SEQUENCE LISTING (242 sequences)

SEQ ID NO: 1 - RNA, 12 nt, Artificial Sequence
  Antisense Compound
cgcgaauucgcg

SEQ ID NO: 2 - RNA, 12 nt, Artificial Sequence
  Antisense Compound
gcgcuuaagcgc

SEQ ID NO: 3 - DNA, 20 bp, Artificial Sequence
  Antisense Compound
ctgctagcctctggatttga

SEQ ID NO: 4 - DNA, 20 bp, Artificial Sequence
  Antisense Compound
ccttccctgaaggttcctcc

SEQ ID NO: 5 - DNA, 20 bp, Artificial Sequence
  Antisense Compound
cttctagcctctggattgga

SEQ ID NO: 6 - DNA, 20 bp, Artificial Sequence
  Antisense Compound
cgttattaacctccgttgaa

SEQ ID NO: 7 - DNA, 20 bp, Artificial Sequence
  Antisense Compound
gagctttgccttcttgccat

SEQ ID NO: 8 - DNA, 20 bp, Artificial Sequence
  Antisense Compound
gcgatttcccgttttcacct

SEQ ID NO: 9 - DNA, 20 bp, Artificial Sequence
  Antisense Compound
  misc_feature 1, 2, 3, 4, 5, 6, 7, 8, 9, 10, 11, 12, 13, 14, 15, 16,17, 18, 19, 20: n = A,T,C or G
nnnnnnnnnnnnnnnnnnnn

SEQ ID NO: 10 - DNA, 20 bp, Artificial Sequence
  Antisense Compound
ttgttgacgttgtactcagc

SEQ ID NO: 11 - DNA, 20 bp, Artificial Sequence
  Antisense Compound
ttgttaacggtgttctcagc

SEQ ID NO: 12 - DNA, 20 bp, Artificial Sequence
  Antisense Compound
tttgtaacggtgttcactga

SEQ ID NO: 13 - DNA, 20 bp, Artificial Sequence
  Antisense Compound
ttcatgaactgcacagaggt

SEQ ID NO: 14 - DNA, 20 bp, Artificial Sequence
  Antisense Compound
tacttgacctacagagtgga

SEQ ID NO: 15 - DNA, 18 bp, Artificial Sequence
  Antisense Compound
tgtgctattctgtgaatt

SEQ ID NO: 16 - DNA, 20 bp, Artificial Sequence
  Antisense Compound
aaccacacttacccatgggc

SEQ ID NO: 17 - DNA, 20 bp, Artificial Sequence
  Antisense Compound
gttggtctcctttgcctgga

SEQ ID NO: 18 - DNA, 20 bp, Artificial Sequence
  Antisense Compound
gttcgtgttctctggctcga

SEQ ID NO: 19 - DNA, 20 bp, Artificial Sequence
  Antisense Compound
tgtcatcgggttcccagcct

SEQ ID NO: 20 - DNA, 20 bp, Artificial Sequence
  Antisense Compound
tccatttattagtctaggaa

SEQ ID NO: 21 - DNA, 20 bp, Artificial Sequence
  Antisense Compound
atgcactcaagaactcggta

SEQ ID NO: 22 - DNA, 20 bp, Artificial Sequence
  Antisense Compound
ttctcatgatgaggtgtacc

SEQ ID NO: 23 - DNA, 20 bp, Artificial Sequence
  Antisense Compound
ccttccctgaaccttcctcc

SEQ ID NO: 24 - DNA, 20 bp, Artificial Sequence
  Antisense Compound
tgttgcaagaatttctcatg

SEQ ID NO: 25 - DNA, 20 bp, Artificial Sequence
  Antisense Compound
gtgtttctgagaacttgtgg

SEQ ID NO: 26 - DNA, 20 bp, Artificial Sequence
  Antisense Compound
atgtccagttttccgccctt

SEQ ID NO: 27 - DNA, 20 bp, Artificial Sequence
  Antisense Compound
tcaaggactgctgatcttcg

SEQ ID NO: 28 - DNA, 20 bp, Artificial Sequence
  Antisense Compound
ggacctgtagccatagccaa

SEQ ID NO: 29 - DNA, 20 bp, Artificial Sequence
  Antisense Compound
ctcgtgaaccagagcaccac

SEQ ID NO: 30 - DNA, 20 bp, Artificial Sequence
  Antisense Compound
tcaagtttctctgtgcccaa

SEQ ID NO: 31 - DNA, 20 bp, Artificial Sequence
  Antisense Compound
tcaagtccttccacacccaa

SEQ ID NO: 32 - DNA, 20 bp, Artificial Sequence
  Antisense Compound
gttcctgtcaaagctcgtgc

SEQ ID NO: 33 - DNA, 20 bp, Artificial Sequence
  Antisense Compound
tcaagtttctccgtgcccaa

SEQ ID NO: 34 - DNA, 20 bp, Artificial Sequence
  Antisense Compound
tcaagtctctccgcgcccaa

SEQ ID NO: 35 - DNA, 20 bp, Artificial Sequence
  Antisense Compound
tcaagtctttccacgcccaa

SEQ ID NO: 36 - skipped

SEQ ID NO: 37 - DNA, 20 bp, Artificial Sequence
  Antisense Compound
gcactttgtggtgccaaggc

SEQ ID NO: 38 - DNA, 20 bp, Artificial Sequence
  Antisense Compound
caggagatgttggccgtggt

SEQ ID NO: 39 - DNA, 20 bp, Artificial Sequence
  Antisense Compound
gctccttccactgatcctgc

SEQ ID NO: 40 - DNA, 19 bp, Artificial Sequence
  Antisense Compound
cgagaggcggacgggaccg

SEQ ID NO: 41 - DNA, 21 bp, Artificial Sequence
  Antisense Compound
cgagaggcggacgggaccgtt

SEQ ID NO: 42 - DNA, 21 bp, Artificial Sequence
  Antisense Compound
ttgctctccgcctgccctggc

SEQ ID NO: 43 - DNA, 18 bp, Artificial Sequence
  Antisense Compound
ctggcacaaagaacagca

SEQ ID NO: 44 - DNA, 18 bp, Artificial Sequence
  Antisense Compound
gaacagcactgactgttt

SEQ ID NO: 45 - DNA, 18 bp, Artificial Sequence
  Antisense Compound
agaacagcactgactgtt

SEQ ID NO: 46 - DNA, 18 bp, Artificial Sequence
  Antisense Compound
aagaacagcactgactgt

SEQ ID NO: 47 - DNA, 18 bp, Artificial Sequence
  Antisense Compound
aaagaacagcactgactg

SEQ ID NO: 48 - DNA, 18 bp, Artificial Sequence
  Antisense Compound
caaagaacagcactgact

SEQ ID NO: 49 - DNA, 18 bp, Artificial Sequence
  Antisense Compound
acaaagaacagcactgac

SEQ ID NO: 50 - DNA, 18 bp, Artificial Sequence
  Antisense Compound
cacaaagaacagcactga

SEQ ID NO: 51 - DNA, 18 bp, Artificial Sequence
  Antisense Compound
gcacaaagaacagcactg

SEQ ID NO: 52 - DNA, 18 bp, Artificial Sequence
  Antisense Compound
ggcacaaagaacagcact

SEQ ID NO: 53 - DNA, 18 bp, Artificial Sequence
  Antisense Compound
tggcacaaagaacagcac

SEQ ID NO: 54 - DNA, 18 bp, Artificial Sequence
  Antisense Compound
gctggcacaaagaacagc

SEQ ID NO: 55 - DNA, 18 bp, Artificial Sequence
  Antisense Compound
ggctggcacaaagaacag

SEQ ID NO: 56 - DNA, 18 bp, Artificial Sequence
  Antisense Compound
tggctggcacaaagaaca

SEQ ID NO: 57 - DNA, 18 bp, Artificial Sequence
  Antisense Compound
ctggctggcacaaagaac

SEQ ID NO: 58 - DNA, 18 bp, Artificial Sequence
  Antisense Compound
cctggctggcacaaagaa

SEQ ID NO: 59 - DNA, 18 bp, Artificial Sequence
  Antisense Compound
tcctggctggcacaaaga

SEQ ID NO: 60 - DNA, 18 bp, Artificial Sequence
  Antisense Compound
gtcctggctggcacaaag

SEQ ID NO: 61 - DNA, 18 bp, Artificial Sequence
  Antisense Compound
tgtcctggctggcacaaa

SEQ ID NO: 62 - DNA, 18 bp, Artificial Sequence
  Antisense Compound
ctgtcctggctggcacaa

SEQ ID NO: 63 - DNA, 18 bp, Artificial Sequence
  Antisense Compound
tctgtcctggctggcaca

SEQ ID NO: 64 - DNA, 20 bp, Artificial Sequence
  Antisense Compound
tccgtcatcgctcctcaggg

SEQ ID NO: 65 - DNA, 20 bp, Artificial Sequence
  Antisense Compound
gtgcgcgcgagcccgaaatc

SEQ ID NO: 66 - DNA, 20 bp, Artificial Sequence
  Antisense Compound
atgcattctgcccccaagga

SEQ ID NO: 67 - DNA, 1004 bp, H. sapiens
gcctcgctcgggcgcccagtggtcctgccgcctggtctcacctcgccatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgcaggggtgcaggcagaagcggggagctatgcccagtcagtgccagcccctc

SEQ ID NO: 68 - DNA, 18 bp, Artificial Sequence
  Antisense Compound
gaccaggcggcaggacca

SEQ ID NO: 69 - DNA, 18 bp, Artificial Sequence
  Antisense Compound
aggtgagaccaggcggca

SEQ ID NO: 70 - DNA, 18 bp, Artificial Sequence
  Antisense Compound
gcagaggcagacgaacca

SEQ ID NO: 71 - DNA, 18 bp, Artificial Sequence
  Antisense Compound
gcaagcagccccagagga

SEQ ID NO: 72 - DNA, 18 bp, Artificial Sequence
  Antisense Compound
ggtcagcaagcagcccca

SEQ ID NO: 73 - DNA, 18 bp, Artificial Sequence
  Antisense Compound
gacagcggtcagcaagca

SEQ ID NO: 74 - DNA, 18 bp, Artificial Sequence
  Antisense Compound
gatggacagcggtcagca

SEQ ID NO: 75 - DNA, 18 bp, Artificial Sequence
  Antisense Compound
ggtggttctggatggaca

SEQ ID NO: 76 - DNA, 18 bp, Artificial Sequence
  Antisense Compound
gcagtgggtggttctgga

SEQ ID NO: 77 - DNA, 18 bp, Artificial Sequence
  Antisense Compound
cacaaagaacagcactga

SEQ ID NO: 78 - DNA, 18 bp, Artificial Sequence
  Antisense Compound
ctggcacaaagaacagca

SEQ ID NO: 79 - DNA, 18 bp, Artificial Sequence
  Antisense Compound
tcctggctggcacaaaga

SEQ ID NO: 80 - DNA, 18 bp, Artificial Sequence
  Antisense Compound
ctgtcctggctggcacaa

SEQ ID NO: 81 - DNA, 18 bp, Artificial Sequence
  Antisense Compound
actctgtgcagtcactca

SEQ ID NO: 82 - DNA, 18 bp, Artificial Sequence
  Antisense Compound
ttcaccgcaaggaaggca

SEQ ID NO: 83 - DNA, 18 bp, Artificial Sequence
  Antisense Compound
ctctgttccaggtgtcta

SEQ ID NO: 84 - DNA, 18 bp, Artificial Sequence
  Antisense Compound
gtgccagccttcttcaca

SEQ ID NO: 85 - DNA, 18 bp, Artificial Sequence
  Antisense Compound
tgcaggacacagctctca

SEQ ID NO: 86 - DNA, 18 bp, Artificial Sequence
  Antisense Compound
aatctgcttgaccccaaa

SEQ ID NO: 87 - DNA, 18 bp, Artificial Sequence
  Antisense Compound
gtatcagaaacccctgta

SEQ ID NO: 88 - DNA, 18 bp, Artificial Sequence
  Antisense Compound
gactgggcagggctcgca

SEQ ID NO: 89 - DNA, 18 bp, Artificial Sequence
  Antisense Compound
cattggagaagaagccga

SEQ ID NO: 90 - DNA, 18 bp, Artificial Sequence
  Antisense Compound
tcgaaagcagatgacaca

SEQ ID NO: 91 - DNA, 18 bp, Artificial Sequence
  Antisense Compound
caggtctttggtctcaca

SEQ ID NO: 92 - DNA, 18 bp, Artificial Sequence
  Antisense Compound
ttttgataaagaccagca

SEQ ID NO: 93 - DNA, 18 bp, Artificial Sequence
  Antisense Compound
gatcgtcgggaaaattga

SEQ ID NO: 94 - DNA, 18 bp, Artificial Sequence
  Antisense Compound
tggagcagcagtgttgga

SEQ ID NO: 95 - DNA, 18 bp, Artificial Sequence
  Antisense Compound
cggttggcatccatgtaa

SEQ ID NO: 96 - DNA, 18 bp, Artificial Sequence
  Antisense Compound
ctgtctctcctgcactga

SEQ ID NO: 97 - DNA, 18 bp, Artificial Sequence
  Antisense Compound
tctggatggacagcggtc

SEQ ID NO: 98 - DNA, 18 bp, Artificial Sequence
  Antisense Compound
ctggtggcagtgtgtctc

SEQ ID NO: 99 - DNA, 18 bp, Artificial Sequence
  Antisense Compound
ggtgcccttctgctggac

SEQ ID NO: 100 - DNA, 18 bp, Artificial Sequence
  Antisense Compound
acaggtgcagatggtgtc

SEQ ID NO: 101 - DNA, 18 bp, Artificial Sequence
  Antisense Compound
gaaacccctgtagcaatc

SEQ ID NO: 102 - DNA, 18 bp, Artificial Sequence
  Antisense Compound
ttggtctcacagcttgtc

SEQ ID NO: 103 - DNA, 18 bp, Artificial Sequence
  Antisense Compound
ctgttgcacaaccaggtc

SEQ ID NO: 104 - DNA, 18 bp, Artificial Sequence
  Antisense Compound
gtcttgtttgtgcctgcc

SEQ ID NO: 105 - DNA, 18 bp, Artificial Sequence
  Antisense Compound
ccacagacaacatcagtc

SEQ ID NO: 106 - DNA, 18 bp, Artificial Sequence
  Antisense Compound
ctggggaccacagacaac

SEQ ID NO: 107 - DNA, 18 bp, Artificial Sequence
  Antisense Compound
tcagccgatcctggggac

SEQ ID NO: 108 - DNA, 18 bp, Artificial Sequence
  Antisense Compound
gtcgggaaaattgatctc

SEQ ID NO: 109 - DNA, 18 bp, Artificial Sequence
  Antisense Compound
ggagccaggaagatcgtc

SEQ ID NO: 110 - DNA, 18 bp, Artificial Sequence
  Antisense Compound
tggcatccatgtaaagtc

SEQ ID NO: 111 - DNA, 18 bp, Artificial Sequence
  Antisense Compound
aactgcctgtttgcccac

SEQ ID NO: 112 - DNA, 18 bp, Artificial Sequence
  Antisense Compound
actgactgggcatagctc

SEQ ID NO: 113 - DNA, 18 bp, Artificial Sequence
  Antisense Compound
gtgggtggttctggatgg

SEQ ID NO: 114 - DNA, 18 bp, Artificial Sequence
  Antisense Compound
gaaggcattccgtttcag

SEQ ID NO: 115 - DNA, 18 bp, Artificial Sequence
  Antisense Compound
gtgtctgtttctgaggtg

SEQ ID NO: 116 - DNA, 18 bp, Artificial Sequence
  Antisense Compound
tggtgtctgtttctgagg

SEQ ID NO: 117 - DNA, 18 bp, Artificial Sequence
  Antisense Compound
ggacacagctctcacagg

SEQ ID NO: 118 - DNA, 18 bp, Artificial Sequence
  Antisense Compound
gagcggtgcaggacacag

SEQ ID NO: 119 - DNA, 18 bp, Artificial Sequence
  Antisense Compound
aagccgggcgagcatgag

SEQ ID NO: 120 - DNA, 18 bp, Artificial Sequence
  Antisense Compound
gctcgcagatggtatcag

SEQ ID NO: 121 - DNA, 18 bp, Artificial Sequence
  Antisense Compound
gatgacacattggagaag

SEQ ID NO: 122 - DNA, 18 bp, Artificial Sequence
  Antisense Compound
gcagatgacacattggag

SEQ ID NO: 123 - DNA, 18 bp, Artificial Sequence
  Antisense Compound
gtttgtgcctgcctgttg

SEQ ID NO: 124 - DNA, 18 bp, Artificial Sequence
  Antisense Compound
caccaccagggctctcag

SEQ ID NO: 125 - DNA, 18 bp, Artificial Sequence
  Antisense Compound
accagcaccaagaggatg

SEQ ID NO: 126 - DNA, 18 bp, Artificial Sequence
  Antisense Compound
tattggttggcttcttgg

SEQ ID NO: 127 - DNA, 18 bp, Artificial Sequence
  Antisense Compound
gggttcctgcttggggtg

SEQ ID NO: 128 - DNA, 18 bp, Artificial Sequence
  Antisense Compound
gtaaagtctcctgcactg

SEQ ID NO: 129 - DNA, 18 bp, Artificial Sequence
  Antisense Compound
ctctttgccatcctcctg

SEQ ID NO: 130 - DNA, 18 bp, Artificial Sequence
  Antisense Compound
cttctgcctgcacccctg

SEQ ID NO: 131 - DNA, 18 bp, Artificial Sequence
  Antisense Compound
ccaggcggcaggaccact

SEQ ID NO: 132 - DNA, 18 bp, Artificial Sequence
  Antisense Compound
cagaggcagacgaaccat

SEQ ID NO: 133 - DNA, 18 bp, Artificial Sequence
  Antisense Compound
ctcaccagtttctgtcct

SEQ ID NO: 134 - DNA, 18 bp, Artificial Sequence
  Antisense Compound
tcactcaccagtttctgt

SEQ ID NO: 135 - DNA, 18 bp, Artificial Sequence
  Antisense Compound
gtgcagtcactcaccagt

SEQ ID NO: 136 - DNA, 18 bp, Artificial Sequence
  Antisense Compound
cagtgaactctgtgcagt

SEQ ID NO: 137 - DNA, 18 bp, Artificial Sequence
  Antisense Compound
attccgtttcagtgaact

SEQ ID NO: 138 - DNA, 18 bp, Artificial Sequence
  Antisense Compound
tggggtcgcagtatttgt

SEQ ID NO: 139 - DNA, 18 bp, Artificial Sequence
  Antisense Compound
ggttggggtcgcagtatt

SEQ ID NO: 140 - DNA, 18 bp, Artificial Sequence
  Antisense Compound
ctaggttggggtcgcagt

SEQ ID NO: 141 - DNA, 18 bp, Artificial Sequence
  Antisense Compound
ctgaggtgcccttctgct

SEQ ID NO: 142 - DNA, 18 bp, Artificial Sequence
  Antisense Compound
ttcacaggtgcagatggt

SEQ ID NO: 143 - DNA, 18 bp, Artificial Sequence
  Antisense Compound
tacagtgccagccttctt

SEQ ID NO: 144 - DNA, 18 bp, Artificial Sequence
  Antisense Compound
gcagggctcgcagatggt

SEQ ID NO: 145 - DNA, 18 bp, Artificial Sequence
  Antisense Compound
tgggcagggctcgcagat

SEQ ID NO: 146 - DNA, 18 bp, Artificial Sequence
  Antisense Compound
gtccaagggtgacatttt

SEQ ID NO: 147 - DNA, 18 bp, Artificial Sequence
  Antisense Compound
cacagcttgtccaagggt

SEQ ID NO: 148 - DNA, 18 bp, Artificial Sequence
  Antisense Compound
gggatcaccaccagggct

SEQ ID NO: 149 - DNA, 18 bp, Artificial Sequence
  Antisense Compound
gaggatggcaaacaggat

SEQ ID NO: 150 - DNA, 18 bp, Artificial Sequence
  Antisense Compound
tggagccaggaagatcgt

SEQ ID NO: 151 - DNA, 18 bp, Artificial Sequence
  Antisense Compound
ggtgcagcctcactgtct

SEQ ID NO: 152 - DNA, 23 bp, Artificial Sequence
  PCR Primer
cagagttcactgaaacggaatgc

SEQ ID NO: 153 - DNA, 23 bp, Artificial Sequence
  PCR Primer
ggtggcagtgtgtctctctgttc

SEQ ID NO: 154 - DNA, 25 bp, Artificial Sequence
  PCR Probe
ttccttgcggtgaaagcgaattcct

SEQ ID NO: 155 - DNA, 19 bp, Artificial Sequence
  PCR Primer
gaaggtgaaggtcggagtc

SEQ ID NO: 156 - DNA, 20 bp, Artificial Sequence
  PCR Primer
gaagatggtgatgggatttc

SEQ ID NO: 157 - DNA, 20 bp, Artificial Sequence
  PCR Probe
caagcttcccgttctcagcc

SEQ ID NO: 158 - DNA, 1004 bp, H. sapeins; identical to SEQ ID NO: 67

SEQ ID NO: 159 - DNA, 18 bp, Artificial Sequence
  Antisense Compound
ccaggcggcaggaccact

SEQ ID NO: 160 - DNA, 18 bp, Artificial Sequence
  Antisense Compound
gaccaggcggcaggacca

SEQ ID NO: 161 - DNA, 18 bp, Artificial Sequence
  Antisense Compound
aggtgagaccaggcggca

SEQ ID NO: 162 - DNA, 18 bp, Artificial Sequence
  Antisense Compound
cagaggcagacgaaccat

SEQ ID NO: 163 - DNA, 18 bp, Artificial Sequence
  Antisense Compound
gcagaggcagacgaacca

SEQ ID NO: 164 - DNA, 18 bp, Artificial Sequence
  Antisense Compound
gcaagcagccccagagga

SEQ ID NO: 165 - DNA, 18 bp, Artificial Sequence
  Antisense Compound
ggtcagcaagcagcccca

SEQ ID NO: 166 - DNA, 18 bp, Artificial Sequence
  Antisense Compound
gacagcggtcagcaagca

SEQ ID NO: 167 - DNA, 18 bp, Artificial Sequence
  Antisense Compound
gatggacagcggtcagca

SEQ ID NO: 168 - DNA, 18 bp, Artificial Sequence
  Antisense Compound
tctggatggacagcggtc

SEQ ID NO: 169 - DNA, 18 bp, Artificial Sequence
  Antisense Compound
ggtggttctggatggaca

SEQ ID NO: 170 - DNA, 18 bp, Artificial Sequence
  Antisense Compound
gtgggtggttctggatgg

SEQ ID NO: 171 - DNA, 18 bp, Artificial Sequence
  Antisense Compound
gcagtgggtggttctgga

SEQ ID NO: 172 - DNA, 18 bp, Artificial Sequence
  Antisense Compound
cacaaagaacagcactga

SEQ ID NO: 173 - DNA, 18 bp, Artificial Sequence
  Antisense Compound
ctggcacaaagaacagca

SEQ ID NO: 174 - DNA, 18 bp, Artificial Sequence
  Antisense Compound
tcctggctggcacaaaga

SEQ ID NO: 175 - DNA, 18 bp, Artificial Sequence
  Antisense Compound
ctgtcctggctggcacaa

SEQ ID NO: 176 - DNA, 18 bp, Artificial Sequence
  Antisense Compound
ctcaccagtttctgtcct

SEQ ID NO: 177 - DNA, 18 bp, Artificial Sequence
  Antisense Compound
tcactcaccagtttctgt

SEQ ID NO: 178 - DNA, 18 bp, Artificial Sequence
  Antisense Compound
gtgcagtcactcaccagt

SEQ ID NO: 179 - DNA, 18 bp, Artificial Sequence
  Antisense Compound
actctgtgcagtcactca

SEQ ID NO: 180 - DNA, 18 bp, Artificial Sequence
  Antisense Compound
cagtgaactctgtgcagt

SEQ ID NO: 181 - DNA, 18 bp, Artificial Sequence
  Antisense Compound
attccgtttcagtgaact

SEQ ID NO: 182 - DNA, 18 bp, Artificial Sequence
  Antisense Compound
gaaggcattccgtttcag

SEQ ID NO: 183 - DNA, 18 bp, Artificial Sequence
  Antisense Compound
ttcaccgcaaggaaggca

SEQ ID NO: 184 - DNA, 18 bp, Artificial Sequence
  Antisense Compound
ctctgttccaggtgtcta

SEQ ID NO: 185 - DNA, 18 bp, Artificial Sequence
  Antisense Compound
ctggtggcagtgtgtctc

SEQ ID NO: 186 - DNA, 18 bp, Artificial Sequence
  Antisense Compound
tggggtcgcagtatttgt

SEQ ID NO: 187 - DNA, 18 bp, Artificial Sequence
  Antisense Compound
ggttggggtcgcagtatt

SEQ ID NO: 188 - DNA, 18 bp, Artificial Sequence
  Antisense Compound
ctaggttggggtcgcagt

SEQ ID NO: 189 - DNA, 18 bp, Artificial Sequence
  Antisense Compound
ggtgcccttctgctggac

SEQ ID NO: 190 - DNA, 18 bp, Artificial Sequence
  Antisense Compound
ctgaggtgcccttctgct

SEQ ID NO: 191 - DNA, 18 bp, Artificial Sequence
  Antisense Compound
gtgtctgtttctgaggtg

SEQ ID NO: 192 - DNA, 18 bp, Artificial Sequence
  Antisense Compound
tggtgtctgtttctgagg

SEQ ID NO: 193 - DNA, 18 bp, Artificial Sequence
  Antisense Compound
acaggtgcagatggtgtc

SEQ ID NO: 194 - DNA, 18 bp, Artificial Sequence
  Antisense Compound
ttcacaggtgcagatggt

SEQ ID NO: 195 - DNA, 18 bp, Artificial Sequence
  Antisense Compound
gtgccagccttcttcaca

SEQ ID NO: 196 - DNA, 18 bp, Artificial Sequence
  Antisense Compound
tacagtgccagccttctt

SEQ ID NO: 197 - DNA, 18 bp, Artificial Sequence
  Antisense Compound
ggacacagctctcacagg

SEQ ID NO: 198 - DNA, 18 bp, Artificial Sequence
  Antisense Compound
tgcaggacacagctctca

SEQ ID NO: 199 - DNA, 18 bp, Artificial Sequence
  Antisense Compound
gagcggtgcaggacacag

SEQ ID NO: 200 - DNA, 18 bp, Artificial Sequence
  Antisense Compound
aagccgggcgagcatgag

SEQ ID NO: 201 - DNA, 18 bp, Artificial Sequence
  Antisense Compound
aatctgcttgaccccaaa

SEQ ID NO: 202 - DNA, 18 bp, Artificial Sequence
  Antisense Compound
gaaacccctgtagcaatc

SEQ ID NO: 203 - DNA, 18 bp, Artificial Sequence
  Antisense Compound
gtatcagaaacccctgta

SEQ ID NO: 204 - DNA, 18 bp, Artificial Sequence
  Antisense Compound
gctcgcagatggtatcag

SEQ ID NO: 205 - DNA, 18 bp, Artificial Sequence
  Antisense Compound
gcagggctcgcagatggt

SEQ ID NO: 206 - DNA, 18 bp, Artificial Sequence
  Antisense Compound
tgggcagggctcgcagat

SEQ ID NO: 207 - DNA, 18 bp, Artificial Sequence
  Antisense Compound
gactgggcagggctcgca

SEQ ID NO: 208 - DNA, 18 bp, Artificial Sequence
  Antisense Compound
cattggagaagaagccga

SEQ ID NO: 209 - DNA, 18 bp, Artificial Sequence
  Antisense Compound
gatgacacattggagaag

SEQ ID NO: 210 - DNA, 18 bp, Artificial Sequence
  Antisense Compound
gcagatgacacattggag

SEQ ID NO: 211 - DNA, 18 bp, Artificial Sequence
  Antisense Compound
tcgaaagcagatgacaca

SEQ ID NO: 212 - DNA, 18 bp, Artificial Sequence
  Antisense Compound
gtccaagggtgacatttt

SEQ ID NO: 213 - DNA, 18 bp, Artificial Sequence
  Antisense Compound
cacagcttgtccaagggt

SEQ ID NO: 214 - DNA, 18 bp, Artificial Sequence
  Antisense Compound
ttggtctcacagcttgtc

SEQ ID NO: 215 - DNA, 18 bp, Artificial Sequence
  Antisense Compound
caggtctttggtctcaca

SEQ ID NO: 216 - DNA, 18 bp, Artificial Sequence
  Antisense Compound
ctgttgcacaaccaggtc

SEQ ID NO: 217 - DNA, 18 bp, Artificial Sequence
  Antisense Compound
gtttgtgcctgcctgttg

SEQ ID NO: 218 - DNA, 18 bp, Artificial Sequence
  Antisense Compound
gtcttgtttgtgcctgcc

SEQ ID NO: 219 - DNA, 18 bp, Artificial Sequence
  Antisense Compound
ccacagacaacatcagtc

SEQ ID NO: 220 - DNA, 18 bp, Artificial Sequence
  Antisense Compound
ctggggaccacagacaac

SEQ ID NO: 221 - DNA, 18 bp, Artificial Sequence
  Antisense Compound
tcagccgatcctggggac

SEQ ID NO: 222 - DNA, 18 bp, Artificial Sequence
  Antisense Compound
caccaccagggctctcag

SEQ ID NO: 223 - DNA, 18 bp, Artificial Sequence
  Antisense Compound
gggatcaccaccagggct

SEQ ID NO: 224 - DNA, 18 bp, Artificial Sequence
  Antisense Compound
gaggatggcaaacaggat

SEQ ID NO: 225 - DNA, 18 bp, Artificial Sequence
  Antisense Compound
accagcaccaagaggatg

SEQ ID NO: 226 - DNA, 18 bp, Artificial Sequence
  Antisense Compound
ttttgataaagaccagca

SEQ ID NO: 227 - DNA, 18 bp, Artificial Sequence
  Antisense Compound
tattggttggcttcttgg

SEQ ID NO: 228 - DNA, 18 bp, Artificial Sequence
  Antisense Compound
gggttcctgcttggggtg

SEQ ID NO: 229 - DNA, 18 bp, Artificial Sequence
  Antisense Compound
gtcgggaaaattgatctc

SEQ ID NO: 230 - DNA, 18 bp, Artificial Sequence
  Antisense Compound
gatcgtcgggaaaattga

SEQ ID NO: 231 - DNA, 18 bp, Artificial Sequence
  Antisense Compound
ggagccaggaagatcgtc

SEQ ID NO: 232 - DNA, 18 bp, Artificial Sequence
  Antisense Compound
tggagccaggaagatcgt

SEQ ID NO: 233 - DNA, 18 bp, Artificial Sequence
  Antisense Compound
tggagcagcagtgttgga

SEQ ID NO: 234 - DNA, 18 bp, Artificial Sequence
  Antisense Compound
gtaaagtctcctgcactg

SEQ ID NO: 235 - DNA, 18 bp, Artificial Sequence
  Antisense Compound
tggcatccatgtaaagtc

SEQ ID NO: 236 - DNA, 18 bp, Artificial Sequence
  Antisense Compound
cggttggcatccatgtaa

SEQ ID NO: 237 - DNA, 18 bp, Artificial Sequence
  Antisense Compound
ctctttgccatcctcctg

SEQ ID NO: 238 - DNA, 18 bp, Artificial Sequence
  Antisense Compound
ctgtctctcctgcactga

SEQ ID NO: 239 - DNA, 18 bp, Artificial Sequence
  Antisense Compound
ggtgcagcctcactgtct

SEQ ID NO: 240 - DNA, 18 bp, Artificial Sequence
  Antisense Compound
aactgcctgtttgcccac

SEQ ID NO: 241 - DNA, 18 bp, Artificial Sequence
  Antisense Compound
cttctgcctgcacccctg

SEQ ID NO: 242 - DNA, 18 bp, Artificial Sequence
  Antisense Compound
actgactgggcatagctc

(19) United States
(12) Patent Application Publication
(10) Pub. No.: US 2005/0142581 A1
(43) Pub. Date: Jun. 30, 2005

(54) Microrna as ligands and target molecules

(76) Inventors: Richard H. Griffey, Vista, CA (US); C. Frank Bennett, Carlsbad, CA (US); David J. Ecker, Encinitas, CA (US); Donna T. Ward, Carlsbad, CA (US); Susan M. Freier, San Diego, CA (US)

Correspondence Address: COZEN O'CONNOR, P.C., 1900 MARKET STREET, PHILADELPHIA, PA, 19103-3508, US

(21) Appl. No.: 10/934,798

(22) Filed: Sep. 3, 2004

Related U.S. Application Data
(60) Provisional application No. 60/500,724, filed on Sep. 4, 2003.
(60) Provisional application No. 60/502,007, filed on Sep. 11, 2003.
(60) Provisional application No. 60/500,732, filed on Sep. 4, 2003.
(60) Provisional application No. 60/502,076, filed on Sep. 11, 2003.
(60) Provisional application No. 60/500,723, filed on Sep. 4, 2003.
(60) Provisional application No. 60/500,824, filed on Sep. 4, 2003.
(60) Provisional application No. 60/500,730, filed on Sep. 4, 2003.
(60) Provisional application No. 60/504,495, filed on Sep. 17, 2003.

(51) Int. Cl. C12Q 1/68

(52) U.S. Cl. 435/6

(57) ABSTRACT

The present invention provides methods for the identification of target molecules that bind to ligands, particularly microRNA ligands and mimics thereof and/or microRNA target molecules and mimics thereof, with as little as millimolar (mM) affinity using mass spectrometry. The methods may be used to determine the mode of binding interaction between two or more of these target molecules to the ligand as well as their relative affinities. Also provided are methods for designing compounds having greater affinity to a ligand by identifying two or more target molecules using mass spectrometry methods of the invention and linking the target molecules together to form a novel compound.

### CROSS-REFERENCE TO RELATED APPLICATIONS

This application claims priority to: 1) U.S. provisional application Ser. No. 60/500,724 filed Sep. 4, 2003; 2) U.S. provisional application Ser. No. 60/502,007 filed Sep. 11, 2003; 3) U.S. provisional application Ser. No. 60/500,732 filed Sep. 4, 2003; 4) U.S. provisional application Ser. No. 60/502,076 filed Sep. 11, 2003; 5) U.S. provisional application Ser. No. 60/500,723 filed Sep. 4, 2003; 6) U.S. provisional application Ser. No. 60/500,824 filed Sep. 4, 2003; 7) U.S. provisional application Ser. No. 60/500,730 filed Sep. 4, 2003; and 8) U.S. provisional application Ser. No. 60/504,495 filed Sep. 17, 2003; each of which is incorporated herein by reference in its entirety.

### FIELD OF THE INVENTION

The present invention is related to mass spectrometry methods for detecting binding interactions of ligands to substrates and, in particular, to methods for determining the mode of binding interaction of microRNA ligands and microRNA substrates, and to structural alterations caused in the target RNA by the interaction of the ligand with the target, so as to cause the target RNA to change from a less folded to more folded conformation, from a more folded to less folded conformation, or from a first folded conformation to a second, alternative, folded conformation, to the automatic generation of oligomeric compounds targeted to a particular nucleic acid sequence via computer-based, iterative robotic synthesis and robotic or robot-assisted analysis of the activities of such compounds, and to use of a cloud algorithm to predict evolutionary mutations and changes in the RNA and/or microRNA of a bioagent.

### BACKGROUND OF THE INVENTION

In many species, introduction of double-stranded RNA (dsRNA) induces potent and specific gene silencing. This phenomenon occurs in both plants and animals and has roles in viral defense and transposon silencing mechanisms. This phenomenon was originally described more than a decade ago by researchers working with the petunia flower. While trying to deepen the purple color of these flowers, Jorgensen et al. introduced a pigment-producing gene under the control of a powerful promoter. Instead of the expected deep purple color, many of the flowers appeared variegated or even white. Jorgensen named the observed phenomenon “cosuppression”, since the expression of both the introduced gene and the homologous endogenous gene was suppressed (Napoli et al., Plant Cell, 1990, 2, 279-289; Jorgensen et al., Plant Mol. Biol., 1996, 31, 957-973).

Cosuppression has since been found to occur in many species of plants, fungi, and has been particularly well characterized in Neurospora crassa, where it is known as “quelling” (Cogoni and Macino, Genes Dev. 2000, 10, 638-643; Guru, Nature, 2000, 404, 804-808).

The first evidence that dsRNA could lead to gene silencing in animals came from work in the nematode, Caenorhabditis elegans. In 1995, researchers Guo and Kemphues were attempting to use antisense RNA to shut down expression of the par-1 gene in order to assess its function. As expected, injection of the antisense RNA disrupted expression of par-1, but curiously, injection of the sense-strand control also disrupted expression (Guo and Kempheus, Cell, 1995, 81, 611-620). This result was a puzzle until Fire et al. injected dsRNA (a mixture of both sense and antisense strands) into C. elegans. This injection resulted in much more efficient silencing than injection of either the sense or the antisense strands alone. Injection of just a few molecules of dsRNA per cell was sufficient to completely silence the homologous gene's expression. Furthermore, injection of dsRNA into the gut of the worm caused gene silencing not only throughout the worm, but also in first generation offspring (Fire et al., Nature, 1998, 391, 806-811).

The potency of this phenomenon led Timmons and Fire to explore the limits of the dsRNA effects by feeding nematodes bacteria that had been engineered to express dsRNA homologous to the C. elegans unc-22 gene. Surprisingly, these worms developed an unc-22 null-like phenotype (Timmons and Fire, Nature 1998, 395, 854; Timmons et al., Gene, 2001, 263, 103-112). Further work showed that soaking worms in dsRNA was also able to induce silencing (Tabara et al., Science, 1998, 282, 430-431). PCT publication WO 01/48183 discloses methods of inhibiting expression of a target gene in a nematode worm involving feeding to the worm a food organism which is capable of producing a double-stranded RNA structure having a nucleotide sequence substantially identical to a portion of the target gene following ingestion of the food organism by the nematode, or by introducing a DNA capable of producing the double-stranded RNA structure (Bogaert et al., 2001).

The posttranscriptional gene silencing defined in C. elegans resulting from exposure to double-stranded RNA (dsRNA) has since been designated as RNA interference (RNAi). This term has come to generally refer to the process of gene silencing involving dsRNA which leads to the sequence-specific reduction of gene expression. In contrast, cosuppression refers to a process in which transgenic DNA leads to silencing of both the transgene and the endogenous gene.

Introduction of exogenous double-stranded RNA (dsRNA) into C. elegans has been shown to specifically and potently disrupt the activity of genes containing homologous sequences. Montgomery et al. suggests that the primary interference effects of dsRNA are post-transcriptional. This conclusion was derived from examination of the primary DNA sequence after dsRNA-mediated interference and a finding of no evidence of alterations, followed by studies assessing the alteration of an upstream operon which had no effect on the activity of its downstream gene. These results argue against an effect on initiation or elongation of transcription. Finally using in situ hybridization they observed that dsRNA-mediated interference produced a substantial, although not complete, reduction in accumulation of nascent transcripts in the nucleus, while cytoplasmic accumulation of transcripts was virtually eliminated. These results indicate that the endogenous mRNA is the primary target for interference and suggest a mechanism that degrades the targeted mRNA before translation can occur. It was also found that this mechanism is not dependent on the SMG system, an mRNA surveillance system in C. elegans responsible for targeting and destroying aberrant messages. The authors further suggest a model of how dsRNA might function as a catalytic mechanism to target homologous mRNAs for degradation. (Montgomery et al., Proc. Natl. Acad. Sci. USA, 1998, 95, 15502-15507).

Recently, the development of a cell-free system from syncytial blastoderm Drosophila embryos that recapitulates many of the features of RNAi has been reported. The interference observed in this reaction is sequence specific, is promoted by dsRNA but not single-stranded RNA, functions by specific mRNA degradation, and requires a minimum length of dsRNA. Furthermore, preincubation of dsRNA potentiates its activity demonstrating that RNAi can be mediated by sequence-specific processes in soluble reactions (Tuschl et al., Genes Dev., 1999, 13, 3191-3197).

In subsequent experiments, Tuschl et al., using the Drosophila in vitro system, demonstrated that 21- and 22-nt RNA fragments are the sequence-specific mediators of RNAi. These fragments, which they termed short interfering RNAs (siRNAs), were shown to be generated by an RNase III-like processing reaction from long dsRNA. They also showed that chemically synthesized siRNA duplexes with overhanging 3′ ends mediate efficient target RNA cleavage in the Drosophila lysate, and that the cleavage site is located near the center of the region spanned by the guiding siRNA. In addition, they suggest that the direction of dsRNA processing determines whether sense or antisense target RNA can be cleaved by the siRNA-protein complex (Elbashir et al., Genes Dev., 2001, 15, 188-200). Further characterization of the suppression of expression of endogenous and heterologous genes caused by the 21-23 nucleotide siRNAs have been investigated in several mammalian cell lines, including human embryonic kidney (293) and HeLa cells (Elbashir et al., Nature, 2001, 411, 494-498).

The Drosophila embryo extract system has been exploited, using green fluorescent protein and luciferase tagged siRNAs, to demonstrate that siRNAs can serve as primers to transform the target mRNA into dsRNA. The nascent dsRNA is degraded to eliminate the incorporated target mRNA while generating new siRNAs in a cycle of dsRNA synthesis and degradation. Evidence is also presented that mRNA-dependent siRNA incorporation to form dsRNA is carried out by an RNA-dependent RNA polymerase activity (RdRP) (Lipardi et al., Cell, 2001, 107, 297-307).

The involvement of an RNA-directed RNA polymerase and siRNA primers as reported by Lipardi et al. (Lipardi et al., Cell, 2001, 107, 297-307) is one of the many intriguing features of gene silencing by RNA interference. This suggests an apparent catalytic nature to the phenomenon. New biochemical and genetic evidence reported by Nishikura et al. also shows that an RNA-directed RNA polymerase chain reaction, primed by siRNA, amplifies the interference caused by a small amount of “trigger” dsRNA (Nishikura, Cell, 2001, 107, 415-418).

Investigating the role of “trigger” RNA amplification during RNA interference (RNAi) in C. elegans, Sijen et al. revealed a substantial fraction of siRNAs that cannot derive directly from input dsRNA. Instead, a population of siRNAs (termed secondary siRNAs) appeared to derive from the action of the previously reported cellular RNA-directed RNA polymerase (RdRP) on mRNAs that are being targeted by the RNAi mechanism. The distribution of secondary siRNAs exhibited a distinct polarity (5′-3′; on the antisense strand), suggesting a cyclic amplification process in which RdRP is primed by existing siRNAs. This amplification mechanism substantially augmented the potency of RNAi-based surveillance, while ensuring that the RNAi machinery focuses on expressed mRNAs (Sijen et al., Cell, 2001, 107, 465-476).

Recently, Tijsterman et al. have shown that single-stranded RNA oligomers of antisense polarity can be potent inducers of gene silencing. As is the case for cosuppression, they showed that antisense RNAs act independently of the RNAi genes rde-1 and rde-4 but require the mutator/RNAi gene mut-7 and a putative DEAD box RNA helicase, mut-14. According to the authors, their data favor the hypothesis that gene silencing is accomplished by RNA primer extension using the mRNA as template, leading to dsRNA that is subsequently degraded suggesting that single-stranded RNA oligomers are ultimately responsible for the RNAi phenomenon (Tijsterman et al., Science, 2002, 295, 694-697).

Several recent publications have described the structural requirements for the dsRNA trigger required for RNAi activity. Recent reports have indicated that ideal dsRNA sequences are 21 nucleotides (nt) in length containing 2-nt 3′-end overhangs (Elbashir et al., EMBO 2001, 20, 6877-6887; Brantl, Biochimica et Biophysica Acta, 2002, 1575, 15-25). In this system, substitution of the 4 nucleosides from the 3′-end with 2′-deoxynucleosides has been demonstrated to not affect activity. On the other hand, substitution with 2′-deoxynucleosides or 2′-OMe-nucleosides throughout the sequence (sense or antisense) was shown to be deleterious to RNAi activity.

Investigation of the structural requirements for RNA silencing in C. elegans has demonstrated modification of the internucleotide linkage (phosphorothioate) to not interfere with activity (Parrish et al., Molecular Cell, 2000, 6, 1077-1087). It was also shown by Parrish et al., that chemical modification like 2′-amino or 5-iodouridine are well tolerated in the sense strand but not the antisense strand of the dsRNA suggesting differing roles for the 2 strands in RNAi. Base modification such as guanine to inosine (where one hydrogen bond is lost) has been demonstrated to decrease RNAi activity independently of the position of the modification (sense or antisense). Some “position independent” loss of activity has been observed following the introduction of mismatches in the dsRNA trigger. Some types of modifications, for example introduction of sterically demanding bases such as 5-iodoU, have been shown to be deleterious to RNAi activity when positioned in the antisense strand, whereas modifications positioned in the sense strand were shown to be less detrimental to RNAi activity. As was the case for the 21-nucleotide dsRNA sequences, RNA-DNA heteroduplexes did not serve as triggers for RNAi. However, dsRNA containing 2′-F-2′-deoxynucleosides appeared to be efficient in triggering RNAi response independent of the position (sense or antisense) of the 2′-F-2′-deoxynucleosides.

In one study, the reduction of gene expression was studied using electroporated dsRNA and a 25-mer morpholino oligomer in post implantation mouse embryos (Mellitzer et al., Mehanisms of Development, 2002, 118, 57-63). The morpholino oligomer did show activity but was not as effective as the dsRNA.

A number of PCT applications have recently been published that relate to the RNAi phenomenon. These include: PCT publication WO 00/44895; PCT publication WO 00/49035; PCT publication WO 00/63364; PCT publication WO 01/36641; PCT publication WO 01/36646; PCT publication WO 99/32619; PCT publication WO 00/44914; PCT publication WO 01/29058; and PCT publication WO 01/75164.

U.S. Pat. Nos. 5,898,031 and 6,107,094, each of which is commonly owned with this application and each of which is herein incorporated by reference, describe certain oligonucleotide having RNA like properties. When hybridized with RNA, these oligonucleotides serve as substrates for a dsRNase enzyme with resultant cleavage of the RNA by the enzyme.

In another recently published paper (Martinez et al., Cell, 2002, 110, 563-574) it was shown that single stranded as well as double stranded siRNA resides in the RNA-induced silencing complex (RISC) together with elF2C1 and elf2C2 (human GERp950) Argonaute proteins. The activity of 5′-phosphorylated single stranded siRNA was comparable to the double stranded siRNA in the system studied. In a related study, the inclusion of a 5′-phosphate moiety was shown to enhance activity of siRNA's in vivo in Drosophilia embryos (Boutla, et al., Curr. Biol., 2001, 11, 1776-1780). In another study, it was reported that the 5′-phosphate was required for siRNA function in human HeLa cells (Schwarz et al., Molecular Cell, 2002, 10, 537-548).

In yet another recently published paper (Chiu et al., Molecular Cell, 2002, 10, 549-561) it was shown that the 5′-hydroxyl group of the siRNA is essential as it is phosphorylated for activity, whereas the 3′-hydroxyl group is not essential and tolerates substitute groups such as biotin. It was further shown that bulge structures in one or both of the sense or antisense strands either abolished or severely lowered the activity relative to the unmodified siRNA duplex. Also shown was severe lowering of activity when psoralen was used to cross link an siRNA duplex.

RNA genes were once considered relics of a primordial “RNA world” that was largely replaced by more efficient proteins. More recently, however, it has become clear that noncoding RNA genes produce functional RNA molecules with important roles in regulation of gene expression, developmental timing, viral surveillance, and immunity. Not only the classic transfer RNAs (tRNAs) and ribosomal RNAs (rRNAs), but also small nuclear RNAs (snRNAs), small nucleolar RNAs (snoRNAs), small interfering RNAs (siRNAs), tiny noncoding RNAs (tncRNAs) and microRNAs (miRNAs) are now known to act in diverse cellular processes such as chromosome maintenance, gene imprinting, pre-mRNA splicing, guiding RNA modifications, transcriptional regulation, and the control of mRNA translation (Eddy, Nat Rev Genet, 2001, 2, 919-929; Kawasaki and Taira, Nature, 2003, 423, 838-842). RNA-mediated processes are now also believed to direct heterochromatin formation, genome rearrangements, and DNA elimination (Cerutti, Trends Genet, 2003, 19, 39-46; Couzin, Science, 2002, 298, 2296-2297).

The process of RNAi can be divided into two general steps: the initiation step occurs when the dsRNA is processed into siRNAs by an RNase III-like dsRNA-specific enzyme known as Dicer, and the effector step, during which the siRNAs are incorporated into a ribonucleoprotein complex, the RNA-induced silencing complex (RISC). RISC is believed to use the siRNA molecules as a guide to identify complementary RNAs, and an endoribonuclease (to date unidentified) cleaves these target RNAs, resulting in their degradation (Cerutti, Trends Genet, 2003, 19, 39-46; Grishok et al., Cell, 2001, 106, 23-34).

In addition to the siRNAs, a large class of small noncoding RNAs known as microRNAs (mRNAs) is now known to act in the RNAi pathway. In nematodes, fruit flies, and humans, mRNAs are predicted to function as endogenous posttranscriptional gene regulators. The founding members of the mRNA family are transcribed by the C. elegans genes let-7 and lin-4, and were first dubbed short temporal RNAs (stRNAs). The let-7 and lin-4 mRNAs act as antisense translational repressors of messenger RNAs that encode proteins crucial to the heterochronic developmental timing pathway in nematode larva. For example, the lin-4 RNA binds to the 3′UTR regions of its targets, the lin-14 and lin-28 mRNAs, and represses synthesis of the LIN-14 and LIN-28 proteins to cause the proper series of stage-specific developmental events in the early larval stages of C. elegans development (Ambros, Cell, 2001, 107, 823-826; Ambros et al., Curr Biol, 2003, 13, 807-818).

Like siRNAs, mRNAs are processed by Dicer and are approximately the same length (21 to 24 nucleotides), and possess the characteristic 5′-phosphate and 3′-hydroxyl termini. The mRNAs are also incorporated into a ribonucleoprotein complex, the miRNP, which is similar, if not identical to the RISC (Bartel and Bartel, Plant Physiol, 2003, 132, 709-717). More than 200 different mRNAs have been identified in plants and animals (Ambros et al., Curr Biol, 2003, 13, 807-818).

In spite of their biochemical and mechanistic similarities, there are also some key differences between siRNAs and mRNAs, based on unique aspects of their biogenesis. Biological siRNAs are generated from the cleavage of long exogenous or endogenous dsRNA molecules, such as very long hairpins or bimolecular duplexes, and numerous siRNAs accumulate from both strands of dsRNA precursors. Mature mRNAs originate from endogenous hairpin (also known as stemloop or foldback) precursor transcripts, usually 50 to 80 nucleotides in length, that can form local hairpin structures. In vivo, these mRNA hairpin precursors are enzymatically processed such that a single-stranded mature mRNA molecule is generated from one arm of the hairpin precursor. Alternatively, a polycistronic mRNA precursor transcript may contain multiple hairpins, each processed into a different, single mRNA. The current model is that either the primary mRNA transcript or the hairpin precursor is cleaved by Dicer to yield a double-stranded intermediate, but only one strand of this short-lived intermediate accumulates as the mature mRNA (Ambros et al., RNA, 2003, 9, 277-279; Bartel and Bartel, Plant Physiol, 2003, 132, 709-717; Shi, Trends Genet, 2003, 19,9-12).

siRNAs and mRNAs can also be functionally distinguished. While siRNAs cause gene silencing by target RNA cleavage and degradation, mRNAs are believed to direct translational repression, primarily. This functional difference may be related to the fact that mRNAs tolerate multiple base pair mismatches whereas siRNAs are perfectly complementary to their target substrates (Ambros et al., Curr Biol, 2003, 13, 807-818; Bartel and Bartel, Plant Physiol, 2003, 132, 709-717; Shi, Trends Genet, 2003, 19, 9-12).

A third class of small noncoding RNAs has also been identified (Ambros et al., Curr Biol, 2003, 13, 807-818). The tiny noncoding RNA (tncRNA) genes produce transcripts similar in length (20-21 nucleotides) to mRNAs, and are also thought to be developmentally regulated but, unlike mRNAs, tncRNAs are reportedly not processed from short hairpin precursors and are not phylogenetically conserved. Although none of these tncRNAs are believed to originate from mRNA hairpin precursors, some are predicted to form potential foldback structures reminiscent of mRNAs; these putative tncRNA precursor structures deviate significantly from the mRNA hairpins in key characteristics, i.e., they exhibit excessive numbers of bulged nucleotides in the stem or have fewer than 16 base pairs involving the small RNA (Ambros et al., Curr Biol, 2003, 13, 807-818).

The list of cellular activities now believed to be regulated by small noncoding RNAs is still growing and is quite diverse. In several plant species, dsRNA can direct methylation of homologous DNA sequences, and connections between RNAi and chromatin and/or genomic DNA modifications are starting to emerge. Some homologues in the polycomb group of proteins, which are generally involved in chromatin repression, have been shown to be required for RNAi under certain experimental conditions (Cerutti, Trends Genet, 2003, 19, 39-46; Matzke et al., Science, 2001, 293, 1080-1083). Recently, several reports have implicated RNAi machinery in heterochromatin formation (Hall et al., Science, 2002, 297, 2232-2237; Volpe et al., Chromosome Res, 2003, 11, 137-146) and genome rearrangements (Mochizuki et al., Cell, 2002, 110, 689-699; Taverna et al., Cell, 2002, 110, 701-711).

RNAi-like processes may operate in the establishment of heterochromatic domains at centromeres and mating-type loci of the fission yeast, as well as during the lineage-specific establishment of silenced chromatin domains during eukaryotic development (Hall et al., Science, 2002, 297, 2232-2237). In plants, animals and fungi, centromeres are heterochromatic regions that consist of arrays of repetitive DNA sequences. In the fission yeast, components of the RNAi machinery (Dicer (Dcr1), Argonaute (Ago1), and RNA-dependent RNA polymerase(Rdp1)) are required to maintain the silent heterochromatic state of functional centromeres, and are believed to be involved in processing transcripts derived from these repeats. Deletion of Dcr1, Ago1, or Rdp1 disrupts histone H3 lysine 9 methylation and recruitment of heterochromatin proteins to the centromere region and results in chromosome missegregation (Reinhart and Bartel, Science, 2002, 297, 1831; Volpe et al., Chromosome Res, 2003, 11, 137-146). Similarly, the mating-type loci of fission yeast appear to have used a repetitive DNA element to organize a highly specialized chromatin structure, and similar RNAi-like processes may influence a variety of chromosomal functions important for preserving genomic integrity, such as prohibition of wasteful transcription and suppression of deleterious recombination between repetitive elements (Hall et al., Science, 2002, 297, 2232-2237).

The unicellular, ciliated eukaryote, Tetrahymena, contains two functionally distinct nuclei: one containing the DNA expressed during the lifetime of the organism, and one carrying the DNA that passes to offspring. During the differentiation of these two nuclei, several thousand internal eliminated sequences (IESs) are precisely excised and deleted from the germline genome, and small RNAs trigger deletion or reshuffling of some DNA sequences as the Tetrahymena divides. RNAi appears to be targeting structures analogous to heterochromatin for elimination. Interestingly, histone H3 lysine 9 methylation is also required for the targeted DNA elimination. (Couzin, Science, 2002, 298, 2296-2297; Mochizuki et al., Cell, 2002, 110, 689-699; Taverna et al., Cell, 2002, 110, 701-711).

It is currently believed that RNAi represents a form of immunity and protection from invasion by exogenous sources of genetic material such as RNA viruses and retrotransposons (Eddy, Nat Rev Genet, 2001, 2, 919-929; Silva et al., Trends Mol Med, 2002, 8, 505-508). In plants, the dsRNA-mediated mechanism of posttranscriptional gene silencing has been linked to viral resistance, and is proposed to represent a primitive immune response. Infection of Arabidopsis by Turnip mosiac virus (TuMV) induces a number of developmental defects which resemble those in mRNA deficient dicer-like1 (dcl1) mutants. A virally encoded RNA-silencing suppressor, P1/HC-Pro, was found to be a part of a counterdefensive mechanism that enables systemic infection by interfering with miR171 (also known as mRNA39), a component of the mRNA-controlled developmental pathways that share components with the antiviral RNA-silencing pathway (Kasschau et al., Dev Cell, 2003, 4, 205-217).

In prokaryotes, antisense-RNA regulated systems have been detected mostly in so-called accessory DNA elements such as plasmids, phage, or transposons, although a few have been found to be of chromosomal origin. Some of these antisense-RNA-mediated mechanisms are remarkably similar to the translation-inhibition mechanisms mediated by mRNAs, and may involve structural elements such as a stemloop (Brantl, Biochim Biophys Acta, 2002, 1575, 15-25). Interestingly, by injection or expression of antiparallel dsRNA in Escherichia coli, a potent and specific RNA-mediated gene-specific silencing effect has been observed (Tchurikov et al., J Biol Chem, 2000, 275, 26523-26529). Furthermore, several groups have recently reported algorithms and screens leading to the identification or computational prediction of novel small noncoding RNA transcripts in bacteria, and although the precise functions of many of them are not fully understood, it is clear that these small noncoding RNAs act as central regulators of gene expression in response to diverse environmental growth conditions (Argaman et al., Curr Biol, 2001, 11, 941-950; Eddy, Nat Rev Genet, 2001, 2, 919-929; Rivas et al., Curr Biol, 2001, 11, 1369-1373; Wassarman, Cell, 2002, 109, 141-144; Wassarman et al., Genes Dev, 2001, 15, 1637-1651).

A total of 201 different expressed RNA sequences potentially encoding novel small non-messenger species (smnRNAs) has been identified from mouse brain cDNA libraries. Based on sequence and structural motifs, several of these have been assigned to the snoRNA class of nucleolar localized molecules known to act as guide RNAs for rRNA modification, whereas others are predicted to direct modification within the U2, U4, or U6 small nuclear RNAs (snRNAs). Some of these newly identified smnRNAs remained unclassified and have no identified RNA targets. It was suggested that some of these RNA species may have novel functions previously unknown for snoRNAs, namely the regulation of gene expression by binding to and/or modifying mRNAs or their precursors via their antisense elements (Huttenhofer et al., Embo J, 2001, 20, 2943-2953).

RNA editing enzymes may also interact with components of the RNAi pathway. Adenosine deaminases that act on RNA (ADARs) are a class of RNA editing enzymes that deaminate adenosines to create inosines in dsRNA. Inosine is read as guanosine during translation, and thus, one function of editing is to generate multiple protein isoforms from the same gene. ADARs bind to dsRNA without sequence specificity, and due to the ability of ADARs to create sequence and structural changes in dsRNA, ADARs could potentially antagonize RNAi by several mechanisms, such as preventing dsRNA from being recognized and cleaved by Dicer, or preventing siRNAs from base-pairing. Recently, it was shown that the editing of dsRNA by ADARs can prevent somatic transgenes from inducing gene silencing via the RNAi pathway (Knight and Bass, Mol Cell, 2002, 10, 809-817).

miRNAs are also believed to be cell death regulators, implicating them in mechanisms of human disease such as cancer. Recently, the Drosophila mir-14 miRNA was identified as a suppressor of apoptotic cell death and is required for normal fat metabolism. While mir-14 mutants are viable, they have elevated levels of the apoptotic effector caspase Drice, are stress sensitive and have a reduced lifespan. Furthermore, deletion of mir-14 results in animals with increased levels of triacylglycerol and diacylglycerol. Deregulation of miRNA expression may contribute to inappropriate survival that occurs in oncogenesis (Xu et al., Curr Biol, 2003, 13, 790-795).

Naturally occurring miRNAs are characterized by imperfect complementarity to their target sequences. Artificially modified miRNAs with sequences completely complementary to their target RNAs have been designed and found to function as siRNAs that inhibit gene expression by reducing RNA transcript levels. Synthetic hairpin RNAs that mimic siRNAs and miRNA precursor molecules were demonstrated to target genes for silencing by degradation and not translational repression (McManus et al., RNA, 2002, 8, 842-850).

Expression of the human mir-30 mRNA specifically blocked the translation in human cells of an mRNA containing artificial mir-30 target sites. Designed miRNAs were excised from transcripts encompassing artificial miRNA precursors and could inhibit the expression of mRNAs containing a complementary target site. These data indicate that novel mRNAs can be readily produced in vivo and can be designed to specifically inactivate the expression of selected target genes in human cells (Zeng et al., Mol Cell, 2002, 9, 1327-1333).

Hes1, a basic helix-loop-helix protein is reported to be a target of microRNA-23 during retinoic-acid-induced neuronal differentiation of human NT2 neuroepithelial cells. Synthetic siRNA-miR-23 and synthetic mutant siRNA-miR-23 were designed and introduced into undifferentiated human NT2; these small interfering RNAs resulted in accumulation of Hes1 and hindered neuronal differentiation (Kawasaki and Taira, Nature, 2003, 423, 838-842).

Disclosed and claimed in PCT Publications WO 03/035667 and WO 03/034985 is a nucleic acid comprising sense and anti-sense nucleic acids, which may be covalently linked to each other, wherein said sense and anti-sense nucleic acids may comprise RNA in the form of a double-stranded interfering RNA, and wherein said sense and anti-sense nucleic acids are substantially complementary to each other and are capable of forming a double stranded nucleic acid and wherein one of said sense or antisense nucleic acids is substantially complementary to a target nucleic acid comprising telomerase RNA or mRNA encoding telomerase reverse transcriptase (TERT). Also claimed is an expression vector comprising the nucleic acid, methods for inhibiting or interfering with telomerase activity, and a pharmaceutical composition. siRNAs for inhibiting telomerase activity are disclosed and claimed (Rowley, 2003; Rowley, 2003).

Disclosed and claimed in PCT Publications WO 03/022052 and WO 03/023015 is a method of expressing an RNA molecule within a cell by transfection of a recombinant retrovirus into a target cell line, wherein the recombinant retrovirus construct comprises an RNA polymerase III promoter region, an RNA coding region and a termination sequence and may comprise a 5′ lentiviral long terminal repeat region, a self-inactivating lentiviral 3′ LTR, wherein the RNA coding region may encode a self-complementary RNA molecule having a sense region, and antisense region and a loop region, and wherein the RNA coding region is at least about 90% identical to a target region of a pathogenic virus genome or genome transcript or a target cell gene involved in the pathogenic virus life cycle. Further claimed is a method of treating a patient infected with HIV. Small interfering RNAs are generally disclosed (Baltimore et al., 2003; Baltimore et al., 2003).

Disclosed and claimed in PCT Publication WO 03/029459 is an isolated nucleic acid molecule comprising a miRNA nucleotide sequence selected from Tables consisting of Drosophila melanogaster, human, and mouse miRNAs or a precursor thereof; a nucleotide sequence which is the complement of said nucleotide sequence which has an identity of at least 80% to said sequence; and a nucleotide sequence which hybridizes under stringent conditions to said sequence. Also claimed is a pharmaceutical composition containing as an active agent at least one of said nucleic acid and optionally a pharmaceutically acceptable carrier, and a method of identifying microRNA molecules or precursor molecules thereof comprising ligating 5′-and 3′-adapter molecules to the ends of a size-fractionated RNA population, reverse transcribing said adapter containing RNA population and characterizing the reverse transcription products (Tuschl et al., 2003).

Disclosed and claimed in PCT Publication WO 03/006477 is an isolated nucleic acid molecule comprising a regulatory sequence operably linked to a nucleic acid sequence that encodes an engineered ribonucleic acid (RNA) precursor, wherein the precursor comprises a first stem portion comprising a sequence of at least 18 nucleotides that is complementary to a sequence of a messenger RNA (mRNA) of a target gene, a second stem portion comprising a sequence of at least 18 nucleotides that is sufficiently complementary to the first stem portion to hybridize with the first stem portion to form a duplex stem, and a loop portion that connects the two stem portions. Also claimed is an engineered RNA precursor comprising a first stem portion comprising a sequence of at least 18 nucleotides that is complementary to a sequence of a messenger RNA (mRNA) of a target gene, a second stem portion comprising a sequence of at least 18 nucleotides that is sufficiently complementary to the first stem portion to hybridize with the first stem portion to form a duplex stem, and a loop portion that connects the two stem portions. Further claimed is a vector comprising said nucleic acid molecule, a host cell, a transgene comprising said nucleic acid, a transgenic, non-human animal, one or more of whose cells comprise a transgene comprising said nucleic acid molecule, wherein the transgene is expressed in one or more cells of the transgenic animal resulting in the animal exhibiting ribonucleic acid interference (RNAi) of the target gene by the engineered RNA precursor, a method of inducing ribonucleic acid interference (RNAi) of a target gene in a cell in an animal, and a method of inducing ribonucleic acid interference (RNAi) of a target gene in a cell, the method comprising obtaining a host cell, culturing the cell, and enabling the cell to express the RNA precursor to form a small interfering ribonucleic acid (siRNA) within the cell, thereby inducing RNAi of the target gene in the cell (Zamore et al., 2003).

Disclosed and claimed in US Patent Application U.S. 2003/0092180 is a process for delivering an siRNA into a cell of a mammal to inhibit nucleic acid expression, comprising making siRNA consisting of a sequence that is complementary to a nucleic acid sequence to be expressed in the mammal, inserting the siRNA into a vessel in the mammal, and delivering the siRNA to the parenchymal cell wherein the nucleic acid expression is inhibited, as well as a process for delivering siRNA to a cell in a mammal to inhibit nucleic acid expression, comprising: inserting the siRNA into a vessel, increasing volume in the mammal to facilitate delivery, delivering the siRNA to the cell, and inhibiting nucleic acid expression (Lewis et al., 2003).

Because RNAi has been demonstrated to suppress gene expression in adult animals, it is hoped that small noncoding RNA-mediated mechanisms might be used in novel therapeutic approaches such as attenuation of viral infection, cancer therapies (Shi, Trends Genet, 2003, 19, 9-12; Silva et al., Trends Mol Med, 2002, 8, 505-508) and in regulation of stem cell differentiation (Kawasaki and Taira, Nature, 2003, 423, 838-842).

Small noncoding RNA-mediated regulation of gene expression is an attractive approach to the treatment of diseases as well as infection by pathogens such as bacteria, viruses and prions. Prion infections resulting in fatal neurodegenerative disorders are associated with an abnormal isoform of the PrPc host-encoded protein. The Prnp gene encoding PrPc has been downregulated in transgenic mice, leading to viable, healthy animals which are resistant to challenge by the infectious agent. Recently, the Prmp mRNA was targeted by RNAi, and a reduction in PrPc levels in transfected cells was demonstrated (Tilly et al., Biochem Biophys Res Commun, 2003, 305, 548-551). Thus, regulation of gene expression using small noncoding RNAs represents a potential means of treating pathogen infection.

There remains a long-felt need for agents which regulate gene expression via the small noncoding RNA-mediated mechanism. Identification of modified miRNAs or miRNA mimics which can increase or decrease gene expression or activity is therefore desirable. Furthermore, because misregulation of genes is known to lead to hyperproliferation and oncogenesis, it is also desirable to target small noncoding RNAs themselves as a means of altering aberrant gene regulation.

Like the RNAse H pathway, the RNA interference pathway for modulation of gene expression is an effective means for modulating the levels of specific gene products and, thus, would be useful in a number of therapeutic, diagnostic, and research applications involving gene silencing. The present invention therefore provides oligomeric compounds useful for modulating gene expression pathways, including those relying on mechanisms of action such as RNA interference and dsRNA enzymes, as well as antisense and non-antisense mechanisms. One having skill in the art, once armed with this disclosure will be able, without undue experimentation, to identify suitable oligonucleotide compounds for these uses.

Drug discovery has evolved from the random screening of natural products into a combinatorial approach of designing large numbers of synthetic molecules as potential bioactive agents (ligands, agonists, antagonists, and inhibitors). Traditionally, drug discovery and optimization have involved the expensive and time-consuming process of synthesis and evaluation of single compounds bearing incremental structural changes. For natural products, the individual components of extracts had to be painstakingly separated into pure constituent compounds prior to biological evaluation. Further, all compounds had to be analyzed and characterized prior to in vitro screening. These screens typically included the evaluation of candidate compounds for binding affinity to their target, competition for the ligand binding site, or efficacy at the target as determined via inhibition, cell proliferation, activation or antagonism end points. Considering all these facets of drug design and screening that slow the process of drug discovery, a number of approaches to alleviate or remedy these matters, have been implemented by those involved in discovery efforts.

The development and use of combinatorial chemistry has radically changed the way diverse chemical compounds are synthesized as potential drug candidates. The high-throughput screening of hundreds of thousands of small molecules against a biological target has become the norm in many pharmaceutical companies. The screening of a combinatorial library of compounds requires the subsequent identification of the active component, which can be difficult and time consuming. In addition, compounds are usually tested as mixtures to efficiently screen large numbers of molecules.

A shortcoming of existing assays relates to the problem of “false positives.” In a typical functional assay, a false positive is a compound that triggers the assay but which compound is not effective in eliciting the desired physiological response. In a typical physical assay, a false positive is a compound that attaches itself to the target but in a non-specific manner (e.g. non-specific binding). False positives are particularly prevalent and problematic when screening higher concentrations of putative ligands because many compounds have non-specific affects at those concentrations. Methods for directly identifying compounds that bind to macromolecules in the presence of those that do not bind to the target could significantly reduce the number of “false positives” and eliminate the need for deconvoluting active mixtures.

In a similar fashion, existing assays are also plagued by the problem of “false negatives,” which result when a compound gives a negative response in the assay but the compound is actually a ligand for the target. False negatives typically occur in assays that use concentrations of test compounds that are either too high (resulting in toxicity) or too low relative to the binding or dissociation constant of the compound to the target.

When a drug discovery scientist screens combinatorial mixtures of compounds, the scientist will conventionally identify an active pool, deconvolute it into its individual members, and identify the active members via re-synthesis and analysis of the discrete compounds. In addition to false positives and false negative, current techniques and protocols for the study of combinatorial libraries against a variety of biologically relevant targets have other shortcomings. These include the tedious nature, high cost, multi-step character, and low sensitivity of many screening technologies. These techniques do not always afford the most relevant structural and binding information, for example, the structure of a target in solution and the nature and the mode of the binding of the ligand with the receptor site. Further, they do not give relevant information as to whether a ligand is a competitive, noncompetitive, concurrent or a cooperative binder of the biological target's binding site.

The screening of diverse libraries of small molecules created by combinatorial synthetic methods is a recent development that has the potential to accelerate the identification of lead compounds in drug discovery. Rapid and direct methods have been developed to identify lead compounds in drug discovery involving affinity selection and mass spectrometry. In this strategy, the receptor or target molecule of interest is used to isolate the active components from the library physically, followed by direct structural identification of the active compounds bound to the target molecule by mass spectrometry. In a drug design strategy, structurally diverse libraries can be used for the initial identification of lead compounds. Once lead compounds have been identified, libraries containing compounds chemically similar to the lead compound can be generated and used to develop a structural activity relationship (SAR) in order to optimize the binding characteristics of the ligand with the target receptor.

One step in the identification of bioactive compounds involves the determination of binding affinity and binding mode of test compounds for a desired biopolymeric or other receptor. For combinatorial chemistry, with its ability to synthesize, or isolate from natural sources, large numbers of compounds for in vitro biological screening, this challenge is greatly magnified. Since combinatorial chemistry generates large numbers of compounds, often isolated as mixtures, there is a need for methods which allow rapid determination of those members of the library or mixture that are most active, those which bind with the highest affinity, and the nature and the mode of the binding of a ligand to a receptor target.

An analysis of the nature and strength of the interaction between a ligand (agonist, antagonist, or inhibitor) and its target can be performed by ELISA (Kemeny and Challacombe, in ELISA and other Solid Phase Immunoassays: Theoretical and Practical Aspects; Wiley, New York, 1988), radioligand binding assays (Berson and Yalow, Clin. Chim. Acta, 1968, 22, 51-60; Chard, in “An Introduction to Radioimmunoassay and Related Techniques,” Elsevier press, Amsterdam/New York, 1982), surface-plasmon resonance (Karlsson, Michaelsson and Mattson, J. Immunol. Methods, 1991, 145, 229; Jonsson et al., Biotechniques, 1991, 11, 620), or scintillation proximity assays (Udenfriend, Gerber and Nelson, Anal. Biochem., 1987, 161, 494-500). Radio-ligand binding assays are typically useful only when assessing the competitive binding of the unknown at the binding site for that of the radio-ligand and also require the use of radioactivity. The surface-plasmon resonance technique is more straightforward to use, but is also quite costly. Conventional biochemical assays of binding kinetics, and dissociation and association constants are also helpful in elucidating the nature of the target-ligand interactions but are limited to the analysis of a few discrete compounds.

A nuclear magnetic resonance (NMR)-based method is described in which small organic molecules that bind to proximal subsites of a protein are identified, optimized, and linked together to produce high-affinity ligands (Shuker et al., Science, 1996, 274, 5252, 1531). The approach is called SAR by NMR because structure-activity relationships (SAR) are obtained from NMR. This technique has several drawbacks for routine screening of a library of compounds. For example, the biological target is required to incorporate a ¹⁵N label. Typically the nitrogen atom of the label is part of amide moiety within the molecule. Because this technique requires deshielding between nuclei of proximal atoms, the ¹⁵N label must also be in close proximity to a biological target's binding site to identify ligands that bind to that site. The binding of a ligand conveys only the approximate location of the ligands. It provides no information about the strength or mode of binding. Moreover none of these methods provide information about changes in the secondary or ternary structure caused or influenced by the intended binding.

Therefore, methods for the screening and identification of complex target/ligand binding and the resultant changes in target conformation are greatly needed. In particular, new methods are needed for the identification of the strength and mode of binding of a ligand to its intended target and the extent to which that binding facilitates a change in target secondary structure are needed. In addition, methods for the screening and identification of complex target/ligand binding, where the ligand is a microRNA and the target is a small molecule, perhaps from a library of small molecules, are greatly needed. In particular, new methods are needed for the identification of the strength and mode of binding of a ligand to its intended target. In addition, new methods that identify and select for directed folding of target RNA are needed.

Synthetic oligonucleotidic compounds may comprise one or more nucleobase sequences sufficient in identity and number to effect specific hybridization or other interactions with a particular (target) nucleic acid. In one instance, because such compounds are complementary to the “sense” strand of nucleic acids that encode polypeptides, they are commonly referred to as “antisense compounds.” A subset of such compounds may be capable of modulating the expression of the target nucleic acid in vivo; such synthetic compounds are described herein as “active oligonucleotide compounds.”

Oligonucleotide compounds are commonly used in vitro as research reagents and diagnostic aids, and in vivo as therapeutic agents. Oligonucleotide compounds can exert their effect by a variety of means. One such means is the antisense-mediated direction of an endogenous nuclease, such as RNase H in eukaryotes or RNase P in prokaryotes, to the target nucleic acid (Chiang et al., J. Biol. Chem., 1991, 266, 18162; Forster et al., Science, 1990, 249, 783). Another means involves covalently linking a synthetic moiety having nuclease activity to an oligonucleotide having an antisense sequence, rather than relying upon recruitment of an endogenous nuclease. Synthetic moieties having nuclease activity include, but are not limited to, enzymatic RNAs, lanthanide ion comlexes, and the like (Haseloff et al., Nature, 1988, 334, 585; Baker et al., J. Am. Chem. Soc., 1997, 119, 8749).

Despite the advances made in utilizing antisense technology to date, it is still preferred to identify sequences amenable to antisense technologies through an empirical approach (Szoka, Nature Biotechnology, 1997, 15, 509). Accordingly, the need exists for systems and methods for efficiently and effectively identifying target nucleotide sequences that are suitable for antisense modulation. The present disclosure answers this need by providing systems and methods for automatically identifying such sequences via in silico and robotic and automated means.

Traditionally, new chemical entities with useful properties are generated by (1) identifying a chemical compound (called a “lead compound”) with some desirable property or activity, (2) creating variants of the lead compound, and (3) evaluating the property and activity of such variant compounds. Although it has been utilized with some degree of success, there are a number of limitations to this approach to lead compound generation, particularly as it pertains to the discovery of bioactive oligonucleotide compounds.

One limitation pertains to the first step of the traditional approach, i.e., the identification of lead compounds. For antisense compounds, although it was a “quite unexpected” finding, active antisense sequences are “dificult to identify” among a pool of candidate antisense sequences (Szoka, Nature Biotechnology, 1997, 15, 509). RNA structure can inhibit duplex formation with antisense compounds, so much so that moving the target nucleotide sequence even a few bases can drastically decrease the activity of such compounds (Lima et al., Biochemistry, 1992, 31, 12055).

Moreover, the search for lead antisense compounds has been limited to the manual synthesis and analysis of such compounds. Consequently, a fundamental limitation of the conventional approach is its dependence upon the availability, number and cost of antisense compounds produced by manual, or at best semi-automated, means. Moreover, the assaying of such compounds has also traditionally been performed by tedious manual techniques. Thus, the traditional approach to generating active antisense compounds is limited by the relatively high cost and long time required to synthesize and screen a relatively small number of candidate antisense compounds.

Accordingly, the need exists for systems and methods for efficiently and effectively generating new active antisense compounds targeted to specific nucleic acids. The present disclosure answers this need by providing systems and methods for automatically generating active antisense compounds via robotic means.

Efforts such as the Human Genome Project are making an enormous amount of nucleotide sequence information available in a variety of forms, e.g., genomic sequences, cDNAs, expression sequence tags (ESTs) and the like. This explosion of information has led one commentator to state that “genome scientists are producing more genes than they can put a function to” (Kahn, Science, 1995, 270, 369). Although some approaches to this problem have been suggested, no solution has yet emerged. For example, methods of looking at gene expression in different disease states or stages of development only provide, at best, an association between a gene and a disease or stage of development (Nowak, Science, 1995, 270, 368). Another approach, looking at the proteins encoded by genes, is developing but “this approach is more complex and big obstacles remain” (Kahn, Science, 1995, 270, 369). Furthermore, neither of these approaches allows one to directly utilize nucleotide sequence information to perform gene function analysis.

In contrast, antisense technology does allow for the direct utilization of nucleotide sequence information for gene function analysis. Once a target nucleic acid sequence has been selected, antisense sequences hybridizable to the sequence can be generated using techniques known in the art. Typically, a large number of candidate antisense oligonucleotides (ASOs) are synthesized having sequences that are more-or-less randomly spaced across the length of the target nucleic acid sequence (e.g., a “gene walk”) and their ability to modulate the expression of the target nucleic acid is assayed. Cells or animals are then treated with one or more active antisense oligonucleotides, and the resulting effects are determined, in order to determine the function(s) of the target gene. Although the practicality and value of the empirical approach to developing active antisense compounds has been acknowledged in the art, it has also been stated that this approach “is beyond the means of most laboratories and is not feasible when a new gene sequence is identified, but whose function and therapeutic potential are unknown” (Scoza, Nature Biotechnology, 1997, 15, 509).

Accordingly, the need exists for systems and methods for efficiently and effectively determining the function of a gene that is uncharacterized except that its nucleotide sequence, or a portion thereof, is known. The present disclosure answers this need by providing systems and methods for automatically generating active antisense compounds to a target nucleotide sequence via robotic means. Such active antisense compounds are contacted with a cell, cell-free extract or animal capable of expressing the gene of interest, and subsequent biochemical or biological parameters are measured. The results are compared to those obtained from a control cell, cell-free extract or animal which has not been contacted with an active antisense compound in order to determine the function of the gene of interest.

Determining the nucleotide sequence of a gene is no longer an end unto itself; rather, it is “merely a means to an end. The critical next step is to validate the gene and its [gene] product as a potential drug target” (Glasser, Genetic Engineering News, 1997, 17, 1). This process, i.e., confirming that modulation of a gene that is suspected of being involved in a disease or disorder actually results in an effect that is consistent with a causal relationship between the gene and the disease or disorder, is known as target validation.

Efforts such as the Human Genome Project are yielding a vast number of complete or partial nucleotide sequences, many of which might correspond to or encode targets useful for new drug discovery efforts. The challenge represented by this plethora of information is how to use such nucleotide sequences to identify and rank valid targets for drug discovery. Antisense technology provides one means by which this might be accomplished; however, the many manual, labor-intensive and costly steps involved in traditional methods of developing active antisense compounds has limited their use in target validation (Szoka, Nature Biotechnology, 1997, 15, 509). Nevertheless, the great target specificity that is characteristic of antisense compounds makes them ideal choices for target validation, especially when the functional roles of proteins that are highly related (in terms of polypeptide sequence, but not at the level of the nucleic acids which encode them) are being investigated (Albert et al., Trends in Pharm. Sci., 1994, 15, 250).

Accordingly, the need exists for systems and methods for efficiently and effectively developing compounds that modulate a gene, wherein such compounds can be directly developed from nucleotide sequence information. Such compounds are needed to confirm that modulation of a gene that is thought to be involved in a disease or disorder will in fact cause an in vitro or in vivo effect that corresponds to the origin, development, spread or growth of the disease or disorder.

The present disclosure answers this need by providing systems and methods for automatically generating active antisense compounds to a target nucleotide sequence via robotic means. Such active antisense compounds are contacted with a cell, cell-free extract or animal capable of expressing the gene of interest, and subsequent biochemical or biological parameters indicative of the origin, development, spread or growth of the disease or disorder are measured. These results are compared to those obtained with a control cell, cell-free extract or animal which has not been contacted with an active antisense compound in order to determine whether or not modulation of the gene of interest will have a therapeutic benefit or not. The resulting active antisense compounds may be used as positive controls when other, non antisense-based agents directed to the same target nucleic acid, or to its gene product, are screened.

It should be noted that embodiments of the invention drawn to gene function analysis and target validation have parameters that are shared with other embodiments of the invention, but also have unique parameters. For example, antisense drug discovery naturally requires that the toxicity of the antisense compounds be minimal or undetectable, whereas, for gene function analysis or target validation, toxicity resulting from the antisense compounds is acceptable unless it interferes with the assay being used to evaluate the effects of treatment with such compounds.

U.S. Pat. No. 5,563,036 reports systems and methods of screening for compounds that inhibit the binding of a transcription factor to a nucleic acid. In one embodiment, an assay portion of the process is stated to be performed by a computer controlled robot.

U.S. Pat. No. 5,708,158 reports systems and methods for identifying pharamacological agents stated to be useful for diagnosing or treating a disease associated with gene the expression of which is modulated by a human nuclear factor of activated T cells. The methods are stated to be particularly suited to high-thoughput screening wherein one or more steps of the process are performed by a computer controlled robot.

U.S. Pat. Nos. 5,693,463 and 5,716,780 report systems and methods for identifying non-oligonucleotide molecules that specifically bind to a DNA molecule based on their ability to compete with a DNA-binding protein that recognizes the DNA molecule.

### SUMMARY OF THE INVENTION

The present invention provides methods for selecting a target molecule that has an affinity for a ligand that is equal to or greater than a baseline affinity comprising: mixing an amount of a standard target with an excess amount of the ligand, wherein the standard target forms a non-covalent binding complex with the ligand and wherein unbound ligand is present in the mixture; introducing the mixture of the standard target and the ligand into a mass spectrometer to obtain a baseline affinity; adjusting the operating performance conditions of the mass spectrometer such that the signal strength of the standard target bound to the ligand is from 1% to about 30% of the signal strength of unbound ligand; introducing at least one target molecule into the test mixture of the ligand and the standard target; introducing the test mixture into a mass spectrometer; and identifying any complexes of the target molecule and the ligand, wherein the presence of a complex is indicated by an affinity that is greater than the baseline affinity, and wherein either one or both of the target molecule and ligand, independently, is a microRNA. The mass spectrometer can be an electrospray mass spectrometer. The ligand can be a microRNA and the target molecule can be microRNA, a microRNA mimic, a protein, an RNA-DNA duplex, an RNA-RNA duplex, a DNA duplex, a polysaccharide, a phospholipid, or a glycolipid; or the target molecule can be a microRNA and the ligand can be a microRNA, a microRNA mimic, a protein, an RNA-DNA duplex, an RNA-RNA duplex, a DNA duplex, a polysaccharide, a phospholipid, or a glycolipid. The ligand and target molecule can both be a microRNA. The ligand or target molecule can be a microRNA mimic. The baseline affinity can be expressed as a dissociation constant is about 50 millimolar. The standard target can be ammonium, a primary amine, a secondary amine, a tertiary amine, an amino acid, or a nitrogen-containing heterocycle. The electrospray mass spectrometer can comprise a desolvation capillary or countercurrent gas and a lens element, and the adjustment of the operating performance conditions can comprise adjustment of the voltage potential across the capillary and the lens element, adjustment of source voltage potential to give a stable electrospray ionization as monitored by the ion abundance of free target molecule, adjustment of the temperature of the desolvation capillary or countercurrent heating gas, or adjustment of the operating gas pressure within the mass spectrometer downstream of the desolvation capillary. The standard target can be an ammonium ion, and the adjustment of the voltage potential across the capillary and the lens element can generate a signal strength of the monoammonium-microRNA complex that is from about 10% to about 20% of the signal strength of unbound microRNA. The microRNA ligand or microRNA target molecule can be from about 10 to about 200 nucleotides in length or from about 15 to about 100 nucleotides in length. The microRNA ligand or microRNA target molecule can comprise an isolated or purified portion of a larger RNA molecule. The microRNA ligand or microRNA target molecule can possess secondary and ternary structure. The electrospray mass spectrometer can comprise a gated ion storage device for effecting thermolysis of the test mixture in the mass spectrometer. The mass spectrometer can comprise mass analysis by a quadrupole, a quadrupole ion trap, a time-of-flight, a FT-ICR, or a hybrid mass detector. The electrospray mass spectrometer can comprise Z-spray, microspray, off-axis spray, or pneumatically assisted electrospray ionization. The Z-spray, microspray, off-axis spray, or pneumatically assisted electrospray ionization can each comprise countercurrent drying gas. The methods may further comprise storing the relative abundance and stoichiometry of the complexes of the ligand and target molecule in a relational database that is cross-indexed to the structure of the target molecule. The target molecule can be a member of a set of target molecules. The members of the set of target molecules, independently, can have a molecular mass less than about 1000 Daltons and fewer than 15 rotatable bonds, or a molecular mass less than about 600 Daltons and fewer than 8 rotatable bonds, or a molecular mass less than about 200 Daltons and fewer than 4 rotatable bonds or no more than one sulfur, phosphorous, or halogen atom. The signal strength can be measured by the relative ion abundance. The methods can further comprise a plurality of target molecules or standard targets.

The present invention also provides methods of selecting those members of group of compounds that can form a non-covalent complex with a ligand and where the affinity of the members for the ligand is greater than a baseline affinity comprising: mixing an amount of a standard compound with an excess amount of the ligand, wherein the standard compound forms a non-covalent binding complex with the ligand and wherein unbound ligand is present in the mixture; introducing the mixture of the standard compound and the ligand into a mass spectrometer to obtain a baseline affinity; adjusting the operating performance conditions of the mass spectrometer such that the signal strength of the standard compound bound to the ligand is from 1% to about 30% of the signal strength of unbound ligand; introducing a sub-set of the group of compounds into a test mixture of the ligand and the standard compound; introducing the test mixture into the mass spectrometer; and identifying the members of the sub-set that form complexes with the ligand, wherein the members of the sub-set have a greater affinity for the ligand than the baseline affinty for the ligand, and wherein either one or both of the group of compounds and ligand, independently, is a microRNA. The signal can be measured as the relative ion abundance. The sub-set can comprise from about 2 to about 8 member compounds. The group of compounds can comprise a collection or library of diverse compounds. The collection or library of diverse compounds can comprise a historical repository of compounds, a collection of natural products, a collection of drug substances, a collection of intermediates produced in forming drug substances, a collection of dye stuffs, a commercial collection of chemical substances, or a combinatorial library of related compounds. The collection or library of diverse compounds can comprise a library of compounds having from 2 to about 100,000 members. The method can further comprise storing the relative abundance and stoichiometry of the complexes of the member compounds and the ligand in a relational database. The method can further comprise cross-indexing the relative abundance and stoichiometry of the complexes to the structures of the member compounds. The members of the group of compounds, independently, can have a molecular mass less than about 1000 Daltons and fewer than 15 rotatable bonds, or a molecular mass less than about 600 Daltons and fewer than 8 rotatable bonds, or a molecular mass less than about 200 Daltons and fewer than 4 rotatable bonds or no more than one sulfur, phosphorous, or halogen atom. The mass spectrometer can be an electrospray mass spectrometer. The ligand or group of compounds can be a microRNA, a microRNA mimic, an RNA, a protein, an RNA-DNA duplex, an RNA-RNA duplex, a DNA duplex, a polysaccharide, a phospholipid, or a glycolipid. The baseline affinity can be expressed as a dissociation constant of about 50 millimolar. The standard compound can be ammonium. The electrospray mass spectrometer can comprise a desolvation capillary and a lens element, and the adjustment of the operating performance conditions comprises adjustment of the voltage across the capillary and the lens element.

The present invention also provides methods of detecting a ligand-target complex having an affinity as expressed as a dissociation constant of from about nanomolar to about 100 millimolar comprising: mixing an amount of a standard target with an excess amount of the ligand such that unbound ligand is present in the mixture, wherein the standard target forms a non-covalent binding complex with the ligand at an affinity of about 50 millimolar as measured as a dissociation constant indicated by an electrospray mass spectrometer; introducing the mixture of the standard target and the ligand into a mass spectrometer; adjusting the operating performance conditions of the mass spectrometer such that the relative ion abundance of the standard target bound to the ligand is from 1% to about 30% of the relative ion abundance of unbound ligand; introducing a set of target molecules into a test mixture of the ligand and the standard target; introducing the test mixture into a mass spectrometer; and identifying the members of the set of target molecules that form complexes with the ligand and have a dissociation constant of from about nanomolar to about 100 millimolar, wherein the ligand-target complex is a microRNA ligand-target complex or a ligand-microRNA target complex. The method can further comprise storing the relative abundance and stoichiometry of the complexes of the member target molecules and the ligand in a relational database. The method can further comprise cross-indexing the relative abundance and stoichiometry of the complexes to the structures of the member target molecules. The target molecules, independently, can have a molecular mass less than about 200 Daltons, or fewer than 4 rotatable bonds. The target molecules, independently, can have no more than one sulfur, no more than one phosphorous, or no more than one halogen atom.

The present invention also provides methods of detecting a ligand-target complex having from about nanomolar to about 100 millimolar affinity as measured as a dissociation constant comprising: mixing an amount of an ionic ammonium standard compound with an excess amount of the ligand such that unbound ligand is present in the mixture; introducing the mixture of the ammonium compound and the ligand into a mass spectrometer; adjusting the operating performance conditions of the mass spectrometer such that the relative ion abundance of ammonium ion bound to the ligand is from 1% to about 30% of the relative ion abundance of unbound ligand; introducing a set of target molecules into a test mixture of the ligand and the ammonium compound; introducing the test mixture into a mass spectrometer; and identifying the members of the set of target molecules that form a complex with the ligand that have from about nanomolar to about 100 millimolar affinity as measured as a dissociation constant, wherein the ligand-target complex is a microRNA ligand-target complex or a ligand-microRNA target complex. The target molecules, independently, can have a molecular mass less than about 200 molecular mass units or fewer than 4 rotatable bonds, or no more than one sulfur, no more than one phosphorous, or no more than one halogen atom.

The present invention also provides methods for determining the relative interaction between at least two target molecules and a ligand comprising: mixing an amount of at least two target molecules with an amount of the ligand to form a mixture; and analyzing the mixture by mass spectrometry to determine the presence or absence of a ternary complex corresponding to simultaneous adduction of two of the target molecules with the ligand, wherein the absence of the ternary complex indicates that binding of the target molecules to the ligand is competitive and the presence of the ternary complex indicates that binding of the target molecules to the ligand is other than competitive, and wherein either one or both of the ligand and two target molecules, independently, is a microRNA. The method can further comprise determining from the mass spectrometry analysis of the mixture, the ion abundance of i) the ternary complex, ii) a first binary complex corresponding to the adduction of a first of the target molecules with the ligand, iii) a second binary complex corresponding to the adduction of a second of the target molecules with the ligand, and iv) the ligand unbound by either the first or second target molecule; determining the relative ion abundance of the contributing binary complexes corresponding to the relative ion abundance of the first binary complex with respect to the unbound ligand multiplied by the absolute ion abundance of the second binary complex and the relative ion abundance of the second binary complex with respect to the unbound ligand multiplied by the absolute ion abundance of the first binary complex; and comparing the absolute ion abundance of the ternary complex with respect to the unbound ligand to the sum of the relative ion abundances of the contributing binary complexes, wherein an equal ion abundance of the ternary complex compared to the sum of the relative ion abundances of the contributing binary complexes indicates a concurrent binding interaction of the target molecules to the ligand, a greater ion abundance of the ternary complex indicates a cooperative binding interaction of the target molecules to the ligand, and a lesser ion abundance of the ternary complex indicates a competitive binding interaction of the target molecules to the ligand. The target molecules can be present in the mixture in molar excess to the ligand. The ligand may not be saturated with the target molecules.

The present invention also provides methods of determining binding interaction between a first target molecule and a second target molecule with respect to a ligand comprising: exposing the ligand to the first and second target molecules to form a mixture comprising i) a ternary complex (LT1T2) of the ligand bound to the first and second target molecules, ii) a first binary complex (LT1) of the first target molecule and the ligand, iii) a second binary complex (LT2) of the second target molecule and the ligand, and iv) ligand (L) unbound by either the first or second target molecule; analyzing the mixture by mass spectrometry to determine the absolute ion abundance of the ternary complex (LT1T2), the first binary complex (LT1), the second binary complex (LT2), and the ligand (L) unbound to the first or second target molecules; and comparing the ion abundance of the first and second binary complexes LT1 and LT2, the ternary complex LT1T2, and the ligand (L) in any of the following formulae: y=LT1T2−LT1×LT2/L−LT2×LT1/L or y=LT1T2−2×(LT1×LT2)/L wherein: when a value for y is zero, the first and second target molecules have a concurrent binding interaction for the ligand; when a value for y is greater than zero, the first and second target molecules have a cooperative binding interaction for the ligand; and when a value for y is less than zero, the first and second target molecules have a competitive binding interaction for the ligand, and wherein either one or both of the ligand and first and second target molecules, independently, is a microRNA. A greater ion abundance of the first binary complex (LT1) compared to the second binary complex (LT2) in the mixture indicates that the first target molecule has greater affinity for the ligand than the second target molecule. The absence of the ternary complex in the mixture indicates that the first and second target molecules bind to the ligand at the same location and the presence of the ternary complex indicates that the first and second target molecules bind to the ligand at a distinct location.

The present invention also provides methods of determining the relative proximity of binding sites for a first target molecule and a second target molecule on a ligand comprising: exposing the ligand to a mixture of the second target molecule and a plurality of derivative compounds of the first target molecule, the first target molecule derivatives comprising the chemical structure of the first target molecule and at least one substituent group pending therefrom; and analyzing the mixture by mass spectrometry to identify a first target molecule derivative that inhibits the binding of the second target molecule to the ligand or that has a competitive binding interaction with the second target molecule for the ligand, and wherein either one or both of the ligand and first and second target molecules, independently, is a microRNA. The substituent groups on the first target molecule binding derivatives can be iteratively lengthened to determine the relative proximity of the second target molecule binding site.

The present invention also provides methods of determining the relative orientation of a first target molecule to a second target molecule when bound to a ligand comprising: exposing the ligand to a mixture of the second target molecule and a plurality of derivative compounds of the first target molecule, the first target molecule derivatives comprising the chemical structure of the first target molecule and having a substituent group pending therefrom; and analyzing the mixture by mass spectrometry to identify a first target molecule derivative that inhibits the binding of the second target molecule to the ligand or that has a competitive binding interaction with the second target molecule for the ligand. The relative orientation of the first and second target molecules when bound to the ligand can be relative to the position at which the substituent group is attached to the chemical structure of the first target molecule. The substituent group can be iteratively attached to different locations on the first target molecule derivatives to determine the relative orientation of the first target molecule binding site to the second target molecule binding site.

The present invention also provides methods for screening target molecules having binding affinity to a ligand comprising: identifying by mass spectrometry in a mixture comprising the target molecules and ligand a first and second target molecule that bind to the ligand non-competitively; and concatenating the first and second target molecule to form a third target molecule having greater binding affinity for the ligand than either the first or second target molecules, and wherein either one or both of the ligand and target molecules, independently, is a microRNA. The relative proximity of the first and second target molecule binding sites can be determined comprising: exposing the ligand to a mixture of the second target molecule and a plurality of derivative compounds of the first target molecule, the first target molecule derivatives comprising the chemical structure of the first target molecule and at least one substituent group pending therefrom; and analyzing the mixture by mass spectrometry to identify a first target molecule derivative that inhibits the binding of the second target molecule to the ligand or that has a competitive binding interaction with the second target molecule for the ligand. The relative orientation of the first and second target molecules when bound to the ligand can be determined comprising: exposing the ligand to a mixture of the second target molecule and a plurality of derivative compounds of the first target molecule, the first target molecule derivatives comprising the chemical structure of the first target molecule and having a substituent group pending therefrom; and analyzing the mixture by mass spectrometry to identify a first target molecule derivative that inhibits the binding of the second target molecule to the ligand or that has a competitive binding interaction with the second target molecule for the ligand. The substituent group can be alkyl, alkenyl, alkynyl, alkoxy, alkoxycarbonyl, acyl, acyloxy, aryl, aralkyl, hydroxyl, hydroxylamino, keto (═O), amino, alkylamino, mercapto, thioalkyl, halogen, nitro, haloalkyl, phosphorous, phosphate, sulfur, or sulfate. The relative proximity of the first and second target molecule binding sites can be determined by in silico calculation or nuclear magnetic resonance. The relative orientation of the first and second target molecules when bound to the ligand can be determined by in silico calculation nuclear magnetic resonance. The third target molecule can comprise the chemical structures of the first and second target molecules covalently linked by a linking group having a length and points of attachment to the target molecules corresponding to the relative proximity and orientation of the substituent group. The linking group can be a bond, alkylene, alkenylene, alkynylene, arylene, ether, alkylene-ester, thioether, alkylene-thioester, aminoalkylene, amine, thioalkylene, or heterocycle.

The present invention also provides methods for modulating the binding affinity of a target molecule for a ligand comprising: exposing the ligand to a first target fragment and a second target fragment; interrogating the ligand exposed to the first and second target fragments in a mass spectrometer to identify binding of the first and second target fragments to the ligand; and concatenating the first and second target fragments together in a structural configuration that improves the binding properties of the first and second target fragments for the ligand, wherein either one or both of the ligand and target molecule is, independently a microRNA. The improvement in binding properties can comprise an increase in binding affinity or a conformational change induced in the ligand, or an increase in binding affinity or a conformational change induced in the ligand. The method can further comprise: modifying the first target fragment by making a structural derivative of the first target fragment to form a modified first target fragment; re-exposing the ligand to the modified first target fragment and the second target fragment; re-interrogating the ligand exposed to the modified first target fragment and the second target fragment in the mass spectrometer to identify binding of the modified first target fragment and the second target fragment to the ligand; and concatenating the modified first target fragment and the second target fragment together in a structural configuration that increases the binding affinity to the ligand. The method can further comprise: modifying the second target fragment by making a structural derivative of the second target fragment to form a modified second target fragment; re-exposing the ligand to the modified first target fragment and the modified second target fragment; re-interrogating the ligand exposed to the modified first target fragment and the modified second target fragment in the mass spectrometer to identify binding of modified target fragments to the ligand; and covalently joining the modified first target fragment and the modified second target fragment together in a structural configuration that mimics the conformation or location of the fragments on the ligand. The first target fragment can be modified by replacing one atom or one substituent group on the first target molecule with a different atom or a different substituent group or by replacing a hydrogen atom with a substituent group. The substituent group can be alkyl, alkenyl, alkynyl, alkoxy, alkoxycarbonyl, acyl, acyloxy, aryl, aralkyl, hydroxyl, hydroxylamino, keto (═O), amino, alkylamino, mercapto, thioalkyl, halogen, nitro, haloalkyl, phosphorous, phosphate, sulfur, or sulfate. The first target fragment can be selected as a target containing a ring and the first target fragment can be modified by expanding or contracting the size of the ring. The second target fragment can be modified by replacing one atom or substituent group on the target with a different atom or different substituent group. The second target fragment can be modified by replacing a hydrogen atom with a substituent group. The substituent group can be alkyl, alkenyl, alkynyl, alkoxy, alkoxycarbonyl, acyl, acyloxy, aryl, aralkyl, hydroxyl, hydroxylamino, keto (═O), amino, alkylamino, mercapto, thioalkyl, halogen, nitro, haloalkyl, phosphorous, phosphate, sulfur, or sulfate. The second target fragment can be selected as a target containing a ring and the target fragment can be modified by expanding or contracting the size of the ring. The method can further comprise refining the binding of a target fragment to the ligand using molecular modeling. The refining can comprise: virtually concatenating the target fragments together to form an in silico 3D model of the concatenated target fragments; positioning the in silico 3D model of the concatenated target fragments on an in silico 3D model of the ligand; scoring the positioning of the in silico 3D model of the concatenated target fragments on the in silico 3D model of the ligand; and refining the positioning of the in silico 3D model of the concatenated target fragments on the in silico 3D model of the ligand using the results of the scoring. The scoring can use one or more hydrophobic, hydrogen-bonding, or electrostatic interactions between the in silico 3D model of the concatenated target fragments and the in silico 3D model of the ligand. The method can further comprise: covalently joining the target fragments together in a structural configuration that mimics the virtually concatenated target fragments; re-exposing the ligand to the covalently joined target fragments; and re-interrogating the ligand exposed to the covalently joined target fragments in the mass spectrometer to identify binding of the covalently joined target fragments and the ligand. The binding can be competitive, concurrent, or cooperative. The target fragments can exhibit either cooperative or concurrent binding with the ligand can be selected for concatenation. The ligand or target molecule can be a microRNA mimic. The ligand or target molecule can be from about 10 to about 200 nucleotides in length, or from about 15 to about 100 nucleotides in length. The ligand or target molecule can compris an isolated or purified portion of a larger RNA molecule. The ligand or target molecule can have secondary and ternary structure. The fragments independently can have a molecular mass of less than 400 or less than 200 or have no more than three rotatable bonds, or have no more than one sulfur, phosphorous, or halogen atom. The ligand or target molecule can be an ammonium salt. The ligand exposed to the target fragments can be introduced into the mass spectrometer via an electrospray ionization source. The electrospray ionization source can be a Z-spray, microspray, off-axis spray, or pneumatically assisted electrospray. The electrospray ionization source can further comprise countercurrent drying gas. The ligand exposed to the target molecules can be interrogated by a mass analyzer, a quadrupole, a quadrupole ion trap, a time-of-flight, a FT-ICR, or a hybrid mass analyzer.

The present invention also provides methods for refining the binding of a target molecule to a ligand comprising: virtually concatenating a first virtual fragment of the target with a second virtual fragment of the target to form an in silico 3D model of the concatenated target fragments; positioning the in silico 3D model of the concatenated target fragments on an in silico 3D model of the ligand; scoring the positioning of the in silico 3D model of the concatenated target fragments on the in silico 3D model of the ligand; and refining the positioning of the in silico 3D model of the concatenated target fragments on the in silico 3D model of the ligand using the results of the scoring, wherein either one or both of the ligand and target molecule is, independently, a microRNA. The scoring can use one or more hydrophobic, hydrogen-bonding, or electrostatic interactions between the in silico 3D model of the concatenated target fragments and the in silico 3D model of the ligand. The method can further comprise: covalently joining a real first target corresponding to the first virtual target fragment with a real second target corresponding to the second virtual target fragment together in a structural configuration that mimics the virtually concatenated target fragments; exposing the ligand to the covalently joined target fragments; and re-interrogating the ligand exposed to the covalently joined target fragments in a mass spectrometer to identify binding of the covalently joined target fragments and the ligand. The method can further comprise: modifying the first virtual target fragment by making a structural derivative of the first virtual target fragment to form a modified first virtual target fragment; virtually concatenating the modified first virtual target fragment and the second virtual target fragment together to form a modified in silico 3D model of the concatenated target fragments; positioning the modified in silico 3D model of the concatenated target fragments on an in silico 3D model of the ligand; scoring the positioning of the modified in silico 3D model of the concatenated target fragments on the in silico 3D model of the ligand; and refining the positioning of the modified in silico 3D model of the concatenated target fragments on the in silico 3D model of the ligand using the results of the scoring. The relative proximity of the first target molecule binding site to the second target molecule binding site can be proportional to the length of the substituent group pending from a first target molecule derivative that inhibits the binding of the second target molecule to the ligand or that has a competitive binding interaction with the second target molecule for the ligand.

In any of the above-described methods, the microRNA mimic can comprise an oligonucleotide comprising from 21 to 24 nucleotides, wherein the oligonucleotide is divided into three regions, and wherein one of the regions comprises a region having at least one first modified nucleotide, wherein the first modified nucleotide comprises a nucleotide that decreases binding affinity for an opposite strand as compared to the binding affinity of an unmodified ribonucleotide to the opposite strand. In addition, at least one of the other of the regions can comprise a region having at least one second modified nucleotide, wherein the second modified nucleotide can comprise a nucleotide that has increased binding affinity to an opposite strand as compared to the binding of an unmodified ribonucleotide to the opposite strand. The other regions can compris a region having at least one second modified nucleotide. The second modified nucleotide can comprise a nucleotide having a 3′-endo configuration, a nucleotide having 4′-deoxy-4′-thio sugar component, a pair of nucleotides linked together with a linkage that has greater binding affinity than the binding affinity of a phosphodiester linkage, or a morpholino nucleotide, a LNA nucleotide, an ENA nucleotide, a hexenyl nucleotide, or PNA nucleotide mimic. The first modified nucleotide can comprise a nucleotide having a heterocylic base that does not hydrogen bond to the heterocyclic bases of RNA and DNA, a purine nucleotide having a substituent group on its 2 or 6 positions and where the substituent is not a hydroxy or amine group, or a pyrimidine nucleotide having a substituent group on its 2 or 4 positions and where the substituent is not a hydroxy or amine group. The oligonucleotide can be 22 nucleotides in length.

The present invention also provides methods of favoring an alternate structure of an oligomer comprising: chemically modifying a first nucleoside of a first portion of the oligomer thereby forming a first modified nucleoside; and chemically modifying a second nucleoside of a second portion of the oligomer thereby forming a second modified nucleoside where the first modified nucleoside and the second modified nucleoside attract each other, energetically favoring the secondary structure. The favored secondary structure can mimic a microRNA.

The present invention also provides methods for identifying a ligand that alters a target compound secondary structure comprising: contacting the target compound with a test ligand to produce a test combination; measuring the conformation of the target in the test combination; and repeating the contacting and measuring steps with a plurality of test ligands to identify ligands that alter the target secondary structure. The measurable change in the target secondary structure can comprise a change in the target secondary structure from less folded to more folded, from more folded to less folded, or from a first folded secondary structure to a second, alternative, secondary structure. The target can be an RNA from about 5 to about 500 nucleotides in length. The measuring step can comprise contacting the test combination and a control combination with an oligonucleotide under conditions in which the oligonucleotide preferentially hybridizes to a predetermined conformation of the target RNA sequence, and measuring the fraction of the target RNA sequence present in hybrids with the oligonucleotide, wherein the fraction measured indicates the fraction of the target RNA in the predetermined conformation. The ligand can be a miRNA, microRNA mimic, siRNA, stRNA, sncRNA, tncRNA, snoRNA, smnRNA, snRNA, other small non-coding RNA, RNA, DNA, proteins, RNA-DNA duplexes, DNA duplexes, polysaccharides, phospholipids, glycolipids, or a mimic thereof, or a combination thereof. The microRNA mimic can comprise from 21 to 24 nucleotides, wherein the microRNA mimic is divided in to three regions, wherein one of the regions comprises a region having at least one first modified nucleotide that decreases binding affinity for an opposite strand as compared to the binding affinity of an unmodified ribonucleotide to the opposite strand. At least one of the other of the regions can comprise a region having at least one second modified nucleotide that has increased binding affinity to an opposite strand as compared to the binding of an unmodified ribonucleotide to the opposite strand. The other regions can comprise a region having at least one second modified nucleotide. The second modified nucleotide can comprise a 3′-endo configuration. The second modified nucleotide can comprise a 4′-deoxy-4′-thio sugar component, or a pair of nucleotides linked together with a linkage that has greater binding affinity than the binding affinity of a phosphodiester linkage, or a morpholino nucleotide, a LNA nucleotide, an ENA nucleotide, a hexenyl nucleotide, or PNA nucleotide mimic. The first modified nucleotide can comprise a nucleotide having a heterocylic base that does not hydrogen bond to the heterocyclic bases of RNA and DNA, or a purine nucleotide having a substituent group on its 2 or 6 positions and where the substituent is not a hydroxy or amine group, or a pyrimidine nucleotide having a substituent group on its 2 or 4 positions and where the substituent is not a hydroxy or amine group. The microRNA mimic can be 22 nucleotides in length.

The present invention also provides methods of determining the relative change in proximity of binding sites for a first ligand and a second ligand on a target substrate influenced by the first ligand comprising: exposing the target substrate to the first ligand under binding conditions, thereby forming a first bound target; exposing the first bound target to a second ligand under binding conditions, therby forming a mixture; and analyzing the mixture by mass spectrometry to determine the relative change in proximity of binding sites for the first ligand and the second ligand. The ligand can be a miRNA, microRNA mimic, siRNA, stRNA, sncRNA, tncRNA, snoRNA, smRNA, snRNA, other small non-coding RNA, RNA, DNA, proteins, RNA-DNA duplexes, DNA duplexes, polysaccharides, phospholipids, glycolipids, or a mimic thereof, or a combination thereof. The microRNA mimic can comprise from 21 to 24 nucleotides, wherein the microRNA mimic is divided in to three regions, wherein one of the regions comprises a region having at least one first modified nucleotide that decreases binding affinity for an opposite strand as compared to the binding affinity of an unmodified ribonucleotide to the opposite strand. At least one of the other of the regions can comprise a region having at least one second modified nucleotide that has increased binding affinity to an opposite strand as compared to the binding of an unmodified ribonucleotide to the opposite strand. The other regions can comprise a region having at least one second modified nucleotide. The second modified nucleotide can comprise a 3′-endo configuration, or a 4′-deoxy-4′-thio sugar component, or a pair of nucleotides linked together with a linkage that has greater binding affinity than the binding affinity of a phosphodiester linkage, or a morpholino nucleotide, a LNA nucleotide, an ENA nucleotide, a hexenyl nucleotide, or PNA nucleotide mimic. The first modified nucleotide can comprise a nucleotide having a heterocylic base that does not hydrogen bond to the heterocyclic bases of RNA and DNA, or a purine nucleotide having a substituent group on its 2 or 6 positions and where the substituent is not a hydroxy or amine group, or a pyrimidine nucleotide having a substituent group on its 2 or 4 positions and where the substituent is not a hydroxy or amine group. The microRNA mimic can be 22 nucleotides in length.

The present invention also provides methods of determining the relative change in proximity of a first binding site for a first binding ligand and a second binding site for a second binding ligand on a target comprising: exposing the target to a first influential ligand that alters the target's secondary folding according to a folding influence; exposing the target to a first binding ligand; exposing the target to a mixture of the second binding ligand and a plurality of derivative compounds of the first binding ligand, wherein the first binding ligand derivatives comprise the chemical structure of the first binding ligand and at least one substituent group pending therefrom; and analyzing the mixture by mass spectrometry to identify a first binding ligand derivative which inhibits the binding of said second binding ligand on the target or has a competitive binding interaction with the second binding ligand for the target. The substituent groups on the first ligand binding derivatives can be iteratively lengthened to determine the relative proximity of the second ligand binding site. The ligand can be a miRNA, microRNA mimic, siRNA, stRNA, sncRNA, tncRNA, snoRNA, smRNA, snRNA, other small non-coding RNA, RNA, DNA, proteins, RNA-DNA duplexes, DNA duplexes, polysaccharides, phospholipids, glycolipids, or a mimic thereof, or a combination thereof. The microRNA mimic can comprise from 21 to 24 nucleotides, wherein the microRNA mimic is divided in to three regions, wherein one of the regions comprises a region having at least one first modified nucleotide that decreases binding affinity for an opposite strand as compared to the binding affinity of an unmodified ribonucleotide to the opposite strand. At least one of the other of the regions comprises a region having at least one second modified nucleotide that has increased binding affinity to an opposite strand as compared to the binding of an unmodified ribonucleotide to the opposite strand. The other regions can comprise a region having at least one second modified nucleotide. The second modified nucleotide can comprise a 3′-endo configuration, or a 4′-deoxy-4′-thio sugar component, or a pair of nucleotides linked together with a linkage that has greater binding affinity than the binding affinity of a phosphodiester linkage, or a morpholino nucleotide, a LNA nucleotide, an ENA nucleotide, a hexenyl nucleotide, or PNA nucleotide mimic. The first modified nucleotide can comprise a nucleotide having a heterocylic base that does not hydrogen bond to the heterocyclic bases of RNA and DNA, or a purine nucleotide having a substituent group on its 2 or 6 positions and where the substituent is not a hydroxy or amine group, or a pyrimidine nucleotide having a substituent group on its 2 or 4 positions and where the substituent is not a hydroxy or amine group. The microRNA mimic can be 22 nucleotides in length.

The present invention also provides methods of determining the relative orientation of a first ligand to a second ligand when bound to a target substrate comprising: exposing the target substrate to a mixture of the second ligand and a plurality of derivative compounds of the first ligand, wherein the first ligand derivatives comprise the chemical structure of the first ligand and have a substituent group pending therefrom; and analyzing the mixture by mass spectrometry to identify a first ligand derivative which inhibits the binding of the second ligand to the target substrate or has a competitive binding interaction with the second ligand for the target substrate. The relative orientation of the first and second ligands when bound to the target substrate can be relative to the position at which the substituent is attached to the chemical structure of the first ligand. The substituent group can be iteratively attached to different locations on the first ligand derivatives to determine the relative orientation of the first ligand binding site to the second ligand binding site. The ligand can be a miRNA, microRNA mimic, siRNA, stRNA, sncRNA, tncRNA, snoRNA, smRNA, snRNA, other small non-coding RNA, RNA, DNA, proteins, RNA-DNA duplexes, DNA duplexes, polysaccharides, phospholipids, glycolipids, or a mimic thereof, or a combination thereof. The microRNA mimic can comprise from 21 to 24 nucleotides, wherein the microRNA mimic is divided in to three regions, wherein one of the regions comprises a region having at least one first modified nucleotide that decreases binding affinity for an opposite strand as compared to the binding affinity of an unmodified ribonucleotide to the opposite strand. At least one of the other of the regions comprises a region having at least one second modified nucleotide that has increased binding affinity to an opposite strand as compared to the binding of an unmodified ribonucleotide to the opposite strand. The other regions can comprise a region having at least one second modified nucleotide. The second modified nucleotide can comprise a 3′-endo configuration, or a 4′-deoxy-4′-thio sugar component, or a pair of nucleotides linked together with a linkage that has greater binding affinity than the binding affinity of a phosphodiester linkage, or a morpholino nucleotide, a LNA nucleotide, an ENA nucleotide, a hexenyl nucleotide, or PNA nucleotide mimic. The first modified nucleotide can comprise a nucleotide having a heterocylic base that does not hydrogen bond to the heterocyclic bases of RNA and DNA, or a purine nucleotide having a substituent group on its 2 or 6 positions and where the substituent is not a hydroxy or amine group, or a pyrimidine nucleotide having a substituent group on its 2 or 4 positions and where the substituent is not a hydroxy or amine group. The microRNA mimic can be 22 nucleotides in length.

The present invention also provides oligomeric compounds comprising a nucleotide sequence at least 80% complementary to a target RNA, wherein the oligomeric compound comprises 21 to 24 nucleotides, and comprises a nucleotide sequence that corresponds to a portion of the nucleotide sequence of a larger oligomeric compound that comprises a stemloop structure. The oligomeric compound can comprise at least one modified nucleotide. The modified nucleotide can have increased binding affinity to an opposite strand as compared to the binding of an unmodified ribonucleotide to the opposite strand. The modified nucleotide can comprise a 3′-endo configuration, or a 4′-deoxy-4′-thio sugar component, or a pair of nucleotides linked together with a linkage that have a greater binding affinity that the binding affinity of a phosphodiester linkage, or a morpholino nucleotide, a LNA nucleotide, an ENA nucleotide, a hexenyl nucleotide, or PNA nucleotide mimic. The oligomeric compound can comprise 22 nucleotides. The oligomeric compound can comprise a nucleotide sequence corresponding to a portion of one of the stems of the stemloop structure of the larger oligomeric compound. The oligomeric compound can comprise a nucleotide sequence corresponding to a portion of the 5′ stem of the larger oligomeric compound. The oligomeric compound can comprise a nucleotide sequence corresponding to a portion of the 3′ stem of the larger oligomeric compound. The larger oligomeric compound can compris 50 to 80 nucleotides and a hairpin, and wherein the larger oligomeric compound is a substrate for DICER protein. The larger oligomeric compound comprises 50 to 70 nucleotides.

The present invention also provides methods of modulating transcription in a cell comprising contacting a target gene with a purified or isolated oligomeric compound comprising 21 to 24 nucleotides and a nucleotide sequence capable of partially hybridizing with the gene, wherein each of the ends of the oligomeric compound hybridize to the gene, and wherein a non-hydrogen binding nucleotide region located in the middle of the oligomeric compound does not hybridize with the gene. Modulation can be suppression of transcription. The oligomeric compound can comprise 22 nucleotides. The non-hydrogen binding nucleotide region can comprise at least one nucleotide having decreased hybridization with the target gene as compared to a normal nucleotide, or a bulge mismatch having at least one nucleotide that does not hydrogen bond to the target gene. The oligomeric compound can comprise at least one modified nucleotide. The modified nucleotide can be located in the non-hydrogen binding nucleotide region. The nucleotide having decreased hybridization with the target gene can comprise a modified nucleotide. At least one of the ends of the oligomeric compound can comprise a modified nucleotide. The ends of the oligomeric compound can comprise a modified nucleotide.

The present invention also provides oligomeric compounds comprising a molecule weight less than 600 daltons and of a shape sufficient to fit into a binding pocket on an RNA that is 50 to 80 nucleotides in length and comprises a hairpin structure, wherein the RNA comprises a substrate for DICER protein, and wherein the oligomeric compound is a modulator of a microRNA.

The present invention also provides methods of modulating translation in a cell comprising: assaying a library of molecules for a molecule that binds to an RNA, wherein the RNA is from 50 to 80 nucleotides in length having a hairpin structure, and wherein the RNA is a substrate for DICER protein; and contacting the RNA in the cell with the molecule to modulate the interaction of the DICER protein and the RNA. Modulation can be suppression of translation.

The present invention also provides methods of modulating conversion of a precursor RNA into a microRNA in a cell comprising: assaying a library of molecules for molecules that binds to the precursor RNA, wherein the precursor RNA is from 50 to 80 nucleotides in length and has a hairpin structure, and wherein the precursor RNA is a substrate for DICER protein; and contacting the precursor RNA in the cell with the molecule to modulate the interaction of the DICER protein and the precursor RNA.

The present invention also provides methods of generating a set of compounds that modulate the expression of a target-nucleic acid molecule comprising generating a library of compounds in silico according to defined criteria, wherein the library is comprised of microRNA, microRNA mimics, or microRNA regulators, or a combination thereof. The target nucleic acid molecule can be a genomic DNA, a cDNA, a product of a polymerase chain reaction, an expressed sequence tag, an mRNA, a microRNA, a microRNA mimic, a microRNA regulator, or a structural RNA. The target nucleic acid molecule can be human.

The present invention also provides methods of generating a set of oligomeric compounds that modulate the expression of a target nucleic acid molecule comprising robotically assaying a plurality of oligomeric compounds for one or more desired physical, chemical, or biological properties, wherein the oligomeric compounds are microRNA, microRNA mimics, or microRNA regulators, or a combination thereof.

The present invention also provides methods of generating a set of compounds that modulate the expression of a target nucleic acid molecule comprising: generating a library of oligomeric compounds in silico according to defined criteria; evaluating in silico a plurality of virtual oligomeric compounds having the nucleobase sequences of the oligomeric compounds generated in silico according to defined criteria; and robotically synthesizing a plurality of oligomeric compounds.

The present invention also provides methods of generating a set of compounds that modulate the expression of a target nucleic acid molecule comprising: generating a library of oligomeric compounds in silico according to defined criteria; evaluating in silico a plurality of virtual oligomeric compounds having the nucleobase sequences of the oligomeric compounds generated in silico according to defined criteria; and robotically assaying a plurality of oligomeric compounds for one or more desired physical, chemical, or biological properties. The step of robotically assaying the plurality of oligomeric compounds can be performed by computer-controlled real-time polymerase chain reaction or by computer-controlled enzyme-linked immunosorbent assay.

The present invention also provides methods of generating a set of compounds that modulate the expression of a target nucleic acid molecule comprising: generating a library of oligomeric compounds in silico according to defined criteria; robotically synthesizing a plurality of oligomeric compounds; and robotically assaying a plurality of oligomeric compounds for one or more desired physical, chemical, or biological properties.

The present invention also provides methods of generating a set of compounds that modulate the expression of a target nucleic acid molecule comprising: evaluating in silico a plurality of virtual oligomeric compounds according to defined criteria; robotically synthesizing a plurality of oligomeric compounds; and robotically assaying a plurality of oligomeric compounds for one or more desired physical, chemical, or biological properties.

The present invention also provides methods of generating a set of compounds that modulate the expression of a target nucleic acid molecule comprising: generating a library of oligomeric compounds in silico according to defined criteria; evaluating in silico a plurality of virtual oligomeric compounds having the nucleobase sequences of oligomeric compounds generated in silico according to defined criteria; robotically synthesizing a plurality of oligomeric compounds; and robotically assaying a plurality of oligomeric compounds for one or more desired physical, chemical, or biological properties.

The present invention also provides methods of generating a set of compounds that modulate the expression of a target nucleic acid molecule comprising: generating a library of oligomeric compounds in silico according to defined criteria; selecting an oligomeric chemistry; robotically synthesizing a set of oligomeric compounds having the nucleobase sequences of oligomeric compounds generated in silico and the oligomeric chemistry; robotically assaying the set of oligomeric compounds for a physical, chemical, or biological activity; and selecting a subset of the set of oligomeric compounds having a desired level of physical, chemical, or biological activity to generate the set of compounds.

The present invention also provides methods of generating a set of compounds that modulate the expression of a target nucleic acid molecule comprising: generating a library of oligomeric compounds in silico according to defined criteria; selecting an oligomeric chemistry; evaluating in silico a plurality of virtual oligomeric compounds having the nucleobase sequences of oligomeric compounds generated in silico and the oligomeric chemistry according to defined criteria, and selecting those having desired characteristics, to generate a set of suitable oligomeric compounds; robotically synthesizing a set of oligomeric compounds having the suitable oligomeric compounds and the oligomeric chemistry; robotically assaying the set of oligomeric compounds for a physical, chemical, or biological activity; and selecting a subset of the set of oligomeric compounds having a desired level of physical, chemical, or biological activity to generate the set of compounds.

The present invention also provides computer formatted media comprising computer readable instructions for identifying active compounds and/or computer readable instructions for performing any of the methods described herein.

The present invention also provides methods of predicting evolutionarily allowed mutations of a microRNA comprising: defining a cloud of evolutionarily allowed mutations as the cloud around a point within the four dimensional space of the microRNA where the point is determined according to the relative percent of each nucleoside within the microRNA; and determining a quantum of modulation permitted for each nucleoside where the combined positional change in the four dimensional space of the microRNA as determined by the permitted mutation does not exceed the boundary defined by the cloud.

The present invention also provides methods of grouping a plurality of biological members according to a grouping criteria comprising: obtaining at least one grouping criteria by which each biological member is grouped; comparing the grouping criteria of at least one biological member with the grouping criteria of at least one other biological member, thereby determining an interrelatedness between the at least one biological member and the at least one other biological member; and grouping the plurality of biological members according to the interrelatedness. The grouping criteria can be a biological constraint. The biological members can have phylum interrelatedness, class interrelatedness, family interrelatedness, genus interrelatedness, or species interrelatedness. The biological constraint can be an evolutionary constraint.

The present invention also provides methods of determining a blur-factor comprising: obtaining a threshold range of variance for each nucleoside within a selected region of a nucleic acid molecule; and altering the percent composition of each nucleoside within the selected region according to a corresponding threshold range, defining thereby a 4-dimensional range of interrelated nucleoside values for the selected region, thereby defining the blur-factor for each nucleoside within the selected region of the nucleic acid molecule. The 4-dimensional range of interrelated nucleoside values for the selected region can define a cloud of allowed nucleoside values for the selected region for a species. The cloud of allowed nucleoside values can be constrained according to evolutionary constraints.

The present invention also provides methods of determining a group of probable mutations for a microRNA comprising: obtaining a threshold range of variance for each nucleoside within a selected region of a microRNA containing nucleic acid molecule; and altering the percent composition of each nucleoside within the selected region according to a corresponding threshold range, defining thereby a 4-dimensional range of interrelated nucleoside values for the selected region, thereby obtaining the group of probable mutations for each nucleoside within the selected region of the nucleic acid.

### BRIEF DESCRIPTION OF THE DRAWINGS

FIG. 1 is a schematic representation of a mass spectrometer employing an electospray ion source.

FIG. 2 is a mass spectrum showing binding of a small molecule ligand (2-amino-4-benzylthio-1,2,4-triazole) to a 27-mer fragment of bacterial 16S A-site ribosomal RNA and ammonium as standard ligand.

FIG. 3 is a mass spectrum showing competitive displacement of glucosamine from the 16S RNA fragment by Ibis-326732.

FIG. 4 is a mass spectrum showing the concurrent binding of 2-DOS and 3,5-diamino-1,2,4-triazole to the 16S RNA fragment.

FIG. 5 is a table of particular amines and carboxylic acids that were conjugated at the R group in all combinations to form a library of amide linked compounds. The amide linked compounds were analyzed by mass spectroscopy to determine their binding affinity to 16S RNA fragment.

FIG. 6 is a mass spectrum showing the binding of a piperazinyl small molecule IBIS-326611 from the amide library to 16S RNA fragment.

FIG. 7 is a mass spectrum showing the binding to 16S RNA fragment of another piperazinyl small molecule IBIS-326645 from the amide library.

FIG. 8 is a mass spectrum showing the enhanced binding to the 16S RNA fragment of concatenated compound IBIS-271583, derived from the structures of IBIS-326611 and IBIS-326645 and sharing the common piperazine moiety of the two parent compounds. The concatenated compound has greater affinity for 16S than either parent compound.

FIG. 9 is a schematic representation of the binding of triazole and 2-deoxystreptamine ligands binding at their respective binding sites on the target 16S RNA fragment and a concatenated compound derived from the two ligands.

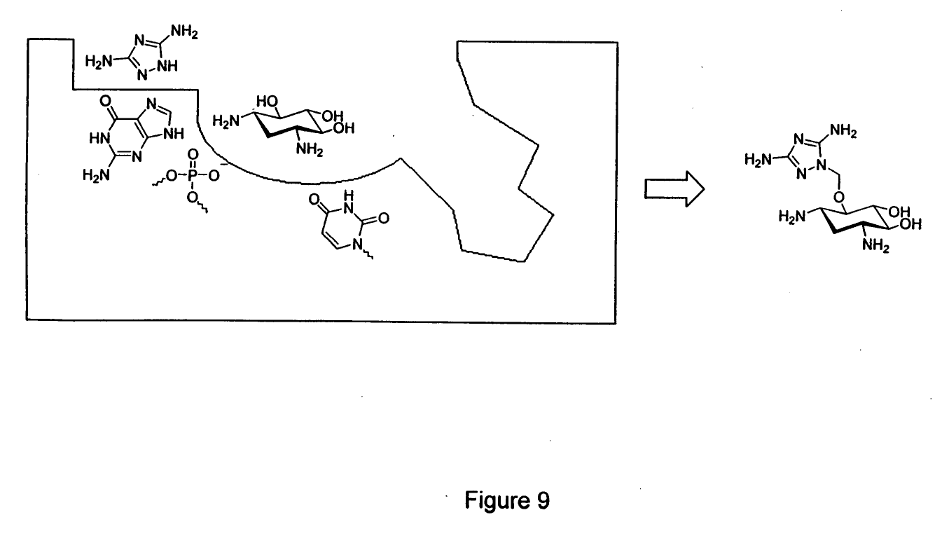

### DETAILED DESCRIPTION OF THE INVENTION

The methods of the present invention are useful for, inter alia, detection, evaluation and optimization of ligands, particularly microRNA ligands, to targets, particularly biological targets, such as microRNA targets. The detection and evaluation of the different binding modes of non-covalently bound ligands to a target are useful for advancing the structure activity relationship (SAR) and for designing ligands with higher binding affinities for their given target sites. The methods and processes of the invention utilize mass spectrometry as the primary tool to accomplish this. Mass spectrometry is described in more detail herein below.

Mass spectrometry is a powerful analytical tool for the study of molecular structure and interaction between small and large molecules. The current state of the art in MS is such that sub-femtomole quantities of material can be readily analyzed to afford information about the molecular contents of the sample. An accurate assessment of the molecular weight of the material may be quickly obtained, irrespective of whether the samples' molecular weight is several hundred, or in excess of a hundred thousand, atomic mass units or Daltons (Da). It has now been found that mass spectrometry can elucidate significant aspects of important biological molecules. One reason for the utility of MS as an analytical tool is the availability of a variety of different MS methods, instruments, and techniques that can provide different pieces of information about the samples.

Mass spectrometry has been used to afford direct and rapid methods to identify lead compounds and to study the interactions between small molecules and biological targets. An advantage of mass spectrometry in identifying lead compounds is the sensitivity of the detection process. Small molecules (ligands) which bind to a target through weak non-covalent interactions, may be missed through conventional screening assays. These non-covalent ligand:target complexes, however, are readily detected by mass spectral analysis using the methods and processes of the invention.

These small molecules include both tight and weak binding ligands that bind to a particular target. In both collections of compounds and in biological samples, tight binding ligands can be present in very low concentrations relative to the weaker binding ligands. A tight binding ligand may be part of a very large library of compounds (e.g. a combinatorial library) or may be present in trace amounts of a tissue extract. In both cases, there is usually a much higher concentration of weaker binding ligands relative to the tight binding ligands.

A tight or a weak binding ligand can bind to a target by a non-covalent bond. These non-covalent interactions include hydrogen-bonding, electrostatic, and hydrophobic contacts that contribute to the binding affinity for the target. The difference between a tight and weak binding ligand is relative, a tight binding ligand has a stronger interaction between a target than does a weak binding ligand. Tight and weak binding non-covalent complexes are in equilibrium with the free ligand and free target. If a target is incubated with a mixture of two ligands, e.g., a tight binding and a weak binding ligand, an equilibrium will be established between the bound and unbound forms of each ligand with the binding site of the biological target. At equilibrium, an equilibrium constant (binding constant) can be calculated and is used as a measure of the binding affinities of the ligands. Binding affinity is a measure of the attraction between a ligand and its target.

A binding site is the specific region of a target where a substrate or a ligand binds to form a complex. For example, an enzyme's active site is where catalysis takes place. In a structured RNA molecule, binding of a ligand at a binding site can result in the disruption of the transcription or translation processes. A ligand is a small molecule that binds to a particular large molecule, a target molecule. Typically the target molecule is a large molecule, as for instance, a biological target such as a protein (enzyme) or a structured RNA or DNA.

In general, a mass spectrometer analyzes charged molecular ions and fragment ions from sample molecules. These ions and fragment ions are then sorted based on their mass to charge ratio (m/z). A mass spectrum is produced from the abundance of these ions and fragment ions that is characteristic of every compound. In the field of biotechnology, mass spectrometry has been used to determine the structure of a biomolecule, as for instance determining the sequence of oligonucleotides, peptides, and oligosaccharides.

In principle, mass spectrometers consist of at least four parts: (1) an inlet system; (2) an ion source; (3) a mass analyzer; and (4) a mass detector/ion-collection system (Skoog, D. A. and West, D. M., Principles of Instrumental Analysis, Saunders College, Philadelphia, Pa., 1980, 477-485). The inlet system permits the sample to be introduced into the ion source. Within the ion source, molecules of the sample are converted into gaseous ions. The most common methods for ionization are electron impact (EI), electrospray ionization (ESI), chemical ionization (CI) and matrix-assisted laser desorption ionization (MALDI). A mass analyzer resolves the ions based on mass-to-charge ratios. Mass analyzers can be based on magnetic means (sector), time-of-flight, quadrupole and Fourier transform mass spectrometry (FTMS). A mass detector collects the ions as they pass through the detector and records the signal. Each ion source can potentially be combined with each type of mass analyzer to generate a wide variety of mass spectrometers.

Mass spectrometry ion sources are well known in the art. Two commonly used ionization methods are electrospray ionization (ESI) and matrix-assisted laser desorption/ionization (MALDI) (Smith et al., Anal. Chem., 1990, 62, 882-899; Snyder, in Biochemical and Biotechnological Applications of Electrospray Ionization Mass Spectrometry, American Chemical Society, Washington, D.C., 1996; and Cole, in Electrospray Ionization Mass Spectrometry: Fundamentals, Instrumentation, Wiley, New York, 1997).

ESI is a gentle ionization method that results in no significant molecular fragmentation and preserves even weakly bound complexes between biopolymers and other molecules so that they are detected intact with mass spectrometry. ESI produces highly charged droplets of the sample being studied by gently nebulizing a solution of the sample in a neutral solvent in the presence of a very strong electrostatic field. This results in the generation of highly charged droplets that shrink due to evaporation of the neutral solvent and ultimately lead to a “coulombic explosion” that affords multiply charged ions of the sample material, typically via proton addition or abstraction, under mild conditions.

Electrospray ionization mass spectrometry (ESI-MS) is particularly useful for very high molecular weight biopolymers such as proteins and nucleic acids greater than 10 kDa in mass, for it affords a distribution of multiply-charged molecules of the sample biopolymer without causing any significant amount of fragmentation. The fact that several peaks are observed from one sample, due to the formation of ions with different charges, contributes to the accuracy of ESI-MS when determining the molecular weight of the biopolymer because each observed peak provides an independent means for calculation of the molecular weight of the sample. Averaging the multiple readings of molecular weight obtained from a single ESI-mass spectrum affords an estimate of molecular weight that is much more precise than would be obtained if a single molecular ion peak were to be provided by the mass spectrometer. Further adding to the flexibility of ESI-MS is the capability of obtaining measurements in either the positive or negative ionization modes.

ESI-MS has been used to study biochemical interactions of biopolymers such as enzymes, proteins and macromolecules such as oligonucleotides and nucleic acids and carbohydrates and their interactions with their ligands, receptors, substrates or inhibitors (Bowers et al., Journal of Physical Chemistry, 1996, 100, 12897-12910; Burlingame et al., J. Anal. Chem., 1998, 70, 647R-716R; Biemann, Ann. Rev. Biochem., 1992, 61, 977-1010; and Crain et al., Curr. Opin. Biotechnol., 1998, 9, 25-34). While interactions that lead to covalent modification of biopolymers have been studied for some time, one of the most significant developments in the field has been the observation, under appropriate solution conditions and analyte concentrations, of specific non-covalently associated macromolecular complexes that have been promoted into the gas-phase intact (Loo, Mass Spectrometry Reviews, 1997, 16, 1-23; Smith et al., Chemical Society Reviews, 1997, 26, 191-202; Ens et al., Standing and Chernushevich, Eds., New Methods for the Study of Biomolecular Complexes, Proceedings of the NATO Advanced Research Workshop, held 16-20 Jun. 1996, in Alberta, Canada, in NATO ASI Ser., Ser. C, 1998, 510, Kluwer, Dordrecht, Netherlands).

A variety of non-covalent complexes of biomolecules have been studied using ESI-MS and reported in the literature (Loo, Bioconjugate Chemistry, 1995, 6, 644-665; Smith et al., J. Biol. Mass Spectrom. 1993, 22, 493-501; Li et al., J. Am. Chem. Soc., 1993, 115, 8409-8413). These include the peptide-protein complexes (Busman et al., Rapid Commun. Mass Spectrom., 1994, 8, 211-216; Loo et al., Biol. Mass Spectrom., 1994, 23, 6-12; Anderegg and Wagner, J. Am. Chem. Soc., 1995, 117, 1374-1377; Baczynskyj et al., Rapid Commun. Mass Spectrom., 1994, 8, 280-286), interactions of polypeptides and metals (Loo et al., J. Am. Soc. Mass Spectrom., 1994, 5, 959-965; Hu and Loo, J. Mass Spectrom., 1995, 30, 1076-1079; Witkowska et al., J. Am. Chem. Soc., 1995, 117, 3319-3324; Lane et al., J. Cell Biol., 1994, 125, 929-943), and protein-small molecule complexes (Ganem and Henion, Chem Tracts-Org. Chem., 1993, 6, 1-22; Henion et al., Ther. Drug Monit., 1993, 15, 563-569; Ganguly et al., Tetrahedron, 1993, 49, 7985-7996, Baca and Kent, J. Am. Chem. Soc., 1992, 114, 3992-3993). Further, the study of the quaternary structure of multimeric proteins (Baca and Kent, J. Am. Chem. Soc., 1992, 114, 3992-3993; Light-Wahl et al., J. Am. Chem. Soc., 1994, 116, 5271-5278; Loo, J. Mass Spectrom., 1995, 30, 180-183, Fitzgerald et al., Proc. Natl. Acad. Sci. USA, 1996, 93, 6851-6856), and of nucleic acid complexes (Light-Wahl et al., J. Am. Chem. Soc., 1993, 115, 803-804; Gale et al., J. Am. Chem. Soc., 1994, 116, 6027-6028; Goodlett et al., Biol. Mass Spectrom., 1993, 22, 181-183; Ganem et al., Tet. Lett., 1993, 34, 1445-1448; Doctycz et al., Anal. Chem., 1994, 66, 3416-3422; Bayer et al., Anal. Chem., 1994, 66, 3858-3863; Greig et al., J. Am. Chem. Soc., 1995, 117, 10765-766), protein-DNA complexes (Cheng et al., Proc. Natl. Acad. Sci. U.S.A., 1996, 93, 7022-7027), multimeric DNA complexes (Griffey et al., Proc. SPIE-Int. Soc. Opt. Eng., 1997, 2985, 82-86), and DNA-drug complexes (Gale et al., JACS, 1994, 116, 6027-6028) are known in the literature.

ESI-MS has also been effectively used for the determination of binding constants of non-covalent macromolecular complexes such as those between proteins and ligands, enzymes and inhibitors, and proteins and nucleic acids. The use of ESI-MS to determine the dissociation constants (K_{D}) for oligonucleotide-bovine serum albumin (BSA) complexes have been reported (Greig et al., J. Am. Chem. Soc., 1995, 117, 10765-10766). The K_{D} values determined by ESI-MS were reported to match solution K_{D} values obtained using capillary electrophoresis.

ESI-MS measurements of enzyme-ligand mixtures under competitive binding conditions in solution afforded gas-phase ion abundances that correlated with measured solution-phase dissociation constants (K_{D}) (Cheng et al., JACS, 1995, 117, 8859-8860). The binding affinities of a 256-member library of modified benzenesulfonamide inhibitors to carbonic anhydrase were ranked. The levels of free and bound ligands and substrates were quantified directly from their relative abundances as measured by ESI-MS and these measurements were used to quantitatively determine molecular dissociation constants that agree with solution measurements. The relative ion abundance of non-covalent complexes formed between D- and L-tripeptides and vancomycin group antibiotics were also used to measure solution binding constants (Jorgensen et al., Anal. Chem., 1998, 70, 4427-4432).

ESI techniques have found application for the rapid and straightforward determination of the molecular weight of certain biomolecules (Feng and Konishi, Anal. Chem., 1992, 64, 2090-2095; Nelson et al., Rapid Commun. Mass Spectrom., 1994, 8, 627-631). These techniques have been used to confirm the identity and integrity of certain biomolecules such as peptides, proteins, oligonucleotides, nucleic acids, glycoproteins, oligosaccharides and carbohydrates. Further, these MS techniques have found biochemical applications in the detection and identification of post-translational modifications on proteins. Verification of DNA and RNA sequences that are less than 100 bases in length has also been accomplished using ESI with FTMS to measure the molecular weight of the nucleic acids (Little et al, Proc. Natl. Acad. Sci. USA, 1995, 92, 2318-2322).

While data generated and conclusions reached from ESI-MS studies for weak non-covalent interactions generally reflect, to some extent, the nature of the interaction found in the solution-phase, it has been pointed out in the literature that control experiments are necessary to rule out the possibility of ubiquitous non-specific interactions (Smith and Light-Wahl, Biol. Mass Spectrom., 1993, 22, 493-501). The use of ESI-MS has been applied to study multimeric proteins because the gentleness of the electrospray/desorption process allows weakly-bound complexes, held together by hydrogen bonding, hydrophobic and/or ionic interactions, to remain intact upon transfer to the gas phase. The literature shows that not only do ESI-MS data from gas-phase studies reflect the non-covalent interactions found in solution, but that the strength of such interactions may also be determined. The binding constants for the interaction of various peptide inhibitors to src SH2 domain protein, as determined by ESI-MS, were found to be consistent with their measured solution phase binding constants (Loo et al., Proc. 43^{rd} ASMS Conf. on Mass Spectrom. and Allied Topics, 1995). ESI-MS has also been used to generate Scatchard plots for measuring the binding constants of vancomycin antibiotics with tripeptide ligands (Lim et al., J. Mass Spectrom., 1995, 30, 708-714).

Similar experiments have been performed to study non-covalent interactions of nucleic acids. ESI-MS has been applied to study the non-covalent interactions of nucleic acids and proteins. Stoichiometry of interaction and the sites of interaction have been ascertained for nucleic acid-protein interactions (Jensen et al., Rapid Commun. Mass Spectrom., 1993, 7, 496-501; Jensen et al., 42^{nd} ASMS Conf. on Mass Spectrom. and Allied Topics, 1994, 923). The sites of interaction are typically determined by proteolysis of either the non-covalent or covalently crosslinked complex (Jensen et al., Rapid Commun. Mass Spectrom., 1993, 7, 496-501; Jensen et al., 42^{nd} ASMS Conf. on Mass Spectrom. and Allied Topics, 1994, 923; Cohen et al., Protein Sci., 1995, 4, 1088-1099). Comparison of the mass spectra with those generated from proteolysis of the protein alone provides information about cleavage site accessibility or protection in the nucleic acid-protein complex and, therefore, information about the portions of these biopolymers that interact in the complex.

Fourier transform ion cyclotron resonance mass spectrometry (FT-ICR MS) is an especially useful analytical technique because of its ability to resolve very small mass differences to make mass measurements with a combination of accuracy and resolution that is superior to other MS detection techniques, in connection with ESI ionization (Amster, J. Mass Spectrom., 1996, 31, 1325-1337, Marshall et al., Mass Spectrom. Rev., 1998, 17, 1-35). FT-ICR MS may be used to obtain high resolution mass spectra of ions generated by any of the other ionization techniques. The basis for FT-ICR MS is ion cyclotron motion, which is the result of the interaction of an ion with a unidirectional magnetic field. The mass-to-charge ratio of an ion (m/q or m/z) is determined by a FT-ICR MS instrument by measuring the cyclotron frequency of the ion.

The insensitivity of the cyclotron frequency to the kinetic energy of an ion is one of the fundamental reasons for the very high resolution achievable with FT-ICR MS. Each small molecule with a unique elemental composition carries an intrinsic mass label corresponding to its exact molecular mass, identifying closely related library members bound to a macromolecular target requires only a measurement of exact molecular mass. The target and potential ligands do not require radio labeling, fluorescent tagging, or deconvolution via single compound re-synthesis. Furthermore, adjustment of the concentration of ligand and target allows ESI-MS assays to be run in a parallel format under competitive or non-competitive binding conditions. Signals can be detected from complexes with dissociation constants ranging from <10 nM to ˜100 mM. FT-ICR MS is an excellent detector in conventional or tandem mass spectrometry, for the analysis of ions generated by a variety of different ionization methods including ESI, or product ions resulting from collisionally activated dissociation.

FT-ICR MS, like ion trap and quadrupole mass analyzers, allows selection of an ion that may actually be a weak non-covalent complex of a large biomolecule with another molecule (Marshall and Grosshans, Anal. Chem., 1991, 63, A215-A229; Beu et al., J. Am. Soc. Mass Spectrom., 1993, 4, 566-577; Winger et al., J. Am. Soc. Mass Spectrom., 1993, 4, 566-577; Huang and Henion, Anal. Chem., 1991, 63, 732-739), or hyphenated techniques such as LC-MS (Bruins et al., Anal. Chem., 1987, 59, 2642-2646; Huang and Henion, J. Am. Soc. Mass Spectrom., 1990, 1, 158-65; Huang and Henion, Anal. Chem., 1991, 63, 732-739) and CE-MS experiments (Cai and Henion, J. Chromatogr., 1995, 703, 667-692). FTICR-MS has also been applied to the study of ion-molecule reaction pathways and kinetics.

The use of ESI-FT-ICR mass spectrometry as a method to determine the structure and relative binding constants for a mixture of competitive inhibitors of the enzyme carbonic anhydrase has been reported (Cheng et al., J. Am. Chem. Soc., 1995, 117, 8859-8860). Using a single ESI-FT-ICR MS experiment these researchers were able to ascertain the relative binding constants for the non-covalent interactions between inhibitors and the enzyme by measuring the relative abundances of the ions of these non-covalent complexes. Further, the K_{D}s so determined for these compounds paralleled their known binding constants in solution. The method was also capable of identifying the structures of tight binding ligands from small mixtures of inhibitors based on the high-resolution capabilities and multistep dissociation mass spectrometry afforded by the FT-ICR technique. A related study (Gao et al., J. Med. Chem., 1996, 39, 1949-55) reports the use of ESI-FT-ICR MS to screen libraries of soluble peptides in a search for tight binding inhibitors of carbonic anhydrase II. Simultaneous identification of the structure of a tight binding peptide inhibitor and determination of its binding constant was performed. The binding affinities determined from mass spectral ion abundance were found to correlate well with those determined in solution experiments. Heretofore, the applicability of this technique to drug discovery efforts is limited by the lack of information generated with regards to sites and mode of such non-covalent interactions between a protein and ligands.

Electrospray ionization has found wide acceptance in the field of analytical mass spectrometry since it is a gentle ionization method which produces multiply charged ions from large molecules with little or no fragmentation and promotes them into the gas phase for direct analysis by mass spectrometry. ESI sources operate in a continuous mode with flow rates ranging from <25 nL/min to 1000 μL/min. The continuous nature of the ion source is well suited for mass spectrometers which employ the m/z scanning, such as quadrupole and sector instruments, as their coupling constitutes a continuous ion source feeding in a nearly continuous mass analyzer. As used in this invention the electrospray ionization source may have any of the standard configurations including but not limited to Z-spray, microspray, off-axis spray or pneumatically assisted electrospray. All of these can be used in conjunction with or without additional countercurrent drying gas. Further the mass spectrometer can include a gated ion storage device for effecting thermolysis of test mixtures.

When the solvated ions generated from electrospray ionization conditions are introduced into the mass spectrometer, the ions are subsequently desolvated in an evaporation chamber and are collected in a rf multi-pole ion reservoir (ion reservoir). A gas pressure around the ion reservoir is reduced to 10⁻³-10⁻⁶ torr by vacuum pumping. The ion reservoir is preferably driven at a frequency that captures the ions of interest and the ensemble of ions are then transported into the mass analyzer by removing or reversing the electric field generated by gate electrodes on either side of the ion reservoir. Mass analysis of the reacted or dissociated ions are then performed. Any type of mass analyzers can be used in effecting the methods and process of the invention. These include, but are not limited to, quadrupole, quadrupole ion trap, linear quadrupole, time-of-flight, FT-ICR and hybrid mass analyzers. A suitable mass analyzer is a FT-ICR mass analyzer.

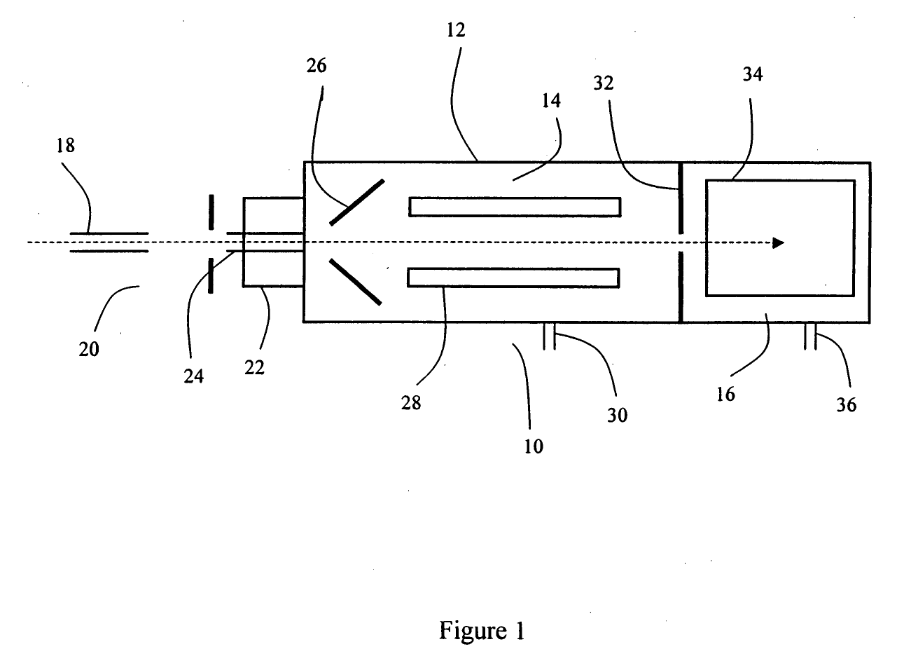

Seen in FIG. 1 is a schematic representation of a mass spectrometer. A review of the mass spectrometer will facilitate understanding of the invention as it includes various component parts that may be included in one or more of the various types of different mass spectrometers. The spectrometer 10 includes a vacuum chamber 12 that is segmented into a first chamber 14 and a second chamber 16. The mass spectrometer 10 is shown as an electrospray mass spectrometer. A metallic micro-electrospray emitter capillary 18 having an electrode 20 is positioned adjacent to the vacuum chamber 12. The electrode/metallic capillary serves as an ion emitter. The capillary 18 is positioned on an X-Y manipulator for movement in two planes.

Adjacent to the capillary 18 and extending from the vacuum chamber 16 is an evaporative chamber 22 having a further capillary 24 extending axially along its length. The X-Y manipulator allows for precise positioning of the capillary 18 with respect to the capillary 24. A plume of ions carried in a solvent is emitted from the emitter capillary 18 towards the evaporator capillary 24. The evaporator capillary 24 serves as an inlet to the interior of vacuum chamber 12 for that portion of the plume directly in line with the evaporator capillary 24.

Within the first chamber 14 is a skimmer cone 26. This skimmer cone 26 serves as a lens element. In line with the skimmer cone 26 is an ion reservoir 28. A port 30 having a valve is connected to a conventional first vacuum source (not shown) for reducing the atmospheric pressure in the first chamber 14 to create a vacuum in that chamber. Separating chambers 14 and 16 is a gate electrode 32.

The ion reservoir 28 can be one of various reservoirs such as a hexapole reservoir. Ions, carried in a solvent, are introduced into chamber 14 via the evaporator capillary 24. Solvent is evaporated from the ions within the interior of capillary 24 of the evaporator chamber 22. Ions travel through skimmer cone 26 towards the electrode 32. By virtue of their charge and a charge placed on the electrode 32 the ions can be held in the reservoir. The electrode 32 includes an opening. Ions are released from the ion reservoir 28 by modifying the potential on the electrode 32. They then can pass through the opening into the second vacuum chamber 16 towards a mass analyzer 34. For use in FT-ICR, positioned with respect to the analyzer 34 is a magnet (not shown). The second vacuum chamber 16 includes port 36 having a valve. As with valve 30 in chamber 14, this valve 36 is attached to an appropriate vacuum pump for creating a vacuum in chamber 16. Chamber 16 may further include a window or lens that is positioned in line with a laser. The laser can be used to excite ions in either the mass analyzer 34 or the ion reservoir. Any of the mass spectrometers described above, for example, can be used to carry out any of the inventions described herein.

In some embodiments of the invention, methods for selecting a target molecule that has an affinity for a ligand that is equal to or greater than a baseline affinity are provided. An amount of a standard target is mixed with an excess amount of the ligand. The standard target forms a non-covalent binding complex with the ligand and the unbound ligand is present in the mixture. The mixture of the standard target and the ligand is introduced into a mass spectrometer to obtain a baseline affinity. The operating performance conditions of the mass spectrometer are adjusted such that the signal strength of the standard target bound to the ligand is from 1% to about 30% of the signal strength of unbound ligand. At least one target molecule is introduced into a test mixture of the ligand and the standard target. The test mixture is introduced into a mass spectrometer. Any complexes of the target molecule and the ligand are identified. A target molecule that has greater affinity for the ligand than the baseline affinity for the ligand is detected. In some embodiments, the ligand and/or the target molecule is a microRNA or mimic thereof.

In other embodiments of the invention, methods of selecting those members of group of compounds that can form a non-covalent complex with a ligand and where the affinity of the members for the ligand is greater than a baseline affinity are provided. An amount of a standard compound is mixed with an excess amount of the ligand. The standard compound forms a non-covalent binding complex with the ligand and the unbound ligand is present in the mixture. The mixture of the standard compound and the ligand is introduced into a mass spectrometer to obtain a baseline affinity. The operating performance conditions of the mass spectrometer are adjusted such that the signal strength of the standard compound bound to the ligand is from 1% to about 30% of the signal strength of unbound ligand. A sub-set of the group of compounds is introduced into a test mixture of the ligand and the standard compound. The test mixture is introduced into the mass spectrometer. The members of the sub-set that form complexes with the ligand are identified. Members of the sub-set that have a greater affinity for the ligand than the baseline affinty for the ligand are detected. In some embodiments, the ligand and/or the group of compounds is a microRNA or mimic thereof.

In other embodiments of the invention, methods of detecting a ligand-target complex having an affinity as expressed as a dissociation constant of from about nanomolar to about 100 millimolar are provided. An amount of a standard target is mixed with an excess amount of the ligand such that unbound ligand is present in the mixture. The standard target forms a non-covalent binding complex with the ligand at an affinity of about 50 millimolar as measured as a dissociation constant indicated by an electrospray mass spectrometer. The mixture of the standard target and the ligand is introduced into a mass spectrometer. The operating performance conditions of the mass spectrometer are adjusted such that the relative ion abundance of the standard target bound to the ligand is from 1% to about 30% of the relative ion abundance of unbound ligand. A set of target molecules is added to a test mixture of the ligand and the standard target. The test mixture is introduced into a mass spectrometer. Members of the set of target molecules that form complexes with the ligand that have an affinity as expressed as a dissociation constant of from about nanomolar to about 100 millimolar are detected. In some embodiments, the ligand and/or the target molecule in the ligand-target complex is a microRNA or mimic thereof.

In other embodiments of the invention, methods of detecting ligand-target complexes having from about nanomolar to about 100 millimolar affinity as measured as a dissociation constant are provided. An amount of an ionic ammonium standard compound is mixed with an excess amount of the ligand such that unbound ligand is present in the mixture. The mixture of the ammonium compound and the ligand is introduced into a mass spectrometer. The operating performance conditions of the mass spectrometer are adjusted such that the relative ion abundance of ammonium ion bound to the ligand is from 1% to about 30% of the relative ion abundance of unbound ligand. A set of target molecules is introduced into a test mixture of the ligand and the ammonium compound. The test mixture is introduced into a mass spectrometer. Members of the set of target molecules that form complexes with the ligand that have from about nanomolar to about 100 millimolar affinity as measured as a dissociation constant are detected. In some embodiments, the ligand and/or the target molecule in the ligand-target complex is a microRNA or mimic thereof.

In other embodiments of the invention, methods for determining the relative interaction between at least two target molecules and a ligand are provided. An amount of at least two target molecules is mixed with an amount of the ligand to form a mixture. The mixture is analyzed by mass spectrometry to determine the presence or absence of a ternary complex corresponding to simultaneous adduction of two of the target molecules with the ligand. The absence of the ternary complex indicates that binding of the target molecules to the ligand is competitive and the presence of the ternary complex indicates that binding of the target molecules to the microRNA ligand is other than competitive. In some embodiments, the ligand and/or the target molecules is a microRNA or mimic thereof.

In other embodiments of the invention, methods of determining binding interaction between a first target molecule and a second target molecule with respect to a ligand are also provided. The ligand is introduced to the first and second target molecules to form a mixture comprising i) a ternary complex (LT1T2) of the ligand bound to the first and second target molecules, ii) a first binary complex (LT1) of the first target molecule and the ligand, iii) a second binary complex (LT2) of the second target molecule and the ligand, and iv) ligand (L) unbound by either the first or second target molecule. The mixture is analyzed by mass spectrometry to determine the absolute ion abundance of the ternary complex (LT1T2), the first binary complex (LT1), the second binary complex (LT2), and the microRNA ligand (L) unbound to the first or second target molecules. The ion abundance of the first and second binary complexes LT1 and LT2, the ternary complex LT1T2, and the ligand (L) are compared to determine if there is a concurrent binding interaction or a competitive binding interaction. In some embodiments, the ligand and/or the target molecules is a microRNA or mimic thereof.

In other embodiments of the invention, methods of determining the relative proximity of binding sites for a first target molecule and a second target molecule on a ligand are also provided. The ligand is exposed to a mixture of the second target molecule and a plurality of derivative compounds of the first target molecule, the first target molecule derivatives comprising the chemical structure of the first target molecule and at least one substituent group pending therefrom. The mixture is analyzed by mass spectrometry to identify a first target molecule derivative that inhibits the binding of the second target molecule to the ligand or that has a competitive binding interaction with the second target molecule for the ligand. In some embodiments, the ligand and/or the target molecules is a microRNA or mimic thereof.

In other embodiments of the invention, methods of determining the relative orientation of a first target molecule to a second target molecule when bound to a ligand are provided. The ligand is exposed to a mixture of the second target molecule and a plurality of derivative compounds of the first target molecule, the first target molecule derivatives comprising the chemical structure of the first target molecule and having a substituent group pending therefrom. The mixture is analyzed by mass spectrometry to identify a first target molecule derivative that inhibits the binding of the second target molecule to the ligand or that has a competitive binding interaction with the second target molecule for the ligand. In some embodiments, the ligand and/or the target molecules is a microRNA or mimic thereof.

In other embodiments of the invention, methods for screening target molecules having binding affinity to a ligand are provided. By mass spectrometry in a mixture comprising the target molecules and ligand, a first and second target molecule that bind to the ligand non-competitively is identified. The first and second target molecules are concatentated to form a third target molecule having greater binding affinity for the ligand than either the first or second target molecules. In some embodiments, the ligand and/or the target molecules is a microRNA or mimic thereof.

In other embodiments of the invention, methods for modulating the binding affinity of a target molecule for a ligand are provided. The ligand is exposed to a first target fragment and a second target fragment. The ligand exposed to the first and second target fragments is interrogated in a mass spectrometer to identify binding of the first and second target fragments to the ligand. The first and second target fragments are concatenated together in a structural configuration that improves the binding properties of the first and second target fragments for the ligand. In some embodiments, the ligand and/or the target molecules is a microRNA or mimic thereof.

In other embodiments of the invention, methods for refining the binding of a target molecule to a ligand are provided. A first virtual fragment of the target is virtually concatenated with a second virtual fragment of the target to form an in silico 3D model of the concatenated target fragments. The in silico 3D model of the concatenated target fragments is positioned on an in silico 3D model of the ligand. The positioning of the in silico 3D model of the concatenated target fragments on the in silico 3D model of the ligand is scored. The positioning of the in silico 3D model of the concatenated target fragments on the in silico 3D model of the ligand is refined using the results of the scoring.

U.S. Ser. No. 09/499,875 is incorporated herein by reference in its entirety.

In each of the above embodiments, an electrospray mass spectrometer is utilized. Electrospray ionization can be accomplished by Z-spray, microspray, off-axis spray or pneumatically assisted electrospray ionization. Further countercurrent drying gas can be used. Mass analyzers for use in identifying the complexes are quadrupole, quadrupole ion trap, time-of-flight, FT-ICR and hybrid mass detectors. A method of measuring signal strength is by the relative ion abundance. The mass spectrometer can also include a gated ion storage device for effecting thermolysis of the test mixtures within the mass spectrometer.

Adjustment of the mass spectrometer operating performance conditions would include adjustment of the source voltage potential across the desolvation capillary and a lens element of the mass spectrometer. This can be monitored by ion abundance of free target molecule. Adjustment of the mass spectrometer operating conditions further can include adjustment of the temperature of the desolvation capillary and adjustment of the operating gas pressure with the mass spectrometer downstream of the desolvation capillary.

In some embodiments, adjustment of the operating performance conditions of the mass spectrometer is effected by adjustment of the voltage potential across the desolvation capillary and a lens element to generate an ion abundance of the ion from a complex of standard ligand with the target of from about 1% to about 30% compared to the abundance of the ion from the target molecule. A range of abundance of the complex of standard ligand with target to the abundance of the ion from the target molecule is from about 10% to about 20%.

Standard targets are those molecules having a baseline affinity for the ligand of about 10 to about 100 millimolar. Standard targets can have a baseline affinity for the ligand of about 50 millimolar as expressed as a dissociation constant. With any ligand, the standard target will typically be selected such that its has a binding affinity, as measured as a dissociation constant, i.e., Kd, of the order of nanomolar to about 100 mM, from 10 to 50 mM, or 50 mM binding affinity for the ligand.

For use with RNA or DNA targets, ammonium (from acetate, chloride, borate or other salts), primary amines (including by not limited to alkyl amines such as methylamine and ethylamine), secondary amines (including but not limited to dialkylamines such as dimethylamine and diethylamine), tertiary amines (including by not limited to trialkyl amines such as triethylamine, trimethylamine and dimethylethyl amine), amino acids (including but not limited to glycine, alanine, tryptophan and serine) and nitrogen containing heterocycles (including but not limited to imidazole, triazole, triazine, pyrimidine and pyridine) are particularly useful as standard targets.

Other standard targets will be used for other target molecules. For use with protein ligands, esters such as formate, acetate and propionate, phosphates, borates, amino acids and nitrogen containing heterocycles (including but not limited to imidazole, triazole, triazine, pyrimidine and pyridine) are particularly useful. As with the above described RNA and DNA ligands, for protein ligands as well as for other ligands, the standard target will typically have a binding affinity, as measured as a dissociation constant, i.e., Kd, of the order of nanomolar to about 100 millimolar for the ligand.

The target molecule or ligand can be one of various target molecules including miRNA, siRNA, stRNA, sncRNA, tncRNA, snoRNA, smRNA, snRNA, other small non-coding RNA, RNA, DNA, proteins, RNA-DNA duplexes, RNA-RNA duplexes, DNA duplexes, polysaccharides, phospholipids and glycolipids. The term “microRNA” shall include any RNA that is a fragment of a larger RNA or is a miRNA, siRNA, stRNA, sncRNA, tncRNA, snoRNA, smRNA, snRNA, other small non-coding RNA.

A target molecule or ligand can be RNA, particularly structured RNA. Structured RNA is a term that refers to definable, relatively local, secondary and tertiary structures such as hairpins, bulges, internal loops, junctions and pseudoknots. Structured RNA can have both base paired and single stranded regions. RNA can be divided into primary, secondary, and tertiary structures and is defined similarly to proteins. Thus, the primary structure is the linear sequence. The secondary structure reflects local intramolecular base pairing to form stems and single stranded loops, bulges, and junctions. The tertiary structure reflects the interactions of secondary structural elements with each other and with single stranded regions. As practiced herein, the target molecule or ligand can, itself, be a fragment of a larger molecule, as for instance, RNA that is a fragment of a larger RNA. Particularly suitable as a target molecule or ligand is RNA, particularly RNA that is a fragment of a larger RNA. Another target molecule is double stranded DNA targeted with ligands that are transcription factors.

Target molecules and ligands can include those having a molecular mass of less than about 1000 Daltons and fewer that 15 rotatable bonds, i.e., covalent bonds linking one atom to a further atom in the molecule and subject to rotation of the respective atoms about the axis of the bond. Target molecules and ligands also include those having a molecular mass of less than about 600 Daltons and fewer than 8 rotatable bonds. Target molecules and ligands also include those have a molecular mass of less than about 200 Daltons and fewer than 4 rotatable bonds. A particularly useful solvent for use in screening target molecules and ligands is dimethylsulfoxide. In one embodiment, the target molecules or ligands are selected as compounds having at least 20 mM solubility in dimethylsulfoxide.

The target molecules and ligands can comprise members of collection or libraries, often categorized by size, structure or function. Collection libraries include historical repositories of compounds, collections of natural products, collections of drug substances or intermediates for such drug substances, collections of dyestuffs, commercial collections of compounds, or combinatorial libraries of compounds. A collection for selecting target molecules or ligands can contain various numbers of members with libraries of from 2 to about 100,000 being suitable. Many universities and pharmaceutical companies maintain historical repositories of all compounds synthesized. These can include drugs substances that have or have not been screened for biological activity, intermediates used in the preparation of such drug substances and derivatives of such drug substances. A typical pharmaceutical company might have millions of such repository samples. Other collections of compounds include collections of natural occurring compounds or derivatives of such natural occurring compounds. Irrespective of the origin of the compounds, the compound collections can be categorized by size, structure, function or other various parameters.

Various microRNA molecules are useful as the ligand or target. In vivo, some microRNAs are enzymatically processed from larger RNA precursor molecules. Thus, microRNA molecules of the invention can be those that are fragments of larger RNA precursor molecules, including larger RNA molecules being from about 10 to about 200 nucleotides in length and having secondary and ternary structure, such as a hairpin or stemloop, for example. Larger microRNA precursor molecules can be from about 15 to about 100 nucleotides in length, from 50 to 80 nucleotides in length, from about 10 to about 25 nucleotides in length, and from 21 to 24 nucleotides in length.

In effecting some embodiments of the present invention, a set of target molecules are probed against a ligand, using the mass spectrometer, to identify those target molecules from the set of target molecules that are “weak” binders with respect to the target molecule. For the purposes of this invention “weak” binding is defined as binding in the millimolar (mM) range. Typically, target molecules will have a binding affinity in the range of 0.2 to 10 nM. As opposed to other techniques, the mass spectrometer will not fail to detect these weak mM interactions. Target molecules and or ligands having binding characteristics with respect to each other are selected. After selection, the binding mode of the ligands and/or target molecules can be determined by re-screening mixtures of target molecules against the ligand. Re-screening is effected by simultaneously exposing a set of target molecules against a ligand. As a result of this screening, target molecules that cannot bind at overlapping sites, competitive binding, are differentiated from those that can bind at remote sites simultaneously, concurrent binding, and those that can bind in a way that traps one compound, cooperative binding, as well as those having “mixed” binding modes.

Ligands and target molecules having selected binding characteristics can be identified and their structure activity relationship (SAR) with respect to binding each other can be probed using the mass spectrometer. Two or more ligands or taget molecules can be joined by concatenation into new structural configurations to create a new ligand or target molecule that will have improved binding characteristics or properties. Thus, starting from small, rigid ligands or target molecules that bind with weak affinity, more complex molecules that bind to specific ligands or target molecules with high affinity can be identified using mass spectrometry. This is effected using the mass spectrometer as the primary tool and does not involve extensive chemical synthesis or extensive molecular modeling.

Concatenation can be effected based on empirical or computational predictions. Thus, concatenation will yield either new synthetic chemical ligands or target molecules having new properties or in silico virtual ligands. In conjunction with molecular modeling tools, the virtual ligands can be used to identify probable binding locations on the target molecule.

In concatenating ligands or target molecules together using the methods and processes of the invention, two ligands or target molecules that have mM (millimolar) affinities might be joined and yield a concatenated ligand or target molecule that might have nM affinity. While we do not wish to be bound by theory, we presently believe this result has multiple contributing factors. There can be a gain in intrinsic binding energy, i.e., loss of translational entropy, when both fragments always bind at the same time. Proper geometry for both fragments can result in a favorable enthalpy of interaction, i.e., no loss of binding enthalpy. Fewer degrees of freedom resulting from two fragments being linked through bonds with limited rotation will result in a loss of rotational entropy that equals a gain in binding energy. And there can be some energy gain (enthalpy and entropy) from desolvation of the target and the ligand fragments. The net result can be a 10³ to a 10⁶ improvement in binding affinity, i.e., a 4-6 kcal/mol gain in binding energy.

Newly synthesized concatenated ligand or target molecules, which retain the best conformations and locations of the ligand fragments with respect to the target or ligand, can be re-probed using the mass spectrometer to ascertain the binding characteristics of the new molecule. Repeated iteration of the process and methods of the invention can improve the binding affinity of these new molecules. The newly synthesized concatenated ligand or target molecules can also be screened using a functional assay that involves the target.

In screening a compound set or target molecule set for potential binding to ligands, sample preparation and certain basic operations of the mass spectrometer can be optimized to preserve the weak non-covalent complexes formed between ligands and the target molecule(s). These include extra care in desalting the target molecule as well as a general reduction of the temperature of the desolvation capillary compared to the temperature that would be used if the only interest was in analyzing the target molecule itself. Also the voltage potential across the capillary exit and the first skimmer cone, i.e., lens element, is optimized to ensure good desolvation. A further consideration is selection of the buffer concentration and solvent to insure good solvation.

The candidate target molecules or ligands can be screened one at a time or in sets. A typical set would have from 2 to 10 members, or from 4 to 8 members. The compound set is screened for members that form non-covalent complexes with the target molecule or ligand using the mass spectrometer. The relative abundances and stoichiometries of the non-covalent complexes with the target molecule or ligand are measured from the integrated ion intensities. These results can be stored in a relational database that is cross-indexed to the structure of the compounds.

Depending on the size of the compound collection used above, from 2 to 10,000 compounds may form complexes with the target or ligand. These compounds are pooled into groups of 4-10 and screened again as a mixture against the target as before. Since all of the compounds have been shown previously to bind to the target or ligand, three possible changes in the relative ion abundances are observed in the mass spectrometry assay. If two compounds bind at the same site, the ion abundance of the target complex for the weaker binder will be decreased through competition for target binding with the higher affinity binder (competitive binding). If two compounds can bind at distinct sites, signals will be observed from the respective binary complexes with the target and from a ternary complex where both compounds bind to the target simultaneously (concurrent binders). If the binding of one compound enhances the binding of a second compound, the ion abundance from the ternary complex will be enhanced relative to the ion abundances from the respective binary complexes (cooperative binding. If the ratio of the relative ion abundances is greater than 1, the binding is considered to be cooperative. These ratios of relative ion abundances are calculated and can be stored in a database for all compounds that bind to the target.

Compounds that bind concurrently are further analyzed. Derivatives of concurrent binders can be prepared with addition of an added moiety, including but not limited to methyl, ethyl, isopropyl, amino, methylamino, dimethylamino, trifluoromethyl, methoxy, thiomethyl or phenyl at different positions around the original compound that binds. These derivatives can be re-screened as a mixture with compounds that bound concurrently to the starting compound. If the additional methyl, ethyl, isopropyl, or phenyl moiety occupies space that the concurrent binder occupied, the two compounds will bind competitively. Observation of this change in the mode of binding using the mass spectrometer indicates the two molecules are spatially proximate as a result of the chemical modification. Correlation of the change in binding mode with the size and position of the chemical modification can be used as a “molecular ruler” to measure the distance between two compounds on the surface of the RNA. Compounds that bind in a cooperative or competitive mode do so by binding in close proximity on the target surface. Locations where addition of a moiety has no effect on the binding mode are potential sites of covalent attachment between the two molecules. This information can be used in conjunction with molecular modeling of the target-ligand complex to generate a pharmacophore map of the chemical groups that bind to the target surface.

In some cases, a 3-dimensional working model of the target structure may be available based on NMR or chemical and enzymatic probing data. These 3-D models of the target can be used with computational programs such as MCSS (MSI, San Diego) or QXP (Thistlesolft, Groton, Conn.) to locate the possible sites of binding with the ligand. MCSS, QCP and similar programs perform a Monte Carlo-based search for sites where the ligand can bind, and rank order the sites based on a scoring scheme. The scoring scheme calculates hydrophobic, hydrogen-bonding, and electrostatic interactions between the ligand and target. The small molecules may bind at many locations along the surface of the target. However, there are some locations that are more suitable than others. These calculations can be performed for molecules that bind competitively or cooperatively, and favorable binding conformations whose proximity is based on the “molecular ruler” as described above can be identified.

In one embodiment of the invention, the QXP program is used to search all interaction space around a RNA target molecule and to cluster the results. From the clustered results the highest probability, low-energy binding sites for binding ligands is identified. All the interaction space around the RNA target is searched for proximate binding sites between ligands. The distances between the ligands are measured to obtain the lengths of linkers required to connect functional group sites on the ligands for best scaffold binding. The search also is used to insure that the lowest energy conformation retains the best binding contacts.

In conjunction with the developers of QXP, the UNIX version of the QXP program designed to run on a SGI computer having 128 processors was ported to a LINUX version that runs on a PC platform having 56 processors. This resulted in an advantage in maximizing the price to performance ratio of the hardware. The computationally intensive nature of identifying global energy minimum for a combinatorial library of small molecule, typically with 8 to 12 rotatable bonds, bound to the receptor is particularly well suited to the “distributed computing” method. The compound library is divided into the number of available computational resources and thus the docking calculations are run in “parallel”. This method exploits the available CPU cycles over a cluster of extremely fast PC boxes networked together in a system commonly referred to as a Beowulf-class cluster. Beowulf-class clusters are described by E. Wilson in Chemical & Engineering News (2000, 78(2):27-31) The PC platform used included 16 PCs, dual Intel pentium II 450 MHz processors, 256 MB RAM and 6.4 GB disk and 12 PCs, dual Intel pentium II 400 MHz processors, 256 MB RAM and 6.4 GB disk totaling 56 processors. A benchmark calculation using 350 MHz Pentium II processors indicated, in terms of speed, that PC boxes clustered together as described would outperform a R5000 SGI O₂ machine.

The same result is reported to be accomplished using the MCSS software, i.e., MCSS/HOOK. As reported by its manufacture, MSI, San Diego, Calif., for proteins, MCSS/HOOK characterizes an active site's ability to bind ligands using energetics calculated via CHARMm. Strongly bound ligands are linked together automatically to provide de novo suggestions for drug candidates. The software is reported to provide a systematic, comprehensive approach to ligand development and de novo ligand design that result in synthetically feasible molecules. Using libraries of functional groups and molecules, MCSS is reported to systematically searches for energetically feasible binding sites in a protein. HOOK is reported to then systematically searches a database for skeletons which logically might connect these binding sites in the presence of the protein. HOOK attempts to link multiple functional groups with molecular templates taken from the its database. The results are potential compounds that are consistent with the geometry and chemistry of the binding site.

Competitive Binding

Ligands bind competitively for a target when the binding of one ligand prevents the binding of the other ligand is the result of the ligands binding to the target at the same location. In this situation, the mixture contains an equilibrium of two binary complexes, one of which being one ligand bound to the target and the other being the other ligand bound to the target. The ligand having the greater affinity for the target will predominate and thus have higher signal intensity for its binary complex with the target compared to the other ligand. Competitive binding interaction between two ligands is determined according to methods of the invention by analyzing the mixture by mass-spectrometry to detect the presence or lack of signal corresponding to a ternary complex where both ligands are bound to the target at the same time. The lack of signal for a ternary complex indicates a competitive binding interaction between the two ligands while the presence of the signal indicates a non-competitive interaction.

Accordingly, in an aspect of the present invention, there is provided a method for determining the relative interaction between at least two ligands with respect to a target substrate. In practicing this method an amount of each of the ligands is mixed with an amount of the target substrate to form a mixture. This mixture is analyzed by mass spectrometry to determine the presence or absence of a ternary complex corresponding to the simultaneous adduction of two of the ligands with the target substrate. The absence of the ternary complex indicates that binding of the ligands to the target substrate is competitive and the presence of the ternary complex indicates that binding of the ligands to the target substrate is other than competitive.

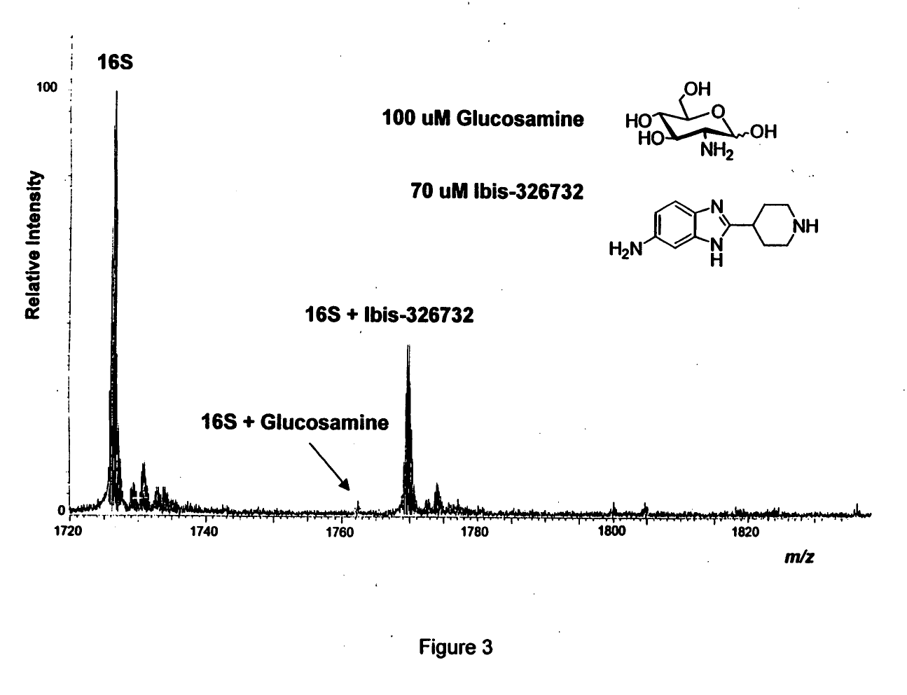

The above method for determining a competitive binding interaction of two ligands is exemplified in FIG. 3 wherein 70 μM of a small molecule Ibis-326732 (4-amino-2-piperidin-4-ylbenzimidazole) was added to a solution of 100 μM glucosamine and 5 μM of a 27 nucleotide fragment of bacterial 16S ribosomal RNA incorporating the A-site. The mass-spectrum trace for the mixture lacks an intensity signal for a ternary complex of the two ligands Ibis-326732 and glucosamine simultaneously bound to the target 16S RNA. This indicates that the two ligands are competitive binders for this target (i.e. bind to the same site). Further, a comparison of the ion abundance of the two binary complexes at approximately 1762 and 1770 m/z indicates that Ibis-326732 binds to the target RNA with greater affinity than glucosamine.

Concurrent Binding

Ligands bind concurrently when the binding of one ligand to the target is unaffected by the binding of the other and is a consequence of the ligands binding to the target at distinct sites. In this situation, a mixture containing two concurrent binding ligands will have an equilibrium of two binary complexes, one being first ligand bound to the target and the other being the second ligand bound to the target as well as a ternary complex of both ligands bound to the target and unbound target substrate. The ligand having the greater affinity for the target will have higher signal intensity for its binary complex with the target compared to the other ligand. Concurrent binding interaction between two ligands is determined according to methods of the invention by analyzing the mixture by mass-spectrometry and comparing the ratios of the ion abundance of the complexes. Particularly, the absolute ion abundance of the ternary complex (TL1L2) is compared to the relative ion abundance of the binary complexes (TL1 and TL2) which contribute to the formation of the ternary complex with respect to the unbound target (TL1×TL2/T). Since there are two binary complexes contributing the formation of the ternary complex, the comparison is with the sum of the two contributing binary complexes i.e. TL1×TL2/T+TL2×TL1/T. If the absolute ion abundance of the ternary complex is equal to the sum of the relative ion abundance of the contributing binary complexes, then the two ligands concurrently bind to the target substrate. Expressed another way, a pair of ligands are concurrent binders for a target if in either of the following equivalent formulae the value of y is equal to zero:

$$y = TL1L2 - TL1 \times \frac{TL2}{T} - TL2 \times \frac{TL1}{T}$$

$$or$$

$$y = TL1L2 - 2 \times \frac{TL1 \times TL2}{T}$$

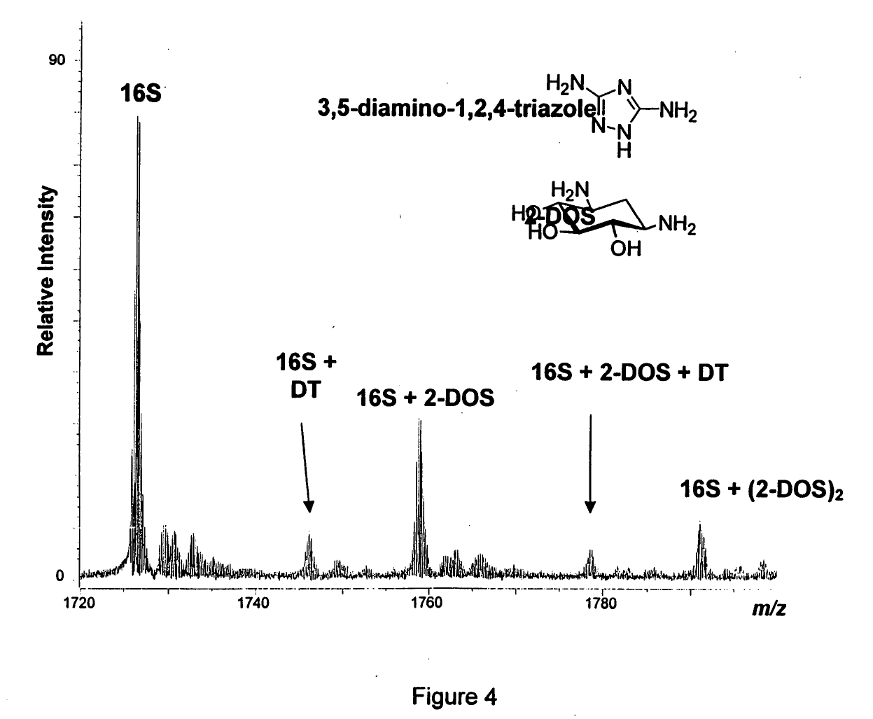

The above method for determining a concurrent binding interaction of two ligands is exemplified in FIG. 4 wherein 3,5-diamino-1,2,4-triazole (DT) and 2-deoxystreptamine (2-DOS) are both ligands for target RNA (a 27-mer fragment of ribosomal RNA comprising the 16S A-site). The mass-spectrum trace shows intensity signals for a ternary complex at approximately 1778 m/z for both ligands bound to the target 16S RNA, a binary complex at about 1758 m/z for 2-DOS bound to 16S RNA, a binary complex at 1746 m/z for DT bound to 16S RNA and another signal at about 1727 m/z for 16S RNA unbound by either ligand. The relative ion abundance of the ternary complex (16S+2−DOS+DT) with respect to the unbound 16S target RNA (16S) is equal, within limits of error, to the sum of the relative ion abundance of the contributing binary complex ((16S+DT)×(16S+2−DOS)) with respect to the unbound target (16S) and the contributing binary complex ((16S+2−DOS)+(16S+DT)) with respect to the unbound target (16S). Expressed in a simplified form of the formula:
y≈(16S+2−DOS+DT)−2×(16S+2−DOS)×(16S+DT)/16S
This indicates a concurrent binding interaction between the two ligands, 2-DOS and DT, for the target 16S RNA. Further, a comparison of the ion abundance of the two binary complexes indicates that 2-DOS has greater binding affinity for the target RNA than DT.
Cooperative Binding

Ligands bind cooperatively when the binding of one ligand to the target enhances the binding of the other, i.e. more of the first ligand will bind to the target in the presence of the second ligand than in its absence. Cooperatively binding ligands may bind to their target at distinct locations. In a mixture containing two cooperatively binding ligands there will be an equilibrium of two binary complexes, a ternary complex and unbound target. The ternary complex is a simultaneous adduction of both ligands to the target. One of the binary complexes is complex of the first ligand bound to the target and the other binary complex is that of the second ligand bound to the target. The ligand having the greater affinity for the target will demonstrate a higher signal intensity for its binary complex with the target compared to the other ligand. Cooperative binding interaction between two ligands is determined according to methods of the invention by analyzing the mixture by mass-spectrometry and comparing the absolute ion abundance of the ternary complex to the sum of the relative ion abundance of the binary complexes contributing to the formation of the ternary complex in the same manner as for concurrent binders. However, in the instance of cooperative binding ligands, the relative ion abundance of the ternary complex (TL1L2/T) is greater than the sum of the relative ion abundances of the contributing binary complexes. Expressed another way, a pair of ligands are concurrent binders for a target if in either of the following equivalent formulae the value of y is greater than zero:

$$y = TL1L2 - TL1 \times \frac{TL2}{T} - TL2 \times \frac{TL1}{T}$$

$$or$$

$$y = TL1L2 - 2 \times \frac{TL1 \times TL2}{T}$$

Mixed Binding

Another scenario can arise when comparing the ion abundances, that is, when the ternary ion abundance is less than the sum of the relative abundances of the contributing binary complexes (i.e., y of the above formulae is less than zero). This indicates a more complex binding situation where there is a combination of interactions resulting from a competitive interaction between the ligands while at the same time another non-competitive interaction (cooperative or concurrent) is also occurring. Stated another way, this indicates a mixed binding mode arising when either or both ligands have more than one binding site on the target that may be detected by a mass-spectrum signal for the multiply bound target. Complex binding interaction of two ligands includes competitive/cooperative, competitive/concurrent, cooperative/concurrent, competitive/cooperative/concurrent or further combinations thereof.

A mixture in which two ligands have both competitive and concurrent binding interactions will exhibit a mass-spec signal for a ternary complex whereas a mixture having only a competitive interaction will exhibit no such signal. A mixture in which two ligands exhibit both a competitive and cooperative interaction will exhibit a mass-spec signal for the ternary complex and the absolute ion abundance for the ternary complex (TL1L2) will be greater than the sum of the relative ion abundance for the contributing binary complexes when the cooperative interaction is predominant. Conversely, the absolute ternary abundance will be less when the competitive interaction is stronger than the cooperative interaction. When there is both competitive and concurrent binding interaction, the absolute ternary ion abundance will be less than the sum of the relative ion abundances for the contributing binary complexes and greater when there is both cooperative and concurrent binding interaction.

Another embodiment of the invention includes methods for determining the relative proximity and orientation of binding sites for a first ligand and a second ligand on a target substrate. The target substrate is exposed to a mixture of the second ligand and at least one derivative compound of the first ligand. Derivative compounds of the first ligand are derivative structures that include the first ligand and have at least one substituent group pendent from the first ligand. The mixture is analyzed by mass spectrometry to identify those first ligand derivatives that inhibits the binding of the second ligand to the target substrate. In this embodiment, the method of determining the mode of binding interaction previously discussed may be used to determine the spatial proximity of ligand binding sites on a target. For example, the knowledge that two ligands are concurrent binders indicates that they have separate and distinct binding sites. In order to determine the distance between these two binding sites, derivatives of one of the ligands are prepared and mixed with the other ligand and the target. The derivatives of the first ligand will have the core chemical structure of the ligand but will also have substituents pending from the structure, the substituents having a diversity of lengths and attachment points to the structure.

A ligand derivative that inhibits the binding of the second ligand to the target, i.e. a derivative that is competitive with the second ligand, provides insight into the proximity and orientation of the binding sites relative to each other. A competitive derivative is identified by mass-spec analysis of the mixture and its particular substituent and attachment point on the parent ligand structure is determined. The point of attachment of the substituent indicates the relative orientation while the length of the substituent indicates the relative proximity of the binding sites. In this way the substituent group serves as a molecular ruler and compass.

An efficient manner of performing the method is by employing combinatorial chemistry techniques to create a library of ligand derivatives having great diversity in substituents. Suitable substituent groups include but are not limited to alkyl (e.g. methyl, ethyl, propyl), alkenyl (e.g. allyl), alkynyl (e.g. propynyl), alkoxy (e.g. methoxy, ethoxy), alkoxycarbonyl, acyl, acyloxy, aryl (e.g. phenyl), aralkyl, hydroxyl, hydroxylamino, keto (═O)amino, alkylamino (e.g. methylamino), mercapto, thioalkyl (e.g. thiomethyl, thioethyl), halogen (e.g. chloro, bromo), nitro, haloalkyl (e.g. trifluoromethyl), phosphorous, phosphate, sulfur and sulfate.

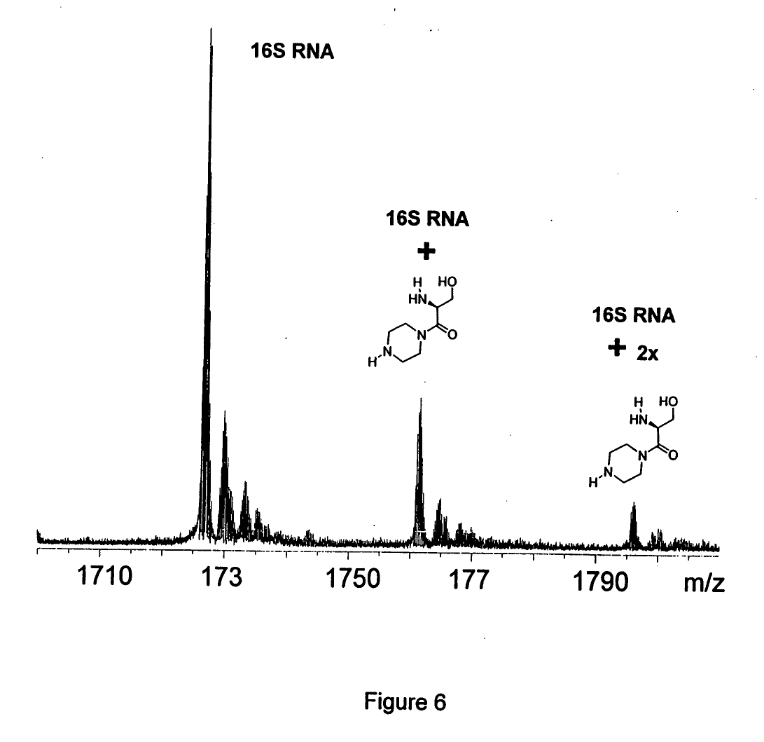

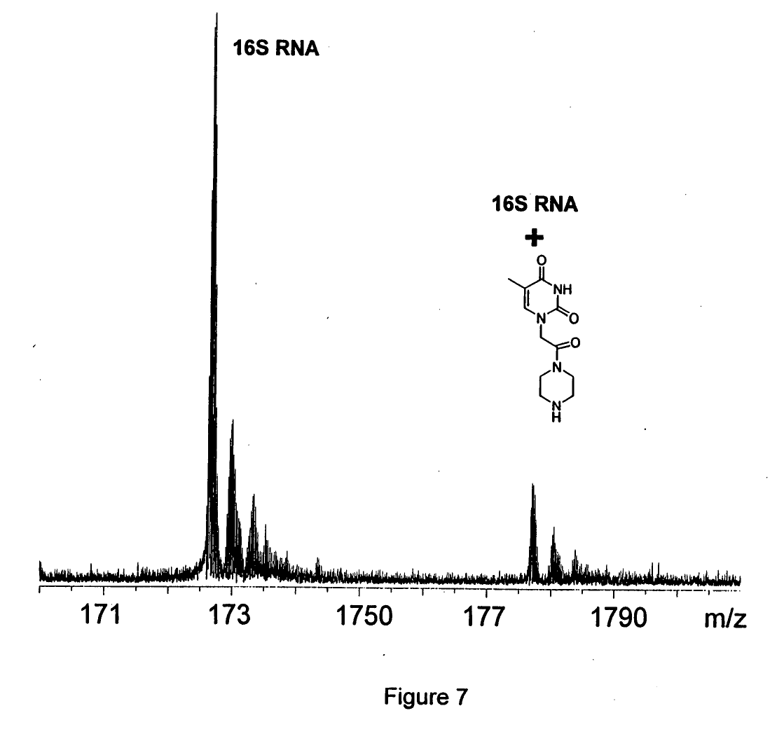

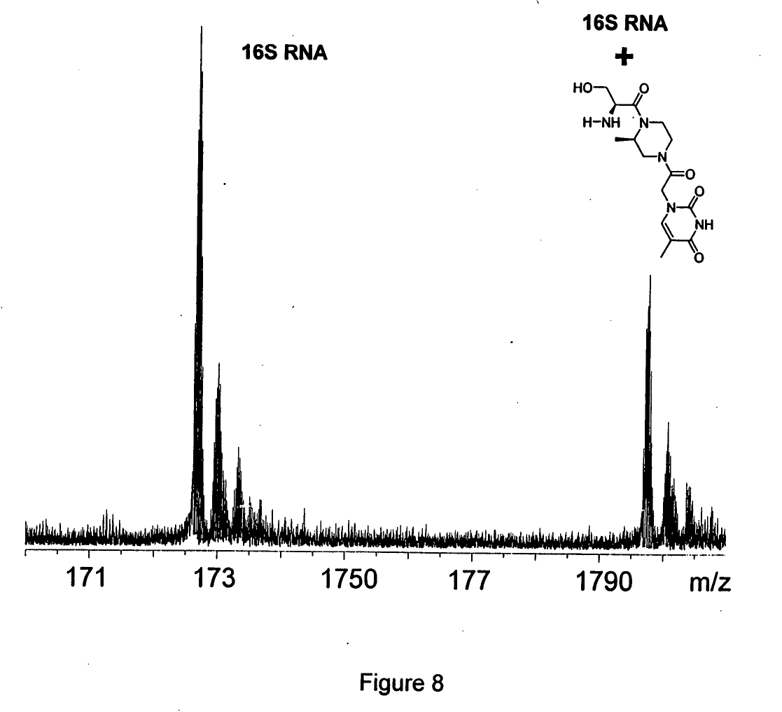

In a further embodiment of the invention, the invention includes a screening method for determining compounds having binding affinity to a target substrate. A mixture of the ligands and the target substrate are analyzed by mass spectrometry. First and second ligand that bind to the target substrate are identified. These first and second ligands are concatenated to form a third ligand having greater binding affinity for the target substrate than either first or second ligand. In this embodiment of the invention, ligands are identified using mass spectrometry methods described herein and are concatenated or linked together to form a new ligand incorporating the chemical structure responsible for binding of the two parent ligands to the target. The new concatenated ligand will have greater binding affinity for the target than either of the two parent ligands. An example of this is illustrated in examples 4 and 5 and FIGS. 6-8 where mass-spec analysis of a library of amide compounds revealed two having binding affinity for a fragment of bacterial 16S ribosomal RNA. The two ligands (IBIS-271583 and IBIS-326611) both incorporated a piperazine moiety and a concatenated compound of the two ligands was prepared having a common piperazine moiety from which the remainder of the ligand structures depend. The concatenated compound (IBIS-326645) is shown in FIG. 8 to bind the target 16S RNA fragment with greater affinity (52.4% of the target) than either of the two parent ligands in FIGS. 6 and 7 (27.8% and 14.7% respectively). In one embodiment, the new concatenated ligand comprises the chemical structure of the first and second ligands linked together by a linking group. Suitable linking groups are well known in the art and depend upon the chemical structure of the ligands and are linked to atoms of the ligand molecule not directly involved in binding to the target.

Linking groups are selected that generally are of a length that results in a reduction in entropy of the ligand target system. Typically a linker will have a length of about 15 Angstroms, less than about 10 Angstroms, or less than 5 Angstroms. Suitable linking groups include, but are not limited to, a direct covalent bond, alkylene (e.g. methylene, ethylene), alkenylene, alkynylene, arylene, ether (e.g. alkylethers), alkylene-esters, thioether, alkylene-thioesters, aminoalkylene (e.g. aminomethylene), amine, thioalkylene and heterocycles (e.g. pyrimidines, piperizine and aralkylene).

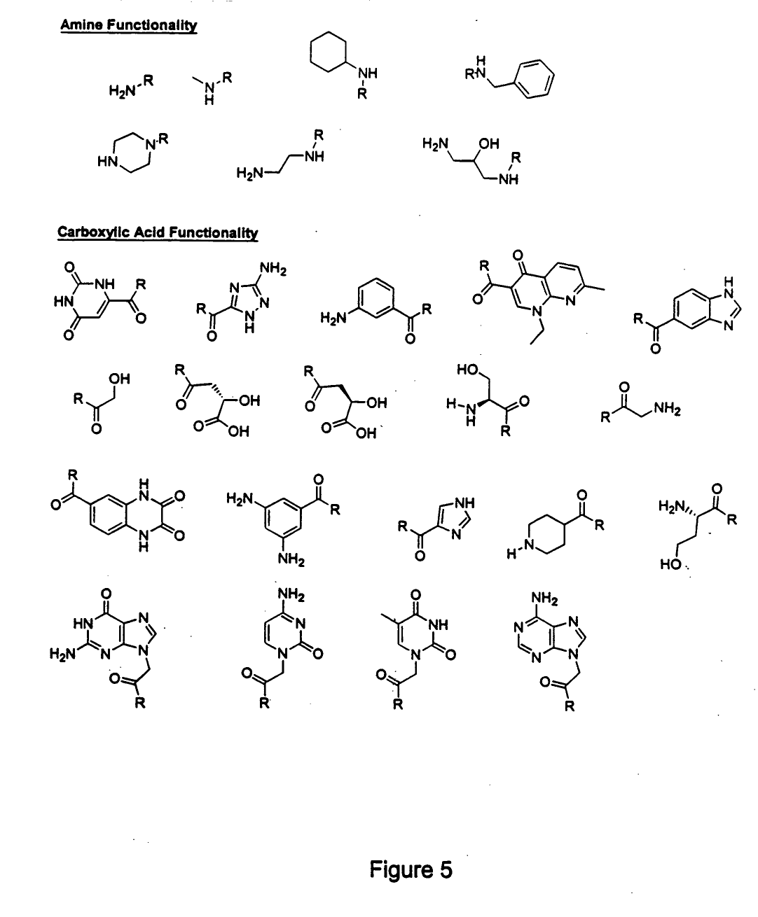

An example of the above method is shown in FIGS. 5 through 7. In separate mixtures, 200 μM of three ligands IBIS-326611 ((2S)-2-amino-3-hydroxy-1-piperazinylpropan-1-one), IBIS-326645 (5-methyl-1-(2-oxo-2-piperazinylethyl)-1,3-dihydropyrimidine-2,4-dione) and a concatenated compound thereof, IBIS-271583 (1-{2-[(3R)-4-((2S)-2-amino-3-hydroxypropanoyl)-3-methylpiperazinyl]-2-oxoethyl}-5-methyl-1,3-dihydropyrimidine-2,4-dione) are each mixed with 5 μM of target 16S RNA fragment and analyzed by mass spectrometry. IBIS-326611 is shown in FIG. 5 to form a binary complex having an ion abundance 27.8% that of the unbound 16S RNA while IBIS-326645 in FIG. 6 forms a binary complex having an ion abundance 14.7% that of the unbound 16S RNA. The concatenated compound IBIS-271483 on the other hand forms a binary complex having 52.4% ion abundance relative to unbound 16S RNA, and therefor has greater affinity for the target 16S RNA than either of the parent compounds.

New concatenated ligands may be screened in the same manner as were the parent ligands, and the affinities of those that bind may be measured through titration of the ligand concentration. The binding location of the new molecule on the target may be determined using a mass spectrometry-based protection assay, infrared multiphoton dissociation, NMR, X-ray crystallography, AFM force microcopy and other known techniques. Suitable concatenated ligands having improved affinity may then be screened in functional assays to demonstrate a biological effect appropriate for a drug molecule. If the biological activity is insufficient, the molecules may be iterated through the process additional times.

In one embodiment, the linking group is chosen based on the relative orientation and proximity of the ligand binding sites by exposing the target substrate to a mixture of the second ligand and a plurality of derivative compounds of the first ligand wherein the first ligand derivatives comprising the chemical structure of the first ligand and at least one substituent group pending therefrom. The mixture is analyzed by mass spectrometry to identify a first ligand derivative that inhibits the binding of said second ligand to the target substrate. In this method, mass spectrometry is used to infer the local environments of ligands. The footprint of one or more of the binding ligands may be increased through addition of substituents such as methyl, ethyl, amino, methylamino, methoxy, ethoxy, thiomethyl, thioethyl, bromo, nitro, chloro, trifluoromethyl and phenyl groups at different positions. This allows a SAR series to be constructed (either virtually or in vitro) for each individual ligand. For example, a methyl group may be added to the first ligand and it is found by the mass-spec screening that the methyl group does not affect the binding of the second ligand. This suggests that a methyl group may be an appropriate point to use for ligation with the second ligand. For example, it was found that first and second ligands bind cooperatively to a target and that a methyl derivative of the first ligand retains the cooperative binding with the second ligand. This indicates that point of attachment of the methyl group on the first ligand may be a suitable point on that ligand for linking to the second ligand. In the instance where the binding sites of the first and second ligand overlap, a concatenated compound comprising a fusion of the two chemical structures that are responsible for binding to the target will have greater affinity to the target than either first or second ligand.

Alternatively, the orientation and proximity of the binding sites may be determined by molecular modeling techniques, i.e., in silico, using programs such as MCSS (LeClerk, 1999) and others that virtually reproduce stacking, hydrogen bonding and electrostatic contacts with the target. Orientation and proximity of the binding sites can be determined by a combination of molecular modeling and the methods employing derivatized ligands in an iterative process wherein each technique provides information useful in performing the other. For example, molecular modeling may predict the orientation of a ligand at its binding site and give insight into the position at which a substituent or linking group may be attached to the ligand. Other techniques may also be used separately or in combination with those mentioned such as X-ray crystallography which provides 3-dimensional orientation and location when bound to its target. Another technique available for determining orientation and proximity of ligands at their binding site for designing linking groups is by NMR. A particular NMR method for determining orientation and proximity is described in patent application WO97/18469 which claims priority from U.S. Ser. No. 08/558,644 (filed 14 Nov. 1995) and Ser. No. 08/678,903 (filed 12 Jul. 1996) each incorporated herein by reference. In this NMR method a target molecule is labeled with ¹⁵N and analyzed by ¹⁵N/¹H NMR correlation spectroscopy when bound by the ligands. This method is particularly useful for targets that are easily labeled with ¹⁵N such as proteins and peptide.

The target molecules that are nucleic acid molecules and/or the ligands that are nucleic acid molecules can have any number of chemistries, which are described in more detail below.

In some embodiments, the nucleic acid molecules (target molecules and/or ligands) can have at least 5 regions that alternate between 3′-endo and 2′-endo in conformational geometry. The nucleoside or nucleosides of a particular region can be modified in a variety of ways to give the region either a 3′-endo or a 2′-endo conformational geometry. The conformational geometry of a selected nucleoside can be modulated in one aspect by modifying the sugar the base or both the sugar and the base. Modifications include attachment of substituent groups or conjugate groups or by directly modifying the base or the sugar.

The sugar conformational geometry (puckering) plays a central role in determining the duplex conformational geometry between an oligonucleotide and its nucleic acid target. By controlling the sugar puckering independently at each position of an oligonucleotide the duplex geometry can be modulated to help maximize desired properties of the resulting chimeric oligomeric compound. Modulation of sugar geometry has been shown to enhance properties such as for example increased lipohpilicity, binding affinity to target nucleic acid (e.g. mRNA), chemical stability and nuclease resistance.

In some embodiments, the nucleic acid molecules (target molecules and/or ligands) comprise a plurality of alternating 3′-endo and 2′-endo (including 2′-deoxy) regions wherein each of the regions are independently from about 1 to about 5 nucleosides in length. The nucleic acid molecules (target molecules and/or ligands) can start and end with either 3′-endo or 2′-endo regions and have from about 5 to about 17 regions in total. The nucleosides of each region can be selected to be uniform such as for example uniform 2′-O-MOE nucleosides for one or more of the 3′-endo regions and 2′-deoxynucleosides for the 2′-endo regions. Alternatively the nucleosides can be mixed such that any nucleoside having 3′-endo conformational geometry can be used in any position of any 3′-endo region and any nucleoside having 2′-endo conformational geometry can be used in any position of any 2′-endo region. In some embodiments a 5′-conjugate group is used as a 5′-cap as a method of increasing the 5′-exonuclease resistance but conjugate groups can be used at any position within the nucleic acid molecules (target molecules and/or ligands).

3′-Endo Regions

In some embodiments of the invention, the nucleic acid molecules (target molecules and/or ligands) have alternating regions wherein one of the alternating regions have 3′-endo conformational geometry. These 3′-endo regions include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA like nucleosides so that particular properties of an oligomeric compound can be enhanced while maintaining the desirable 3′-endo conformational geometry. Properties that are enhanced by using more stable 3′-endo nucleosides include but are not limited to modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. The present invention provides regions of nucleosides modified in such a way as to favor a C3′-endo type conformation.

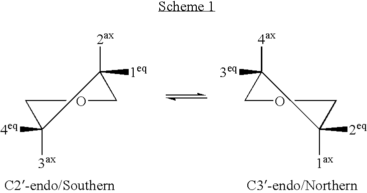

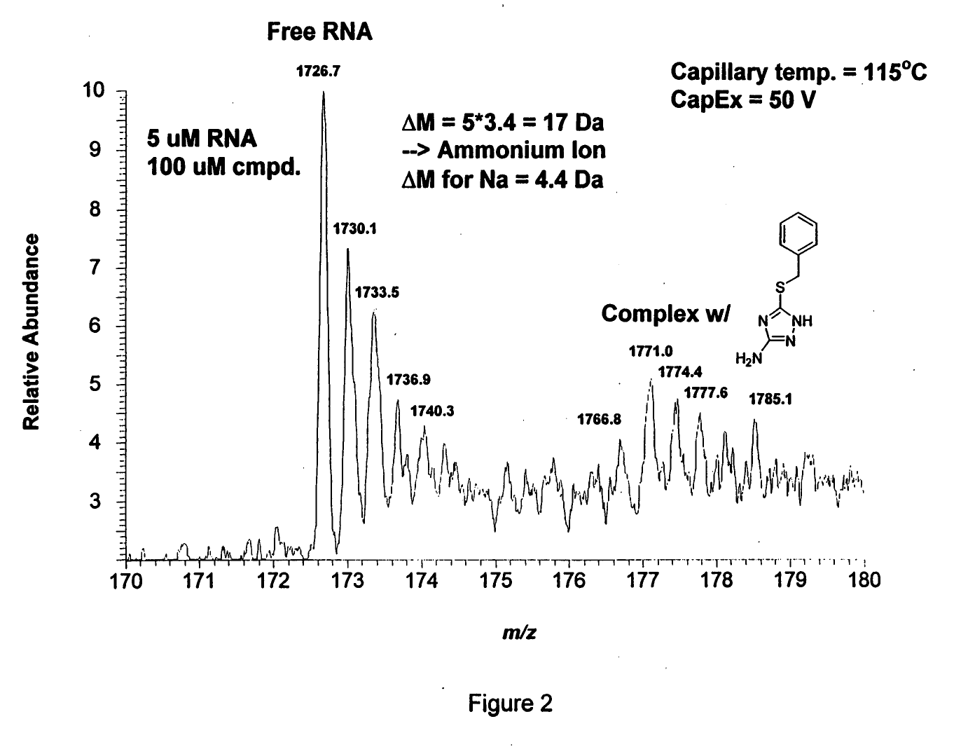

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag). Modification of the 2′ position to favor the 3′-endo conformation can be achieved while maintaining the 2′-OH as a recognition element, as illustrated in FIG. 2, below (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754).

Alternatively, preference for the 3′-endo conformation can be achieved by deletion of the 2′-OH as exemplified by 2′deoxy-2′F-nucleosides (Kawasaki et al., J. Med. Chem. (1993), 36, 831-841), which adopts the 3′-endo conformation positioning the electronegative fluorine atom in the axial position. Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Along similar lines, 3′-endo regions can include one or more nucleosides modified in such a way that conformation is locked into a C3′-endo type conformation, i.e. Locked Nucleic Acid (LNA, Singh et al, Chem. Commun. (1998), 4, 455-456), and ethylene bridged Nucleic Acids (ENA, Morita et al, Bioorganic & Medicinal Chemistry Letters (2002), 12, 73-76.).

Examples of modified nucleosides amenable to the present invention are shown below in Table 1. These examples are meant to be representative and not exhaustive.

TABLE 1

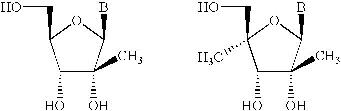

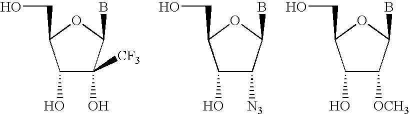

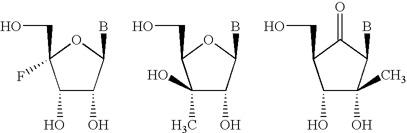

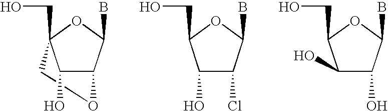

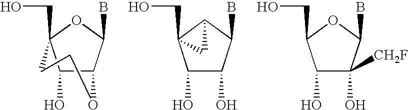

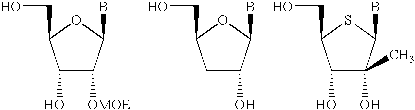

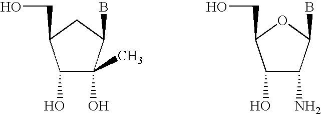

The preferred conformation of modified nucleosides and their oligomers can be estimated by various methods such as molecular dynamics calculations, nuclear magnetic resonance spectroscopy and CD measurements. Hence, modifications predicted to induce RNA like conformations, A-form duplex geometry in an oligomeric context, are selected for use in the modified oligoncleotides of the present invention. The synthesis of numerous of the modified nucleosides amenable to the present invention are known in the art (see for example, Chemistry of Nucleosides and Nucleotides Vol 1-3, ed. Leroy B. Townsend, 1988, Plenum press., and the examples section below). Nucleosides known to be inhibitors/substrates for RNA dependent RNA polymerases (for example HCV NS5B).

The terms used to describe the conformational geometry of homoduplex nucleic acids are “A Form” for RNA and “B Form” for DNA. The respective conformational geometry for RNA and DNA duplexes was determined from X-ray diffraction analysis of nucleic acid fibers (Arnott and Hukins, Biochem. Biophys. Res. Comm., 1970, 47, 1504.) In general, RNA:RNA duplexes are more stable and have higher melting temperatures (Tms) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). As used herein, B-form geometry is inclusive of both C2′-endo pucker and O4′-endo pucker. This is consistent with Berger et. al., Nucleic Acids Research, 1998, 26, 2473-2480, who pointed out that in considering the furanose conformations which give rise to B-form duplexes consideration should also be given to a O4′-endo pucker contribution.

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of the duplex formed between a target RNA and a synthetic sequence is central to therapies such as but not limited to antisense and RNA interference as these mechanisms require the binding of a synthetic oligonucleotide strand to an RNA target strand. In the case of antisense, effective inhibition of the mRNA requires that the antisense DNA have a very high binding affinity with the mRNA. Otherwise the desired interaction between the synthetic oligonucleotide strand and target mRNA strand will occur infrequently, resulting in decreased efficacyl.

One routinely used method of modifying the sugar puckering is the substitution of the sugar at the 2′-position with a substituent group that influences the sugar geometry. The influence on ring conformation is dependant on the nature of the substituent at the 2′-position. A number of different substituents have been studied to determine their sugar puckering effect. For example, 2′-halogens have been studied showing that the 2′-fluoro derivative exhibits the largest population (65%) of the C3′-endo form, and the 2′-iodo exhibits the lowest population (7%). The populations of adenosine (2′-OH) versus deoxyadenosine (2′-H) are 36% and 19%, respectively. Furthermore, the effect of the 2′-fluoro group of adenosine dimers (2′-deoxy-2′-fluoroadenosine-2′-deoxy-2′-fluoro-adenosine) is further correlated to the stabilization of the stacked conformation.

As expected, the relative duplex stability can be enhanced by replacement of 2′-OH groups with 2′-F groups thereby increasing the C3′-endo population. It is assumed that the highly polar nature of the 2′-F bond and the extreme preference for C3′-endo puckering may stabilize the stacked conformation in an A-form duplex. Data from UV hypochromicity, circular dichroism, and ¹H NMR also indicate that the degree of stacking decreases as the electronegativity of the halo substituent decreases. Furthermore, steric bulk at the 2′-position of the sugar moiety is better accommodated in an A-form duplex than a B-form duplex. Thus, a 2′-substituent on the 3′-terminus of a dinucleoside monophosphate is thought to exert a number of effects on the stacking conformation: steric repulsion, furanose puckering preference, electrostatic repulsion, hydrophobic attraction, and hydrogen bonding capabilities. These substituent effects are thought to be determined by the molecular size, electronegativity, and hydrophobicity of the substituent. Melting temperatures of complementary strands is also increased with the 2′-substituted adenosine diphosphates. It is not clear whether the 3′-endo preference of the conformation or the presence of the substituent is responsible for the increased binding. However, greater overlap of adjacent bases (stacking) can be achieved with the 3′-endo conformation.

One synthetic 2′-modification that imparts increased nuclease resistance and a very high binding affinity to nucleotides is the 2-methoxyethoxy (2′-MOE, 2′-OCH₂CH₂OCH₃) side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligonucleotides having the 2′-O-methoxyethyl substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926). Relative to DNA, the oligonucleotides having the 2′-MOE modification displayed improved RNA affinity and higher nuclease resistance. Chimeric oligomeric compounds having 2′-MOE substituents in the wing nucleosides and an internal region of deoxy-phosphorothioate nucleotides (also termed a gapped oligonucleotide or gapmer) have shown effective reduction in the growth of tumors in animal models at low doses. 2′-MOE substituted oligonucleotides have also shown outstanding promise as antisense agents in several disease states. One such MOE substituted oligonucleotide is presently being investigated in clinical trials for the treatment of CMV retinitis.

To better understand the higher RNA affinity of 2′-O-methoxyethyl substituted RNA and to examine the conformational properties of the 2′-O-methoxyethyl substituent, two dodecamer oligonucleotides were synthesized having SEQ ID NO:1 (CGCGAAUUCGCG) and SEQ ID NO:2 (GCGCUUAAGCGC). These self-complementary strands have every 2′-position modified with a 2′-O-methoxyethyl. The duplex was crystallized at a resolution of 1.7 Ångstrom and the crystal structure was determined. The conditions used for the crystallization were 2 mM oligonucleotide, 50 mM Na Hepes pH 6.2-7.5, 10.50 mM MgCl₂, 15% PEG 400. The crystal data showed: space group C2, cell constants a=41.2 Å, b=34.4 Å, c=46.6 Å, .=92.4°. The resolution was 1.7 Å at −170° C. The current R=factor was 20% (R_{free} 26%).

This crystal structure is believed to be the first crystal structure of a fully modified RNA oligonucleotide analogue. The duplex adopts an overall A-form conformation and all modified sugars display C3′-endo pucker. In most of the 2′-O-substituents, the torsion angle around the A′-B′ bond, as depicted in Structure II below, of the ethylene glycol linker has a gauche conformation. For 2′-O-MOE, A′ and B′ of Structure II below are methylene moieties of the ethyl portion of the MOE and R′ is the methoxy portion.

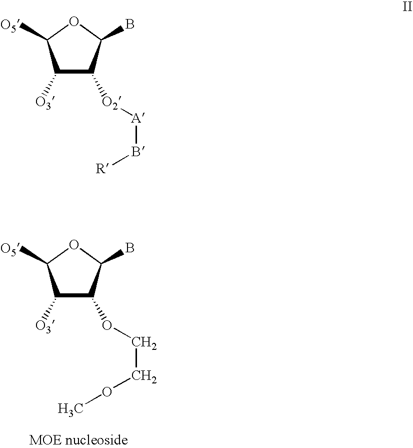

In the crystal, the 2′-O-MOE RNA duplex adopts a general orientation such that the crystallographic 2-fold rotation axis does not coincide with the molecular 2-fold rotation axis. The duplex adopts the expected A-type geometry and all of the 24 2′-O-MOE substituents were visible in the electron density maps at full resolution. The electron density maps as well as the temperature factors of substituent atoms indicate flexibility of the 2′-O-MOE substituent in some cases.

Most of the 2′-O-MOE substituents display a gauche conformation around the C—C bond of the ethyl linker. However, in two cases, a trans conformation around the C—C bond is observed. The lattice interactions in the crystal include packing of duplexes against each other via their minor grooves. Therefore, for some residues, the conformation of the 2′-O-substituent is affected by contacts to an adjacent duplex. In general, variations in the conformation of the substituents (e.g. g⁺ or g⁻ around the C—C bonds) create a range of interactions between substituents, both inter-strand, across the minor groove, and intra-strand. At one location, atoms of substituents from two residues are in van der Waals contact across the minor groove. Similarly, a close contact occurs between atoms of substituents from two adjacent intra-strand residues.

Previously determined crystal structures of A-DNA duplexes were for those that incorporated isolated 2′-O-methyl T residues. In the crystal structure noted above for the 2′-O-MOE substituents, a conserved hydration pattern has been observed for the 2′-O-MOE residues. A single water molecule is seen located between O2′, O3′ and the methoxy oxygen atom of the substituent, forming contacts to all three of between 2.9 and 3.4 Å. In addition, oxygen atoms of substituents are involved in several other hydrogen bonding contacts. For example, the methoxy oxygen atom of a particular 2′-O-substituent forms a hydrogen bond to N3 of an adenosine from the opposite strand via a bridging water molecule.

In several cases a water molecule is trapped between the oxygen atoms O2′, O3′ and OC′ of modified nucleosides. 2′-O-MOE substituents with trans conformation around the C—C bond of the ethylene glycol linker are associated with close contacts between OC′ and N2 of a guanosine from the opposite strand, and, water-mediated, between OC′ and N3(G). When combined with the available thermodynamic data for duplexes containing 2′-O-MOE modified strands, this crystal structure allows for further detailed structure-stability analysis of other modifications.

In extending the crystallographic structure studies, molecular modeling experiments were performed to study further enhanced binding affinity of oligonucleotides having 2′-O-modifications of the invention. The computer simulations were conducted on compounds of SEQ ID NO:1, above, having 2′-O-modifications of the invention located at each of the nucleoside of the oligonucleotide. The simulations were performed with the oligonucleotide in aqueous solution using the AMBER force field method (Cornell et al., J. Am. Chem. Soc., 1995, 117, 5179-5197)(modeling software package from UCSF, San Francisco, Calif.). The calculations were performed on an Indigo2 SGI machine (Silicon Graphics, Mountain View, Calif.).

Further 2′-O-modifications that will have a 3′-endo sugar influence include those having a ring structure that incorporates a two atom portion corresponding to the A′ and B′ atoms of Structure II. The ring structure is attached at the 2′ position of a sugar moiety of one or more nucleosides that are incorporated into an oligonucleotide. The 2′-oxygen of the nucleoside links to a carbon atom corresponding to the A′ atom of Structure II. These ring structures can be aliphatic, unsaturated aliphatic, aromatic or heterocyclic. A further atom of the ring (corresponding to the B′ atom of Structure II), bears a further oxygen atom, or a sulfur or nitrogen atom. This oxygen, sulfur or nitrogen atom is bonded to one or more hydrogen atoms, alkyl moieties, or haloalkyl moieties, or is part of a further chemical moiety such as a ureido, carbamate, amide or amidine moiety. The remainder of the ring structure restricts rotation about the bond joining these two ring atoms. This assists in positioning the “further oxygen, sulfur or nitrogen atom” (part of the R position as described above) such that the further atom can be located in close proximity to the 3′-oxygen atom (O3′) of the nucleoside.

Another suitable 2′-sugar substituent group that gives a 3′-endo sugar conformational geometry is the 2′-OMe group. 2′-Substitution of guanosine, cytidine, and uridine dinucleoside phosphates with the 2′-OMe group showed enhanced stacking effects with respect to the corresponding native (2′-OH) species leading to the conclusion that the sugar is adopting a C3′-endo conformation. In this case, it is believed that the hydrophobic attractive forces of the methyl group tend to overcome the destabilizing effects of its steric bulk.

The ability of oligonucleotides to bind to their complementary target strands is compared by determining the melting temperature (Tₘ) of the hybridization complex of the oligonucleotide and its complementary strand. The melting temperature (Tₘ), a characteristic physical property of double helices, denotes the temperature (in degrees centigrade) at which 50% helical (hybridized) versus coil (unhybridized) forms are present. Tₘ is measured by using the UV spectrum to determine the formation and breakdown (melting) of the hybridization complex. Base stacking, which occurs during hybridization, is accompanied by a reduction in UV absorption (hypochromicity). Consequently, a reduction in UV absorption indicates a higher Tₘ. The higher the Tₘ, the greater the strength of the bonds between the strands.

Freier and Altmann, Nucleic Acids Research, (1997) 25:4429-4443, have previously published a study on the influence of structural modifications of oligonucleotides on the stability of their duplexes with target RNA. In this study, the authors reviewed a series of oligonucleotides containing more than 200 different modifications that had been synthesized and assessed for their hybridization affinity and Tm. Sugar modifications studied included substitutions on the 2′-position of the sugar, 3′-substitution, replacement of the 4′-oxygen, the use of bicyclic sugars, and four member ring replacements. Several nucleobase modifications were also studied including substitutions at the 5, or 6 position of thymine, modifications of pyrimidine heterocycle and modifications of the purine heterocycle. Modified internucleoside linkages were also studied including neutral, phosphorus and non-phosphorus containing internucleoside linkages.

Increasing the percentage of C3′-endo sugars in a modified oligonucleotide targeted to an RNA target strand should preorganize this strand for binding to RNA. Of the several sugar modifications that have been reported and studied in the literature, the incorporation of electronegative substituents such as 2′-fluoro or 2′-alkoxy shift the sugar conformation towards the 3′ endo (northern) pucker conformation. This preorganizes an oligonucleotide that incorporates such modifications to have an A-form conformational geometry. This A-form conformation results in increased binding affinity of the oligonucleotide to a target RNA strand.

Molecular modeling experiments were performed to study further enhanced binding affinity of oligonucleotides having 2′-O-modifications. Computer simulations were conducted on compounds having SEQ ID NO:3, r(CGC GAA UUC GCG), having 2′-O-modifications of the invention located at each of the nucleoside of the oligonucleotide. The simulations were performed with the oligonucleotide in aqueous solution using the AMBER force field method (Cornell et al., J. Am. Chem. Soc., 1995, 117, 5179-5197)(modeling software package from UCSF, San Francisco, Calif.). The calculations were performed on an Indigo2 SGI machine (Silicon Graphics, Mountain View, Calif.).

In addition, for 2′-substituents containing an ethylene glycol motif, a gauche interaction between the oxygen atoms around the O—C≡C—O torsion of the side chain may have a stabilizing effect on the duplex (Freier ibid.). Such gauche interactions have been observed experimentally for a number of years (Wolfe et al., Acc. Chem. Res., 1972, 5, 102; Abe et al., J. Am. Chem. Soc., 1976, 98, 468). This gauche effect may result in a configuration of the side chain that is favorable for duplex formation. The exact nature of this stabilizing configuration has not yet been explained. While we do not want to be bound by theory, it may be that holding the O—C—C—O torsion in a single gauche configuration, rather than a more random distribution seen in an alkyl side chain, provides an entropic advantage for duplex formation.

Representative 2′-substituent groups amenable to the present invention that give A-form conformational properties (3′-endo) to the resultant duplexes include 2′-O-alkyl, 2′-O-substituted alkyl and 2′-fluoro substituent groups. Suitable substituent groups are various alkyl and aryl ethers and thioethers, amines and monoalkyl and dialkyl substituted amines. It is further intended that multiple modifications can be made to one or more nucleosides and or internucleoside linkages within an oligonucleotide of the invention to enhance activity of the oligonucleotide. Tables 2 through 8 list nucleoside and internucleotide linkage modifications/replacements that have been shown to give a positive ∈Tm per modification when the modification/replacement was made to a DNA strand that was hybridized to an RNA complement.

TABLE 2
Modified DNA strand having 2′-substituent groups that
gave an overall increase in Tm against an RNA complement:

Positive εTm/mod
2′-substituents 2′-OH
2′-O—C₁—C₄ alkyl
2′-O—(CH₂)₂CH₃
2′-O—CH₂CH═CH₂
2′-F
2′-O—(CH₂)₂—O—CH₃
2′-[O—(CH₂)₂]₂—O—CH₃
2′-[O—(CH₂)₂]₃—O—CH₃
2′-[O—(CH₂)₂]₄—O—CH₃
2′-[O—(CH₂)₂]₃—O—(CH₂)₈CH₃
2′-O—(CH₂)₂CF₃
2′-O—(CH₂)₂OH
2′-O—(CH₂)₂F
2′-O—CH₂CH(CH₃)F
2′-O—CH₂CH(CH₂OH)OH
2′-O—CH₂CH(CH₂OCH₃)OCH₃
2′-O—CH₂CH(CH₃)OCH₃
2′-O—CH₂—C₁₄H₇O₂(—C₁₄H₇O₂ = Anthraquinone)
2′-O—(CH₂)₃—NH₂*
2′-O—(CH₂)₄—NH₂*
*These modifications can increase the Tm of oligonucleotides but can also decrease the Tm depending on positioning and number (motiff dependant).

TABLE 3
Modified DNA strand having modified sugar ring (see structure x)
that give an overall increase in Tm against an RNA complement:

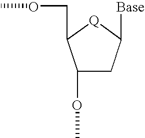

| | Positive ∈Tm/mod |
|---|---|
| Q | —S— |
| | —CH₂— |
| Note: | |
| In general ring oxygen substitution with sulfur or methylene had only a minor effect on Tm for the specific motiffs studied. Substitution at the 2′-position with groups shown to stabilize the duplex were destabilizing when CH₂ replaced the ring O. | |
| # This is thought to be due to the necessary gauche interaction between the ring O with particular 2′-substituents (for example —O—CH₃ and —(O—CH₂CH₂)₃—O—CH₃. | |

TABLE 4
Modified DNA strand having modified sugar ring that
give an overall increase in Tm against an RNA complement:

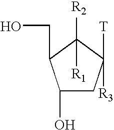

| | Positive ∈Tm/mod |
|---|---|
| —C(H)R₁ effects | OH |
| (R₂, R₃ both = H) | CH₃* |
| | CH₂OH* |
| | OCH₃* |

*These modifications can increase the Tm of oligonucleotides but can also decrease the Tm depending on positioning and number (motiff dependant).

TABLE 5
Modified DNA strand having bicyclic substitute sugar modifications
that give an overall increase in Tm against an RNA complement:

| Formula | Positive ∈Tm/mod |
|---|---|
| I | + |
| II | + |

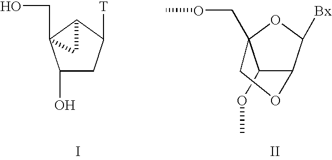

TABLE 6
Modified DNA strand having modified heterocyclic base moieties
that give an overall increase in Tm against an RNA complement:

| Modification/Formula | Positive ∈Tm/mod |
|---|---|
| Heterocyclic base | 2-thioT |
| modifications | 2′-O-methylpseudoU |
| | 7-halo-7-deaza purines |
| | 7-propyne-7-deaza purines |
| | 2-aminoA(2,6-diaminopurine) |
| Modification/Formula | Positive ∈Tm/mod |
| 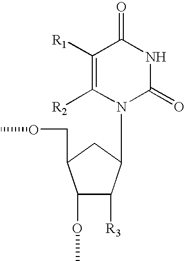 | |
| (R₂, R₃ = H), R = | Br |
| | C/C—CH₃ |
| | (CH₂)₃NH₂ |
| | CH₃ |
| Motiffs-disubstitution | |
| R₁ = C/C—CH₃, R₂ = H, R₃ = | F |
| R₁ = C/C—CH₃, R₂ = H | R₃ = O—(CH₂)₂—O—CH₃ |
| R₁ = O—CH₃, R₂ = H, | R₃ = O—(CH₂)₂—O—CH₃* |

*This modification can increase the Tm of oligonucleotides but can also decrease the Tm depending on positioning and number (motiff dependant).

Substitution at R₁ can be stabilizing, substitution at R₂ is generally greatly destabilizing (unable to form anti conformation), motiffs with stabilizing 5 and 2′-substituent groups are generally additive e.g. increase stability.

Substitution of the O4 and O2 positions of 2′-O-methyl uridine was greatly duplex destabilizing as these modifications remove hydrogen binding sites that would be an expected result. 6-Aza T also showed extreme destabilization as this substitution reduces the pKₐ and shifts the nucleoside toward the enol tautomer resulting in reduced hydrogen bonding.

TABLE 7
DNA strand having at least one modified phosphorus
containing internucleoside linkage and the effect
on the Tm against an RNA complement:

| εTm/mod + | εTm/mod − |
|---|---|
| | phosphorothioate¹ |
| | phosphoramidate¹ |
| | methyl phosphonates¹ |
| | (¹one of the non-bridging oxygen atoms |
| | replaced with S, N(H)R or —CH₃) |
| phosphoramidate (the 3′-bridging | |
| atom replaced with an N(H)R | |
| group, stabilization effect | |
| enhanced when also have 2′-F) | |

TABLE 8
DNA strand having at least one non-phosphorus
containing internucleoside linkage and the effect
on the Tm against an RNA complement:

Positive εTm/mod
—CH₂C(═O)NHCH₂—*
—CH₂C(═O)N(CH₃)CH₂—*
—CH₂C(═O)N(CH₂CH₂CH₃)CH₂—*
—CH₂C(═O)N(H)CH₂— (motiff with 5′-propyne on T's)
—CH₂N(H)C(═O)CH₂—*
—CH₂N(CH₃)OCH₂—*
—CH₂N(CH₃)N(CH₃)CH₂—*
*This modification can increase the Tm of oligonucleotides but can also decrease the Tm depending on positioning and number (motiff dependant).

Notes: In general carbon chain internucleotide linkages were destabilizing to duplex formation. This destabilization was not as severe when double and tripple bonds were utilized. The use of glycol and flexible ether linkages were also destabilizing.

Suitable ring structures of the invention for inclusion as a 2′-O modification include cyclohexyl, cyclopentyl and phenyl rings as well as heterocyclic rings having spacial footprints similar to cyclohexyl, cyclopentyl and phenyl rings. Particularly suitable 2′-O-substituent groups of the invention are listed below including an abbreviation for each:
2′-O-(trans 2-methoxy cyclohexyl)—2′-O-(TMCHL)
2′-O-(trans 2-methoxy cyclopentyl)—2′-O-(TMCPL)
2′-O-(trans 2-ureido cyclohexyl)—2′-O-(TUCHL)
2′-O-(trans 2-methoxyphenyl)—2′-O-(2MP)

Structural details for duplexes incorporating such 2-O-substituents were analyzed using the described AMBER force field program on the Indigo2 SGI machine. The simulated structure maintained a stable A-form geometry throughout the duration of the simulation. The presence of the 2′ substitutions locked the sugars in the C3′-endo conformation.

The simulation for the TMCHL modification revealed that the 2′-O-(TMCHL) side chains have a direct interaction with water molecules solvating the duplex. The oxygen atoms in the 2′-O-(TMCHL) side chain are capable of forming a water-mediated interaction with the 3′ oxygen of the phosphate backbone. The presence of the two oxygen atoms in the 2′-O-(TMCHL) side chain gives rise to favorable gauche interactions. The barrier for rotation around the O—C—C—O torsion is made even larger by this novel modification. The preferential preorganization in an A-type geometry increases the binding affinity of the 2′-O-(TMCHL) to the target RNA. The locked side chain conformation in the 2′-O-(TMCHL) group created a more favorable pocket for binding water molecules. The presence of these water molecules played a key role in holding the side chains in the preferable gauche conformation. While not wishing to be bound by theory, the bulk of the substituent, the diequatorial orientation of the substituents in the cyclohexane ring, the water of hydration and the potential for trapping of metal ions in the conformation generated will additionally contribute to improved binding affinity and nuclease resistance of oligonucleotides incorporating nucleosides having this 2′-O-modification.

As described for the TMCHL modification above, identical computer simulations of the 2′-O-(TMCPL), the 2′-O-(2MP) and 2′-O-(TUCHL) modified oligonucleotides in aqueous solution also illustrate that stable A-form geometry will be maintained throughout the duration of the simulation. The presence of the 2′ substitution will lock the sugars in the C3′-endo conformation and the side chains will have direct interaction with water molecules solvating the duplex. The oxygen atoms in the respective side chains are capable of forming a water-mediated interaction with the 3′ oxygen of the phosphate backbone. The presence of the two oxygen atoms in the respective side chains give rise to the favorable gauche interactions. The barrier for rotation around the respective O—C—C—O torsions will be made even larger by respective modification. The preferential preorganization in A-type geometry will increase the binding affinity of the respective 2′-O-modified oligonucleotides to the target RNA. The locked side chain conformation in the respective modifications will create a more favorable pocket for binding water molecules. The presence of these water molecules plays a key role in holding the side chains in the preferable gauche conformation. The bulk of the substituent, the diequatorial orientation of the substituents in their respective rings, the water of hydration and the potential trapping of metal ions in the conformation generated will all contribute to improved binding affinity and nuclease resistance of oligonucleotides incorporating nucleosides having these respective 2′-O-modification.

Ribose conformations in C2′-modified nucleosides containing S-methyl groups were examined. To understand the influence of 2′-O-methyl and 2′-S-methyl groups on the conformation of nucleosides, we evaluated the relative energies of the 2′-O- and 2′-S-methylguanosine, along with normal deoxyguanosine and riboguanosine, starting from both C2′-endo and C3′-endo conformations using ab initio quantum mechanical calculations. All the structures were fully optimized at HF/6-31G* level and single point energies with electron-correlation were obtained at the MP2/6-31G*//HF/6-31G* level. As shown in Table 9, the C2′-endo conformation of deoxyguanosine is estimated to be 0.6 kcal/mol more stable than the C3′-endo conformation in the gas-phase. The conformational preference of the C2′-endo over the C3′-endo conformation appears to be less dependent upon electron correlation as revealed by the MP2/6-31G*//HF/6-31G* values which also predict the same difference in energy. The opposite trend is noted for riboguanosine. At the HF/6-31G* and MP2/6-31G*//HF/6-31G* levels, the C3′-endo form of riboguanosine is shown to be about 0.65 and 1.41 kcal/mol more stable than the C2′endo form, respectively.

TABLE 9
Relative energies* of the C3′-endo and C2′-endo
conformations of representative nucleosides.

| HF/6-31G | MP2/6-31-G | CONTINUUM | AMBER | MODEL |
|---|---|---|---|---|
| dG | 0.60 | 0.56 | 0.88 | 0.65 |
| rG | −0.65 | −1.41 | −0.28 | −2.09 |
| 2′-O—MeG | −0.89 | −1.79 | −0.36 | −0.86 |
| 2′-S—MeG | 2.55 | 1.41 | 3.16 | 2.43 |

*energies are in kcal/mol relative to the C2′-endo conformation

Table 9 also includes the relative energies of 2′-O-methylguanosine and 2′-S-methylguanosine in C2′-endo and C3′-endo conformation. This data indicates the electronic nature of C2′-substitution has a significant impact on the relative stability of these conformations. Substitution of the 2′-O-methyl group increases the preference for the C3′-endo conformation (when compared to riboguanosine) by about 0.4 kcal/mol at both the HF/6-31G* and MP2/6-31G*//HF/6-31G* levels. In contrast, the 2′-S-methyl group reverses the trend. The C2′-endo conformation is favored by about 2.6 kcal/mol at the HF/6-31G* level, while the same difference is reduced to 1.41 kcal/mol at the MP2/6-31G*//HF/6-31G* level. For comparison, and also to evaluate the accuracy of the molecular mechanical force-field parameters used for the 2′-O-methyl and 2′-S-methyl substituted nucleosides, we have calculated the gas phase energies of the nucleosides. The results reported in Table 9 indicate that the calculated relative energies of these nucleosides compare qualitatively well with the ab initio calculations.

Additional calculations were also performed to gauge the effect of solvation on the relative stability of nucleoside conformations. The estimated solvation effect using HF/6-31G* geometries confirms that the relative energetic preference of the four nucleosides in the gas-phase is maintained in the aqueous phase as well (Table 9). Solvation effects were also examined using molecular dynamics simulations of the nucleosides in explicit water. From these trajectories, one can observe the predominance of C2′-endo conformation for deoxyriboguanosine and 2′-S-methylriboguanosine while riboguanosine and 2′-O-methylriboguanosine prefer the C3′-endo conformation. These results are in much accord with the available NMR results on 2′-S-methylribonucleosides. NMR studies of sugar puckering equilibrium using vicinal spin-coupling constants have indicated that the conformation of the sugar ring in 2′-S-methylpyrimidine nucleosides show an average of >75% S-character, whereas the corresponding purine analogs exhibit an average of >90% Spucker (Fraser, A., Wheeler, P., Cook, P. D. and Sanghvi, Y. S., J. Heterocycl. Chem., 1993, 30, 1277-1287). It was observed that the 2′-S-methyl substitution in deoxynucleoside confers more conformational rigidity to the sugar conformation when compared with deoxyribonucleosides.

Structural features of DNA:RNA, OMe-DNA:RNA and SMe-DNA:RNA hybrids were also observed. The average RMS deviation of the DNA:RNA structure from the starting hybrid coordinates indicate the structure is stabilized over the length of the simulation with an approximate average RMS deviation of 1.0 Å. This deviation is due, in part, to inherent differences in averaged structures (i.e. the starting conformation) and structures at thermal equilibrium. The changes in sugar pucker conformation for three of the central base pairs of this hybrid are in good agreement with the observations made in previous NMR studies. The sugars in the RNA strand maintain very stable geometries in the C3′-endo conformation with ring pucker values near 0°. In contrast, the sugars of the DNA strand show significant variability.

The average RMS deviation of the OMe-DNA:RNA is approximately 1.2 Å from the starting A-form conformation; while the SMe-DNA:RNA shows a slightly higher deviation (approximately 1.8 Å) from the starting hybrid conformation. The SMe-DNA strand also shows a greater variance in RMS deviation, suggesting the S-methyl group may induce some structural fluctuations. The sugar puckers of the RNA complements maintain C3′-endo puckering throughout the simulation. As expected from the nucleoside calculations, however, significant differences are noted in the puckering of the OMe-DNA and SMe-DNA strands, with the former adopting C3′-endo, and the latter, C1′-exo/C2′-endo conformations.

An analysis of the helicoidal parameters for all three hybrid structures has also been performed to further characterize the duplex conformation. Three of the more important axis-basepair parameters that distinguish the different forms of the duplexes, X-displacement, propeller twist, and inclination, are reported in Table 10. Usually, an X-displacement near zero represents a B-form duplex; while a negative displacement, which is a direct measure of deviation of the helix from the helical axis, makes the structure appear more A-like in conformation. In A-form duplexes, these values typically vary from −4 Å to −5 Å. In comparing these values for all three hybrids, the SMe_DNA:RNA hybrid shows the most deviation from the A-form value, the OMe_DNA:RNA shows the least, and the DNA:RNA is intermediate. A similar trend is also evident when comparing the inclination and propeller twist values with ideal A-form parameters. These results are further supported by an analysis of the backbone and glycosidic torsion angles of the hybrid structures. Glycosidic angles (X) of A-form geometries, for example, are typically near −159° C. while B form values are near −102° C. These angles are found to be −162° C., −133° C., and −108° C. for the OMe-DNA, DNA, and SMe-DNA strands, respectively. All RNA complements adopt an X angle close to −160°. In addition, “crankshaft” transitions were also noted in the backbone torsions of the central UpU steps of the RNA strand in the SMe-DNA:RNA and DNA:RNA hybrids. Such transitions suggest some local conformational changes may occur to relieve a less favorable global conformation. Taken overall, the results indicate the amount of A-character decreases as OMe-DNA:RNA>DNA:RNA>SMe-DNA:RNA, with the latter two adopting more intermediate conformations when compared to A- and B-form geometries.

TABLE 10
Average helical parameters derived from the last 500 ps of simulation
time. (canonical A-and B-form values are given for comparison)

| Helicoidal | B-DNA | B-DNA | A-DNA | | | |
|---|---|---|---|---|---|---|
| Parameter | (x-ray) | (fibre) | (fibre) | DNA:RNA | OMe_DNA:RNA | SMe_DNA:RNA |
| X-disp | 1.2 | 0.0 | −5.3 | −4.5 | −5.4 | −3.5 |
| Inclination | −2.3 | 1.5 | 20.7 | 11.6 | 15.1 | 0.7 |
| Propeller | −16.4 | −13.3 | −7.5 | −12.7 | −15.8 | −10.3 |

Stability of C2′-modified DNA:RNA hybrids was determined. Although the overall stability of the DNA:RNA hybrids depends on several factors including sequence-dependencies and the purine content in the DNA or RNA strands DNA:RNA hybrids are usually less stable than RNA:RNA duplexes and, in some cases, even less stable than DNA:DNA duplexes. Available experimental data attributes the relatively lowered stability of DNA:RNA hybrids largely to its intermediate conformational nature between DNA:DNA (B-family) and RNA:RNA (A-family) duplexes. The overall thermodynamic stability of nucleic acid duplexes may originate from several factors including the conformation of backbone, base-pairing and stacking interactions. While it is difficult to ascertain the individual thermodynamic contributions to the overall stabilization of the duplex, it is reasonable to argue that the major factors that promote increased stability of hybrid duplexes are better stacking interactions and more favorable groove dimensions for hydration. The C2′-S-methyl substitution has been shown to destabilize the hybrid duplex. The notable differences in the rise values among the three hybrids may offer some explanation. While the 2′-S-methyl group has a strong influence on decreasing the base-stacking through high rise values (˜3.2 Å), the 2′-O-methyl group makes the overall structure more compact with a rise value that is equal to that of A-form duplexes (˜2.6 Å). Despite its overall A-like structural features, the SMe_DNA:RNA hybrid structure possesses an average rise value of 3.2 Å which is quite close to that of B-family duplexes. In fact, some local base-steps (CG steps) may be observed to have unusually high rise values (as high as 4.5 Å). Thus, the greater destabilization of 2′-S-methyl substituted DNA:RNA hybrids may be partly attributed to poor stacking interactions.

It has been postulated that RNase H binds to the minor groove of RNA:DNA hybrid complexes, requiring an intermediate minor groove width between ideal A- and B-form geometries to optimize interactions between the sugar phosphate backbone atoms and RNase H. A close inspection of the averaged structures for the hybrid duplexes using computer simulations reveals significant variation in the minor groove width dimensions as shown in Table 11. Whereas the O-methyl substitution leads to a slight expansion of the minor groove width when compared to the standard DNA:RNA complex, the S-methyl substitution leads to a general contraction (approximately 0.9 Å). These changes are most likely due to the preferred sugar puckering noted for the antisense strands which induce either A- or B-like single strand conformations. In addition to minor groove variations, the results also point to potential differences in the steric makeup of the minor groove. The O-methyl group points into the minor groove while the S-methyl is directed away towards the major groove. Essentially, the S-methyl group has flipped through the bases into the major groove as a consequence of C2′-endo puckering.

TABLE 11
Minor groove widths averaged over the last 500 ps of simulation time

| Phosphate | | | | DNA:RNA | RNA:RNA |
|---|---|---|---|---|---|
| Distance | DNA:RNA | OMe_DNA:RNA | SMe_DNA:RNA | (B-form) | (A-form) |
| P5-P20 | 15.27 | 16.82 | 13.73 | 14.19 | 17.32 |
| P6-P19 | 15.52 | 16.79 | 15.73 | 12.66 | 17.12 |
| P7-P18 | 15.19 | 16.40 | 14.08 | 11.10 | 16.60 |
| P8-P17 | 15.07 | 16.12 | 14.00 | 10.98 | 16.14 |
| P9-P16 | 15.29 | 16.25 | 14.98 | 11.65 | 16.93 |
| P10-P15 | 15.37 | 16.57 | 13.92 | 14.05 | 17.69 |

In addition to the modifications described above, the nucleotides of the chimeric oligomeric compounds of the invention can have a variety of other modification so long as these other modifications do not significantly detract from the properties described above. Thus, for nucleotides that are incorporated into oligonucleotides of the invention, these nucleotides can have sugar portions that correspond to naturally-occurring sugars or modified sugars. Representative modified sugars include carbocyclic or acyclic sugars, sugars having substituent groups at their 2′ position, sugars having substituent groups at their 3′ position, and sugars having substituents in place of one or more hydrogen atoms of the sugar. Other altered base moieties and altered sugar moieties are disclosed in U.S. Pat. No. 3,687,808 and PCT application PCT/US89/02323.

2′-Endo Regions

A number of different nucleosides can be used independently or exclusively to create one or more of the C2′-endo regions to prepare chimeric oligomeric compounds of the present invention. For the purpose of the present invention the terms 2′-endo and C2′-endo are meant to include O4′-endo and 2′-deoxy nucleosides. 2′-Deoxy nucleic acids prefer both C2′-endo sugar pucker and 04′-endo sugar, i.e., also known as Southern pucker, which is thought to impart a less stable B-form geometry (Sanger, W. (1984) Principles of Nucleic Acid Structure, Springer-Verlag, New York, N.Y. and Berger, et. al., Nucleic Acids Research, 1998, 26, 2473-2480). The 2′-deoxyribonucleoside is one suitable nucleoside for the 2′-endo regions but all manner of nucleosides known in the art that have a preference for 2′-endo sugar conformational geometry are amenable to the present invention. Such nucleosides include without limitation 2′-modified ribonucleosides such as for example: 2′-SCH₃, 2′-NH₂, 2′-NH(C₁-C₂ alkyl), 2′-N(C₁-C₂ alkyl)₂, 2′-CF₃, 2′=CH₂, 2′=CHF, 2′=CF₂, 2′-CH₃, 2′-C₂H₅, 2′-CH═CH₂ or 2′-C≡CH. Also amenable to the present invention are modified 2′-arabinonucleosides including without limitation: 2′-CN, 2′-F, 2′-Cl, 2′-Br, 2′-N₃ (azido), 2′-OH, 2′-O—CH₃ or 2′-dehydro-2′-CH₃.

Sugar modifications for the 2′-endo regions of the present invention include without limitation 2′-deoxy-2′-S-methyl, 2′-deoxy-2′-methyl, 2′-deoxy-2′-amino, 2′-deoxy-2′-mono or dialkyl substituted amino, 2′-deoxy-2′-fluoromethyl, 2′-deoxy-2′-difluoromethyl, 2′-deoxy-2′-trifluoromethyl, 2′-deoxy-2′-methylene, 2′-deoxy-2′-fluoromethylene, 2′-deoxy-2′-difluoromethylene, 2′-deoxy-2′-ethyl, 2′-deoxy-2′-ethylene and 2′-deoxy-2′-acetylene. These nucleotides can alternately be described as 2′-SCH₃ ribonucleotide, 2′-CH₃ ribonucleotide, 2′-NH₂ ribonucleotide 2′-NH(C₁-C₂ alkyl) ribonucleotide, 2′-N(C₁-C₂ alkyl)₂ ribonucleotide, 2′-CH₂F ribonucleotide, 2′-CHF₂ ribonucleotide, 2′-CF₃ ribonucleotide, 2′=CH₂ ribonucleotide, 2′=CHF ribonucleotide, 2′=CF₂ ribonucleotide, 2′-C₂H₅ ribonucleotide, 2′-CH═CH₂ ribonucleotide, 2′-CCH ribonucleotide. A further useful sugar modification is one having a ring located on the ribose ring in a cage-like structure including 3′,O,4′-C-methyleneribonucleotides. Such cage-like structures will physically fix the ribose ring in the desired conformation.

Additionally, sugar modifications for the 2′-endo regions of the present invention include without limitation are arabino nucleotides having 2′-deoxy-2′-cyano, 2′-deoxy-2′-fluoro, 2′-deoxy-2′-chloro, 2′-deoxy-2′-bromo, 2′-deoxy-2′-azido, 2′-methoxy and the unmodified arabino nucleotide (that includes a 2′-OH projecting upwards towards the base of the nucleotide). These arabino nucleotides can alternately be described as 2′-CN arabino nucleotide, 2′-F arabino nucleotide, 2′-Cl arabino nucleotide, 2′-Br arabino nucleotide, 2′-N₃ arabino nucleotide, 2′-O—CH₃ arabino nucleotide and arabino nucleotide.

Such nucleotides are linked together via phosphorothioate, phosphorodithioate, boranophosphate or phosphodiester linkages.

Internucleoside Linkages

Specific examples of ligands and/or target molecules useful in this invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

Modified internucleoside linkages containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, certain of which are commonly owned with this application, and each of which is herein incorporated by reference.

In other embodiments of the invention, chimeric oligomeric compounds include one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type internucleoside linkages are disclosed in the above referenced U.S. Pat. No. 5,489,677. Suitable amide internucleoside linkages are disclosed in the above referenced U.S. Pat. No. 5,602,240.

Modified internucleoside linkages that do not include a phosphorus atom therein include those formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Conjugate Groups

A further substitution that can be appended to the oligomeric compounds of the invention involves the linkage of one or more moieties or conjugates which enhance the activity, cellular distribution or cellular uptake of the resulting oligomeric compounds. In one embodiment, such modified oligomeric compounds are prepared by covalently attaching conjugate groups to functional groups such as hydroxyl or amino groups. Conjugate groups of the invention include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugates groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992 the entire disclosure of which is incorporated herein by reference. Conjugate moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

The ligand and/or target molecules of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999) which is incorporated herein by reference in its entirety.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, certain of which are commonly owned with the instant application, and each of which is herein incorporated by reference.

Oligomeric Compounds

In the context of the present invention, the term “oligomeric compound” refers to a polymeric structure capable of hybridizing a region of a nucleic acid molecule. This term includes oligonucleotides, oligonucleosides, oligonucleotide analogs, oligonucleotide mimetics and combinations of these. Oligomeric compounds routinely prepared linearly but can be joined or otherwise prepared to be circular and may also include branching. Oligomeric compounds can hybridized to form double stranded compounds which can be blunt ended or may include overhangs. In general, an oligomeric compound comprises a backbone of linked momeric subunits where each linked momeric subunit is directly or indirectly attached to a heterocyclic base moiety. The linkages joining the monomeric subunits, the sugar moieties or surrogates and the heterocyclic base moieties can be independently modified giving rise to a plurality of motifs for the resulting oligomeric compounds including hemimers, gapmers and chimeras.

As is known in the art, a nucleoside is a base-sugar combination. The base portion of the nucleoside is normally a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. The respective ends of this linear polymeric structure can be joined to form a circular structure by hybridization or by formation of a covalent bond, however, open linear structures are generally suitable. Within the oligonucleotide structure, the phosphate groups are commonly referred to as forming the internucleoside linkages of the oligonucleotide. The normal internucleoside linkage of RNA and DNA is a 3′ to 5′ phosphodiester linkage.

In the context of this invention, the term “oligonucleotide” refers to an oligomer or polymer of ribonucleic acid (RNA) or deoxyribonucleic acid (DNA). This term includes oligonucleotides composed of naturally-occurring nucleobases, sugars and covalent internucleoside linkages. The term “oligonucleotide analog” refers to oligonucleotides that have one or more non-naturally occurring portions which function in a similar manner to oligonulceotides. Such non-naturally occurring oligonucleotides are often favored over the naturally occurring forms because of desirable properties such as, for example, enhanced cellular uptake, enhanced affinity for nucleic acid target and increased stability in the presence of nucleases.

In the context of this invention, the term “oligonucleoside” refers to nucleosides that are joined by internucleoside linkages that do not have phosphorus atoms. Internucleoside linkages of this type include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, certain of which are commonly owned with this application, and each of which is herein incorporated by reference.

Further included in the present invention are oligomeric compounds such as antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these oligomeric compounds may be introduced in the form of single-stranded, double-stranded, circular or hairpin oligomeric compounds and may contain structural elements such as internal or terminal bulges or loops. Once introduced to a system, the oligomeric compounds of the invention may elicit the action of one or more enzymes or structural proteins to effect modification of the target nucleic acid.

One non-limiting example of such an enzyme is RNAse H, a cellular endonuclease which cleaves the RNA strand of an RNA:DNA duplex. It is known in the art that single-stranded antisense oligomeric compounds which are “DNA-like” or have DNA like regions elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While one form of antisense acting chimeric oligomeric compound is a single-stranded chimeric oligonucleotide, in many species the introduction of double-stranded structures, such as double-stranded RNA (dsRNA) molecules, has been shown to induce potent and specific antisense-mediated reduction of the function of a gene or its associated gene products. This phenomenon occurs in both plants and animals and is believed to have an evolutionary connection to viral defense and transposon silencing.

In addition to the modifications described above, the nucleosides of the oligomeric compounds of the invention can have a variety of other modifications so long as these other modifications either alone or in combination with other nucleosides enhance one or more of the desired properties described above. Thus, for nucleotides that are incorporated into oligonucleotides of the invention, these nucleotides can have sugar portions that correspond to naturally-occurring sugars or modified sugars. Representative modified sugars include carbocyclic or acyclic sugars, sugars having substituent groups at one or more of their 2′, 3′ or 4′ positions and sugars having substituents in place of one or more hydrogen atoms of the sugar. Additional nucleosides amenable to the present invention having altered base moieties and or altered sugar moieties are disclosed in U.S. Pat. No. 3,687,808 and PCT application PCT/US89/02323.

The oligomeric compounds in accordance with this invention comprise from about 10 to about 200 nucleobases (i.e. from about 10 to about 200 linked nucleosides). One of ordinary skill in the art will appreciate that the invention embodies oligomeric compounds of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 nucleobases in length, or any range therewithin.

In another embodiment, the oligomeric compounds of the invention are 15 to 100 nucleobases in length. One having ordinary skill in the art will appreciate that this embodies oligomeric compounds of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleobases in length, or any range therewithin.

In another embodiment, the oligomeric compounds of the invention are 15 to 50 nucleobases in length. One having ordinary skill in the art will appreciate that this embodies oligomeric compounds of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length, or any range therewithin.

In another embodiment, the oligomeric compounds of the invention are 15 to 30 nucleobases in length. One having ordinary skill in the art will appreciate that this embodies oligomeric compounds of 15, 16, 17, 18, 19, 20, 21, 22, 23, 24, 25, 26, 27, 28, 29, or 30 nucleobases in length, or any range therewithin.

In another embodiment, the oligomeric compounds of the invention are 17 to 25 nucleobases in length. One having ordinary skill in the art will appreciate that this embodies oligomeric compounds of 17, 18, 19, 20, 21, 22, 23, 24, or 25 nucleobases in length, or any range therewithin.

Oligomer Synthesis

Oligomerization of modified and unmodified nucleosides is performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA:Protein Interactions, Ed. Smith (1998), 1-36. Gallo et al., Tetrahedron (2001), 57, 5707-5713) synthesis as appropriate. In addition specific protocols for the synthesis of oligomeric compounds of the invention are illustrated in the examples below.

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The present invention is also useful for the preparation of oligomeric compounds incorporating at least one 2′-O-protected nucleoside. After incorporation and appropriate deprotection the 2′-O-protected nucleoside will be converted to a ribonucleoside at the position of incorporation. The number and position of the 2-ribonucleoside units in the final oligomeric compound can vary from one at any site or the strategy can be used to prepare up to a full 2′-OH modified oligomeric compound. All 2′-O-protecting groups amenable to the synthesis of oligomeric compounds are included in the present invention. In general a protected nucleoside is attached to a solid support by for example a succinate linker. Then the oligonucleotide is elongated by repeated cycles of deprotecting the 5′-terminal hydroxyl group, coupling of a further nucleoside unit, capping and oxidation (alternatively sulfurization). In a more frequently used method of synthesis the completed oligonucleotide is cleaved from the solid support with the removal of phosphate protecting groups and exocyclic amino protecting groups by treatment with an ammonia solution. Then a further deprotection step is normally required for the more specialized protecting groups used for the protection of 2′-hydroxyl groups which will give the fully deprotected oligonucleotide.

A large number of 2′-O-protecting groups have been used for the synthesis of oligoribonucleotides but over the years more effective groups have been discovered. The key to an effective 2′-O-protecting group is that it is capable of selectively being introduced at the 2′-O-position and that it can be removed easily after synthesis without the formation of unwanted side products. The protecting group also needs to be inert to the normal deprotecting, coupling, and capping steps required for oligoribonucleotide synthesis. Some of the protecting groups used initially for oligoribonucleotide synthesis included tetrahydropyran-1-yl and 4-methoxytetrahydropyran-4-yl. These two groups are not compatible with all 5′-O-protecting groups so modified versions were used with 5′-DMT groups such as 1-(2-fluorophenyl)-4-methoxypiperidin-4-yl (Fpmp). Reese has identified a number of piperidine derivatives (like Fpmp) that are useful in the synthesis of oligoribonucleotides including 1-[(chloro-4-methyl)phenyl]-4′-methoxypiperidin-4-yl (Reese et al., Tetrahedron Lett., 1986, (27), 2291). Another approach was to replace the standard 5′-DMT (dimethoxytrityl) group with protecting groups that were removed under non-acidic conditions such as levulinyl and 9-fluorenylmethoxycarbonyl. Such groups enable the use of acid labile 2′-protecting groups for oligoribonucleotide synthesis. Another more widely used protecting group initially used for the synthesis of oligoribonucleotides was the t-butyldimethylsilyl group (Ogilvie et al., Tetrahedron Lett., 1974, 2861; Hakimelahi et al., Tetrahedron Lett., 1981, (22), 2543; and Jones et al., J. Chem. Soc. Perkin I., 2762). The 2′-O-protecting groups can require special reagents for their removal such as for example the t-butyldimethylsilyl group is normally removed after all other cleaving/deprotecting steps by treatment of the oligomeric compound with tetrabutylammonium fluoride (TBAF).

One group of researchers examined a number of 2′-O-protecting groups (Pitsch, S., Chimia, 2001, (55), 320-324.) The group examined fluoride labile and photolabile protecting groups that are removed using moderate conditions. One photolabile group that was examined was the [2-(nitrobenzyl)oxy]methyl(nbm) protecting group (Schwartz et al., Bioorg. Med. Chem. Lett., 1992, (2), 1019.) Other groups examined included a number structurally related formaldehyde acetal-derived, 2′-O-protecting groups. Also prepared were a number of related protecting groups for preparing 2′-O-alkylated nucleoside phosphoramidites including 2′-O-[(triisopropylsilyl)oxy]methyl(2′-O—CH₂—O—Si(iPr)₃, TOM). One 2′-O-protecting group that was prepared to be used orthogonally to the TOM group was 2′-O-[(R)-1-(2-nitrophenyl)ethyloxy)methyl]((R)-mnbm).

Another strategy using a fluoride labile 5′-O-protecting group (non-acid labile) and an acid labile 2′-O-protecting group has been reported (Scaringe, Stephen A., Methods, 2001, (23) 206-217). A number of possible silyl ethers were examined for 5′-O-protection and a number of acetals and orthoesters were examined for 2′-O-protection. The protection scheme that gave the best results was 5′-O-silyl ether-2′-ACE (5′-O-bis(trimethylsiloxy)cyclododecyloxysilyl ether (DOD)-2′-O-bis(2-acetoxyethoxy)methyl (ACE). This approach uses a modified phosphoramidite synthesis approach in that some different reagents are required that are not routinely used for RNA/DNA synthesis.

Although a lot of research has focused on the synthesis of oligoribonucleotides the main RNA synthesis strategies that are presently being used commercially include 5′-O-DMT-2′-O-t-butyldimethylsilyl (TBDMS), 5′-O-DMT-2′-O-[1(2-fluorophenyl)-4-methoxypiperidin-4-yl](FPMP), 2′-O-[(triisopropylsilyl)oxy]methyl(2′-O—CH₂—O—Si(iPr)₃ (TOM), and the 5′-O-silyl ether-2′-ACE (5′-O-bis(trimethylsiloxy)cyclododecyloxysilyl ether (DOD)-2′-O-bis(2-acetoxyethoxy)methyl (ACE). A current list of some of the major companies currently offering RNA products include Pierce Nucleic Acid Technologies, Dharmacon Research Inc., Ameri Biotechnologies Inc., and Integrated DNA Technologies, Inc. One company, Princeton Separations, is marketing an RNA synthesis activator advertised to reduce coupling times especially with TOM and TBDMS chemistries. Such an activator would also be amenable to the present invention.

The primary groups being used for commercial RNA synthesis are:
TBDMS=5′-O-DMT-2′-O-t-butyldimethylsilyl;
TOM=2′-O-[(triisopropylsilyl)oxy]methyl;
DOD/ACE=(5′-O-bis(trimethylsiloxy)cyclododecyloxysilyl ether-2′-O-bis(2-acetoxyethoxy)methyl
FPMP=5′-O-DMT-2′-O-[1 (2-fluorophenyl)-4-methoxypiperidin-4-yl].

All of the aforementioned RNA synthesis strategies are amenable to the present invention. Strategies that would be a hybrid of the above e.g. using a 5′-protecting group from one strategy with a 2′-O-protecting from another strategy is also amenable to the present invention.

The preparation of ribonucleotides and oligomeric compounds having at least one ribonucleoside incorporated and all the possible configurations falling in between these two extremes are encompassed by the present invention. The corresponding oligomeric comounds can be hybridized to further oligomeric compounds including oligoribonucleotides having regions of complementarity to form double-stranded (duplexed) oligomeric compounds. Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The methods of preparing oligomeric compounds of the present invention can also be applied in the areas of drug discovery and target validation. The present invention comprehends the use of the oligomeric compounds and suitable targets identified herein in drug discovery efforts to elucidate relationships that exist between proteins and a disease state, phenotype, or condition. These methods include detecting or modulating a target peptide comprising contacting a sample, tissue, cell, or organism with the oligomeric compounds of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a particular disease, condition, or phenotype.

Effect of nucleoside modifications on RNAi activity is evaluated according to existing literature (Elbashir et al., Nature (2001), 411, 494-498; Nishikura et al., Cell (2001), 107, 415-416; and Bass et al., Cell (2000), 101, 235-238.)

Oligomer Mimetics (Oligonucleotide Mimics)

Another group of oligomeric compounds amenable to the present invention includes oligonucleotide mimetics. The term mimetic as it is applied to oligonucleotides is intended to include oligomeric compounds wherein only the furanose ring or both the furanose ring and the internucleotide linkage are replaced with novel groups, replacement of only the furanose ring is also referred to in the art as being a sugar surrogate. The heterocyclic base moiety or a modified heterocyclic base moiety is maintained for hybridization with an appropriate target nucleic acid. One such oligomeric compound, an oligonucleotide mimetic that has been shown to have excellent hybridization properties, is referred to as a peptide nucleic acid (PNA). In PNA oligomeric compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA oligomeric compounds include, but are not limited to, U.S. Pat. Nos. 5,539,082; 5,714,331; and 5,719,262, each of which is herein incorporated by reference. Further teaching of PNA oligomeric compounds can be found in Nielsen et al., Science, 1991, 254, 1497-1500.

One oligonucleotide mimetic that has been reported to have excellent hybridization properties, is peptide nucleic acids (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

PNA has been modified to incorporate numerous modifications since the basic PNA structure was first prepared. The basic structure is shown below: 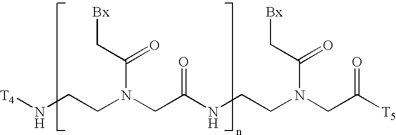
wherein
Bx is a heterocyclic base moiety;
T₄ is hydrogen, an amino protecting group, —C(O)R₅, substituted or unsubstituted C₁-C₁₂ alkyl, substituted or unsubstituted C₂-C₁₂ alkenyl, substituted or unsubstituted C₂-C₁₂ alkynyl, alkylsulfonyl, arylsulfonyl, a chemical functional group, a reporter group, a conjugate group, a D or L α-amino acid linked via the α-carboxyl group or optionally through the co-carboxyl group when the amino acid is aspartic acid or glutamic acid or a peptide derived from D, L or mixed D and L amino acids linked through a carboxyl group, wherein the substituent groups are selected from hydroxyl, amino, alkoxy, carboxy, benzyl, phenyl, nitro, thiol, thioalkoxy, halogen, alkyl, aryl, alkenyl and alkynyl;
T₅ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group or a conjugate group;
Z₁ is hydrogen, C₁-C₆ alkyl, or an amino protecting group;
Z₂ is hydrogen, C₁-C₆ alkyl, an amino protecting group, —C(═O)—(CH₂)ₙ-J-Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;
Z₃ is hydrogen, an amino protecting group, —C₁-C₆ alkyl, —C(═O)—CH₃, benzyl, benzoyl, or —(CH₂)ₙ—N(H)Z₁;
each J is O, S or NH;
R₅ is a carbonyl protecting group; and
n is from 2 to about 50.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups have been selected to give a non-ionic oligomeric compound. The non-ionic morpholino-based oligomeric compounds are less likely to have undesired interactions with cellular proteins. Morpholino-based oligomeric compounds are non-ionic mimics of oligonucleotides which are less likely to form undesired interactions with cellular proteins (Dwaine A. Braasch and David R. Corey, Biochemistry, 2002, 41(14), 4503-4510). Morpholino-based oligomeric compounds are disclosed in U.S. Pat. No. 5,034,506, issued Jul. 23, 1991. The morpholino class of oligomeric compounds have been prepared having a variety of different linking groups joining the monomeric subunits.

Morpholino nucleic acids have been prepared having a variety of different linking groups (L₂) joining the monomeric subunits. The basic formula is shown below: 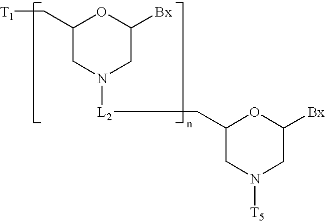
wherein
T₁ is hydroxyl or a protected hydroxyl;
T₅ is hydrogen or a phosphate or phosphate derivative;
L₂ is a linking group; and
n is from 2 to about 50.

A further class of oligonucleotide mimetic is referred to as cyclohexenyl nucleic acids (CeNA). The furanose ring normally present in an DNA/RNA molecule is replaced with a cyclohenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. coli RNase resulting in cleavage of the target RNA strand.

The general formula of CeNA is shown below: 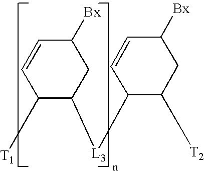
wherein
each Bx is a heterocyclic base moiety;
T₁ is hydroxyl or a protected hydroxyl; and
T2 is hydroxyl or a protected hydroxyl.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566) and would have the general formula:

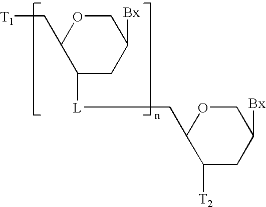

Another group of modifications includes nucleosides having sugar moieties that are bicyclic thereby locking the sugar conformational geometry. The most studied of these nucleosides having a bicyclic sugar moiety is locked nucleic acid or LNA. As can be seen in the structure below the 2′-O— has been linked via a methylene group to the 4′ carbon. This bridge attaches under the 3′ bonds forcing the sugar ring into a locked 3′-endo conformation geometry. The linkage can be a methylene (—CH₂—)ₙ group bridging the 2′ oxygen atom and the 4′ carbon atom wherein n is 1 for LNA. LNA and LNA analogs display very high duplex thermal stabilities with complementary DNA and RNA (Tm=+3 to +10 C), stability towards 3′-exonucleolytic degradation and good solubility properties.

An LNA analog that also has been looked at is ENA wherein an additional methylene group has been added to the bridge between the 2′ and the 2′ carbons (4′-CH₂—CH₂—O-2′, Kaneko et al., United States Patent Application Publication No.: U.S. 2002/0147332, Singh et al., Chem. Commun., 1998, 4, 455-456, also see Japanese Patent Application HEI-11-33863, Feb. 12, 1999).

In another publication a large genus of nucleosides having bicyclic sugar moieties is disclosed. The bridging group is variable as are the points of attachment (United States Patent Application Publication No.: U.S. 2002/0068708).

The basic structure of LNA showing the bicyclic ring system is shown below:

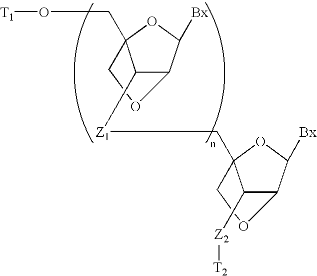

The conformations of LNAs determined by 2D NMR spectroscopy have shown that the locked orientation of the LNA nucleotides, both in single-stranded LNA and in duplexes, constrains the phosphate backbone in such a way as to introduce a higher population of the N-type conformation (Petersen et al., J. Mol. Recognit., 2000, 13, 44-53). These conformations are associated with improved stacking of the nucleobases (Wengel et al., Nucleosides Nucleotides, 1999, 18, 1365-1370).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands.

Novel types of LNA-oligomeric compounds, as well as the LNAs, are useful in a wide range of diagnostic and therapeutic applications. Among these are antisense applications, PCR applications, strand-displacement oligomers, substrates for nucleic acid polymerases and generally as nucleotide based drugs.

Potent and nontoxic antisense oligonucleotides containing LNAs have been described (Wahlestedt et al., Proc. Natl. Acad. Sci. U.S.A., 2000, 97, 5633-5638.) The authors have demonstrated that LNAs confer several desired properties to antisense agents. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished.

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

The first analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., PCT International Application WO 98-DK393 19980914). Furthermore, synthesis of 2′-amino-LNA, a novel conformationally restricted high-affinity oligonucleotide analog with a handle has been described in the art (Singh et al., J. Org. Chem., 1998, 63, 10035-10039). In addition, 2′-Amino- and 2′-methylamino-LNA's have been prepared and the thermal stability of their duplexes with complementary RNA and DNA strands has been previously reported.

One group has added an additional methlene group to the LNA 2′,4′-bridging group (e.g. 4′-CH₂—CH₂—O-2′ (ENA), Kaneko et al., United States Patent Application Publication No.: U.S. 2002/0147332, also see Japanese Patent Application HEI-11-33863, Feb. 12, 1999).

Further oligonucleotide mimetics have been prepared to incude bicyclic and tricyclic nucleoside analogs having the formulas (amidite monomers shown): 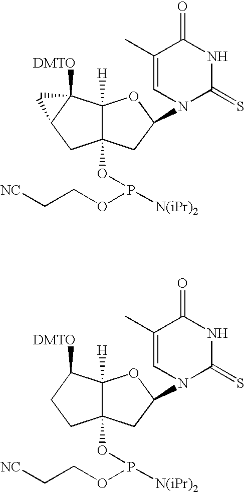
(see Steffens et al., Helv. Chim. Acta, 1997, 80, 2426-2439; Steffens et al., J. Am. Chem. Soc., 1999, 121, 3249-3255; and Renneberg et al., J. Am. Chem. Soc., 2002, 124, 5993-6002). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids incorporate a phosphorus group in a backbone the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

The general formula (for definitions of Markush variables see: U.S. Pat. Nos. 5,874,553 and 6,127,346 herein incorporated by reference in their entirety) is shown below.

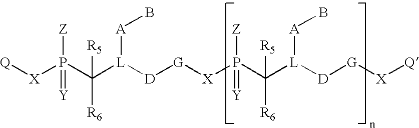

Another oligonucleotide mimetic has been reported wherein the furanosyl ring has been replaced by a cyclobutyl moiety.

Modified Sugars

Oligomeric compounds of the invention may also contain one or more substituted sugar moieties. Suitable oligomeric compounds comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₂ alkyl or C₂ to C₁₂ alkenyl and alkynyl. Particularly suitable are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise a sugar substituent group selected from: C₁ to C₁₂ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Another modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other sugar substituent groups include methoxy (—O—CH₃), aminopropoxy (—OCH₂CH₂CH₂NH₂), allyl(—CH₂—CH═CH₂), —O-allyl(—O—CH₂—CH═CH₂) and fluoro (F). 2′-Sugar substituent groups may be in the arabino (up) position or ribo (down) position. A suitable 2′-arabino modification is 2′-F. Similar modifications may also be made at other positions on the oligomeric compoiund, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, certain of which are commonly owned with the instant application, and each of which is herein incorporated by reference in its entirety.

Further representative sugar substituent groups include groups of formula Iₐ or IIₐ: wherein: 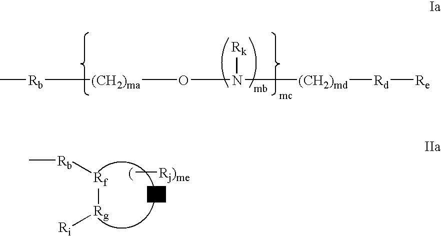
R_{b} is O, S or NH;
R_{d} is a single bond, O, S or C(═O);
Rₑ is C₁-C₁₂ alkyl, N(Rₖ)(Rₘ), N(Rₖ)(Rₙ), N═C(Rₚ)(R_{q}), N═C(Rₚ)(R_{f}) or has formula IIIₐ; 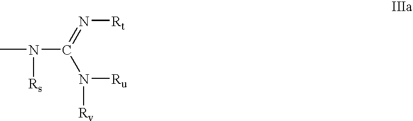
Rₚ and R_{q} are each independently hydrogen or C₁-C₁₂ alkyl;
Rᵣ is —Rₓ—R_{y};
each R₅, R₁, Rᵤ and Rᵥ is, independently, hydrogen, C(O)R_{w}, substituted or unsubstituted C₁-C₁₂ alkyl, substituted or unsubstituted C₂-C₁₂ alkenyl, substituted or unsubstituted C₂-C₁₂ alkynyl, alkylsulfonyl, arylsulfonyl, a chemical functional group or a conjugate group, wherein the substituent groups are selected from hydroxyl, amino, alkoxy, carboxy, benzyl, phenyl, nitro, thiol, thioalkoxy, halogen, alkyl, aryl, alkenyl and alkynyl;
or optionally, Rᵤ and Rᵥ, together form a phthalimido moiety with the nitrogen atom to which they are attached;
each R_{w} is, independently, substituted or unsubstituted C₁-C₁₂alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl or aryl;
Rₖ is hydrogen, a nitrogen protecting group or —Rₓ—R_{y};
Rₚ is hydrogen, a nitrogen protecting group or —Rₓ—R_{y};
Rₓ is a bond or a linking moiety;
R_{y} is a chemical functional group, a conjugate group or a solid support medium;
each Rₘ and Rₙ is, independently, H, a nitrogen protecting group, substituted or unsubstituted C₁-C₁₂ alkyl, substituted or unsubstituted C₂-C₁₂ alkenyl, substituted or unsubstituted C₂-C₁₂ alkynyl, wherein the substituent groups are selected from hydroxyl, amino, alkoxy, carboxy, benzyl, phenyl, nitro, thiol, thioalkoxy, halogen, alkyl, aryl, alkenyl, alkynyl; NH₃⁺, N(Rᵤ)(Rᵥ) guanidino and acyl where said acyl is an acid amide or an ester;
or Rₘ and Rₙ, together, are a nitrogen protecting group, are joined in a ring structure that optionally includes an additional heteroatom selected from N and O or are a chemical functional group;
R₁ is OR_{z}, SR_{z}, or N(R_{z})₂;
each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;
R_{f}, R_{g} and Rₕ comprise a ring system having from about 4 to about 7 carbon atoms or having from about 3 to about 6 carbon atoms and 1 or 2 heteroatoms wherein said heteroatoms are selected from oxygen, nitrogen and sulfur and wherein said ring system is aliphatic, unsaturated aliphatic, aromatic, or saturated or unsaturated heterocyclic;
Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ)ORₖ, halo, SRₖ or CN;
mₐ is 1 to about 10;
each mb is, independently, 0 or 1;
mc is 0 or an integer from 1 to 10;
md is an integer from 1 to 10;
me is from 0, 1 or 2; and
provided that when mc is 0, md is greater than 1.

Representative substituents groups of Formula I are disclosed in U.S. patent application Ser. No. 09/130,973, filed Aug. 7, 1998, entitled “Capped 2′-Oxyethoxy Oligonucleotides,” hereby incorporated by reference in its entirety.

Representative cyclic substituent groups of Formula II are disclosed in U.S. patent application Ser. No. 09/123,108, filed Jul. 27, 1998, entitled “RNA Targeted 2′-Oligomeric compounds that are Conformationally Preorganized,” hereby incorporated by reference in its entirety.

Particularly sugar substituent groups include O[(CH₂)ₙO]ₘCH₃, O(CH₂)ₙOCH₃, O(CH₂)ₙNH₂, O(CH₂)ₙCH₃, O(CH₂)ₙONH₂ and O(CH₂)ₙON[(CH₂)ₙCH₃)]₂, where n and m are from 1 to about 10.

Representative guanidino substituent groups that are shown in formula III and IV are disclosed in co-owned U.S. patent application Ser. No. 09/349,040, entitled “Functionalized Oligomers”, filed Jul. 7, 1999, hereby incorporated by reference in its entirety.

Representative acetamido substituent groups are disclosed in U.S. Pat. No. 6,147,200 which is hereby incorporated by reference in its entirety.

Representative dimethylaminoethyloxyethyl substituent groups are disclosed in International Patent Application PCT/US99/17895, entitled “2′-O-Dimethylaminoethyloxyethyl-Oligomeric compounds”, filed Aug. 6, 1999, hereby incorporated by reference in its entirety.

Modified Nucleobases/Naturally Occurring Nucleobases

Chimeric oligomeric compounds of the invention may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl(—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are suitable base substitutions, even more particularly when combined with 2′-O-methoxyethyl sugar modifications.

In one aspect of the present invention chimeric oligomeric compounds are prepared having polycyclic heterocyclic compounds in place of one or more heterocyclic base moieties. A number of tricyclic heterocyclic comounds have been previously reported. These compounds are routinely used in antisense applications to increase the binding properties of the modified strand to a target strand. The most studied modifications are 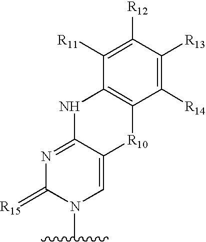
targeted to guanosines hence they have been termed G-clamps or cytidine analogs. Many of these polycyclic heterocyclic compounds have the general formula:

Representative cytosine analogs that make 3 hydrogen bonds with a guanosine in a second strand include 1,3-diazaphenoxazine-2-one (R₁₀=O, R₁₁-R₁₄=H) [Kurchavov, et al., Nucleosides and Nucleotides, 1997, 16, 1837-1846], 1,3-diazaphenothiazine-2-one (R₁₀=S, R₁₁-R₁₄=H), [Lin, K.-Y.; Jones, R. J.; Matteucci, M. J. Am. Chem. Soc. 1995, 117, 3873-3874] and 6,7,8,9-tetrafluoro-1,3-diazaphenoxazine-2-one (R₁₀=O, R₁₁-R₁₄=F) [Wang, J.; Lin, K.-Y., Matteucci, M. Tetrahedron Lett. 1998, 39, 8385-8388]. Incorporated into oligonucleotides these base modifications were shown to hybridize with complementary guanine and the latter was also shown to hybridize with adenine and to enhance helical thermal stability by extended stacking interactions (also see U.S. patent application entitled “Modified Peptide Nucleic Acids” filed May 24, 2002, Ser. No. 10/155,920; and U.S. patent application entitled “Nuclease Resistant Chimeric oligomeric compounds” filed May 24, 2002, Ser. No. 10/013,295, both of which are commonly owned with this application and are herein incorporated by reference in their entirety).

Further helix-stabilizing properties have been observed when a cytosine analog/substitute has an aminoethoxy moiety attached to the rigid 1,3-diazaphenoxazine-2-one scaffold (R₁₀=O, R₁₁=—O—(CH₂)₂—NH₂, R₁₂₋₁₄=H) [Lin, K.-Y.; Matteucci, M. J. Am. Chem. Soc. 1998, 120, 8531-8532]. Binding studies demonstrated that a single incorporation could enhance the binding affinity of a model oligonucleotide to its complementary target DNA or RNA with a ΔTₘ of up to 18° relative to 5-methyl cytosine (dC5^{me}′, which is the highest known affinity enhancement for a single modification, yet. On the other hand, the gain in helical stability does not compromise the specificity of the oligonucleotides. The Tₘ data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in United States patent Serial U.S. Pat. No. 6,028,183, which issued on May 22, 2000, and United States patent Serial U.S. Pat. No. 6,007,992, which issued on Dec. 28, 1999, the contents of both are commonly assigned with this application and are incorporated herein in their entirety.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity [Lin, K-Y; Matteucci, M. J. Am. Chem. Soc. 1998, 120, 8531-8532]. The activity enhancement was even more pronounced in case of G-clamp, as a single substitution was shown to significantly improve the in vitro potency of a 20mer 2′-deoxyphosphorothioate oligonucleotides [Flanagan, W. M.; Wolf, J. J.; Olson, P.; Grant, D.; Lin, K.-Y.; Wagner, R. W.; Matteucci, M. Proc. Natl. Acad. Sci. USA, 1999, 96, 3513-3518]. Nevertheless, to optimize oligonucleotide design and to better understand the impact of these heterocyclic modifications on the biological activity, it is important to evaluate their effect on the nuclease stability of the oligomers.

Further modified polycyclic heterocyclic compounds useful as heterocyclcic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and Unites States patent application Ser. No. 09/996,292 filed Nov. 28, 2001, certain of which are commonly owned with the instant application, and each of which is herein incorporated by reference.

Activated Phosphorus Groups

The ligands and/or target molecules of the present invention can have activated phosphorus compositions (e.g. compounds having activated phosphorus-containing substituent groups) in coupling reactions. As used herein, the term “activated phosphorus composition” includes monomers and oligomers that have an activated phosphorus-containing substituent group that is reactive with a hydroxyl group of another monomeric or oligomeric compound to form a phosphorus-containing internucleotide linkage. Such activated phosphorus groups contain activated phosphorus atoms in P^{III} valence state and are known in the art and include, but are not limited to, phosphoramidite, H-phosphonate, phosphate triesters and chiral auxiliaries. One synthetic solid phase synthesis utilizes phosphoramidites as activated phosphates. The phosphoramidites utilize P^{III} chemistry. The intermediate phosphite compounds are subsequently oxidized to the P^{V} state using known methods to yield, in another embodiment, phosphodiester or phosphorothioate internucleotide linkages. Additional activated phosphates and phosphites are disclosed in Tetrahedron Report Number 309 (Beaucage and Iyer, Tetrahedron, 1992, 48, 2223-2311).

Activated phosphorus groups are useful in the preparation of a wide range of oligomeric compounds including but not limited to oligonucleosides and oligonucleotides as well as oligonucleotides that have been modified or conjugated with other groups at the base or sugar or both. Also included are oligonucleotide mimetics including but not limited to peptide nucleic acids (PNA), morpholino nucleic acids, cyclohexenyl nucleic acids (CeNA), anhydrohexitol nucleic acids, locked nucleic acids (LNA and ENA), bicyclic and tricyclic nucleic acids, phosphonomonoester nucleic acids and cyclobutyl nucleic acids. A representative example of one type of oligomer synthesis that utilizes the coupling of an activated phosphorus group with a reactive hydroxyl group is the widely used phosphoramidite approach. A phosphoramidite synthon is reacted under appropriate conditions with a reactive hydroxyl group to form a phosphite linkage that is further oxidized to a phosphodiester or phosphorothioate linkage. This approach commonly utilizes nucleoside phosphoramidites of the formula: 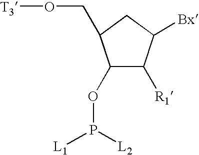
wherein
each Bx′ is an optionally protected heterocyclic base moiety;
each R_{1′} is, independently, H or an optionally protected sugar substituent group;
T_{3′} is H, a hydroxyl protecting group, a nucleoside, a nucleotide, an oligonucleoside or an oligonucleotide;
L₁ is N(R₁)R₂;
each R₂ and R₃ is, independently, C₁-C₁₂ straight or branched chain alkyl;
or R₂ and R₃ are joined together to form a 4- to 7-membered heterocyclic ring system including the nitrogen atom to which R₂ and R₃ are attached, wherein said ring system optionally includes at least one additional heteroatom selected from O, N and S;
L₂ is Pg-O—, Pg-S—, C₁-C₁₂ straight or branched chain alkyl, CH₃(CH₂)₀₋₁₀—O— or —NR₅R₆;
   Pg is a protecting/blocking group; and
each R₅ and R₆ is, independently, hydrogen, C₁-C₁₂ straight or branched chain alkyl, cycloalkyl or aryl;
or optionally, R₅ and R₆, together with the nitrogen atom to which they are attached form a cyclic moiety that may include an additional heteroatom selected from O, S and N; or
L₁ and L₂ together with the phosphorus atom to which L₁ and L₂ are attached form a chiral auxiliary.

Groups that are attached to the phosphorus atom of internucleotide linkages before and after oxidation (L₁ and L₂) can include nitrogen containing cyclic moieties such as morpholine. Such oxidized internucleoside linkages include a phosphoromorpholidothioate linkage (Wilk et al., Nucleosides and nucleotides, 1991, 10, 319-322). Further cyclic moieties amenable to the present invention include mono-, bi- or tricyclic ring moieties which may be substituted with groups such as oxo, acyl, alkoxy, alkoxycarbonyl, alkyl, alkenyl, alkynyl, amino, amido, azido, aryl, heteroaryl, carboxylic acid, cyano, guanidino, halo, haloalkyl, haloalkoxy, hydrazino, ODMT, alkylsulfonyl, nitro, sulfide, sulfone, sulfonamide, thiol and thioalkoxy. One bicyclic ring structure that includes nitrogen is phthalimido.

Unless otherwise defined herein, alkyl means C₁-C₁₂, C₁-C₈, or C₁-C₆, straight or (where possible) branched chain aliphatic hydrocarbyl.

Unless otherwise defined herein, heteroalkyl means C₁-C₁₂, C₁-C₈, or C₁-C₆, straight or (where possible) branched chain aliphatic hydrocarbyl containing at least one or about 1 to about 3 hetero atoms in the chain, including the terminal portion of the chain. Suitable heteroatoms include N, O and S.

Unless otherwise defined herein, cycloalkyl means C₃-C₁₂, C₃-C₈, or C₃-C₆, aliphatic hydrocarbyl ring.

Unless otherwise defined herein, alkenyl means C₂-C₁₂, C₂-C₈, or C₂-C₆ alkenyl, which may be straight or (where possible) branched hydrocarbyl moiety, which contains at least one carbon-carbon double bond.

Unless otherwise defined herein, alkynyl means C₂-C₁₂, C₂-C₈, or C₂-C₆ alkynyl, which may be straight or (where possible) branched hydrocarbyl moiety, which contains at least one carbon-carbon triple bond.

Unless otherwise defined herein, heterocycloalkyl means a ring moiety containing at least three ring members, at least one of which is carbon, and of which 1, 2 or three ring members are other than carbon. The number of carbon atoms can vary from 1 to about 12 or from 1 to about 6, and the total number of ring members can vary from three to about 15 or from about 3 to about 8. Suitable ring heteroatoms are N, O and S. Suitable heterocycloalkyl groups include morpholino, thiomorpholino, piperidinyl, piperazinyl, homopiperidinyl, homopiperazinyl, homomorpholino, homothiomorpholino, pyrrolodinyl, tetrahydrooxazolyl, tetrahydroimidazolyl, tetrahydrothiazolyl, tetrahydroisoxazolyl, tetrahydropyrrazolyl, furanyl, pyranyl, and tetrahydroisothiazolyl.

Unless otherwise defined herein, aryl means any hydrocarbon ring structure containing at least one aryl ring. Suitable aryl rings have about 6 to about 20 ring carbons. Suitable aryl rings also include phenyl, napthyl, anthracenyl, and phenanthrenyl.

Unless otherwise defined herein, hetaryl means a ring moiety containing at least one fully unsaturated ring, the ring consisting of carbon and non-carbon atoms. The ring system can contain about 1 to about 4 rings. The number of carbon atoms can vary from 1 to about 12 or from 1 to about 6, and the total number of ring members can vary from three to about 15 or from about 3 to about 8. Suitable ring heteroatoms are N, O and S. Suitable hetaryl moieties include, but are not limited to, pyrazolyl, thiophenyl, pyridyl, imidazolyl, tetrazolyl, pyridyl, pyrimidinyl, purinyl, quinazolinyl, quinoxalinyl, benzimidazolyl, benzothiophenyl, etc.

Unless otherwise defined herein, where a moiety is defined as a compound moiety, such as hetarylalkyl (hetaryl and alkyl), aralkyl (aryl and alkyl), etc., each of the sub-moieties is as defined herein.

Unless otherwise defined herein, an electron withdrawing group is a group, such as the cyano or isocyanato group that draws electronic charge away from the carbon to which it is attached. Other electron withdrawing groups of note include those whose electronegativities exceed that of carbon, for example halogen, nitro, or phenyl substituted in the ortho- or para-position with one or more cyano, isothiocyanato, nitro or halo groups.

Unless otherwise defined herein, the terms halogen and halo have their ordinary meanings. Suitable halo (halogen) substituents are Cl, Br, and I.

The aforementioned optional substituents are, unless otherwise herein defined, suitable substituents depending upon desired properties. Included are halogens (Cl, Br, I), alkyl, alkenyl, and alkynyl moieties, NO₂, NH₃ (substituted and unsubstituted), acid moieties (e.g. —CO₂H, —OSO₃H₂, etc.), heterocycloalkyl moieties, hetaryl moieties, aryl moieties, etc.

In all the preceding formulae, the squiggle (˜) indicates a bond to an oxygen or sulfur of the 5′-phosphate.

Phosphate protecting groups include those described in US patents No. U.S. Pat. No. 5,760,209, U.S. Pat. No. 5,614,621, U.S. Pat. No. 6,051,699, U.S. Pat. No. 6,020,475, U.S. Pat. No. 6,326,478, U.S. Pat. No. 6,169,177, U.S. Pat. No. 6,121,437, U.S. Pat. No. 6,465,628 each of which is expressly incorporated herein by reference in its entirety.

Hybridization

In the context of this invention, “hybridization” means the pairing of complementary strands of oligomeric compounds. In the present invention, one mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An oligomeric compound is specifically hybridizable when binding of the compound to the target nucleic acid interferes with the normal function of the target nucleic acid to cause a loss of activity, and there is a sufficient degree of complementarity to avoid non-specific binding of the antisense oligomeric compound to non-target nucleic acid sequences under conditions in which specific binding is desired, i.e., under physiological conditions in the case of in vivo assays or therapeutic treatment, and under conditions in which assays are performed in the case of in vitro assays.

In the present invention the phrase “stringent hybridization conditions” or “stringent conditions” refers to conditions under which an oligomeric compound of the invention will hybridize to its target sequence, but to a minimal number of other sequences. Stringent conditions are sequence-dependent and will vary with different circumstances and in the context of this invention, “stringent conditions” under which oligomeric compounds hybridize to a target sequence are determined by the nature and composition of the oligomeric compounds and the assays in which they are being investigated.

“Complementary,” as used herein, refers to the capacity for precise pairing of two nucleobases regardless of where the two are located. For example, if a nucleobase at a certain position of an oligomeric compound is capable of hydrogen bonding with a nucleobase at a certain position of a target nucleic acid, the target nucleic acid being a DNA, RNA, or oligonucleotide molecule, then the position of hydrogen bonding between the oligonucleotide and the target nucleic acid is considered to be a complementary position. The oligomeric compound and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of a chimeric oligomeric compound compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). The chimeric oligomeric compounds of the present invention can comprise at least 70%, at least 80%, at least 90%, at least 95%, or at least 99% sequence complementarity to a target region within the target nucleic acid sequence to which they are targeted. For example, a chimeric oligomeric compound in which 18 of 20 nucleobases are complementary to a target region, which specifically hybridizes, would represent 90 percent complementarity. In this example, the remaining noncomplementary nucleobases may be clustered or interspersed with complementary nucleobases and need not be contiguous to each other or to complementary nucleobases. As such, a chimeric oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of a chimeric oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

In some embodiments, the term “ligand” can refer to an agent that binds a target RNA. The agent may bind the target RNA when the target RNA is in a native or alternative conformation, or when it is partially or totally unfolded or denatured. According to the present invention, a ligand can be an agent that binds anywhere on the target RNA. Therefore, the ligands of the present invention encompass agents that in and of themselves may have no apparent biological function, beyond their ability to bind to the target RNA.

In some embodiments, the term “test ligand” refers to an agent, comprising a compound, molecule or complex, which is being tested for its ability to bind to a target RNA. Test ligands can be virtually any agent including, without limitation, metals, peptides, proteins, lipids, polysaccharides, small organic molecules, nucleotides (including non-naturally occurring ones) and combinations thereof. Small organic molecules have a molecular weight of more than 50 yet less than about 2,500 daltons or less than about 400 daltons. Test ligands may or may not be oligonucleotides. Complex mixtures of substances such as natural product extracts, which may include more than one test ligand, can also be tested, and the component that binds the target RNA can be purified from the mixture in a subsequent step.

Test ligands may be derived from large libraries of synthetic or natural compounds. For example, synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from Pan Labs (Bothell, Wash.) or MycoSearch (NC), or are readily producible. Additionally, natural and synthetically produced libraries and compounds are readily modified through conventional chemical, physical, and biochemical means. For example, the compounds may be modified to enhance efficacy, stability, pharmaceutical compatibility, and the like. For example, once a peptide ligand has been identified using the present invention, it may be modified in a variety of ways to enhance its stability, such as using an unnatural amino acid, such as a D-amino acid, particularly D-alanine, or by functionalizing the amino or carboxyl terminus, e.g., for the amino group, acylation or alkylation, and for the carboxyl group, esterification or amidification, or through constraint of the peptide chain in a cyclic form, or through other strategies well known to those skilled in the art.

In some embodiments, the term “target RNA” refers to a RNA sequence for which identification of a ligand or binding partner is desired. Target RNAs include, without limitation, sequences known or believed to be involved in the etiology of a given disease, condition or pathophysiological state, or in the regulation of physiological function. Target RNAs may be derived from any living organism, such as a vertebrate, particularly a mammal and even more particularly a human, or from a virus, bacterium, fungus, protozoan, parasite or bacteriophage. Target RNA may comprise wild type sequences, or, alternatively, mutant or variant sequences, including those with altered stability, activity, or other variant properties, or hybrid sequences to which heterologous sequences have been added. Furthermore, target RNA includes RNA that has been chemically modified, such as, for example, by conjugation of biotin, peptides, fluorescent molecules, and the like.

Target RNA sequences for use in the present invention are typically from about 5 to about 500, from about 30 to about 100, or from about 20 to about 30 nucleobases in length. Target RNAs may be isolated from native sources, or can be synthesized in vitro using conventional polymerase-directed cell-free systems such as those employing T7 RNA polymer.

In some embodiments, “test combination” refers to the combination of a test ligand and a target RNA. “Control combination” refers to the target RNA in the absence of a test ligand. As used herein, the “folded state” of a target RNA refers to a native or alternative conformation of the sequence in the absence of denaturing conditions. The folded state of an RNA encompasses both particular patterns of intramolecular base-pairing, as well as particular higher-order structures. Without wishing to be bound by theory, it is believed that certain target RNAs may achieve one of several alternative folded states depending upon experimental conditions (including buffer, temperature, presence of ligands, and the like) including binding interactions with one or more than one ligand.

In some embodiments, the “unfolded state” of a target RNA refers to a situation in which the RNA has been rendered partially or completely single-stranded relative to its folded state(s) or otherwise lacks elements of its structure that are present in its folded state. The term “unfolded state” encompasses partial or total denaturation and loss of structure.

As used herein, a “measurable change” in RNA conformation refers to a quantity that is empirically determined and that will vary depending upon the method used to monitor RNA conformation. The present invention encompasses any difference between the test and control combinations in any measurable physical parameter, where the difference is greater than expected due to random statistical variation.

The present invention provides high-throughput screening methods for identifying a ligand that binds a target RNA. If the target RNA to which the test ligand binds is associated with or causative of a disease or condition, the ligand may be useful for diagnosing, preventing or treating the disease or condition. A ligand identified by the present method can also be one that is used in a purification or separation method, such as a method that results in purification or separation of the target RNA from a mixture. The present invention also relates to ligands identified by the present method and their therapeutic uses (for diagnostic, preventive or treatment purposes) and uses in purification and separation methods.

A ligand for a target RNA can be identified by its ability to influence the extent or pattern of intramolecular folding or the rate of folding or unfolding of the target RNA. Experimental conditions are chosen so that the target RNA is subjected to unfolding or rearrangement. If the test ligand binds to the target RNA under these conditions, the relative amount of folded:unfolded target RNA, the relative amounts of one or another of multiple alternative folded states of the target RNA, or the rate of folding or unfolding of the target RNA in the presence of the test ligand will be different, i.e., higher or lower, than that observed in the absence of the test ligand. Thus, the present method encompasses incubating the target RNA in the presence and absence of a test ligand. This is followed by analysis of the absolute or relative amounts of folded vs. unfolded target RNA, the relative amounts of specific folded conformations, or of the rate of folding or unfolding of the target RNA.

One feature of the present invention is that it may detect any compound that binds to any region of the target RNA, not only to discrete regions that are intimately involved in a biological activity or function.

The test ligand can be combined with a target RNA, and the mixture maintained under appropriate conditions and for a sufficient time to allow binding of the test ligand to the target RNA. Experimental conditions are determined empirically for each target RNA. When testing multiple test ligands, incubation conditions are chosen so that most ligand:target RNA interactions would be expected to proceed to completion. In general, the test ligand is present in molar excess relative to the target RNA. As discussed in more detail below, the target RNA can be in a soluble form, or, alternatively, can be bound to a solid phase matrix.

The time necessary for binding of target RNA to ligand will vary depending on the test ligand, target RNA and other conditions used. In some cases, binding will occur instantaneously (e.g., essentially simultaneous with combination of test ligand and target RNA), while in others, the test ligand-target RNA combination is maintained for a longer time e.g. up to 12-16 hours, before binding is detected. When many test ligands are employed, an incubation time is chosen that is sufficient for most RNA:ligand interactions, typically about one hour. The appropriate time will be readily determined by one skilled in the art.

Other experimental conditions that are optimized for each RNA target include pH, reaction temperature, salt concentration and composition, divalent cation concentration and composition, amount of RNA, reducing agent concentration and composition, and the inclusion of non-specific protein and/or nucleic acid in the assay. One consideration when screening chemical or natural product libraries is the response of the assay to organic solvents (e.g., dimethyl sulfoxide, methanol or ethanol) commonly used to resuspend such materials. Accordingly, each RNA is tested in the presence of varying concentrations of each of these organic solvents. Finally, the assay may be particularly sensitive to certain types of compounds, such as intercalating agents, that commonly appear in chemical and especially natural product libraries. These compounds can often have potent, but non-specific, inhibitory activity. Some of the buffer components and their concentrations will be specifically chosen in anticipation of this problem. For example, bovine serum albumin will react with radicals and minimize surface adsorption. The addition of non-specific DNA or RNA may also be necessary to minimize the effect of nucleic acid-reactive molecules (such as, for example, intercalating agents) that would otherwise score as “hits” in the assay.

Binding of a test ligand to the target RNA is assessed by comparing the absolute amount of folded or unfolded target RNA in the absence and presence of test ligand, or, alternatively, by determining the ratio of folded:unfolded target RNA or change in the folded state of the target RNA, or the rate of target RNA folding or unfolding in the absence and presence of test ligand. If a test ligand binds the target RNA (i.e., if the test ligand is a ligand for the target RNA), there may be significantly more folded, and less unfolded, target RNA (and, thus, a higher ratio of folded to unfolded target RNA) than is present in the absence of a test ligand. Alternatively, binding of the test ligand may result in significantly less folded, and more unfolded, target RNA than is present in the absence of a test ligand. Another possibility is that binding of the test ligand changes the pattern or properties of alternative RNA folded structures. Similarly, binding of the test ligand may cause the rate of target RNA folding or unfolding to change significantly or may change the rate of acquisition of an alternative structure.

In either case, determination of the absolute amounts of folded and unfolded target RNA, the folded:unfolded ratio, or the rates of folding or unfolding, may be carried out using any method, including without limitation hybridization with complementary oligonucleotides, treatment with conformation-specific nucleases, binding to matrices specific for single-stranded or double-stranded nucleic acids, and fluorescence energy transfer between adjacent fluorescence probes. Other physico-chemical techniques may also be used, either alone or in conjunction with the above methods; these include without limitation measurements of circular dichroism, ultraviolet and fluorescence spectroscopy, and calorimetry. However, it will be recognized by those skilled in the art that each target RNA may have unique properties that make a particular detection method most suitable in a particular application.

The present invention may be practiced using any of a large number of detection methods well-known in the art. For example, an oligonucleotide (whether DNA or RNA) can be designed so that it will hybridize to a particular RNA target only when the RNA is in an unfolded conformation or to single-stranded regions in an otherwise folded conformation. In some embodiments, hybridization of an oligonucleotide to a target RNA is allowed to proceed in the absence and presence of test ligands (i.e., in control and test combinations, respectively), after which the extent of hybridization is measured using any of the methods well-known in the art. Typically, an increase or decrease in hybridization that is greater than that expected due to random statistical variation in the test vs. control combination indicates that the test ligand binds the target RNA. Other useful methods to measure the extent of folding of the target RNA include without limitation intramolecular fluorescence energy transfer, digestion with conformation-specific nucleases, binding to materials specific for either single-stranded or double-stranded nucleic acids (such as, nitrocellulose or hydroxylapatite), measurement of biophysical properties indicative of RNA folded structure (such as UV, Raman, or CD spectrum, intrinsic fluorescence, sedimentation rate, or viscosity), measurement of the stability of a folded RNA structure to heat and/or formamide denaturation (using methods such as, spectroscopy or nuclease susceptibility), and measurement of protein binding to adjacent reporter RNA. Examples of these methods are disclosed in the following articles: Kan et al., Eur. J. Biochem., 1987, 168, 635; Edy et al., Eur. J. Biochem., 1976, 61, 563; Yeh et al., J. Biol. Chem., 1988, 263, 18213; Clever et al., J. Virol., 1995, 69, 2101; and Vigne et al., J. Mol. Evol., 1977, 10, 77; Millar, Biochim. Biophy. Acta, 1969, 174, 32, (thermal melting, fluorescence polarization); and Zimmerman, Biochem. Z., 1966, 344, 386; and Dupont et al., Acad. Sci. Hebd. Seances Acad. Sci. D., 1968, 266, 2234 (viscosity).

Examples of RNA targets to which the present invention can be applied are shown in the following table:

| Area | RNA Targets |
|---|---|
| Antivirals | HBV epsilon sequence; HCV 5′ untranslated |
| | region; HIV packaging sequence, RRE, |
| | TAR; picornavirus internal translation enhancer |
| Antibacterials | RNAse P, tRNA, rRNA (16 S and 23 S), 4.5 S RNA |
| Antifungals | Similar RNA targets as for antibacterials |
| Rheumatoid | Alternative splicing of CD23 |
| Arthritis | |
| Cancer | Metastatic behavior is conferred by alternatively- |
| | spliced CD44; mRNAs encode proto-oncogenes |
| CNS | RNA editing alters glutamate receptor-B, changing |
| | calcium ion permeability |
| | Neurofibromatosis RNA editing introduces stop codon at |
| | 5′ end of NFl type I |
| | GAP-related domain to inactivate NFl epigenetically |
| Cardiovascular | RNA editing influences amount of ApoB-100, |
| | strongly associated with atherosclerosis |

The present invention also provides novel chimeric oligomeric compounds comprising regions that alternate between 3′-endo sugar conformational geometry (3′-endo regions) and 2′-endo/O4′-endo sugar conformational geometry (2′-endo regions). Each of the alternating regions comprise from 1 to about 5 nucleosides. The chimeric oligomeric compounds can start (5′-end) or end (3′-end) with either of the 2 regions and can have from about 5 to about 20 separate regions. One or more of the nucleosides of the chimeric oligomeric compound can further comprise a conjugate group. Chimeric oligomeric compounds can have the formula:
T₁-(3′-endo region)-[(2′-endo region)-(3′-endo region)]ₙ-T₂
wherein n is at least two and each T₁ and T₂ is independently an optional conjugate group.

Each of the regions can range from 1 to about 5 nucleosides in length allowing for a plurality of motifs for oligonucleotides having the same length. Such as for example a chimeric oligomeric compound of the present invention having a length of 20 base pairs (bp) would include such motifs as 3-3-2-4-2-3-3, 3-4-1-4-1-4-3 and 4-3-1-4-1-3-4 where each motif has the same number and orientation of regions (bold and underlined numbers are 3′-endo regions, unbold and not underlined numbers are 2′-endo regions and the number corresponding to each region representing the number of base pairs for that particular region).

A plurality of motifs for the chimeric oligomeric compounds of the present invention have been prepared and have shown activity in a plurality of assays against various targets. In addition to in vitro assays some posative data has also been obtained by in vivo assay. A list of motifs that have been prepared is shown below. This list is meant to be representative and not limiting. Refer to the figures for activity data for the various targets.

Motifs

#=number of 3′-endo nucleosides in the region

#=number of 2′-deoxy ribonucleotides in the region

| # bp's | Regions | Motif |
|---|---|---|
| 20 mer | 5 | 3-5-4-5-3 |
| 20 mer | 5 | 3-6-1-7-3 |
| 20 mer | 5 | 3-7-1-6-3 |
| 20 mer | 7 | 3-3-2-4-2-3-3 |
| 20 mer | 7 | 3-4-1-4-1-4-3 |
| 20 mer | 7 | 4-3-1-4-1-3-4 |
| 18 mer | 9 | 2-2-1-3-1-2-1-3-3 |
| 20 mer | 9 | 3-2-1-3-1-3-1-3-3 |
| 20 mer | 9 | 3-2-1-3-1-2-1-3-4 |
| 18 mer | 9 | 3-3-1-2-1-3-1-2-2 |
| 20 mer | 9 | 3-3-1-2-1-3-1-3-3 |
| 20 mer | 9 | 3-3-1-3-1-2-1-2-4 |
| 20 mer | 9 | 3-3-1-3-1-2-1-3-3 |
| 20 mer | 9 | 5-2-1-2-1-2-1-1-5 |
| 20 mer | 11 | 3-2-2-1-2-1-2-1-1-2-3 |
| 20 mer | 11 | 3-1-3-1-2-1-2-1-2-1-3 |
| 20 mer | 11 | 3-1-2-1-2-1-2-1-2-1-4 |
| 20 mer | 11 | 3-2-1-2-1-2-1-2-1-2-3 |
| 20 mer | 11 | 3-2-1-2-1-3-1-2-1-1-3 |
| 20 mer | 15 | 1-1-1-1-1-1-1-1-1-1-1-1-1-1-1-1-1-1-2 |
| 20 mer | 15 | 2-1-1-2-1-1-1-1-1-1-1-1-1-3-2 |
| 20 mer | 15 | 3-1-1-1-1-1-1-1-1-1-1-1-1-1-4 |
| 20 mer | 19 | 1-1-1-1-1-1-1-1-1-1-1-1-1-1-1-1-1-1-2 |

Chimeric Oligomeric Compounds/Synthetic Sequences

A representative list of chimeric oligomeric compounds prepared to sequence specific targets includes:

Target/SEQ ID NO:/ISIS NO:/Sequence 5′-3′

```
PTEN/3/334270/CTGCTAGCCTCTGGATTTGA (b.END cells)
                            3-6-1-7-3 (5)
PTEN/3/334271/CTGCTAGCCTCTGGATTTGA (b.END cells)
                            3-7-1-6-3 (5)
PTEN/3/334272/CTGCTAGCCTCTGGATTTGA (b.END cells)
                            3-4-1-4-1-4-3 (7)
PTEN/3/334273/CTGCTAGCCTCTGGATTTGA (b.END cells)
                            3-3-1-2-1-3-1-3-3 (9)
PTEN/3/334274/CTGCTAGCCTCTGGATTTGA (b.END cells)
                            3-3-1-3-1-2-1-3-3 (9)
PTEN/3/334275/CTGCTAGCCTCTGGATTTGA (b.END cells)
                            3-2-1-2-1-2-1-2-1-2-3 (11)
PTEN/3/116847/CTGCTAGCCTCTGGATTTGA (b.END cells)
                            5-10-5 gapmer control
PTEN/3/334269/CTGCTAGCCTCTGGATTTGA (b.END cells)
                            3-14-3 gapmer control
PTEN/3/334276/CCTTCCCTGAAGGTTCCTCC (b.END cells)
                            full 2′-MOE
PTEN/4/141923/CCTTCCCTGAAGGTTCCTCC (b.END cells)
                            5-10-5 gapmer mismatch control
PTEN/5/284346/CTTCTAGCCTCTGGATTGGA (b.END cells)
                            5-10-5 gapmer mismatch control
PTEN/6/129686/CGTTATTAACCTCCGTTGAA (b.END cells)
                            5-10-5 gapmer mismatch control
PTEN/3/337217/CTGCTAGCCTCTGGATTTGA (b.END cells)
                            3-2-2-1-2-1-2-1-1-2-3 (11)
PTEN/3/337218/CTGCTAGCCTCTGGATTTGA (b.END cells)
                            3-1-3-1-2-1-2-1-2-1-3 (11)
PTEN/3/337219/CTGCTAGCCTCTGGATTTGA (b.END cells)
                            3-1-2-1-2-1-2-1-2-1-4 (11)
PTEN/3/337220/CTGCTAGCCTCTGGATTTGA (b.END cells)
                            3-1-1-1-1-1-1-1-1-1-1-1-1-1-4 (15)
PTEN/3/337221/CTGCTAGCCTCTGGATTTGA (b.END cells)
                            1-1-1-1-1-1-1-1-1-1-1-1-1-1-1-1-1-
                            1-2 (19)
```

Note: all internucleoside linkages are phosphorothioate, bold underlined nucleosides are 2′-MOE (2′-O—CH₂CH₂—O—CH₃) and all C nucleosides are 5-methyl-C nucleosides.

Target/SEQ ID NO:/ISIS NO:/Sequence 5′-3′

```
Murine Glugagon Receptor/
7/300861/GAGCTTTGCCTTCTTGCCAT
                  3-2-1-3-1-3-1-3-3 (9)
Murine Glugagon Receptor/
7/180475/GAGCTTTGCCTTCTTGCCAT
                  5-10-5 (Gapmer control)
Murine Glugagon Receptor/
8/298682/GCGATTTCCCGTTTTCACCT
                  5-10-5 (Mismatch gapmer control)
Murine Glugagon Receptor/
7/298683/GAGCTTTGCCTTCTTGCCAT
                  (Full 2′-MOE)
Murine Glugagon Receptor/
9/29848/NNNNNNNNNNNNNNNNNNNN
                5-10-5 (Randomer control)
```

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| Fatty Acid | 10 | 304170 | TTGTTGACGTTGTACTCAGC |
| Synthase (Rat) | | | 3-2-1-2-1-2-1-2-1-2-3 (11) |
| Fatty Acid | 10 | 256899 | TTGTTGACGTTGTACTCAGC |
| Synthase (Rat) | | | 5-10-5 (gapmer control) |
| Fatty Acid | 11 | 319237 | TTGTTAACGGTGTTCTCAGC |
| Synthase (Rat) | | | 5-10-5 (3 bp mismatch gapmer) |
| Fatty Acid | 12 | 319238 | TTTGTAACGGTGTTCACTGA |
| Synthase (Rat) | | | 5-10-5 (8 bp mismatch gapmer) |
| Fatty Acid | 13 | 319239 | TTCATGAACTGCACAGAGGT |
| Synthase (Rat) | | | 3-2-1-2-1-2-1-2-1-2-3 (11) |
| Fatty Acid | 13 | 148529 | TTCATGAACTGCACAGAGGT |
| Synthase (Rat) | | | 5-10-5 (gapmer control) |
| Fatty Acid | 14 | 319240 | TACTTGACCTACAGAGTGGA |
| Synthase (Rat) | | | 5-10-5 (7 bp mismatch gapmer) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| Murine Survivin | 15 | 299228 | TGTGCTATTCTGTGAATT |
| Human | | | 2-2-1-3-1-2-1-3-3 (9) |
| Murine Survivin | 15 | 299229 | TGTGCTATTCTGTGAATT |
| Human | | | 3-3-1-2-1-3-1-2-2 (9) |
| Murine Survivin | 16 | 299230 | AACCACACTTACCCATGGGC |
| Mouse | | | 3-2-1-3-1-2-1-3-4 (9) |
| Murine Survivin | 17 | 299231 | GTTGGTCTCCTTTGCCTGGA |
| Mouse | | | 3-2-1-3-1-2-1-3-4 (9) |
| Murine Survivin | 17 | 114905 | GTTGGTCTCCTTTGCCTGGA |
| Mouse | | | 5-10-5 (gapmer control) |
| Murine Survivin | 18 | 303767 | GTTCGTGTTCTCTGGCTCGA |
| Mouse | | | 5-10-5 (6 bp gapmer mismatch) |
| Murine Survivin | 19 | 299232 | TGTCATCGGGTTCCCAGCCT |
| Mouse | | | 3-2-1-3-1-2-1-3-4 (9) |

Note: all internucleoside linkages are phosphorothioate, bold underlined nucleoside are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| Murine DGAT 2 | 20 | 310515 | TCCATTTATTAGTCTAGGAA |
| primary hepatocytes | | | 3-2-1-2-1-2-1-2-1-2-3 (11) |
| Murine DGAT 2 | 20 | 217376 | TCCATTTATTAGTCTAGGAA |
| primary hepatocytes | | | 5-10-5 (gapmer control) |
| Murine DGAT 2 | 21 | 310514 | ATGCACTCAAGAACTCGGTA |
| primary hepatocytes | | | 3-2-1-2-1-2-1-2-1-2-3 (11) |
| Murine DGAT 2 | 21 | 337205 | ATGCACTCAAGAACTCGGTA |
| primary hepatocytes | | | 3-16-3 (gapmer) |
| Murine DGAT 2 | 21 | 337206 | ATGCACTCAAGAACTCGGTA |
| primary hepatocytes | | | 3-6-1-7-3 (5) |
| Murine DGAT 2 | 21 | 337207 | ATGCACTCAAGAACTCGGTA |
| primary hepatocytes | | | 3-7-1-6-3 (5) |
| Murine DGAT 2 | 21 | 337208 | ATGCACTCAAGAACTCGGTA |
| primary hepatocytes | | | 3-4-1-4-1-4-3 (7) |
| Murine DGAT 2 | 21 | 337209 | ATGCACTCAAGAACTCGGTA |
| primary hepatocytes | | | 3-3-1-2-1-3-1-3-3 (9) |
| Murine DGAT 2 | 21 | 337210 | ATGCACTCAAGAACTCGGTA |
| primary hepatocytes | | | 3-3-1-3-1-2-1-3-3 (9) |
| Murine DGAT 2 | 21 | 337211 | ATGCACTCAAGAACTCGGTA |
| primary hepatocytes | | | full deoxy |
| Murine DGAT 2 | 21 | 337212 | ATGCACTCAAGAACTCGGTA |
| primary hepatocytes | | | 3-2-2-1-2-1-2-1-1-2-3 (11) |
| Murine DGAT 2 | 21 | 337213 | ATGCACTCAAGAACTCGGTA |
| primary hepatocytes | | | 3-1-3-1-2-1-2-1-2-1-3 (11) |
| Murine DGAT 2 | 21 | 337214 | ATGCACTCAAGAACTCGGTA |
| primary hepatocytes | | | 3-1-2-1-2-1-2-1-2-1-4 (11) |
| Murine DGAT 2 | 21 | 337215 | ATGCACTCAAGAACTCGGTA |
| primary hepatocytes | | | 3-1-1-1-1-1-1-1-1-1-1-1-1-1-4 (15) |
| Murine DGAT 2 | 21 | 337216 | ATGCACTCAAGAACTCGGTA |
| primary hepatocytes | | | 1-(1-1-)₈-1-2 (19) |
| Murine DGAT 2 | 21 | 337222 | ATGCACTCAAGAACTCGGTA |
| primary hepatocytes | | | full 2′-MOE |
| Murine DGAT 2 | 21 | 217352 | ATGCACTCAAGAACTCGGTA |
| primary hepatocytes | | | 5-10-5 (gapmer control) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| Murine HSD1 | 22 | 310516 | TTCTCATGATGAGGTGTACC |
| primary mouse hepatocytes | | | 3-2-1-2-1-2-1-2-1-2-3 (11) |
| Murine HSD1 | 22 | 146038 | TTCTCATGATGAGGTGTACC |
| primary mouse hepatocytes | | | 5-10-5 (gapmer control) |
| Murine HSD1 | 23 | 141923 | CCTTCCCTGAACCTTCCTCC |
| primary mouse hepatocytes | | | 5-10-5 (gapmer mismatch control) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| Murine HSD1 | 24 | 310517 | TGTTGCAAGAATTTCTCATG |
| primary rat hepatocytes | | | 3-2-1-2-1-2-1-2-1-2-3 (11) |
| Murine HSD1 | 24 | 146039 | TGTTGCAAGAATTTCTCATG |
| primary rat hepatocytes | | | 5-10-5 (gapmer control) |
| Murine HSD1 | 23 | 141923 | CCTTCCCTGAACCTTCCTCC |
| primary rat hepatocytes | | | 5-10-5 (gapmer mismatch control) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| Murine SCD1 | 25 | 312844 | GTGTTTCTGAGAACTTGTGG |
| primary mouse hepatocytes | | | 3-2-1-2-1-2-1-2-1-2-3 (11) |
| Murine SCD1 | 25 | 244504 | GTGTTTCTGAGAACTTGTGG |
| primary mouse hepatocytes | | | 5-10-5 (gapmer control) |
| Murine SCD1 | 26 | 244541 | ATGTCCAGTTTTCCGCCCTT |
| primary mouse hepatocytes | | | 5-10-5 (gapmer mismatch control) |
| Murine SCD1 | 23 | 141923 | CCTTCCCTGAACCTTCCTCC |
| primary mouse hepatocytes | | | 5-10-5 (gapmer mismatch control) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| ACS1 | 27 | 319162 | TCAAGGACTGCTGATCTTCG |
| primary mouse hepatocytes | | | 3-2-1-2-1-2-1-2-1-2-3 (11) |
| ACS1 | 27 | 291452 | TCAAGGACTGCTGATCTTCG |
| primary mouse hepatocytes | | | 5-10-5 (gapmer control) |
| ACS1 | 23 | 141923 | CCTTCCCTGAAGGTTCCTCC |
| primary mouse hepatocytes | | | 5-10-5 (gapmer control) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| NaDC1 | 28 | 312837 | GGACCTGTAGCCATAGCCAA |
| primary mouse hepatocytes | | | 3-2-1-2-1-2-1-2-1-2-3 (11) |
| NaDC1 | 28 | 249375 | GGACCTGTAGCCATAGCCAA |
| primary mouse hepatocytes | | | 5-10-5 (gapmer) |
| NaDC1 | 29 | 249386 | CTCGTGAACCAGAGCACCAC |
| primary mouse hepatocytes | | | 5-10-5 (gapmer) |
| NaDC1 | 23 | 141923 | CCTTCCCTGAAGGTTCCTCC |
| primary mouse hepatocytes | | | 5-10-5 (gapmer control) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| CD86 mRNA | 30 | 306058 | TCAAGTTTCTCTGTGCCCAA |
| MHS cells | | | 3-2-1-2-1-3-1-2-1-1-3 (11) |
| CD86 mRNA | 30 | 121874 | TCAAGTTTCTCTGTGCCCAA |
| MHS cells | | | 5-10-5 (gapmer) |
| CD86 mRNA | 31 | 131906 | TCAAGTCCTTCCACACCCAA |
| MHS cells | | | 5-10-5 (7 bp mismatch gapmer) |
| CD86 mRNA | 32 | 121875 | GTTCCTGTCAAAGCTCGTGC |
| MHS cells | | | 5-10-5 (gapmer) |
| CD86 mRNA | 33 | 131903 | TCAAGTTTCTCCGTGCCCAA |
| MHS cells | | | 5-10-5 (gapmer) |
| CD86 mRNA | 34 | 131904 | TCAAGTCTCTCCGCGCCCAA |
| MHS cells | | | 5-10-5 (mismatch, gapmer) |
| CDB6 mRNA | 35 | 131905 | TCAAGTCTTTCCACGCCCAA |
| MHS cells | | | 5-10-5 (mismatch, gapmer) |

Note: all internucleoside linkages are phosphorothioate, bold underlined nucleosides are 2′-MOE (2′-O—CH₂CH₂—O—CH₃) and all C nucleosides are 5-methyl-C nucleosides.

Murine Glucagon Receptor SAR study

| | | | |
|---|---|---|---|
| SEQ ID NO: | ISIS NO: | Sequence 5′-3′ | motif |

| | | | |
|---|---|---|---|
| 3 | 180475 | GAGCTTTGCCTTCTTGCCAT | 5-10-5 (gapmer) |
| 3 | 300861 | GAGCTTTGCCTTCTTGCCAT | 3-2-1-3-1-3-1-3-3 (9) |
| 3 | 332864 | GAGCTTTGCCTTCTTGCCAT | 4-3-1-4-1-3-4 (7) |
| 3 | 332865 | GAGCTTTGCCTTCTTGCCAT | 3-2-1-2-1-2-1-2-1-2-3 (11) |
| 3 | 332866 | GAGCTTTGCCTTCTTGCCAT | 3-5-4-5-3 (5) |
| 3 | 332867 | GAGCTTTGCCTTCTTGCCAT | 3-14-3 (gapmer) |
| 3 | 332868 | GAGCTTTGCCTTCTTGCCAT | 3-3-2-4-2-3-3 (7) |
| 3 | 332869 | GAGCTTTGCCTTCTTGCCAT | 3-1-1-1-1-1-1-1-1-1-1-1-1-1-4 (15) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| TRADD | 28 | 338177 | CGCTCGTACTCGTAGGCCAG |
| | | | 3-5-4-5-3 (5) |
| TRADD | 28 | 338179 | CGCTCGTACTCGTAGGCCAG |
| | | | 3-3-2-4-2-3-3 (7) |
| TRADD | 28 | 338175 | CGCTCGTACTCGTAGGCCAG |
| | | | 4-3-1-4-1-3-4 (7) |
| TRADD | 28 | 338176 | CGCTCGTACTCGTAGGCCAG |
| | | | 3-2-1-2-1-2-1-2-1-2-3 (11) |
| TRADD | 28 | 338180 | CGCTCGTACTCGTAGGCCAG |
| | | | 3-1-1-1-1-1-1-1-1-1-1-1-1-1-4 (15) |
| TRADD | 28 | 338173 | CGCTCGTACTCGTAGGCCAG |
| | | | 5-10-5 (gapmer) |
| TRADD | 28 | 338178 | CGCTCGTACTCGTAGGCCAG |
| | | | 3-14-3 (gapmer) |
| TRADD | 28 | 338174 | CGCTCGTACTCGTAGGCCAG |
| | | | full MOE |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| Toxicity study | 29 | 194563 | CCTGCTCCCTCTAATGCTGC |
| Serum transaminases in Lean Mice | | | 2-1-1-2-1-1-1-1-1-1-1-1-1-3-2 (15) |
| Toxicity study | 29 | 129605 | CCTGCTCCCTCTAATGCTGC |
| Serum transaminases in Lean Mice | | | 5-10-5 (gapmer) |
| Toxicity study | 30 | 118929 | TCTACAGTCATGCTGAGTAA |
| Serum transaminases in Lean Mice | | | 5-10-5 (gapmer) |
| Toxicity study | 31 | 148548 | TTGTTGACATTGTACTCGGC |
| Serum transaminases in Lean Mice | | | 5-10-5 (gapmer) |
| Murine Glugagon | 5 | 29848 | NNNNNNNNNNNNNNNNNNNN |
| Receptor | | | 5-10-5 (Randomer control) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| Toxicity study | 29 | 199042 | CCTGCTCCCTCTAATGCTGC |
| Serum transaminases in Lean Mice | | | 5-2-1-2-1-2-1-1-5 (9) |
| Toxicity study | 29 | 129605 | CCTGCTCCCTCTAATGCTGC |
| Serum transaminases in Lean Mice | | | 5-10-5 (gapmer) |
| Toxicity study | 29 | 189525 | CCTGCTCCCTCTAATGCTGC |
| Serum transaminases in Lean Mice | | | 5-10-5 (gapmer-no 5-MeC's) |
| Toxicity study | 29 | 199041 | CCTGCTCCCTCTAATGCTGC |
| Serum transaminases in Lean Mice | | | full 2′-MOE |
| Toxicity study | 29 | 199043 | CCTGCTCCCTCTAATGCTGC |
| Serum transaminases in Lean Mice | | | full 2′-deoxy |
| Toxicity study | 29 | 199044 | CCTGCTCCCTCTAATGCTGC |
| Serum transaminases in Lean Mice | | | 5-10-5 (underlined = 2′-O-methyl) |
| Toxicity study | 29 | 199046 | C*C*T*G*CTCCCTCTAATG*C*T*G*C |
| Serum transaminases in Lean Mice | | | 5-10-5 (gapmer, * = P=O linkage) |
| Toxicity study | 32 | 199047 | CCTGATCCCTCTAATGATGC |
| Serum transaminases in Lean Mice | | | 5-10-5 (mismatch, gapmer) |
| Toxicity study | 33 | 199048 | CCTGCTCACTCTAATGCTGC |
| Serum transaminases in Lean Mice | | | 5-10-5 (mismatch, gapmer) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| Fatty Acid | 31 | 304171 | TTGTTGACATTGTACTCGGC |
| Synthase (Murine) | | | 3-2-1-2-1-2-2-1-1-2-3 (11) |
| Fatty Acid | 31 | 148548 | TTGTTGACATTGTACTCGGC |
| Synthase (Murine) | | | 5-10-5 (gapmer control) |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| GCGR (Human) | 37 | 332522 | GCACTTTGTGGTGCCAAGGC |
| | | | 3-2-1-2-1-2-1-2-1-2-3 (11) |
| GCGR (Human) | 37 | 310457 | GCACTTTGTGGTGCCAAGGC |
| | | | 5-10-5 (gapmer control) |
| GCGR (Human) | 37 | 332520 | GCACTTTGTGGTGCCAAGGC |
| | | | Uniform 2′-MOE |
| GCGR (Human) | 37 | 332521 | GCACTTTGTGGTGCCAAGGC |
| | | | Uniform deoxy |
| GCGR (Human) | 38 | 333024 | CAGGAGATGTTGGCCGTGGT |
| | | | 3-2-1-2-1-2-1-2-1-2-3 (11) |
| GCGR (Human) | 38 | 310456 | CAGGAGATGTTGGCCGTGGT |
| | | | 5-10-5 (gapmer control) |
| GCGR (Human) | 38 | 333022 | CAGGAGATGTTGGCCGTGGT |
| | | | Uniform 2′-MOE |
| GCGR (Human) | 38 | 333023 | CAGGAGATGTTGGCCGTGGT |
| | | | Uniform deoxy |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| GCGR | 7 | 300861 | GAGCTTTGCCTTCTTGCCAT |
| db/db mice (fasted plasma levels) | | | 3-2-1-3-1-3-1-3-3 (9) |
| GCGR | 7 | 180475 | GAGCTTTGCCTTCTTGCCAT |
| db/db mice (fasted plasma levels) | | | 5-10-5 (Gapmer control) |
| PTEN | 3 | 116847 | CTGCTAGCCTCTGGATTTGA |
| db/db mice (fasted plasma levels) | | | 5-10-5 gapmer control |
| PTEN | 23 | 141923 | CCTTCCCTGAAGGTTCCTCC |
| db/db mice (fasted plasma levels) | | | 5-10-5 gapmer mismatch control |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| GCGR | 7 | 300861 | GAGCTTTGCCTTCTTGCCAT |
| db/db mice (liver mRNA) | | | 3-2-1-3-1-3-1-3-3 (9) |
| GCGR | 7 | 180475 | GAGCTTTGCCTTCTTGCCAT |
| db/db mice (liver mRNA) | | | 5-10-5 (Gapmer control) |
| PTEN | 3 | 116847 | CTGCTAGCCTCTGGATTTGA |
| db/db mice (liver mRNA) | | | 5-10-5 gapmer control |
| PTEN | 22 | 141923 | CCTTCCCTGAAGGTTCCTCC |
| db/db mice (liver mRNA) | | | 5-10-5 gapmer mismatch control |

Note: all internucleoside linkages are phosphorothioate, bold underlined nucleosides are 2′-MOE (2′-O—CH₂CH₂—O—CH₃) and all C nucleosides are 5-methyl-C nucleosides.

| | | | |
|---|---|---|---|
| Target | SEQ ID NO: | ISIS NO: | Sequence 5′-3′ |

| | | | |
|---|---|---|---|
| PTP-1B mRNA | 39 | 113715 | GCTCCTTCCACTGATCCTGC |
| | | | 3-3-1-3-1-2-1-2-4 (9) |
| PTP-1B mRNA | 39 | 166659 | GCTCCTTCCACTGATCCTGC |
| primary mouse hepatocytes | | | 5-10-5 (gapmer) |
| PTP-1B mRNA | 39 | 283586 | GCTCCTTCCACTGATCCTGC |
| primary mouse hepatocytes | | | full 2′-MOE |

Note: all internucleoside linkages are phosphorothioate, bold underlined nucleosides are 2′-MOE (2′-O—CH₂CH₂—O—CH₃) and all C nucleosides are 5-methyl-C nucleosides.

The chimeric oligomeric compounds of the present invention can be targeted to nucleic acid targets in a sequence dependent manner. A suitable nucleic acid target is messenger RNA. More specifically, chimeric oligomeric compounds of the invention will modulate gene expression by hybridizing to a nucleic acid target resulting in loss of normal function of the target nucleic acid. As used herein, the term “target nucleic acid” or “nucleic acid target” is used for convenience to encompass any nucleic acid capable of being targeted including without limitation DNA, RNA (including pre-mRNA and mRNA or portions thereof) transcribed from such DNA, and also cDNA derived from such RNA. In one embodiment of the invention the target nucleic acid is a messenger RNA. The inhibition of the target is typically based upon hydrogen bonding-based hybridization of the chimeric oligomeric compound strands or segments such that at least one strand or segment is cleaved, degraded, or otherwise rendered inoperable. In this regard, it is presently suitable to target specific nucleic acid molecules and their functions for such inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the desired form of modulation of expression and mRNA is often a desired target nucleic acid.

In one aspect, the present invention is directed to chimeric oligomeric compounds that are prepared having enhanced activity against nucleic acid targets. As used herein the phrase “enhanced activity” can indicate upregulation or downregulation of a system. A target and a mechanism for its modulation is determined. An oligonucleotide is selected having an effective length and sequence that is complementary to a portion of the target sequence. The selected sequence is divided into regions and the nucleosides of each region is modified to enhance the desired properties of the respective region. Consideration is also given to the 5′ and 3′-termini as there are often advantageous modifications that can be made to one or more of the terminal nucleosides. Further modifications are also considered such as internucleoside linkages, conjugate groups, substitute sugars or bases, substitution of one or more nucleosides with nucleoside mimetics and any other modification that can enhance the selected sequence for its intended target.

“Targeting” an oligomeric compound to a particular nucleic acid molecule, in the context of this invention, can be a multistep process. The process usually begins with the identification of a target nucleic acid whose levels, expression or function is to be modulated. This target nucleic acid may be, for example, a mRNA transcribed from a cellular gene whose expression is associated with a particular disorder or disease state, a small non-coding RNA or its precursor, or a nucleic acid molecule from an infectious agent.

The targeting process usually also includes determination of at least one target region, segment, or site within the target nucleic acid for the interaction to occur such that the desired effect, e.g., modulation of levels, expression or function, will result. Within the context of the present invention, the term “region” is defined as a portion of the target nucleic acid having at least one identifiable sequence, structure, function, or characteristic. Within regions of target nucleic acids are segments. “Segments” are defined as smaller or sub-portions of regions within a target nucleic acid. “Sites,” as used in the present invention, are defined as specific positions within a target nucleic acid. The terms region, segment, and site can also be used to describe an oligomeric compound of the invention such as for example a gapped oligomeric compound having three separate segments.

Targets of the present invention include both coding and non-coding nucleic acid sequences. For coding nucleic acid sequences, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon.” A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding a nucleic acid target, regardless of the sequence(s) of such codons. It is also known in the art that a translation termination codon (or “stop codon”) of a gene may have one of three sequences, i.e., 5′-UAA, 5′-UAG and 5′-UGA (the corresponding DNA sequences are 5′-TAA, 5′-TAG and 5′-TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the oligomeric compounds of the present invention.

The open reading frame (ORF) or “coding region,” which is known in the art to refer to the region between the translation initiation codon and the translation termination codon, is also a region which may be targeted effectively. Within the context of the present invention, a further suitable region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also suitable to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also target sites. mRNA transcripts produced via the process of splicing of two (or more) mRNAs from different gene sources are known as “fusion transcripts.” It is also known that introns can be effectively targeted using oligomeric compounds targeted to, precursor molecules for example, pre-mRNA.

It is also known in the art that alternative RNA transcripts can be produced from the same genomic region of DNA. These alternative transcripts are generally known as “variants.” More specifically, “pre-mRNA variants” are transcripts produced from the same genomic DNA that differ from other transcripts produced from the same genomic DNA in either their start or stop position and contain both intronic and exonic sequences.

Upon excision of one or more exon or intron regions, or portions thereof, during splicing, pre-mRNA variants produce smaller “mRNA variants.” Consequently, mRNA variants are processed pre-mRNA variants and each unique pre-mRNA variant must always produce a unique mRNA variant as a result of splicing. These mRNA variants are also known as “alternative splice variants.” If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also target nucleic acids.

Certain non-coding RNA genes are known to produce functional RNA molecules with important roles in diverse cellular processes. Such non-translated, non-coding RNA molecules can include ribosomal RNAs, tRNAs, snRNAs, snoRNAs, tncRNAs, rasiRNAs, short hairpin RNAs (shRNAs), short temporal RNAs (stRNAs), short hairpin RNAs (shRNAs), siRNAs, miRNAs and smnRNAs. These non-coding RNA genes and their products are also suitable targets of the compounds of the invention. Such cellular processes include transcriptional regulation, translational regulation, developmental timing, viral surveillance, immunity, chromosome maintenance, ribosomal structure and function, gene imprinting, subcellular compartmentalization, pre-mRNA splicing, and guidance of RNA modifications. RNA-mediated processes are now also believed to direct heterochromatin formation, genome rearrangements, cellular differentiation and DNA elimination.

A total of 201 different expressed RNA sequences potentially encoding novel small non-messenger species (smnRNAs) has been identified from mouse brain cDNA libraries. Based on sequence and structural motifs, several of these have been assigned to the snoRNA class of nucleolar localized molecules known to act as guide RNAs for rRNA modification, whereas others are predicted to direct modification within the U2, U4, or U6 small nuclear RNAs (snRNAs). Some of these newly identified smnRNAs remained unclassified and have no identified RNA targets. It was suggested that some of these RNA species may have novel functions previously unknown for snoRNAs, namely the regulation of gene expression by binding to and/or modifying mRNAs or their precursors via their antisense elements (Huttenhofer et al., Embo J., 2001, 20, 2943-2953). Therefore, these smnRNAs are also suitable targets for the compounds of the present invention.

The locations on the target nucleic acid to which compounds and compositions of the invention hybridize are herein referred to as “suitable target segments.” As used herein the term “suitable target segment” is defined as at least an 8-nucleobase portion of a target region to which oligomeric compound is targeted.

Once one or more targets, target regions, segments or sites have been identified, oligomeric compounds are designed to be sufficiently complementary to the target, i.e., hybridize sufficiently well and with sufficient specificity, to give the desired effect. The desired effect may include, but is not limited to modulation of the levels, expression or function of the target.

In accordance with the present invention, a series of single stranded oligomeric compounds can be designed to target or mimic one or more specific small non-coding RNAs. These oligomeric compounds can be of a specified length, for example from 8 to 80, 12 to 50, 13 to 80, 15 to 30, 70 to 450, 110 to 430, 110 to 280, 50 to 110, 60 to 80, 15 to 49, 17 to 25 or 19 to 23 nucleotides long and have one or more modifications.

In accordance with one embodiment of the invention, a series of double-stranded oligomeric compounds (duplexes) comprising, as the antisense strand, the single-stranded oligomeric compounds of the present invention, and the fully or partially complementary sense strand, can be designed to modulate the levels, expression or function of one or more small non-coding RNAs or small non-coding RNA targets. One or both termini of the duplex strands may be modified by the addition of one or more natural or modified nucleobases to form an overhang. The sense strand of the duplex may be designed and synthesized as the complement of the antisense strand and may also contain modifications or additions to either terminus. For example, in one embodiment, both strands of the duplex would be complementary over the central region of the duplex, each having overhangs at one or both termini.

For the purposes of this invention, the combination of an antisense strand and a sense strand, each of which can be of a specified length, for example from 8 to 80, 12 to 50, 13 to 80, 15 to 30, 15 to 49, 17 to 25 or 19 to 23 subunits long, is identified as a complementary pair of oligomeric compounds. This complementary pair of oligonucleotides can include additional nucleotides on either of their 5′ or 3′ ends. They can include other molecules or molecular structures on their 3′ or 5′ ends, such as a phosphate group on the 5′ end, or non-nucleic acid moieties conjugated to either terminus of either strand or both strands. One group of compounds of the invention includes a phosphate group on the 5′ end of the antisense strand compound. Other compounds also include a phosphate group on the 5′ end of the sense strand compound. Some compounds include additional nucleotides such as a two base overhang on the 3′ end as well as those lacking overhangs.

For example, a complementary pair of oligomeric compounds may comprise an antisense strand oligomeric compound having the sequence CGAGAGGCGGACGGGACCG (SEQ ID NO:40), having a two-nucleobase overhang of deoxythymidine (dT) and its complement sense strand. This complementary pair of oligomeric compounds would have the following structure:

| | | |
|---|---|---|
| cgagaggcggacgggaccgTT | Antisense Strand | (SEQ ID NO:41) |
| \|\|\|\|\|\|\|\|\|\|\|\|\|\|\|\|\|\|\| | | |
| TTgctctccgcctgccctggc | Complement Sense Strand | (SEQ ID NO:42) |

In some embodiments, a single-stranded oligomeric compound may be designed comprising the antisense portion as a first region and the sense portion as a second region. The first and second regions can be linked together by either a nucleotide linker (a string of one or more nucleotides that are linked together in a sequence) or by a non-nucleotide linker region or by a combination of both a nucleotide and non-nucleotide structure. In any of these structures, the oligomeric compound, when folded back on itself, would form at least a partially complementary structure at least between a portion of the first region, the antisense portion, and a portion of the second region, the sense portion.

The desired RNA strand(s) of the duplex can be synthesized by methods disclosed herein or purchased from various RNA synthesis companies such as for example Dharmacon Research Inc., (Lafayette, Colo.) (see also the section on RNA synthesis below). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of the buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA compound is 20 uM. This solution can be stored frozen (−20° C.) and freeze-thawed up to 5 times.

Once prepared, the desired synthetic duplexs are evaluated for their ability to modulate target expression. When cells reach 80% confluency, they are treated with synthetic duplexs comprising at least one oligomeric compound of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired dsRNA compound at a final concentration of 200 nM. After 5 hours of treatment, the medium is replaced with fresh medium. Cells are harvested 16 hours after treatment, at which time RNA is isolated and target reduction measured by RT-PCR.

In a further embodiment, the “suitable target segments” identified herein may be employed in a screen for additional oligomeric compounds that modulate the expression of a target. “Modulators” are those oligomeric compounds that decrease or increase the expression of a nucleic acid molecule encoding a target and which comprise at least an 8-nucleobase portion which is complementary to a suitable target segment. The screening method comprises the steps of contacting a suitable target segment of a nucleic acid molecule encoding a target with one or more candidate modulators, and selecting for one or more candidate modulators which decrease or increase the expression of a nucleic acid molecule encoding a target. Once it is shown that the candidate modulator or modulators are capable of modulating (e.g. either decreasing or increasing) the expression of a nucleic acid molecule encoding a target, the modulator may then be employed in further investigative studies of the function of a target, or for use as a research, diagnostic, or therapeutic agent in accordance with the present invention.

The suitable target segments of the present invention may also be combined with their respective complementary chimeric oligomeric compounds of the present invention to form stabilized double-stranded (duplexed) oligonucleotides.

The suitable target segments of the present invention may also be combined with their respective complementary chimeric oligomeric compounds of the present invention to form stabilized double-stranded (duplexed) oligonucleotides. Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The oligomeric compounds of the present invention can also be applied in the areas of drug discovery and target validation. The present invention comprehends the use of the oligomeric compounds and targets identified herein in drug discovery efforts to elucidate relationships that exist between proteins and a disease state, phenotype, or condition. These methods include detecting or modulating a target peptide comprising contacting a sample, tissue, cell, or organism with the oligomeric compounds of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a particular disease, condition, or phenotype.

Effect of nucleoside modifications on RNAi activity is evaluated according to existing literature (Elbashir et al., Nature (2001), 411, 494-498; Nishikura et al., Cell (2001), 107, 415-416; and Bass et al., Cell (2000), 101, 235-238.).

The oligomeric compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the oligomeric compounds of the present invention, either alone or in combination with other oligomeric compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more chimeric oligomeric compounds are compared to control cells or tissues not treated with chimeric oligomeric compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds and or oligomeric compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The oligomeric compounds of the invention are useful for research and diagnostics, because these oligomeric compounds hybridize to nucleic acids encoding proteins. For example, oligonucleotides that are shown to hybridize with such efficiency and under such conditions as disclosed herein as to be effective protein inhibitors will also be effective primers or probes under conditions favoring gene amplification or detection, respectively. These primers and probes are useful in methods requiring the specific detection of nucleic acid molecules encoding proteins and in the amplification of the nucleic acid molecules for detection or for use in further studies. Hybridization of the chimeric oligomeric compounds, particularly the primers and probes, of the invention with a nucleic acid can be detected by means known in the art. Such means may include conjugation of an enzyme to the oligonucleotide, radiolabelling of the oligonucleotide or any other suitable detection means. Kits using such detection means for detecting the level of selected proteins in a sample may also be prepared.

The specificity and sensitivity of antisense is also harnessed by those of skill in the art for therapeutic uses. Antisense oligomeric compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense oligomeric compounds can be useful therapeutic modalities that can be configured to be useful in treatment regimes for the treatment of cells, tissues and animals, especially humans.

For therapeutics, an animal, such as a human, suspected of having a disease or disorder which can be treated by modulating the expression of a selected protein is treated by administering chimeric oligomeric compounds in accordance with this invention. For example, in one non-limiting embodiment, the methods comprise the step of administering to the animal in need of treatment, a therapeutically effective amount of a protein inhibitor. The protein inhibitors of the present invention effectively inhibit the activity of the protein or inhibit the expression of the protein. In one embodiment, the activity or expression of a protein in an animal can be inhibited by about 10% or more, by about 20% or more, by about 30% or more, by about 40% or more, by about 50% or more, by about 60% or more, by about 70% or more, by about 80% or more, by about 90% or more, by about 95% or more, or by about 99% or more.

For example, the reduction of the expression of a protein may be measured in serum, adipose tissue, liver or any other body fluid, tissue or organ of the animal. The cells contained within the fluids, tissues or organs being analyzed can contain a nucleic acid molecule encoding a protein and/or the protein itself.

The oligomeric compounds and compositions of the invention can be utilized in pharmaceutical compositions by adding an effective amount of the compound or composition to a suitable pharmaceutically acceptable diluent or carrier. Use of the oligomeric compounds and methods of the invention may also be useful prophylactically.

The oligomeric compounds and compositions of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative U.S. patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The oligomeric compounds and compositions of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the oligomeric compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligomeric compounds of the invention can be prepared as SATE ((S-acetyl-2-thioethyl) phosphate) derivatives according to the methods disclosed in WO 93/24510 to Gosselin et al., published Dec. 9, 1993 or in WO 94/26764 and U.S. Pat. No. 5,770,713 to Imbach et al. Larger oligomeric compounds that are processed to supply, as cleavage products, compounds capable of modulating the function or expression of small non-coding RNAs or their downstream targets are also considered prodrugs.

The term “pharmaceutically acceptable salts” refers to physiologically and pharmaceutically acceptable salts of the compounds and compositions of the invention: i.e., salts that retain the desired biological activity of the parent compound and do not impart undesired toxicological effects thereto. Suitable examples include, but are not limited to, sodium and postassium salts. For oligonucleotides, examples of pharmaceutically acceptable salts and their uses are further described in U.S. Pat. No. 6,287,860, which is incorporated herein in its entirety.

The present invention also includes pharmaceutical compositions and formulations that include the oligomeric compounds and compositions of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Oligomeric compounds may be formulated for delivery in vivo in an acceptable dosage form, e.g. as parenteral or non-parenteral formulations. Parenteral formulations include intravenous (IV), subcutaneous (SC), intraperitoneal (IP), intravitreal and intramuscular (IM) formulations, as well as formulations for delivery via pulmonary inhalation, intranasal administration, topical administration, etc. Non-parenteral formulations include formulations for delivery via the alimentary canal, e.g. oral administration, rectal administration, intrajejunal instillation, etc. Rectal administration includes administration as an enema or a suppository. Oral administration includes administration as a capsule, a gel capsule, a pill, an elixir, etc.

In some embodiments, an oligomeric compound can be administered to a subject via an oral route of administration. The subject may be an animal or a human (man). An animal subject may be a mammal, such as a mouse, a rat, a dog, a guinea pig, a monkey, a non-human primate, a cat or a pig. Non-human primates include monkeys and chimpanzees. A suitable animal subject may be an experimental animal, such as a mouse, rat, mouse, a rat, a dog, a monkey, a non-human primate, a cat or a pig.

In some embodiments, the subject may be a human. In certain embodiments, the subject may be a human patient. In certain embodiments, the subject may be in need of modulation of expression of one or more genes as discussed in more detail herein. In some particular embodiments, the subject may be in need of inhibition of expression of one or more genes as discussed in more detail herein. In particular embodiments, the subject may be in need of modulation, i.e. inhibition or enhancement, of a nucleic acid target in order to obtain therapeutic indications discussed in more detail herein.

In some embodiments, non-parenteral (e.g. oral) oligomeric compound formulations according to the present invention result in enhanced bioavailability of the compound. In this context, the term “bioavailability” refers to a measurement of that portion of an administered drug which reaches the circulatory system (e.g. blood, especially blood plasma) when a particular mode of administration is used to deliver the drug. Enhanced bioavailability refers to a particular mode of administration's ability to deliver oligonucleotide to the peripheral blood plasma of a subject relative to another mode of administration. For example, when a non-parenteral mode of administration (e.g. an oral mode) is used to introduce the drug into a subject, the bioavailability for that mode of administration may be compared to a different mode of administration, e.g. an IV mode of administration. In some embodiments, the area under a compound's blood plasma concentration curve (AUC₀) after non-parenteral (e.g. oral, rectal, intrajejunal) administration may be divided by the area under the drug's plasma concentration curve after intravenous (i.v.) administration (AUCᵢᵥ) to provide a dimensionless quotient (relative bioavailability, RB) that represents the fraction of compound absorbed via the non-parenteral route as compared to the IV route. A composition's bioavailability is said to be enhanced in comparison to another composition's bioavailability when the first composition's relative bioavailability (RB₁) is greater than the second composition's relative bioavailability (RB₂).

In general, bioavailability correlates with therapeutic efficacy when a compound's therapeutic efficacy is related to the blood concentration achieved, even if the drug's ultimate site of action is intracellular (van Berge-Henegouwen et al., Gastroenterol., 1977, 73, 300). Bioavailability studies have been used to determine the degree of intestinal absorption of a drug by measuring the change in peripheral blood levels of the drug after an oral dose (DiSanto, Chapter 76 In: Remington's Pharmaceutical Sciences, 18th Ed., Gennaro, ed., Mack Publishing Co., Easton, Pa., 1990, pages 1451-1458).

In general, an oral composition's bioavailability is said to be “enhanced” when its relative bioavailability is greater than the bioavailability of a composition substantially consisting of pure oligonucleotide, i.e. oligonucleotide in the absence of a penetration enhancer.

Organ bioavailability refers to the concentration of compound in an organ. Organ bioavailability may be measured in test subjects by a number of means, such as by whole-body radiography. Organ bioavailability may be modified, e.g. enhanced, by one or more modifications to the oligomeric compound, by use of one or more carrier compounds or excipients. In general, an increase in bioavailability will result in an increase in organ bioavailability.

Oral oligomeric compound compositions according to the present invention may comprise one or more “mucosal penetration enhancers,” also known as “absorption enhancers” or simply as “penetration enhancers.” Accordingly, some embodiments of the invention comprise at least one oligomeric compound in combination with at least one penetration enhancer. In general, a penetration enhancer is a substance that facilitates the transport of a drug across mucous membrane(s) associated with the desired mode of administration, e.g. intestinal epithelial membranes. Accordingly it is desirable to select one or more penetration enhancers that facilitate the uptake of one or more oligomeric compounds, without interfering with the activity of the compounds, and in such a manner the compounds can be introduced into the body of an animal without unacceptable side-effects such as toxicity, irritation or allergic response.

Embodiments of the present invention provide compositions comprising one or more pharmaceutically acceptable penetration enhancers, and methods of using such compositions, which result in the improved bioavailability of oligomeric compounds administered via non-parenteral modes of administration. Heretofore, certain penetration enhancers have been used to improve the bioavailability of certain drugs. See Muranishi, Crit. Rev. Ther. Drug Carrier Systems, 1990, 7, 1 and Lee et al., Crit. Rev. Ther. Drug Carrier Systems, 1991, 8, 91. It has been found that the uptake and delivery of oligonucleotides can be greatly improved even when administered by non-parenteral means through the use of a number of different classes of penetration enhancers.

In some embodiments, compositions for non-parenteral administration include one or more modifications from naturally-occurring oligonucleotides (i.e. full-phosphodiester deoxyribosyl or full-phosphodiester ribosyl oligonucleotides). Such modifications may increase binding affinity, nuclease stability, cell or tissue permeability, tissue distribution, or other biological or pharmacokinetic property. Modifications may be made to the base, the linker, or the sugar, in general, as discussed in more detail herein with regards to oligonucleotide chemistry. In some embodiments of the invention, compositions for administration to a subject, and in particular oral compositions for administration to an animal or human subject, will comprise modified oligonucleotides having one or more modifications for enhancing affinity, stability, tissue distribution, or other biological property.

Suitable modified linkers include phosphorothioate linkers. In some embodiments according to the invention, the oligomeric compound has at least one phosphorothioate linker. Phosphorothioate linkers provide nuclease stability as well as plasma protein binding characteristics to the compound. Nuclease stability is useful for increasing the in vivo lifetime of oligomeric compounds, while plasma protein binding decreases the rate of first pass clearance of oligomeric compound via renal excretion. In some embodiments according to the present invention, the oligomeric compound has at least two phosphorothioate linkers. In some embodiments, wherein the oligomeric compound has exactly n nucleosides, the oligomeric compound has from one to n−1 phosphorothioate linkages. In some embodiments, wherein the oligomeric compound has exactly n nucleosides, the oligomeric compound has n−1 phosphorothioate linkages. In other embodiments wherein the oligomeric compound has exactly n nucleoside, and n is even, the oligomeric compound has from 1 to n/2 phosphorothioate linkages, or, when n is odd, from 1 to (n−1)/2 phosphorothioate linkages. In some embodiments, the oligomeric compound has alternating phosphodiester (PO) and phosphorothioate (PS) linkages. In other embodiments, the oligomeric compound has at least one stretch of two or more consecutive PO linkages and at least one stretch of two or more PS linkages. In other embodiments, the oligomeric compound has at least two stretches of PO linkages interrupted by at least one PS linkage.

In some embodiments, at least one of the nucleosides is modified on the ribosyl sugar unit by a modification that imparts nuclease stability, binding affinity or some other beneficial biological property to the sugar. In some cases, the sugar modification includes a 2′-modification, e.g. the 2′-OH of the ribosyl sugar is replaced or substituted. Suitable replacements for 2′-OH include 2′-F and 2′-arabino-F. Suitable substitutions for OH include 2′-O-alkyl, e.g. 2′-O-methyl, and 2′-O-substituted alkyl, e.g. 2′-O-methoxyethyl, 2′-O-aminopropyl, etc. In some embodiments, the oligomeric compound contains at least one 2′-modification. In some embodiments, the oligomeric compound contains at least 2 2′-modifications. In some embodiments, the oligomeric compound has at least one 2′-modification at each of the termini (i.e. the 3′- and 5′-terminal nucleosides each have the same or different 2′-modifications). In some embodiments, the oligomeric compound has at least two sequential 2′-modifications at each end of the compound. In some embodiments, oligomeric compounds further comprise at least one deoxynucleoside. In particular embodiments, oligomeric compounds comprise a stretch of deoxynucleosides such that the stretch is capable of activating RNase (e.g. RNase H) cleavage of an RNA to which the oligomeric compound is capable of hybridizing. In some embodiments, a stretch of deoxynucleosides capable of activating RNase-mediated cleavage of RNA comprises about 8 to about 16, e.g. about 8 to about 16 consecutive deoxynucleosides. In further embodiments, oligomeric compounds are capable of eliciting cleaveage by dsRNAse enzymes.

Oral compositions for administration of non-parenteral oligomeric compounds and compositions of the present invention may be formulated in various dosage forms such as, but not limited to, tablets, capsules, liquid syrups, soft gels, suppositories, and enemas. The term “alimentary delivery” encompasses e.g. oral, rectal, endoscopic and sublingual/buccal administration. A common requirement for these modes of administration is absorption over some portion or all of the alimentary tract and a need for efficient mucosal penetration of the nucleic acid(s) so administered.

Delivery of a drug via the oral mucosa, as in the case of buccal and sublingual administration, has several desirable features, including, in many instances, a more rapid rise in plasma concentration of the drug than via oral delivery (Harvey, Chapter 35 In: Remington's Pharmaceutical Sciences, 18th Ed., Gennaro, ed., Mack Publishing Co., Easton, Pa., 1990, page 711).

Endoscopy may be used for delivery directly to an interior portion of the alimentary tract. For example, endoscopic retrograde cystopancreatography (ERCP) takes advantage of extended gastroscopy and permits selective access to the biliary tract and the pancreatic duct (Hirahata et al., Gan To Kagaku Ryoho, 1992, 19(10 Suppl.), 1591). Pharmaceutical compositions, including liposomal formulations, can be delivered directly into portions of the alimentary canal, such as, e.g., the duodenum (Somogyi et al., Pharm. Res., 1995, 12, 149) or the gastric submucosa (Akamo et al., Japanese J. Cancer Res., 1994, 85, 652) via endoscopic means. Gastric lavage devices (Inoue et al., Artif. Organs, 1997, 21, 28) and percutaneous endoscopic feeding devices (Pennington et al., Ailment Pharmacol. Ther., 1995, 9, 471) can also be used for direct alimentary delivery of pharmaceutical compositions.

In some embodiments, oligomeric compound formulations may be administered through the anus into the rectum or lower intestine. Rectal suppositories, retention enemas or rectal catheters can be used for this purpose and may be desired when patient compliance might otherwise be difficult to achieve (e.g., in pediatric and geriatric applications, or when the patient is vomiting or unconscious). Rectal administration can result in more prompt and higher blood levels than the oral route. (Harvey, Chapter 35 In: Remington's Pharmaceutical Sciences, 18th Ed., Gennaro, ed., Mack Publishing Co., Easton, Pa., 1990, page 711). Because about 50% of the drug that is absorbed from the rectum will bypass the liver, administration by this route significantly reduces the potential for first-pass metabolism (Benet et al., Chapter 1 In: Goodman & Gilman's The Pharmacological Basis of Therapeutics, 9th Ed., Hardman et al., eds., McGraw-Hill, New York, N.Y., 1996).

Some embodiments of the present invention employ various penetration enhancers in order to effect transport of oligomeric compounds and compositions across mucosal and epithelial membranes. Penetration enhancers may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Penetration enhancers and their uses are described in U.S. Pat. No. 6,287,860, which is incorporated herein in its entirety. Accordingly, some embodiments comprise oral oligomeric compound compositions comprising at least one member of the group consisting of surfactants, fatty acids, bile salts, chelating agents, and non-chelating surfactants. Further embodiments comprise oral oligomeric compound comprising at least one fatty acid, e.g. capric or lauric acid, or combinations or salts thereof. Other embodiments comprise methods of enhancing the oral bioavailability of an oligomeric compound, the method comprising co-administering the oligomeric compound and at least one penetration enhancer.

Other excipients that may be added to oral oligomeric compound compositions include surfactants (or “surface-active agents”), which are chemical entities which, when dissolved in an aqueous solution, reduce the surface tension of the solution or the interfacial tension between the aqueous solution and another liquid, with the result that absorption of oligomeric compounds through the alimentary mucosa and other epithelial membranes is enhanced. In addition to bile salts and fatty acids, surfactants include, for example, sodium lauryl sulfate, polyoxyethylene-9-lauryl ether and polyoxyethylene-20-cetyl ether (Lee et al., Critical Reviews in Therapeutic Drug Carrier Systems, 1991, page 92); and perfluorohemical emulsions, such as FC-43 (Takahashi et al., J. Pharm. Phamacol., 1988, 40, 252).

Fatty acids and their derivatives which act as penetration enhancers and may be used in compositions of the present invention include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glyceryl 1-monocaprate, 1-dodecylazacycloheptan-2-one, acylcarnitines, acylcholines and mono- and di-glycerides thereof and/or physiologically acceptable salts thereof (i.e., oleate, laurate, caprate, myristate, palmitate, stearate, linoleate, etc.) (Lee et al., Critical Reviews in Therapeutic Drug Carrier Systems, 1991, page 92; Muranishi, Critical Reviews in Therapeutic Drug Carrier Systems, 1990, 7, 1; El-Hariri et al., J. Pharm. Pharmacol., 1992, 44, 651).

In some embodiments, oligomeric compound compositions for oral delivery comprise at least two discrete phases, which phases may comprise particles, capsules, gel-capsules, microspheres, etc. Each phase may contain one or more oligomeric compounds, penetration enhancers, surfactants, bioadhesives, effervescent agents, or other adjuvant, excipient or diluent. In some embodiments, one phase comprises at least one oligomeric compound and at least one penetration enhancer. In some embodiments, a first phase comprises at least one oligomeric compound and at least one penetration enhancer, while a second phase comprises at least one penetration enhancer. In some embodiments, a first phase comprises at least one oligomeric compound and at least one penetration enhancer, while a second phase comprises at least one penetration enhancer and substantially no oligomeric compound. In some embodiments, at least one phase is compounded with at least one degradation retardant, such as a coating or a matrix, which delays release of the contents of that phase. In some embodiments, a first phase comprises at least one oligomeric compound, at least one penetration enhancer, while a second phase comprises at least one penetration enhancer and a release-retardant. In particular embodiments, an oral oligomeric compound comprises a first phase comprising particles containing an oligomeric compound and a penetration enhancer, and a second phase comprising particles coated with a release-retarding agent and containing penetration enhancer.

A variety of bile salts also function as penetration enhancers to facilitate the uptake and bioavailability of drugs. The physiological roles of bile include the facilitation of dispersion and absorption of lipids and fat-soluble vitamins (Brunton, Chapter 38 In: Goodman & Gilman's The Pharmacological Basis of Therapeutics, 9th Ed., Hardman et al., eds., McGraw-Hill, New York, N.Y., 1996, pages 934-935). Various natural bile salts, and their synthetic derivatives, act as penetration enhancers. Thus, the term “bile salt” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (CDCA, sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1; Yamamoto et al., J. Pharm. Exp. Ther., 1992, 263, 25; Yamashita et al., J. Pharm. Sci., 1990, 79, 579).

In some embodiments, penetration enhancers useful in some embodiments of present invention are mixtures of penetration enhancing compounds. One such penetration enhancer is a mixture of UDCA (and/or CDCA) with capric and/or lauric acids or salts thereof e.g. sodium. Such mixtures are useful for enhancing the delivery of biologically active substances across mucosal membranes, in particular intestinal mucosa. Other penetration enhancer mixtures comprise about 5-95% of bile acid or salt(s) UDCA and/or CDCA with 5-95% capric and/or lauric acid. Particular penetration enhancers are mixtures of the sodium salts of UDCA, capric acid and lauric acid in a ratio of about 1:2:2 respectively. Anther such penetration enhancer is a mixture of capric and lauric acid (or salts thereof) in a 0.01:1 to 1:0.01 ratio (mole basis). In particular embodiments capric acid and lauric acid are present in molar ratios of e.g. about 0.1:1 to about 1:0.1, in particular about 0.5:1 to about 1:0.5.

Other excipients include chelating agents, i.e. compounds that remove metallic ions from solution by forming complexes therewith, with the result that absorption of oligomeric compounds through the alimentary and other mucosa is enhanced. With regard to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315). Chelating agents of the invention include, but are not limited to, disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1; Buur et al., J. Control Rel., 1990, 14, 43).

As used herein, non-chelating non-surfactant penetration enhancers may be defined as compounds that demonstrate insignificant activity as chelating agents or as surfactants but that nonetheless enhance absorption of oligomeric compounds through the alimentary and other mucosal membranes (Muranishi, Critical Reviews in Therapeutic Drug Carrier Systems, 1990, 7, 1). This class of penetration enhancers includes, but is not limited to, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621).

Agents that enhance uptake of oligomeric compounds at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), can be used.

Some oral oligomeric compound compositions also incorporate carrier compounds in the formulation. As used herein, “carrier compound” or “carrier” can refer to a nucleic acid, or analog thereof, which may be inert (i.e., does not possess biological activity per se) or may be necessary for transport, recognition or pathway activation or mediation, or is recognized as a nucleic acid by in vivo processes that reduce the bioavailability of an oligomeric compound having biological activity by, for example, degrading the biologically active oligomeric compound or promoting its removal from circulation. The coadministration of a oligomeric compound and a carrier compound, typically with an excess of the latter substance, can result in a substantial reduction of the amount of oligomeric compound recovered in the liver, kidney or other extracirculatory reservoirs, presumably due to competition between the carrier compound and the oligomeric compound for a common receptor. For example, the recovery of a partially phosphorothioate oligomeric compound in hepatic tissue can be reduced when it is coadministered with polyinosinic acid, dextran sulfate, polycytidic acid or 4-acetamido-4′isothiocyano-stilbene-2,2′-disulfonic acid (Miyao et al., Antisense Res. Dev., 1995, 5, 115; Takakura et al., Antisense & Nucl. Acid Drug Dev., 1996, 6, 177).

A “pharmaceutical carrier” or “excipient” may be a pharmaceutically acceptable solvent, suspending agent or any other pharmacologically inert vehicle for delivering one or more oligomeric compounds to an animal. The excipient may be liquid or solid and is selected, with the planned manner of administration in mind, so as to provide for the desired bulk, consistency, etc., when combined with an oligomeric compound and the other components of a given pharmaceutical composition. Typical pharmaceutical carriers include, but are not limited to, binding agents (e.g., pregelatinised maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, EXPLOTAB); and wetting agents (e.g., sodium lauryl sulphate, etc.).

Oral oligomeric compound compositions may additionally contain other adjunct components conventionally found in pharmaceutical compositions, at their art-established usage levels. Thus, for example, the compositions may contain additional, compatible, pharmaceutically-active materials such as, for example, antipuritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers. However, such materials, when added, should not unduly interfere with the biological activities of the components of the compositions of the present invention.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The oligomeric compounds and compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations.

Emulsions are typically heterogenous systems of one liquid dispersed in another in the form of droplets usually exceeding 0.1 μm in diameter. Emulsions may contain additional components in addition to the dispersed phases, and the active drug that may be present as a solution in either the aqueous phase, oily phase or itself as a separate phase. Microemulsions are included as an embodiment of the present invention. Emulsions and their uses are well known in the art and are described in U.S. Pat. No. 6,287,860, which is incorporated herein in its entirety.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged nucleic acid molecules to form a stable complex. Liposomes that are pH-sensitive or negatively-charged are believed to entrap nucleic acids rather than complex with it. Both cationic and noncationic liposomes have been used to deliver nucleic acids and oligomeric compounds to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are described in U.S. Pat. No. 6,287,860, which is incorporated herein in its entirety.

The pharmaceutical formulations and compositions of the present invention may also include surfactants. The use of surfactants in drug products, formulations and in emulsions is well known in the art. Surfactants and their uses are described in U.S. Pat. No. 6,287,860, which is incorporated herein in its entirety.

One of skill in the art will recognize that formulations are routinely designed according to their intended use, i.e. route of administration.

Formulations for topical administration include those in which the oligomeric compounds of the invention are in admixture with a topical delivery agent such as lipids, liposomes, fatty acids, fatty, acid esters, steroids, chelating agents and surfactants. Lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligomeric compounds and compositions of the invention may be encapsulated within liposomes or may form complexes thereto, in particular to cationic liposomes. Alternatively, they may be complexed to lipids, in particular to cationic lipids. Topical formulations are described in detail in U.S. patent application Ser. No. 09/315,298 filed on May 20, 1999, which is incorporated herein by reference in its entirety.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Oral formulations are those in which oligomeric compounds of the invention are administered in conjunction with one or more penetration enhancers surfactants and chelators. A particularly suitable combination is the sodium salt of lauric acid, capric acid and UDCA. Penetration enhancers also include polyoxyethylene-9-lauryl ether, polyoxyethylene-20-cetyl ether. Compounds and compositions of the invention may be delivered orally, in granular form including sprayed dried particles, or complexed to form micro or nanoparticles. Certain oral formulations for oligonucleotides and their preparation are described in detail in U.S. application Ser. No. 09/108,673 (filed Jul. 1, 1998), Ser. No. 09/315,298 (filed May 20, 1999) and U.S. Application Publication 20030027780, each of which is incorporated herein by reference in their entirety.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more of the compounds and compositions of the invention and one or more other chemotherapeutic agents that function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the oligomeric compounds of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of oligomeric compounds and compositions of the invention and other drugs are also within the scope of this invention. Two or more combined compounds such as two oligomeric compounds or one oligomeric compound combined with further compounds may be used together or sequentially.

In another embodiment, compositions of the invention may contain one or more of the compounds and compositions of the invention targeted to a first nucleic acid target and one or more additional oligomeric compounds targeted to a second nucleic acid target. Alternatively, compositions of the invention may contain two or more oligomeric compounds and compositions targeted to different regions, segments or sites of the same target. Two or more combined compounds may be used together or sequentially.

The formulation of therapeutic compounds and compositions of the invention and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligomeric compounds, and can generally be estimated based on EC₅₀s found to be effective in in vitro and in vivo animal models. In general, dosage is from 0.01 μg to 100 g per kg of body weight, from 0.1 μg to 10 g per kg of body weight, from 1.0 μg to 1 g per kg of body weight, from 10.0 μg to 100 mg per kg of body weight, from 100 μg to 10 mg per kg of body weight, or from 1 mg to 5 mg per kg of body weight, and may be given once or more daily, weekly, monthly or yearly, or even once every 2 to 20 years. Persons of ordinary skill in the art can easily determine repetition rates for dosing based on measured residence times and concentrations of the drug in bodily fluids or tissues. Following successful treatment, it may be desirable to have the patient undergo maintenance therapy to prevent the recurrence of the disease state, wherein the oligomeric compound is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight, once or more daily, to once every 20 years. The effects of treatments with therapeutic compositions can be assessed following collection of tissues or fluids from a patient or subject receiving said treatments. It is known in the art that a biopsy sample can be procured from certain tissues without resulting in detrimental effects to a patient or subject. In certain embodiments, a tissue and its constituent cells comprise, but are not limited to, blood (e.g., hematopoietic cells, such as human hematopoietic progenitor cells, human hematopoietic stem cells, CD34⁺ cells CD4⁺ cells), lymphocytes and other blood lineage cells, bone marrow, breast, cervix, colon, esophagus, lymph node, muscle, peripheral blood, oral mucosa and skin. In other embodiments, a fluid and its constituent cells comprise, but are not limited to, blood, urine, semen, synovial fluid, lymphatic fluid and cerebro-spinal fluid. Tissues or fluids procured from patients can be evaluated for expression levels of a target small non-coding RNA, mRNA or protein. Additionally, the mRNA or protein expression levels of other genes known or suspected to be associated with the specific disease state, condition or phenotype can be assessed. mRNA levels can be measured or evaluated by real-time PCR, Northern blot, in situ hybridization or DNA array analysis.

The present invention also provides methods as described below.

Target Nucleic Acid Selection

The target selection process provides a target nucleotide sequence that is used to help guide subsequent steps of the process. It is generally desired to modulate the expression of the target nucleic acid for any of a variety of purposes, such as, e.g., drug discovery, target validation and/or gene function analysis.

One of the primary objectives of the target selection process is to identify molecular targets that represent significant therapeutic opportunities, provide new medicines to the medical community to fill therapeutic voids or improve upon existing therapies, to provide new and efficacious means of drug discovery and to determine the function of genes that are uncharacterized except for nucleotide sequence. To meet these objectives, genes are classified based upon specific sets of selection criteria.

One such set of selection criteria concerns the quantity and quality of target nucleotide sequence. There must be sufficient target nucleic acid sequence information available for oligonucleotide design. Moreover, such information must be of sufficient quality, e.g., not containing too many missing or incorrect base entries. In the case of a target sequence that encodes a polypeptide, such errors can be detected by virtually translating all three reading frames of the sense strand of the target sequence and confirming the presence of a continuous polypeptide sequence having predictable attributes (e.g., encoding a polypeptide of known size, or encoding a polypeptide that is about the same length as a homologous protein). In any event, only a very high frequency of sequence errors will frustrate the method of the invention; most oligonucleotides to the target sequence will avoid such errors unless such errors occur frequently throughout the entire target sequence.

Another criterion is that appropriate culturable cell lines should be available. Such cell lines express, or can be induced to express, the gene comprising the target nucleic acid sequence. The oligonucleotide compounds generated by the process of the invention are assayed using such cell lines and, if such assaying is performed robotically, the cell line is tractable to robotic manipulation and growth in 96 well plates. Those skilled in the art will recognize that if an appropriate cell line does not exist, it will nevertheless be possible to construct an appropriate cell line. For example, a cell line can be transfected with an expression vector comprising the target gene in order to generate an appropriate cell line for assay purposes.

For gene function analysis, a selection criterion is a lack of information regarding, or incomplete characterization of, the biological function(s) of the target nucleic acid or its gene product. A target nucleic acid for gene function analysis might be absolutely uncharacterized, or might be thought to have a function based only on minimal data or homology to another gene. By application of the process of the invention to such a target, active compounds that modulate the expression of the gene can be developed and applied to cells. The resulting cellular, biochemical or molecular biological responses are observed, and this information is used by those skilled in the art to elucidate the function of the target gene.

For target validation and drug discovery, another selection criterion is disease association. Candidate target genes are placed into one of several broad categories of known or deduced disease association. Level 1 Targets are target nucleic acids for which there is a strong correlation with disease. This correlation can come from multiple scientific disciplines including, but not limited to, epidemiology, wherein frequencies of gene abnormalities are associated with disease incidence; molecular biology, wherein gene expression and function are associated with cellular events correlated with a disease; and biochemistry, wherein the in vitro activities of a gene product are associated with disease parameters. Because there is a strong therapeutic rationale for focusing on Level 1 Targets, these targets are most suitable for drug discovery and/or target validation. Level 2 Targets are nucleic acid targets for which the combined epidemiological, molecular biological, and/or biochemical correlation with disease is more tenuous. Level 3 Targets are targets for which there is little or no data to directly link the target with a disease process, but there is indirect evidence for such a link (i.e., homology with a Level 1 or Level 2 target nucleic acid sequence or with the gene product thereof). In order to not prejudice the target selection process, and to ensure that the maximum number of nucleic acids actually involved in the causation, potentiation, aggravation, spread, continuance or after-effects of disease states are investigated, it is desirable to examine a balanced mix of Level 1, 2 and 3 target nucleic acids.

In order to carry out drug discovery, experimental systems and reagents must be available in order for one to evaluate the therapeutic potential of active compounds generated by the process of the invention. Such systems may be operable in vitro (e.g., in vitro models of cell:cell association) or in vivo (e.g., animal models of disease states). It is also desirable, but not obligatory, to have available animal model systems which can be used to evaluate drug pharmacology.

Candidate targets nucleic acids can also classified by biological processes. For example, programmed cell death (“apoptosis”) has recently emerged as an important biological process that is perturbed in a wide variety of diseases. Accordingly, nucleic acids that encode factors that play a role in the apoptotic process are identified as candidate targets. Similarly, potential target nucleic acids can be classified as being involved in inflammation, autoimmune disorders, cancer, or other pathological or dysfunctional processes.

Moreover, genes can often be grouped into families based on sequence homology and biological function. Individual family members can act in either a redundantly, or provide specificity through diversity of interactions with down stream effectors, or specificity through expression being restricted to specific cell types. When one member of a gene family is associated with a disease process then the rationale for targeting other members of the same family is reasonably strong. Therefore, members of such gene families are suitable target nucleic acids to which the methods and systems of the invention may be applied. Indeed, the potent specificity of antisense compounds for different gene family members makes the invention particularly suited for such targets (Albert et al., Trends Pharm. Sci., 1994, 15, 250). Those skilled in the art will recognize that a partial or complete nucleotide sequence of such family members can be obtained using the polymerase chain reaction (PCR) and “universal” primers, i.e., primers designed to be common to all members of a given gene family.

PCR products generated from universal primers can be cloned and sequenced or directly sequenced using techniques known in the art. Moreover, as is known in the art, PCR can be used to directly sequence RNAs. Thus, although nucleotide sequences from cloned DNAs, or from complementary DNAs (cDNAs) derived from mRNAs, may be used in the process of the invention, there is no requirement that the target nucleotide sequence be isolated from a cloned nucleic acid. Any nucleotide sequence, no matter how determined, of any nucleic acid, isolated or prepared in any fashion, may be used as a target nucleic acid in the process of the invention. One potentially fertile source of design information may be in microRNA, such as RNAi, siRNA, miRNA, tncRNA and others. These microRNA, including modified mimics thereof may be used as a target nucleic acid in the process of the invention.

Furthermore, although polypeptide-encoding nucleic acids provide the target nucleotide sequences in one embodiment of the invention, other nucleic acids may be targeted as well. Thus, for example, the nucleotide sequences of structural or enzymatic RNAs may be utilized for drug discovery and/or target validation when such RNAs are associated with a disease state, or for gene function analysis when their biological role is not known.

Assembly of Target Nucleotide Sequence

The ease of the oligonucleotide design process is dependent upon the availability of accurate RNA sequence information. Because of limitations of automated genome sequencing technology, gene sequences are often accumulated in fragments. Further, because individual genes are often being sequenced by independent laboratories using different sequencing strategies, sequence information corresponding to different fragments is often deposited in different databases. The target nucleic acid assembly process takes advantage of computerized homology search algorithms and sequence fragment assembly algorithms to search available databases for related sequence information and incorporate available sequence information into the best possible representation of the target RNA molecule. This representation of a unique RNA transcript from a target gene is then used to design oligonucleotides, which are eventually tested for biological activity.

In the case of genes directing the synthesis of multiple transcripts, i.e., by alternative splicing, each distinct transcript is a unique target nucleic acid. In one embodiment of the invention, if active compounds specific for a given transcript isoform are desired, the target nucleotide sequence is limited to those sequences that are unique to that transcript isoform. In another embodiment of the invention, if it is desired to modulate two or more transcript isoforms in concert, the target nucleotide sequence is limited to sequences that are shared between the two or more transcripts.

In the case of a polypeptide-encoding nucleic acid, it is generally suitable that full-length cDNA be used in the oligonucleotide design. Although full-length cDNA is suitable, it is possible to design oligonucleotides using partial sequence information. Therefore it is not necessary for the assembly process to generate a complete cDNA sequence. Further in some cases it may be desirable to design oligonucleotides targeting introns. In this case the process can be used to identify individual introns.

The process is initiated by entering initial sequence information on a selected molecular target. In the case of a polypeptide-encoding nucleic acid, the full-length cDNA sequence is generally suitable for use in oligonucleotide design strategies. The first step is to determine if the initial sequence information represents the full-length cDNA. In the case where the full-length cDNA sequence is available the process advances directly to the oligonucleotide. When the full-length cDNA sequence is not available, databases are searched for additional sequence information.

The algorithm used is Gapped BLAST, usually referred to as “BLAST” (Altschul et al., Nucl. Acids Res., 1997, 25, 3389). BLAST is database search tool based on sequence homology used to identify related sequences in a sequence database. The BLAST search parameters are set to only identify closely related sequences. The databases searched by BLAST are a combination of public domain and proprietary databases. The databases, their contents, and sources are listed in Table 12.

When genomic sequence information is available, introns and exons are identified. Introns are removed and exons are assembled into continuous sequence representing the cDNA sequence. Exon assembly occurs using the Phragment Assembly Program “Phrap” (Copyright University of Washington Genome Center, Seattle, Wash.). The Phrap algorithm analyzes sets of overlapping sequences and assembles them into one continuous sequence referred to as a “contig”. The resulting contig is used to search databases for additional sequence information. When genomic information is not available the results are analyzed for individual exons. Exons are frequently recorded individually in databases. If multiple complete exons are identified, they are assembled into a contig using Phrap. If multiple complete exons are not identified, then sequences are analyzed for partial sequence information. ESTs identified in the database dbEST are examples of such partial sequence information. If additional partial information is not found, then the process is advanced. If partial sequence information is found then that information is advanced.

These process and decision steps define a loop designed to iteratively extend the amount of sequence information available for targeting. At the end of each iteration of this loop, the results are analyzed. If no new information is found then the process advances. If there is an unexpectedly large amount of sequence information identified, then the process is cycled back one iteration and that sequence is advanced. If a small amount of new sequence information is identified, then the loop is iterated by taking the 100 most 5-prime and 100 most 3-prime bases and interating them through the BLAST homology search. New sequence information is added to the existing contig.

This loop is iterated until either no new sequence information is identified, or an unexpectedly large amount of new information is found, suggesting that the process moved outside the boundary of the gene into repetitive genomic sequence. In either of these cases, iteration of this loop will be stopped and the process will advance to the oligonucleotide design.

In an alternative embodiment of the invention, each possible oligonucleotide chemistry is first assigned to each possible oligonucleotide sequence. Then, each combination of oligonucleotide chemistry and sequence is evaluated according to the previous parameters. This embodiment has the desirable feature of taking into account the effect of alternate oligonucleotide chemistries on such parameters. For example, substitution of 5-methyl cytosine (m5c) for cytosine in an antisense compound may enhance the stability of a duplex formed between that compound and its target nucleic acid. Other oligonucleotide chemistries that enhance oligonucleotide:[target nucleic acid] duplexes are known in the art (see for example, Frier et al., Nucleic Acids Research, 1997, 25, 4429). As will be appreciated by those skilled in the art, different oligonucleotide chemistries may be desired for different target nucleic acids. That is, the optimal oligonucleotide chemistry for a target DNA might be suboptimal for a target RNA having the same nucleotide sequence.

TABLE 12
Database Sources of Target Sequences

| Database | Contents | Source |
|---|---|---|
| NR | All non-redundant | National Center for Bio- |
| | GenBank, EMBL, DDBJ | technology Information at |
| | and PDB sequences | the National Institutes of |
| | | Health |
| Month | All new or revised | National Center for Bio- |
| | GenBank, EMBL, DDBJ | technology Information at |
| | and PDB sequences | the National Institutes |
| | released in the last | of Health |
| | 30 days | |
| Dbest | Non-redundant data- | National Center for Bio- |
| | base of GenBank, EMBL, | technology Information at |
| | DDBJ and EST divisions | the National Institutes |
| | | of Health |
| Dbsts | Non-redundant database | National Center for Bio- |
| | of GenBank, EMBL, DDBJ | technology Information at |
| | and STS divisions | the National Institutes |
| | | of Health |
| Htgs | High throughput genomic | National Center for Bio- |
| | sequences | technology Information at |
| | | the National Institutes |
| | | of Health |

In Silico Generation of a Set of Nucleobase Sequences and Virtual Oligonucleotides

From a target nucleic acid sequence assembled, a list of oligonucleotide sequences is generated. The desired oligonucleotide length is chosen. In one embodiment, oligonucleotide length is between from about 8 to about 30 or from about 12 to about 25 nucleotides. All possible oligonucleotide sequences of the desired length capable of hybridizing to the target sequence obtained are generated. In this step, a series of oligonucleotide sequences are generated, simply by determining the most 5′ oligonucleotide possible and “walking” the target sequence in increments of one base until the 3′ most oligonucleotide possible is reached.

A virtual oligonucleotide chemistry is applied to the nucleobase sequences in order to yield a set of virtual oligonucleotides that can be evaluated in silico. Default virtual oligonucleotide chemistries include those that are well-characterized in terms of their physical and chemical properties, e.g., 2′-deoxyribonucleic acid having naturally occurring bases (A, T, C and G), unmodified sugar residues and a phosphodiester backbone.

In Silico Evaluation of Thermodynamic Properties of Virtual Oligonucleotides

A series of thermodynamic, sequence, and homology scores are calculated for each virtual oligonucleotide obtained. The desired thermodynamic properties are selected. This will typically include calculation of the free energy of the target structure. These steps correspond to calculation of the free energy of intramolecular oligonucleotide interactions, intermolecular interactions and duplex formation. In addition, a free energy of oligonucleotide-target binding is calculated.

Other thermodynamic and kinetic properties may be calculated for oligonucleotides. Such other thermodynamic and kinetic properties may include melting temperatures, association rates, dissociation rates, or any other physical property that may be predictive of oligonucleotide activity.

The free energy of the target structure is defined as the free energy needed to disrupt any secondary structure in the target binding site of the targeted nucleic acid. This region includes any nucleotide base pairs that need to be disrupted in order for an oligonucleotide to bind to its complementary base pairs. The effect of this localized disruption of secondary structure is to provide accessibility by the oligonucleotide. Such structures will include double helices, terminal unpaired and mismatched nucleotides, loops, including hairpin loops, bulge loops, internal loops and multibranch loops (Serra et al., Methods in Enzymology, 1995, 259, 242).

The intermolecular free energies refer to inherent energy due to the most stable structure formed by two oligonucleotides; such structures would include dimer formation. Intermolecular free energies should be taken into account when, for example, two or more oligonucleotides are going to be administered to the same cell in an assay.

The intramolecular free energies refer to the energy needed to disrupt the most stable secondary structure within a single oligonucleotide. Such structures include, for example, hairpin loops, bulges and internal loops. The degree of intramolecular base pairing is indicative of the energy needed to disrupt such base pairing.

The free energy of duplex formation is the free energy of denatured oligonucleotide binding to its denatured target sequence. The oligonucleotide-target binding is the total binding involved, and includes the energies involved in opening up intra- and intermolecular oligonucleotide structures, opening up target structure, and duplex formation.

The most stable RNA structure is predicted based on nearest neighbor analysis (Serra et al., Methods in Enzymology, 1995, 259, 242). This analysis is based on the assumption that stability of a given base pair is determined by the adjacent base pair. For each possible nearest neighbor combination, thermodynamic properties have been determined and are provided. For double helical regions, two additional factors need to be considered, an entropy change required to initiate a helix and a entropy change associated with self-complementary strands only. Thus, the free energy of a duplex can be calculated using the equation:
ΔG°_{T}=ΔH°−TΔS°
where:
ΔG is the free energy of duplex formation,
ΔH is the enthalpy change for each nearest neighbor,
ΔS is the entropy change for each nearest neighbor, and
T is temperature.
   The ΔH and ΔS for each possible nearest neighbor combination have been experimentally determined and these are available in published tables. For terminal unpaired and mismatched nucleotides, enthalpy and entropy measurements for each possible nucleotide combination are also available in published tables. Such results are added directly to values determined for duplex formation. For loops, while the available data is not as complete or accurate as for base pairing, one known model determines the free energy of loop formation as the sum of free energy based on loop size, the closing base pair, the interactions between the first mismatch of the loop with the closing base pair, and additional factors including being closed by AU or UA or a first mismatch of GA or UU. Such equations may also be used for oligoribonucleotide-target RNA interactions.

The stability of DNA duplexes is used in the case of intra- or intermolecular oligodeoxyribonucleotide interactions. DNA duplex stability is calculated using similar equations as RNA stability, except experimentally determined values differ between nearest neighbors in DNA and RNA and helix initiation tends to be more favorable in DNA than in RNA (SantaLucia et al., Biochemistry, 1996, 35, 3555).

Additional thermodynamic parameters are used in the case of RNA/DNA hybrid duplexes. This would be the case for an RNA target and oligodeoxynucleotide. Such parameters were determined by Sugimoto et al. (Biochemistry, 1995, 34, 11211). In addition to values for nearest neighbors, differences were seen for values for enthalpy of helix initiation.

In Silico Evaluation of Target Accessibility

Target accessibility is believed to be an important consideration in selecting oligonucleotides. Such a target site will possess minimal secondary structure and thus, will require minimal energy to disrupt such structure. In addition, secondary structure in oligonucleotides, whether inter- or intra-molecular, is undesirable due to the energy required to disrupt such structures. Oligonucleotide-target binding is dependent on both these factors. It is desirable to minimize the contributions of secondary structure based on these factors. The other contribution to oligonucleotide-target binding is binding affinity. Favorable binding affinities based on tighter base pairing at the target site is desirable.

Following the calculation of thermodynamic properties, the desired sequence properties to be scored are selected. These properties include the number of strings of four guanosine residues in a row or three guanosines in a row, the length of the longest string of adenosines, cytosines or uridines or thymidines, the length of the longest string of purines or pyrimidines, the percent composition of adenosine, cytosine, guanosine or uridines or thymidines, the percent composition of purines or pyrimidines, the number of CG dinucleotide repeats, CA dinucleotide repeats or UA or TA dinucleotide repeats. In addition, other sequence properties may be used as found to be relevant and predictive of antisense efficacy.

These sequence properties may be important in predicting oligonucleotide activity, or lack thereof. For example, U.S. Pat. No. 5,523,389 discloses oligonucleotides containing stretches of three or four guanosine residues in a row. Oligonucleotides having such sequences may act in a sequence-independent manner. For an antisense approach, such a mechanism is not desired. In addition, high numbers of dinucleotide repeats may be indicative of low complexity regions which may be present in large numbers of unrelated genes. Unequal base composition, for example, 90% adenosine, can also give non-specific effects. From a practical standpoint, it may be desirable to remove oligonucleotides that possess long stretches of other nucleotides due to synthesis considerations. Other sequences properties, either listed above or later found to be of predictive value may be used to select oligonucleotide sequences.

The homology scores to be calculated are selected. Homology to nucleic acids encoding protein isoforms of the target may be desired. For example, oligonucleotides specific for an isoform of protein kinase C can be selected. Also, oligonucleotides can be selected to target multiple isoforms of such genes. Homology to analogous target sequences may also be desired. For example, an oligonucleotide can be selected to a region common to both humans and mice to facilitate testing of the oligonucleotide in both species. Homology to splice variants of the target nucleic acid may be desired. In addition, it may be desirable to determine homology to other sequence variants as necessary.

Once scores were obtained in each selected parameter, a desired range is selected to select the most promising oligonucleotides. Typically, only several parameters will be used to select oligonucleotide sequences. As structure prediction improves, additional parameters may be used. Once the desired score ranges are chosen, a list of all oligonucleotides having parameters falling within those ranges will be generated.

Targeting Oligonucleotides to Functional Regions of a Nucleic Acid

It may be desirable to target oligonucleotide sequences to specific functional regions of the target nucleic acid. A decision is made whether to target such regions. If it is desired to target functional regions, then the desired functional regions are selected. Such regions include the transcription start site or 5′ cap, the 5′ untranslated region, the start codon, the coding region, the stop codon, the 3′ untranslated region, 5′ or 3′ splice sites, specific exons or specific introns, mRNA stabilization signal, mRNA destabilization signal, polyadenylation signal, poly-A addition site, poly-A tail, or the gene sequence 5′ of known pre-mRNA. In addition, additional functional sites may be selected.

Many functional regions are important to the proper processing of the gene and are attractive targets for antisense approaches. For example, the AUG start codon is commonly targeted because it is necessary to initiate translation. In addition, splice sites are thought to be attractive targets because these regions are important for processing of the mRNA. Other known sites may be more accessible because of interactions with protein factors or other regulatory molecules.

After the desired functional regions are selected and determined, then a subset of all previously selected oligonucleotides are selected based on hybridization to only those desired functional regions.

Uniform Distribution of Oligonucleotides

Whether or not targeting functional sites is desired, a large number of oligonucleotide sequences may result from the process thus far. In order to reduce the number of oligonucleotide sequences to a manageable number, a decision is made whether to uniformly distribute selected oligonucleotides along the target. A uniform distribution of oligonucleotide sequences will aim to provide complete coverage throughout the complete target nucleic acid or the selected functional regions. A utility is used to automate the distribution of sequences. Such a utility factors in parameters such as length of the target nucleic acid, total number of oligonucleotide sequences desired, oligonucleotide sequences per unit length, number of oligonucleotide sequences per functional region. Manual selection of oligonucleotide sequences is also provided. In some cases, it may be desirable to manually select oligonucleotide sequences. For example, it may be useful to determine the effect of small base shifts on activity. Once the desired number of oligonucleotide sequences is obtained, then oligonucleotide chemistries are assigned.

Assignment of Actual Oligonucleotide Chemistry

Once a set of select nucleobase sequences has been generated according to the preceding process and decision steps, actual oligonucleotide chemistry is assigned to the sequences. An “actual oligonucleotide chemistry” or simply “chemistry” is a chemical motif that is common to a particular set of robotically synthesized oligonucleotide compounds. Suitable chemistries include, but are not limited to, oligonucleotides in which every linkage is a phosphorothioate linkage, and chimeric oligonucleotides, in which a defined number of 5′ and/or 3′ terminal residues have a 2′-methoxyethoxy modification.

Chemistries are assigned to the nucleobase. Chemistry assignment can be effected by assignment directly into a word processing program, via an interactive word processing program or via automated programs and devices. In each of these instances, the output file is selected to be in a format that can serve as an input file to automated synthesis devices.

Oligonucleotide Compounds

In the context of this invention, in reference to oligonucleotides, the term “oligonucleotide” is used to refer to an oligomer or polymer of ribonucleic acid (RNA) or deoxyribonucleic acid (DNA) or mimetics thereof. Thus this term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often desired over native forms, i.e., phosphodiester linked A, C, G, T and U nucleosides, because of desirable properties such as, for example, enhanced cellular uptake, enhanced affinity for nucleic acid target and increased stability in the presence of nucleases.

The oligonucleotide compounds in accordance with this invention can be of various lengths depending on various parameters, including but not limited to those discussed above in reference to the selection criteria of general procedure 300. Normally oligonucleotides used for binding interact with a target as antisense compounds are from about 8 to about 30 nucleobases in length. Particularly desired are antisense oligonucleotides comprising from about 8 to about 30 nucleobases (i.e. from about 8 to about 30 linked nucleosides). A discussion of antisense oligonucleotides and some desirable modifications can be found in De Mesmaeker et al., Acc. Chem. Res., 1995, 28, 366. Other lengths of oligonucleotides might be selected for non-antisense targeting strategies as for instance using the oligonucleotides as ribozymes. Such ribozymes normally require oligonucleotides of longer length as is known in the art.

A nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a normal (where normal is defined as being found in RNA and DNA) pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally suitable. Within the oligonucleotide structure, the phosphate groups are commonly referred to as forming the intersugar backbone of the oligonucleotide. The normal linkage or backbone of RNA and DNA is a 3′ to 5′ phosphodiester linkage.

Specific examples of oligonucleotides useful in this invention include oligonucleotides containing modified backbones or non-natural intersugar linkages. As defined in this specification, oligonucleotides having modified backbones include those that retain a phosphorus atom in the backbone and those that do not have a phosphorus atom in the backbone. Whether for the purposes of this specification, and as sometimes referenced in the art, modified oligonucleotides that do not have a phosphorus atom in their intersugar backbone can also be considered to be oligonucleosides.

Selection of Oligonucleotide Chemistries

For each nucleoside position, the user or automated devices is interrogated first for a base assignment, followed by a sugar assignment, a linker assignment and finally a conjugate assignment. Thus for each nucleoside a base is selected. In selecting the base, base chemistry 1 can be selected or one or more alternate bases are selected. After base selection is effected, the sugar portion of the nucleoside is selected. Thus for each nucleoside, a sugar is selected that together with the select base will complete the nucleoside. In selecting the sugar, sugar chemistry 1 can be selected or one or more alternate sugars are selected. For each two adjacent nucleoside units, the internucleoside linker is selected. The linker chemistry for the internucleoside linker can be linker chemistry 1 selected or one or more alternate internucleoside linker chemistries are selected.

In addition to the base, sugar and internucleoside linkage, at each nucleoside position, one or more conjugate groups can be attached to the oligonucleotide via attachment to the nucleoside or attachment to the internucleoside linkage. The addition of a conjugate group is integrated and the assignment of the conjugate group is effected.

For each of the base, the sugar, the internucleoside linkers, or the conjugate, chemistries 1 though n are illustrated. As described in this specification, it is understood that the number of alternate chemistries between chemistry 1 and alternate chemistry n, for each of the base, the sugar, the internucleoside linkage and the conjugate, is variable and includes, but is not limited to, each of the specific alternate bases, sugar, internucleoside linkers and conjugates identified in this specification as well as equivalents known in the art.

Description of Automated Oligonucleotide Synthesis

In the next step of the overall process, oligonucleotides are synthesized on an automated synthesizer. The synthesizer is a variation of the synthesizer described in U.S. Pat. Nos. 5,472,672 and 5,529,756, the entire contents of which are herein incorporated by reference. The synthesizer of those patents was modified to include movement in along the Y axis in addition to movement along the X axis. As so modified, a 96-well parallel array of compounds can be synthesized by the synthesizer. The synthesizer further includes temperature control and the ability to maintain an inert atmosphere during all phases of a synthesis. The reagent array delivery format employs orthogonal X-axis motion of a matrix of reaction vessels and Y-axis motion of an array of reagents. Each reagent has its own dedicated plumbing system to eliminate the possibility of cross-contamination of reagents and line flushing and/or pipette washing. This in combination with a high delivery speed obtained with a reagent mapping system allows for the extremely rapid delivery of reagents. This further allows long and complex reaction sequences to be performed in a facile manner. The software, which operates the synthesizer, allows for the straightforward programming of the parallel synthesis of a large number of compounds. The software utilizes a general synthetic procedure in the form of a command (.cmd) file, which calls upon certain reagents to be added to certain wells via lookup in a sequence (.seq) file. The bottle position, flow rate, and concentration of each reagent is stored in a lookup table (.tab) file. Thus, once any synthetic method has been outlined, a plate of compounds are made by permutating a set of reagents, and writing the resulting output to a text file, which is directly used for synthesis. The synthesizer is interfaced with a relational database allowing data output related to the synthesized compounds to be registered in a highly efficient manner.

Thus as a part of the general oligonucleotide synthesis procedure, for each linker chemisty, a synthesis file, i.e., a .cmd file, is built. This file can be built fresh to reflect a completely new set of machine commands reflecting a set of chemical synthesis steps or it can modify an existing file stored by editing that stored file. The .cmd files are built using a word processor and a command set of instructions as outlined below.

In a like manner to the building the .cmd files, .tab files are built to reflect the necessary reagents used in the automatic synthesizer for the particular chemistries that have been selected for the bases, sugars and conjugate chemistries. Thus for each of a set of these chemistries, a .tab file is built and stored. As with the .cmd files, an existing tab file can be edited.

Both the .cmd files and the tab files are linked together and stored for later retrievable in an appropriate sample database. Linking can be as simple as using like file names to associate a .cmd file to its appropriate tab file, e.g., synthesis_{—}1.cmd is liked to synthesis_{—}1.tab by use of the same preamble in their names.

The automated, multi well parallel array synthesizer employs a reagent array delivery format, in which each reagent utilized has a dedicated plumbing system. An inert atmosphere is maintained during all phases of a synthesis. Temperature is controlled via a thermal transfer plate, which holds an injection molded reaction block. The reaction plate assembly slides in the X-axis direction, while eight nozzle blocks holding the reagent lines slide in the Y-axis direction, allowing for the extremely rapid delivery of any of 64 reagents to 96 wells. In addition, there are six banks of fixed nozzle blocks which deliver the same reagent or solvent to eight wells at once, for a total of 72 possible reagents. In synthesizing oligonucleotides for screening, the target reaction vessels, a 96 well plate (a 2-dimensional array), moves in one direction along the X axis, while the series of independently controlled reagent delivery nozzles move along the Y-axis relative to the reaction vessel. As the reaction plate and reagent nozzles can be moved independently at the same time, this arrangement facilitated the extremely rapid delivery of up to 72 reagents independently to each of the 96 reaction vessels.

The system software allows the straightforward programming of the synthesis of a large number of compounds by supplying the general synthetic procedure in the form of the command file to call upon certain reagents to be added to specific wells via lookup in the sequence file with the bottle position, flow rate, and concentration of each reagent being stored in the separate reagent table file. Compounds can be synthesized on various scales. For Oligonucleotide, a 200 nmole scale is selected while for other compounds larger scales, as for example a 10 μmole scale (3-5 mg), might be utilized. The resulting crude compounds are generally >80% pure, and are utilized directly for high throughput screening assays. Alternately, prior to use the plates can be subjected to quality control (see general procedure 600 and Example 9) to ascertain their exact purity. Use of the synthesizer results in a very efficient means for the parallel synthesis of compounds for screening.

The software inputs accept tab delimited text files from any text editor. A typical command file, a .cmd file, a typical sequence files, seq files, and a typical reagent file, a tab file, are shown below. 2′-O-(methoxyethyl) modified nucleoside are utilized in a first region (a wing) of the oligonucleotide, followed by a second region (a gap) of 2′-deoxy nucleotides and finally a third region (a further wing) that has the same chemistry as the first region. Typically some of the wells of the 96 well plate may be left empty (depending on the number of oligonucleotides to be made during an individual synthesis) or some of the well may have oligonucleotides that will serve as standards for comparison or analytical purposes.

Prior to loading reagents, moisture sensitive reagent lines are purged with argon for 20 minutes. Reagents are dissolved to appropriate concentrations and installed on the synthesizer. Large bottles are used for wash solvents and the delivery of general activators, trityl group cleaving reagents and other reagents that may be used in multiple wells during any particular synthesis. Small septa are utilized to contain individual nucleotide amidite precursor compounds. This allows for anhydrous preparation and efficient installation of multiple reagents by using needles to pressurize the bottle, and as a delivery path. After all reagents are installed, the lines are primed with reagent, flow rates measured, then entered into the reagent table (.tab file). A dry resin loaded plate is removed from vacuum and installed in the machine for the synthesis.

The modified 96 well polypropylene plate is utilized as the reaction vessel. The working volume in each well is approximately 700 μl. The bottom of each well is provided with a pressed-fit 20 μm polypropylene frit and a long capillary exit into a lower collection chamber as is illustrated in FIG. 5 of the above referenced U.S. Pat. No. 5,372,672. The solid support for use in holding the growing oligonucleotide during synthesis is loaded into the wells of the synthesis plate by pipetting the desired volume of a balanced density slurry of the support suspended in an appropriate solvent, typically acetonitrile-methylene chloride mixtures. Reactions can be run on various scales as for instance the above noted 200 nmole and 10 μmol scales. For oligonucldotide synthesis, a CPG support is suitable however other medium loading polystyrene-PEG supports such as TentaGel™ or ArgoGel™ can also be used.

The synthesis plate is transported back and forth in the X-direction under an array of 8 moveable banks of 8 nozzles (64 total) in the Y-direction, and 6 banks of 48 fixed nozzles, so that each well can receive the appropriate amounts of reagents and/or solvents from any reservoir (large bottle or smaller septa bottle). A sliding balloon-type seal surrounds this nozzle array and joins it to the reaction plate headspace. A slow sweep of nitrogen or argon at ambient pressure across the plate headspace is used to preserve an anhydrous environment.

The liquid contents in each well do not drip out until the headspace pressure exceeds the capillary forces on the liquid in the exit nozzle. A slight positive pressure in the lower collection chamber can be added to eliminate residual slow leakage from filled wells, or to effect agitation by bubbling inert gas through the suspension. In order to empty the wells, the headspace gas outlet valve is closed and the internal pressure raised to about 2 psi. Normally, liquid contents are blown directly to waste 566. However, a 96 well microtiter plate can be inserted into the lower chamber beneath the synthesis plate in order to collect the individual well eluents for spectrophotometric monitoring (trityl, etc.) of reaction progress and yield.

The basic plumbing scheme for the machine is the gas-pressurized delivery of reagents. Each reagent is delivered to the synthesis plate through a dedicated supply line, solenoid valve and nozzle. Reagents never cross paths until they reach the reaction well. Thus, no line needs to be washed or flushed prior to its next use and there is no possibility of cross-contamination of reagents. The liquid delivery velocity is sufficiently energetic to thoroughly mix the contents within a well to form a homogeneous solution, even when employing solutions having drastically different densities. With this mixing, once reactants are in homogeneous solution, diffusion carries the individual components into and out of the solid support matrix where the desired reaction takes place. Each reagent reservoir can be plumbed to either a single nozzle or any combination of up to 8 nozzles. Each nozzle is also provided with a concentric nozzle washer to wash the outside of the delivery nozzles in order to eliminate problems of crystallized reactant buildup due to slow evaporation of solvent at the tips of the nozzles. The nozzles and supply lines can be primed into a set of dummy wells directly to waste at any time.

The entire plumbing system is fabricated with teflon tubing, and reagent reservoirs are accessed via syringe needle/septa or direct connection into the higher capacity bottles. The septum vials are held in removable 8-bottle racks to facilitate easy setup and cleaning. The priming volume for each line is about 350 μl. The minimum delivery volume is about 2 μl, and flow rate accuracy is ±5%. The actual amount of material delivered depends on a timed flow of liquid. The flow rate for a particular solvent will depend on its viscosity and wetting characteristics of the teflon tubing. The flow rate (typically 200-350 μl per sec) is experimentally determined, and this information is contained in the reagent table setup file.

Heating and cooling of the reaction block is effected utilizing a recirculating heat exchanger plate, similar to that found in PCR thermocyclers, that nests with the polypropylene synthesis plate to provide good thermal contact. The liquid contents in a well can be heated or cooled at about 10° C. per minute over a range of +5 to +80° C., as polypropylene begins to soften and deform at about 80° C. For temperatures greater than this, a non-disposable synthesis plate machined from stainless steel or monel with replaceable frits might be utilized.

The hardware controller is designed around a set of three 1 MHz 86332 chips. This controller is used to drive the single x-axis and 8 y-axis stepper motors as well as provide the timing functions for a total of 154 solenoid valves. Each chip has 16 bidirectional timer I/O and 8 interrupt channels in its timer processing unit (TPU). These are used to provide the step and direction signals, and to read 3 encoder inputs and 2 limit switches for controlling up to three motors per chip. Each 86332 chip also drives a serial chain of 8 UNC5891A darlington array chips to provide power to 64 valves with msec resolution. The controller communicates with the Windows software interface program running on a PC via a 19200 Hz serial channel, and uses an elementary instruction set to communicate valve_number and time_open, and motor_number and position_data.

The three components of the software program that run the array synthesizer, the generalized procedure or command (.cmd) file which specifies the synthesis instructions to be performed, the sequence (.seq) file which specifies the scale of the reaction and the order in which variable groups will be added to the core synthon, and the reagent table (.tab) file which specifies the name of a chemical, its location (bottle number), flow rate, and concentration are utilized in conjunction with a basic set of command instructions. The basic set of command instructions are:

| ADD | | |
|---|---|---|
| IF | {block of instructions} | END_IF |
| REPEAT | {block of instructions} | END_REPEAT |

PRIME, NOZZLE_WASH
WAIT, DRAIN
LOAD, REMOVE
NEXT_SEQUENCE
LOOP_BEGIN, LOOP_END

The ADD instruction has two forms, and is intended to have the look and feel of a standard chemical equation. Reagents are specified to be added by a molar amount if the number proceeds the name identifier, or by an absolute volume in microliters if the number follows the identifier. The number of reagents to be added is a parsed list, separated by the “+” sign. For variable reagent identifiers, the key word, <seq>, means look in the sequence table for the identity of the reagent to be added, while the key word, <act>, means add the reagent which is associated with that particular <seq>. Reagents are delivered in the order specified in the list.

Thus:
ADD ACN 300
means: Add 300 μl of the named reagent ACN to each well of active synthesis
ADD <seq>300
means: If the sequence pointer in the .seq file is to a reagent in the list of reagents, independent of scale, add 300 μl of that particular reagent specified for that well.
ADD 1.1 PYR+1.0<seq>+1.1<act1>
means: If the sequence pointer in the seq file is to a reagent in the list of acids in the Class ACIDS_{—}1, and PYR is the name of pyridine, and ethyl chloroformate is defined in the .tab file to activate the class, ACIDS_{—}1, then this instruction means:
   Add 1.1 equiv. pyridine
1.0 equiv. of the acid specified for that well and
1.1 equiv. of the activator, ethyl chloroformate
   The IF command allows one to test what type of reagent is specified in the <seq> variable and process the succeeding block of commands accordingly.

Thus:

| | |
|---|---|
| ACYLATION | {the procedure name} |

BEGIN

IF CLASS = ACIDS_1

ADD 1.0 <seq> + 1.1 <act1> + 1.1 PYR
WAIT 60

ENDIF
IF CLASS = ACIDS_2
ADD 1.0 <seq> + 1.2 <act1> + 1.2 TEA
ENDIF
WAIT 60
DRAIN 10

END

means: Operate on those wells for which reagents contained in the Acid_{—}1 class are specified, WAIT 60 sec, then operate on those wells for which reagents contained in the Acid_{—}2 class are specified, then WAIT 60 sec longer, then DRAIN the whole plate. Note that the Acid_{—}1 group has reacted for a total of 120 sec, while the Acid_{—}2 group has reacted for only 60 sec.

The REPEAT command is a simple way to execute the same block of commands multiple times.

Thus:

| | |
|---|---|
| WASH_1 | {the procedure name} |

BEGIN

REPEAT 3

ADD ACN 300
DRAIN 15

END_REPEAT

END

means: repeats the add acetonitrile and drain sequence for each well three times.

The PRIME command will operate either on specific named reagents or on nozzles which will be used in the next associated <seq> operation. The μl amount dispensed into a prime port is a constant that can be specified in a config.dat file.

The NOZZLE_WASH command for washing the outside of reaction nozzles free from residue due to evaporation of reagent solvent will operate either on specific named reagents or on nozzles which have been used in the preceding associated <seq> operation. The machine is plumbed such that if any nozzle in a block has been used, all the nozzles in that block will be washed into the prime port.

The WAIT and DRAIN commands are by seconds, with the drain command applying a gas pressure over the top surface of the plate in order to drain the wells. The LOAD and REMOVE commands are instructions for the machine to pause for operator action.

The NEXT_SEQUENCE command increments the sequence pointer to the next group of substituents to be added in the sequence file. The general form of a seq file entry is the definition:
Well_No Well_ID Scale Sequence

The sequence information is conveyed by a series of columns, each of which represents a variable reagent to be added at a particular position. The scale (μmole) variable is included so that reactions of different scale can be run at the same time if desired. The reagents are defined in a lookup table (the .tab file), which specifies the name of the reagent as referred to in the sequence and command files, its location (bottle number), flow rate, and concentration. This information is then used by the controller software and hardware to determine both the appropriate slider motion to position the plate and slider arms for delivery of a specific reagent, as well as the specific valve and time required to deliver the appropriate reagents. The adept classification of reagents allows the use of conditional IF loops from within a command file to perform addition of different reagents differently during a ‘single step’ performed across 96 wells simultaneously. The special class ACTIVATORS defines certain reagents that always get added with a particular class of reagents (for example tetrazole during a phosphitylation reaction in adding the next nucleotide to a growing oligonucleotide).

The general form of the .tab file is the definition:
Class Bottle Reagent Name Flow_rate Conc.

The LOOP_BEGIN and LOOP_END commands define the block of commands which will continue to operate until a NEXT_SEQUENCE command points past the end of the longest list of reactants in any well.

Not included in the command set is a MOVE command. For all of the above commands, if any plate or nozzle movement is required, this is automatically executed in order to perform the desired solvent or reagent delivery operation. This is accomplished by the controller software and hardware, which determines the correct nozzle(s) and well(s) required for a particular reagent addition, then synchronizes the position of the requisite nozzle and well prior to adding the reagent.

A MANUAL mode is also utilized in which the synthesis plate and nozzle blocks can be “homed” or moved to any position by the operator, the nozzles primed or washed, the various reagent bottles depressurized or washed with solvent, the chamber pressurized, etc. The automatic COMMAND mode can be interrupted at any point, MANUAL commands executed, and then operation resumed at the appropriate location. The sequence pointer can be increment to restart a synthesis anywhere within a command file.

The queue of oligonucleotides for synthesis can be rearrange or grouped for optimization of synthesis. The oligonucleotides are grouped according to a factor on which to base the optimization of synthesis. As illustrated in the Examples below, one such factor is the 3′ most nucleoside of the oligonucleotide. Using the amidite approach for oligonucleotide synthesis, a nucleotide bearing a 3′ phosphoramite is added to the 5′ hydroxyl group of the a growing nucleotide chain. The first nucleotide (at the 3′ terminus of the oligonucleotide—the 3′ most nucleoside) is first connected to a solid support. This is normally done batch wise on a large scale as is practice during standard oligonucleotide synthesis. Such solid supports pre-loaded with a nucleoside are commercially available. In utilizing the multi well format for oligonucleotide synthesis, for each oligonucleotide to be synthesized, an aliquot of a solid support bearing the proper nucleoside thereon is added to the well for synthesis. Prior to loading the sequence of oligonucleotides to be synthesized in the seq file, they are sorted by the 3′ terminus nucleotide. Based on that sort, all of the oligonucleotide sequences terminating with a “A” nucleoside at their 3′ end are grouped together, those with a “C” nucleoside are grouped together as are those with “G” and “T” nucleosides. Thus in loading the nucleoside bearing solid support in to the synthesis wells, machine movements are conserved.

The oligonucleotides can be group by the above described parameter or other parameters that facilitate the synthesis of the oligonucleotides. Thus, sorting is noted as being effect by some parameter of type 1, as for instance the above described 3′ most nucleoside, or other types of parameters from type 2 to type n. Since synthesis will be from the 3′ end of the oligonucleotides to the 5′ end, the oligonucleotide sequences are reverse sorted to read 3′ to 5′. The oligonucleotides are entered in the the seq file in this form, i.e., reading 3′ to 5′.

Once sorted in to types, the position of the oligonucleotides on the synthesis plates is specified by the creation of a seq file as describe above. The seq file is associated with the respective .cmd and .tab files needed for synthesis of the particular chemistries specified for the oligonucleotides by retrieval of the .cmd and .tab files from the sample database. These files are then input into the multi well synthesizer for oligonucleotide synthesis. Once physically synthesized, library of oligonucleotides again enters the general procedure.

Quality Control

In an optional step, quality control is performed on the oligonucleotides after a decision is made to perform quality control. Although optional, quality control may be desired when there is some reason to doubt that some aspect of the synthetic process has been compromised. Alternatively, samples of the oligonucleotides may be taken and stored in the event that the results of assays conducted using the oligonucleotides yield confusing results or suboptimal data. In the latter event, for example, quality control might be performed if no oligonucleotides with sufficient activity are identified. In either event, the decision step follows quality control step process. If one or more of the oligonucleotides do not pass quality control, the process step can be repeated, i.e., the oligonucleotides are synthesized for a second time.

Sterile, double-distilled water is robotically transferred by an automated liquid handler to each well of a multi-well plate containing a set of lyophilized antisense oligonucleotides. The automated liquid handler reads the barcode sticker on the multi-well plate to obtain the plate's identification number. Automated liquid handler then queries Sample Database (which resides in Database Server) for the quality control assay instruction set for that plate and executes the appropriate steps. Three quality control processes are available.

The first process quantitates the concentration of oligonucleotide in each well. Thus, a “YES” entry in Sample Database under the field “Determine Oligonucleotide Concentration” in the record of Plate Number x causes the Sample Database to send the appropriate instruction set to an automated liquid handler to remove an aliquot from each well of the master plate and generate a replicate daughter plate for transfer to the UV spectrophotometer. The UV spectrophotometer then measures the optical density of each well at a wavelength of 260 nanometers. Using standardized conversion factors, a microprocessor within UV spectrophotometer then calculates a concentration value from the measured absorbance value for each well and output the results to Sample Database.

The second available quality control process quantitates the percent of total oligonucleotide in each well that is full length. Thus, a “YES” entry in Sample Database under the field “Determine % Full Length Oligonucleotide Product” in the record of Plate Number x causes the Sample Database to send the appropriate instruction set to an automated liquid handler to remove an aliquot from each well of the master plate and generate a replicate daughter plate for transfer to the multichannel capillary gel electrophoresis apparatus. The apparatus electrophoretically resolves in capillary tube gels the oligonucleotide product in each well. As the product reaches the distal end of the tube gel during electrophoresis, a detection window dynamically measures the optical density of the product that passes by it. Following electrophoresis, the value of percent product that passed by the detection window with respect to time is utilized by a built in microprocessor to calculate the relative size distribution of oligonucleotide product in each well. These results are then output to the Sample Database.

The third available quality control process quantitates the mass of total oligonucleotide in each well that is full length. Thus, a “YES” entry in Sample Database under the field “Determine Mass of Oligonucleotide Product” in the record of Plate Number x causes the Sample Database to send the appropriate instruction set to an automated liquid handler to remove an aliquot from each well of the master plate and generate a replicate daughter plate for transfer to the multichannel liquid electrospray mass spectrometer. The apparatus then uses electrospray technology to inject the oligonucleotide product into the mass spectrometer. A built in microprocessor calculates the mass-to-charge ratio to arrive at the mass of oligonucleotide product in each well. The results are then output to Sample Database.

Following completion of the selected quality control processes, the output data is manually examined and a decision is made as to whether or not the plate receives “Pass” or “Fail” status. The current criteria for acceptance is that at least 85% of the oligonucleotides in a multi-well plate must be 85% or greater full length product as measured by both capillary gel electrophoresis and mass spectrometry. A manual input is then made into Sample Database as to the pass/fail status of the plate. If a plate fails, the process cycles back, and a new plate of the same oligonucleotides is automatically placed in the plate synthesis request queue. If a plate receives “Pass” status, Sample Database then instructs an automated liquid handler to remove appropriate aliquots from each well of the master plate and generate two replicate daughter plates in which the oligonucleotide in each well is at a concentration of 30 micromolar. The plate then moves on for oligonucleotide activity evaluation.

Cell Lines for Assaying Oligonucleotide Activity

The effect of antisense compounds on target nucleic acid expression can be tested in any of a variety of cell types provided that the target nucleic acid, or its gene product, is present at measurable levels. This can be routinely determined using, for example, PCR or Northern blot analysis. The cell types are described above.

Treatment of Cells with Candidate Compounds

When cells reach about 80% confluency, they are treated with oligonucleotide. For cells grown in 96-well plates, wells are washed once with 200 μl Opti-MEM-1 reduced-serum medium (Life Technologies) and then treated with 130 μl of Opti-MEM-1 containing 3.75 μg/ml LIPOFECTIN (Life Technologies) and the desired oligonucleotide at a final concentration of 150 nM. After 4 hours of treatment, the medium was replaced with fresh medium. Cells were harvested 16 hours after oligonucleotide treatment.

Assaying Oligonucleotide Activity

Oligonucleotide-mediated modulation of expression of a target nucleic acid can be assayed in a variety of ways known in the art.

For example, target RNA levels can be quantitated by, e.g., Northern blot analysis, competitive PCR, or real-time PCR (RT-PCR). RNA analysis can be performed on total cellular RNA or, preferably in the case of polypeptide-encoding nucleic acids, poly(A)+ mRNA. For RT-PCR, poly(A)+ mRNA is suitable. Methods of RNA isolation are taught in, for example, Ausubel et al. (Short Protocols in Molecular Biology, 2nd Ed., pp. 4-1 to 4-13, Greene Publishing Associates and John Wiley & Sons, New York, 1992). Northern blot analysis is routine in the art (Id., pp. 4-14 to 4-29). Real-time polymerase chain reaction (RT-PCR) can be conveniently accomplished using the commercially available ABI PRISM 7700 Sequence Detection System (PE-Applied Biosystems, Foster City, Calif.) according to manufacturer's instructions. Other methods of PCR are also known in the art.

Target protein levels can be quantitated in a variety of ways well known in the art, such as immunoprecipitation, Western blot analysis (immunoblotting), Enzyme-linked immunosorbent assay (ELISA) or fluorescence-activated cell sorting (FACS). Antibodies directed to a protein encoded by a target nucleic acid can be identified and obtained from a variety of sources, such as the MSRS catalog of antibodies, (Aerie Corporation, Birmingham, Mich. or via the internet at www.ANTIBODIES-PROBES.com/), or can be prepared via conventional antibody generation methods. Methods for preparation of polyclonal, monospecific (“antipeptide”) and monoclonal antisera are taught by, for example, Ausubel et al. (Short Protocols in Molecular Biology, 2nd Ed., pp. 11-3 to 11-54, Greene Publishing Associates and John Wiley & Sons, New York, 1992).

Immunoprecipitation methods are standard in the art and are described by, for example, Ausubel et al. (Id., pp. 10-57 to 10-63). Western blot (immunoblot) analysis is standard in the art (Id., pp. 10-32 to 10-10-35). Enzyme-linked immunosorbent assays (ELISA) are standard in the art (Id., pp. 11-5 to 11-17).

Because it is desired to assay the compounds of the invention in a batchwise fashion, i.e., in parallel to the automated synthesis process described above, one means of assaying are suitable for use in 96 well plates and with robotic means. Accordingly, automated RT-PCR is suitable for assaying target nucleic acid levels, and automated ELISA is suitable for assaying target protein levels.

After an appropriate cell line is selected, a decision is made as to whether real-time PCR (RT-PCR) will be the only method by which the activity of the compounds is evaluated. In some instances, it is desirable to run alternate assay methods; for example, hen it is desired to assess target polypeptide levels as well as target RNA levels, an immunoassay such as an ELISA is run in parallel with the RT-PCR assays. Such assays can be tractable to semi-automated or robotic means.

When RT-PCR is used to evaluate the activities of the compounds, cells are plated into multi-well plates (typically, 96 well plates) in process step and treated with test or control oligonucleotides. Then, the cells are harvested and lysed and the lysates are introduced into an apparatus where RT-PCR is carried out. A raw data file is generated, and the data is downloaded and compiled. Spreadsheet files with data charts are generated, and the experimental data is analyzed. Based on the results, a decision is made as to whether it is necessary to to repeat the assays and, if so, the process begins again with step. In any event, data from all the assays on each oligonucleotide is complied and statistical parameters are automatically determined.

Classification of Compounds Based on Their Activity

Following assaying, oligonucleotide compounds are classified according to one or more desired properties. Typically, three classes of compounds are used: active compounds, marginally active (or “marginal”) compounds and inactive compounds. To some degree, the selection criteria for these classes varies from target to target, and members to one or more classes may not be present for a given set of oligonucleotides.

However, some criteria are constant. For example, inactive compounds will typically comprise those compounds having 5% or less inhibition of target expression (relative to basal levels). Active compounds will typically cause at least 30% inhibition of target expression, although lower levels of inhibition are acceptable in some instances. Marginal compounds will have activities intermediate between active and inactive compounds, with marginal compounds having activities more like those of active compounds.

Optimization of Lead Compounds by Sequence

One means by which oligonucleotide compounds are optimized for activity is by varying their nucleobase sequences so that different regions of the target nucleic acid are targeted. Some such regions will be more accessible to oligonucleotide compounds than others, and “sliding” a nucleobase sequence along a target nucleic acid only a few bases can have significant effects on activity. Accordingly, varying or adjusting the nucleobase sequences of the compounds of the invention is one means by which suboptimal compounds can be made optimal, or by which new active compounds can be generated.

The operation of the gene walk process follows. As used herein, the term “gene walk” is defined as the process by which a specified oligonucleotide sequence x that binds to a specified nucleic acid target y is used as a frame of reference around which a series of new oligonucleotides sequences capable of hybridizing to nucleic acid target y are generated that are frame shift increments of oligonucleotide sequence x.

The user manually enters the identification number of the oligonucleotide sequence around which it is desired to execute gene walk process and the name of the corresponding target nucleic acid. The user then enters the scope of the gene walk at step, by which is meant the number of oligonucleotide sequences that it is desired to generate. The user then enters in step a positive integer value for the frame shift increment. Once this data is generated, the gene walk is effected. This causes a subroutine to be executed that automatically generates the desired list of sequences by walking along the target sequence. At that point, the user proceeds to process to assign chemistries to the selected oligonucleotides.

For example, if it was desired to execute the gene walk process using a CD40 antisense oligonucleotide having SEQ ID NO:43 (5′-CTGGCACAAAGAACAGCA; see the Examples below) one could enter the following parameters:

| Gene Walk Parameter | Entered value |
|---|---|
| Oligonucleotide Sequence ID: | ISIS 19225 |
| Name of Gene Target: | CD40 |
| Scope of Gene Walk: | 20 |
| Frame Shift Increment: | 1 |

Entering these values and effecting the gene walk clicking causes the following list to be automatically generated:

| | |
|---|---|
| SEQ ID NO: | Sequence |

| | |
|---|---|
| 44 | GAACAGCACTGACTGTTT |
| 45 | AGAACAGCACTGACTGTT |
| 46 | AAGAACAGCACTGACTGT |
| 47 | AAAGAACAGCACTGACTG |
| 48 | CAAAGAACAGCACTGACT |
| 49 | ACAAAGAACAGCACTGAC |
| 50 | CACAAAGAACAGCACTGA |
| 51 | GCACAAAGAACAGCACTG |
| 52 | GGCACAAAGAACAGCACT |
| 53 | TGGCACAAAGAACAGCAC |
| 54 | GCTGGCACAAAGAACAGC |
| 55 | GGCTGGCACAAAGAACAG |
| 56 | TGGCTGGCACAAAGAACA |
| 57 | CTGGCTGGCACAAAGAAC |
| 58 | CCTGGCTGGCACAAAGAA |
| 59 | TCCTGGCTGGCACAAAGA |
| 60 | GTCCTGGCTGGCACAAAG |
| 61 | TGTCCTGGCTGGCACAAA |
| 62 | CTGTCCTGGCTGGCACAA |
| 63 | TCTGTCCTGGCTGGCACA |

The list shown above contains 20 oligonucleotide sequences directed against the CD40 nucleic acid sequence. They are ordered by the position along the CD40 sequence at which the 5′ terminus of each oligonucleotide hybridizes. Thus, the first ten oligonucleotides are single-base frame shift sequences directed against the CD40 sequence upstream of ISIS 19225 and the latter ten are single-base frame shift sequences directed against the CD40 sequence downstream of ISIS 19225.

In subsequent steps, this new set of nucleobase sequences is used to direct the automated synthesis of a second set of candidate oligonucleotides. These compounds are then taken through subsequent process steps to yield active compounds or reiterated as necessary to optimize activity of the compounds.

Optimization of Lead Compounds by Chemistry

Another means by which oligonucleotoide compounds of the invention are optimized is by reiterating portions of the process of the invention using marginal compounds from the first iteration and selecting additional chemistries to the nucleobase sequences thereof.

Thus, for example, an oligonucleotide chemistry different from than that of the first set of oligoncuelotides is assigned. The nucleobase sequences of marginal compounds are used to direct the synthesis of a second set of oligonucleotides having the second assigned chemistry. The resulting second set of oligonucleotide compounds is assayed in the same manner as the first set and the results are examined to determine if compounds having sufficient activity have been generated.

Identification of Sites Amenable to Antisense Technologies

In a related process, a second oligonucleotide chemistry is assigned to the nucleobase sequences of all of the oligonucleotides (or, at least, all of the active and marginal compounds) and a second set of oligonucleotides is synthesized having the same nucleobase sequences as the first set of compounds. The resulting second set of oligonucleotide compounds is assayed in the same manner as the first set and active and marginal compounds are identified.

In order to identify sites on the target nucleic acid that are amenable to a variety of antisense technologies, the following mathematically simple steps are taken. The sequences of active and marginal compounds from two or more such automated syntheses/assays are compared and a set of nucleobase sequences that are active, or marginally so, in both sets of compounds is identified. The reverse complements of these nucleobase sequences corresponds to sequences of the target nucleic acid that are tractable to antisense and other sequence-based technologies. These antisense-sensitive sites are assembled into contiguous sequences (contigs) using the procedures described for assembling target nucelotide sequences.

Systems for Executing the Process of the Invention

In this embodiment, four main computer servers are provided. Firstly, a large database server stores all chemical structure, sample tracking and genomic, assay, quality control, and program state data. Further, this database server provides serves as the platform for a document management system. Secondly, a compute engine runs computational programs including RNA folding, oligonucleotide walking, and genomic searching. Thirdly, a file server allows raw instrument output storage and sharing of robot instructions. Fourthly, a groupware server enhances staff communication and process scheduling.

A redundant high-speed network system is provided between the main servers and the. These bridges provide reliable network access to the many workstations and instruments deployed for this process. The instruments selected to support this embodiment are all designed to sample directly from standard 96 well microtiter plates, and include an optical density reader, a combined liquid chromatography and mass spectroscopy instrument, a gel fluorescence and scintillation imaging system, a capillary gel electrophoreses system and a real-time PCR system.

Most liquid handling is accomplished automatically using robots with individually controllable robotic pipetters as well a 96 well pipette system for duplicating plates. Windows NT or Macintosh workstations are deployed for instrument control, analysis and productivity support.

Relational Database

Data is stored in an appropriate database. For use with the methods of the invention, a relational database is suitable. Various elements of data are segregated among linked storage elements of the database.

The present invention also provides a cloud algorithm is used to account for mutations and evolutionary changes. Expected base counts can be blurred according to the natural principles of biological mutations, customizing the specific blurring to the biological constraints of each amplified region. Each amplified region of a particular bioagent is constrained in some fashion by its biological purpose (i.e., RNA structure, protein coding, etc.). For example, protein coding regions are constrained by amino acid coding considerations, whereas a ribosome is mostly constrained by base pairing in stems and sequence constraints in unpaired loop regions. Moreover, different regions of the ribosome might have significant preferences that differ from each other. One embodiment of the cloud algorithm is described in Example 1. By collecting all likely species amplicons from a primer set and enlarging the set to include all biologically likely variant amplicons using the cloud algorithm, a suitable cluster region of base count space is defined for a particular species of bioagent. The regions of base count space in which groups of related species are clustered are referred to as “bioclusters.” When a biocluster is constructed, every base count in the biocluster region is assigned a percentage probability that a species variant will occur at that base count. To form a probability density distribution of the species over the biocluster region, the entire biocluster probability values are normalized to one. Thus, if a particular species is present in a sample, the probability of the species biocluster integrated over all of base count space is equal to one. At this point in the ranking procedure, proposed target species to be detected are taken into account. These generally are the bioagents that are of primary importance in a particular detection scenario. For example, if Yersinia pestis (the causative agent of bubonic and pneumonic plague) were the target, the Yersinia pestis species biocluster identified as described above, would be the “target biocluster.” To complete the example, assume that all other database species serve as the scenario background. The discrimination metric in this case is defined as the sum total of all the biocluster overlap from other species into the Yersinia pestis biocluster. In this example, the Yersinia pestis biocluster overlap is calculated as follows. A probability of detection of 99% (PD=0.99) is defined, although this value can be altered as needed. The “detection range” is defined as the set of biocluster base counts, of minimal number, that encloses 99% of the entire target biocluster. For each other bacterial species in the database, the amount of biocluster probability density that resides in the base counts in the defined detection range is calculated and is the effective biocluster overlap between that background species and the target species. The sum of the biocluster overlap over all background species serves as the designation for measuring the discrimination ability of a defined target by a proposed primer set. Mathematically, because the most discriminating primer sets will have minimal biocluster overlap, a designation Φ is defined, Φ=Σθᵢ where i=all bioclusters and where the sum is taken over the individual biocluster overlap values θᵢ from all N background species bioclusters (i=1, . . . , N). Using the inverse figure of merit minimization criteria, also known as the biocluster designation, defined above, the result is that primer set number 4 provides the best discrimination of any of the individual primer sets in the master list. This set of biocluster designation criteria also can be applied to combinations of primer sets. The respective four-dimensional base count spaces from each primer set can be dimensionally concatenated to form a (4×N)-dimensional base count space for N primer sets. Nowhere in the biocluster definition is it necessary that the biocluster reside in a four-dimensional space, thus the biocluster analysis seamlessly adapts to any arbitrary dimensionality. As a result, a master list of primer sets can be searched and ranked according to the biocluster designation of any combination of primer sets with any arbitrary number of primer sets making up the combination. An improved discrimination is achieved through use of an increasing number of primers. For each number of primers value on the x-axis, the plotted inverse figure of merit value is that obtained from the most discriminating group (that group with the minimum figure of merit for that number of primer sets simultaneously used for discrimination). The result is that after the best groups of 3 and 4 primer sets are found, the inverse figure of merit and the potential to differentiate samples according to biocluster designation approaches one and goes no further. That means that there is the equivalent of one background species biocluster overlapping into the target biocluster. In this example it is the Yersinia pseudotuberculosis species biocluster, which cannot be discriminated from Yersinia pestis by any combination of the 16 primer sets in the example. Thus, using the “best” 3 or 4 primer sets in the master list, Yersinia pestis is essentially discriminated from all other species bioclusters, regardless of envisioned or engineered mutations. Moreover, an analysis of examples from any specific species that fill their respective biocluster space, a probability map is derived wherein is defined likely mutational directions for the species, according to evolutionary guidance. Additionally, each biocluster has a level of species specificity whereby allowed mutations further define the species from which the sample relates. The application of this cloud algorithm or cloud logic is not limited to any particular nucleic acid but may be applied to RNA from any source and of any nucleotide length or secondary structure, including RNAs that act in the RNAi mechanism, small nuclear RNAs (snRNAs), small nucleolar RNAs (snoRNAs), small interfering RNAs (siRNAs), tiny noncoding RNAs (tncRNAs) and microRNAs (miRNAs) and synthesized mimics or alterations thereof.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

General

All MS experiments were performed by using an Apex II 70e ESI-FT-ICR MS (Bruker Daltonics, Billerica, Mass.) with an actively shielded 7 tesla superconducting magnet. RNA solutions were prepared in 50 mM NH₄OAc (pH 7), mixed with 10% isopropanol to aid desolvation, and infused at a rate of 1.5 μL/min by using a syringe pump. Ions were formed in a modified electrospray source (Analytica, Branford, Conn.) by using an off-axis grounded electrospray probe positioned about 1.5 cm from the metallized terminus of the glass desolvation capillary biased at 5,000 V. A countercurrent flow of dry oxygen gas heated to 150° C. was used to assist in the desolvation process. Ions were accumulated in an external ion reservoir comprised of a radio frequency-only hexapole, a skimmer cone, and an auxiliary electrode for 1,000 ms before transfer into the trapped ion cell for mass analysis. Each spectrum was the result of the co-addition of 64 transients comprised of 524,288 data points acquired over a 217,391-kHz bandwidth, resulting in a 1.2-sec detection interval. All aspects of pulse sequence control, data acquisition, and postacquisition processing were performed by using a Bruker Daltonics data station running XMASS Version 4.0 on a Silicon Graphics (Mountain View, Calif.) R5000 computer.

Several of the following examples are directed to using mass spectrometry to identify compounds that have affinity for an RNA target molecule. These examples can be easily adapted by one skilled in the art to use mass spectrometry to identify target molecules that have an affinity for a microRNA ligand.

### Example 1

### Mass Spectrometry-Based Selection of Compounds with Affinity for RNA

RNA binding ligands are selected from a set of compounds using mass spectrometry. The RNA used for the target molecule is an RNA whose electrospray ionization properties have been optimized in conjunction with optimization of the electrospray ionization and desolvation conditions. A set of compounds that contains members with molecular mass less than 200, 3 or fewer rotatable bonds, no more than one sulfur, phosphorous, or halogen atom, and at least 20 mM solubility in dimethylsulfoxide is used. A 50 μM stock solution of the RNA is purified, and dialyzed to remove sodium and potassium ions.

The compound set is pooled into mixtures of 8 members, each present at 1-10 mM in DMSO. A collection of these mixtures is diluted 1:50 into an aqueous solution containing 50-150 mM ammonium acetate buffer at pH 7.0, 1-5 μM RNA target, and 10-50% isopropanol, ethanol, or methanol to create the screening sample. The aqueous solution contains 100 μM each of 8 compounds, 50 mM ammonium acetate, 5 μM RNA target, and 25% isopropanol. These screening samples are arrayed in a 96-well microtiter plate, or added to individual vials for queuing into an automated robotic liquid hander under computer control by the mass spectrometer.

The source voltage potentials are adjusted to give stable electrospray ionization by monitoring the ion abundance of the free RNA. The temperature of the desolvation capillary is next reduced incrementally and the voltage potential between the capillary and the first skimmer lens element of the mass spectrometer is adjusted until adducts of ammonia with the RNA can be observed. If available on the mass spectrometers, the partial gas pressure beyond the desolvation capillary is adjusted by throttling the pumping speed. This gas pressure may also be altered to optimize the ion abundance and observation of the ammonium ion adducts. After instrument performance has been optimized, the voltage potential between the capillary and skimmer lens is increase to reduce the abundance of the ion from the monoammonium-RNA complex to ˜10% of the abundance of the ion from the RNA. These instrument parameters are used for detection of complexes between the RNA and compound set.

The compound set is screened for members that form non-covalent complexes with the RNA. The relative abundances and stoichiometries of the non-covalent complexes with the RNA are measured from the integrated ion intensities, and the results are stored in a relational database cross-indexed to the structure of the compounds.

FIG. 2 shows the resulting spectrum obtained after adjustment of operating performance conditions of the mass spectrometer for detection of weak affinity complexes. Free target RNA is seen at 1726.7 m/z in the spectrum. Ions associated with adducts of ammonium with the RNA target can be observed and are easily differentiated from sodium ion adducts based on the combined molecular mass of the ammonium/RNA adducts. Ions associated with an adduct of a triazole ligand (2-amino-4-benzylthio-1,2,4-triazole) are also seen. The RNA target is present at a concentration 5 micromolar and the triazole ligand at a concentration of 100 micromolar and the relative abundances of the ion peaks are normalized to that of the target RNA.

### Example 2

### Chemical Optimization of Compounds that Form Complexes with the RNA Target

In a second step, compounds are obtained with structures derived from those selected in Example 1. These compounds may be simple derivatives with additional methyl, amino, or hydroxyl groups, or derivatives where the composition and size of rings and side chains have been varied. These derivatives are screened as in Example 1 to obtain SAR information and to optimize the binding affinity with the RNA target.

### Example 3

### Determination of the Mode of Binding for Compounds Forming Complexes with the RNA Target

In the compound collection used in Example 1, those compounds that formed complexes with the RNA target are pooled into groups of 4-10 and screened again as a mixture against the RNA target as outlined in Example 1. Since all of the compounds have been shown previously to bind to the RNA, three possible changes in the relative ion abundance are observed in the mass spectrometry assay. If two compounds bind at the same site, the ion abundance of the RNA complex for the weaker binder will be decreased through competition for RNA binding with the higher affinity binder (competitive binding). An example is presented in FIG. 3, where the ion abundance from a glucosamine-RNA complex is reduced as glucosamine is displaced from the RNA by addition of a benzimidazole compound. If two compounds can bind at distinct sites, signals will be observed from the respective binary complexes with the RNA and from the ternary complex where both compounds bind to the RNA simultaneously (concurrent binders). If the binding of one compound enhances the binding of a second compound, the ion abundance from the ternary complex will be enhanced relative to the ion abundance from the respective binary complexes (cooperative binding). An example of cooperative binding between 2-deoxystreptamine (2-DOS) and 3,5-diaminotriazole (3,5-DT) is presented in FIG. 4. The relative ion abundance from the secondary complex for 3,5-DT to the free RNA is measured, as is the relative ion abundance from the ternary complex between 3,5-DT, 2-DOS, and RNA and the binary complex. If the ratio of the relative ion abundance is greater than 1, the binding is considered to be cooperative. The ratios of relative ion abundance are calculated and stored in a database for all compounds that bind to this RNA.

### Example 4

### Amide Library Synthesis—General Procedures

Operations involving resin were carried out in a Quest 210 automated synthesizer (Argonaut Technologies, San Carlos, Calif.). HPLC/MS spectra were obtained on a HP 1100 MSD system (Hewlett-Packard, Palo Alto, Calif.) equipped with a SEDEX (Sedere) evaporative light scattering detector (ELSD). A 4.6×50 mm Zorbax XDB-C18 reversed phase column (Hewlett-Packard, Palo Alto, Calif.) was operated using a linear gradient of 5% A to 100% B over 4 min at 2 mL/min flow rate (A=10 mM aqueous ammonium acetate+1% v/v acetic acid, B=10 mM ammonium acetate in 95:5 v/v acetonitrile/water+1% v/v acetic acid. The flow was split 3:1 after the column, with 0.5 mL/min flowing to the MSD mass detector, and 1.5 mL/min flowing to the ELSD detector. Quantitation was based on integration of the ELSD peak corresponding to product, which was identified by the corresponding mass spectrum of the eluting peak. ¹H NMR spectra for all compounds were recorded either at 399.94 MHz on a Varian Unity 400 NMR spectrometer or at 199.975 MHz on a Varian Gemini 200 NMR spectrometer.

General Procedure for Synthesis of Secondary Amine Resins: Preparation of AG-MB-Benzylamine Resin

2-methoxy-4-alkoxy-benzaldehyde PEG-PS resin (ArgoGel-MB-CHO, Argonaut Technologies, San Carlos, Calif., 10 g, 0.4 mmole/g) was slurried in 30 ml dry trimethylorthoformate (TMOF). Benzylamine (0.52 ml, 4.8 mmole) was added and the slurry swirled gently on a shaker table under dry nitrogen overnight. A solution of 40 ml dry methanol, acetic acid (0.46 ml, 8.0 mmole) and borane-pyridine complex (1.0 ml, 8.0 mmole) was added, and the slurry swirled overnight. The resin was filtered, and washed several times with methanol, DMF, CH₂Cl₂, and finally methanol. Gel-phase NMR showed complete conversion from the aldehyde to secondary benzylamine derivative. Gel-phase ¹³C NMR (C₆D₆) δ 40.9, 48.1, 53.0, 54.8, 67.7, 70.9 (PEG linker), 99.5, 104.7, 121.3, 127.0, 127.8 (poly-styrene beads), 128.5, 130.5, 141.2, 159.0, 159.8.

The supports AG-MB-cyclohexylamine and AG-MB-methylamine, were similarly prepared using cyclohexyl and methylamine (used as a methanol solution available from Aldrich), respectively. The following are the resins employed and the resulting amine functionality of the library compounds.

| resin | amine functionality |
|---|---|
| 1,2-diaminoethane-PS | 1,2-diaminoethane |
| 2-OH-1,3-diaminopropane-PS | 2-OH-1,3-diaminopropane |
| AG-MB-benzylamine | benzylamine |
| AG-MB-cyclohexylamine | cyclohexylamine |
| AG-MB-methylamine | methylamine |
| AG-Rink-NH—Fmoc | amino |
| PS-trityl-piperazine | piperazine |

General Procedure for Synthesis of Amide Motifs

The desired carboxylic acid (1 eq.) was suspended in dry DMF (5 mL/mmole), and HATU (Perseptive Biosystems, 1 eq.) and collidine (3 eq.) was added. The suspension was stirred for 15 min, and if a suspension still existed, diisopropylethylamine (1 eq.) was added, and stirring continued. At this point all acids were in solution. This 0.2 M (5 eq. per eq. of amine on the resin) solution of activated acid was added to the appropriate resin containing a primary or secondary amine, and the mixture was agitated overnight at 65° C. The resins were either purchased from Novabiochem, Argonaut Technologies, or prepared via the general procedure. The mixture was filtered, and the resin washed with DMF (3×), MeOH (3×), CH₂Cl₂ (3×), DMF (3×) and CH₂Cl₂ (3×) and dried with a flow of inert gas. To the resulting resin, trifluoroacetic acid (7 mL/g dry resin) containing 5% v/v triisopropylsilane was added, and the suspension agitated for 4 h. The mixture was filtered, and the resin washed with trifluoroacetic acid (3×). The combined filtrates were concentrated to afford the desired products. The products were characterized by HPLC/MS and were generally sufficiently pure for testing.

The following are the carboxylic acids each of which were coupled with each of the resin bound amines listed above. The corresponding amide functionality of the resulting library compounds are listed thereafter.
carboxylic acid
(R)-(−)-2,2-dimethyl-5-oxo-1,3-dioxolane-4-acetic acid
(S)-(+)-2,2-dimethyl-5-oxo-1,3-dioxolane-4-acetic acid
2,3-dihydroxyquinoxaline-6-carboxylic acid
2-N-Bhoc-guanine-1-acetic acid
4-N-Bhoc-cytosine-1-acetic acid
6-N-Bhoc-adenine-1-acetic acid
bis(BOC-3,5-diaminobenzoic acid)
BOC-3-ABZ-OH
BOC-benzimidazole-5-carboxylic acid
BOC-glycine
BOC-imidazole-4-carboxylic acid
BOC-isonipecotic acid
BOC-SER(tBu)-OH
FMOC-3-amino-1,2,4-triazole-5-carboxylic acid
nalidixic acid
N-BOC-L-homoserine
orotic acid
t-butoxyacetic acid
thymine-1-acetic acid
amide functionality
(R)-3-hydroxy-3-carboxypropionyl
(S)-3-hydroxy-3-carboxypropionyl
2,3-dihydroxyquinoxaline-6-carboxyl
guanine-1-acetyl
cytosine-1-acetyl
adenine-1-acetyl
3,5-diaminobenzoyl
3-aminobenzoyl
5-carboxy-benzimidazole
1-aminoacetyl
imidazole-4-carboxyl
isonipecotyl
(2S)-2-amino-3-hydroxypropionyl
3-amino-1,2,4-triazole-5-carboxyl
nalidixoyl
(2S)-2-amino-4-hydroxybutyryl
orotyl
hydroxyacetyl
thymine-1-acetyl

### Example 5

### (2S)-2-Amino-3-hydroxy-1-piperazinylpropan-1-one

According to the general procedure, the title compound was prepared using PS-trityl-piperazine resin (Novabiochem) and BOC-(tBu)-Serine (Bachem): HPLC/MS M+H 174 fnd., (0.25 min, 100%).

### Thymine-1-acetylpiperazine

According to the general procedure, the title compound was prepared using PS-trityl-piperazine resin (Novabiochem) and thymine-1-acetic acid (Aldrich): HPLC/MS M+H=253 fnd., (0.29 min, 100%).

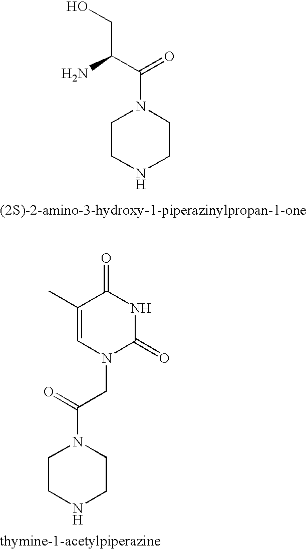

### 1-{2-[(3R)-4-((2S)-2-Amino-3-hydroxypropanoyl)-3-methylpiperazinyl]-2-oxoethyl}-5-methyl-1,3-dihydropyrimidine-2,4-dione

HATU (1.1 g, 2.7 mmol) and DIEA (4.7 mL, 27 mmol) were added sequentially to a solution of Boc-Ser(tBu)-OH (0.71 g, 2.7 mmol) in DMF (10 mL). The mixture was stirred at room temperature for about 30 min then was added to a solution of (R)-(−)-2-methylpiperazine (0.3 g, 3 mmol) in DMF (5 mL). The mixture was stirred for 12 h and was diluted with a mixture of sat. NaHCO₃/EtOAc (200 mL, v/v, 50:50). The aqueous layer was extracted with more EtOAc (2×30 mL). The combined organic layer was dried (Na₂SO₄), filtered, and concentrated in vacuo to give a colorless oily residue, which was used in the next step without purification.

HATU (0.38 g, 1.0 mmol) and 2,4,6-collidine (0.73 mL, 5.5 mmol) were added sequentially to a solution of thymine-1-acetic acid (0.19 g, 1 mmol) in DMF (5 mL). The mixture was stirred at room temperature for about 30 min then was added to a solution of the residue prepared above in DMF (5 mL). The mixture was stirred for 12 h and was diluted with a mixture of sat. NaHCO₃/EtOAc (100 mL, v/v, 50:50). The aqueous layer was extracted with more EtOAc (2×10 mL). The combined organic layer was dried (Na₂SO₄), filtered, and concentrated in vacuo to give a colorless oily residue. Purification of the residue by flash column chromatography (gradient elution 3-5% MeOH/CH₂CL₂) provided N-BOC-O-t-butyl protected derivative (38 mg, 8% yield in two step): TLC (R_{f}=0.4; 10% MeOH/CH₂Cl₂); ¹³CNMR (DMSO-d₆) δ 169.8, 165.4, 164.4, 155.2, 151.0, 142.2, 107.9, 78.2, 72.7, 61.5, 50.3, 48.2, 45.1, 28.1, 27.1, 11.8; HRMS (MALDI) m/z 532.2736 (M+Na)⁺ (C₂₄H₃₉N₅O₇ requires 532.2747).

A solution of the protected derivative (23.4 mg, 0.046 mmol) in concentrated aqueous HCl (2 mL) was stirred at room temperature for 12 h. The reaction mixture was evaporated to give the title compound (20 mg, quantitative yield). ¹³C NMR (CD₃OD) δ 167.3, 167.0, 153.2, 143.9, 111.0, 73.6, 72.4, 62.2, 60.8, 54.4, 47.1, 46.5, 43.8, 12.3.

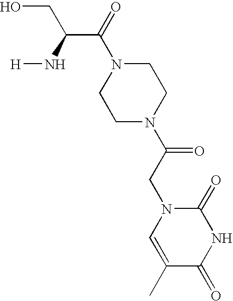

### Example 6

### 2-Deoxy-1,3-diazido-4-[(5-bromo-3-nitro-1,2,4-triazolyl)methyl]-5,6-di-O-acetylstreptamine

Dry hydrogen chloride is passed through a solution of 2-deoxy-1,3-diazido-5,6-di-O-acetylstreptamine (296 mg, 1 mmole, prepared according to the method of Wong et. al., J. Am. Chem. Soc. 1999, 121, 6527-6541) and paraformaldehyde (45 mg, 1.5 mmole) in dichlorethane at 0° C. for 6 h. Solid CaCl₂ is added, the mixture filtered, then concentrated in vacuo. The syrup is azeotroped three times with dry acetonitrile to provide the chloromethyl derivative. Separately, a suspension of 5-bromo-3-nitro-1,2,4-triazole (386 mg, 2 mmole) is stirred with sodium hydride (60% w/w, 80 mg, 2 mmole) for 0.5 h in acetonitrile (20 mL). This suspension is then added directly to the chloromethyl derivative, and the mixture stirred overnight at room temperature. Water and ethyl acetate were added, the organic layer collected, dried over magnesium sulfate, concentrated, and chromatographed (20% ethyl acetate/hexanes) to provide the title compound.

### 2-Deoxy-1,3-diazido-4-[(5-amino-3-nitro-1, 2, 4-triazolyl)methyl]streptamine

2-Deoxy-1,3-diazido-4-[(5-bromo-3-nitro-1,2,4-triazolyl)methyl]-5,6-di-O-acetylstreptamine is dissolved in 3:1 dioxane/28% aqueous ammonia, and the solution stirred at 60° C. in a sealed vessel overnight. The solvent is removed, and the residue chromatographed (10% methanol/chloroform) to provide the title compound.

### 2-Deoxy-4-[(3,5-diamino-1,2,4-triazolyl)methyl]streptamine

2-Deoxy-1,3-diazido-4-[(5-amino-3-nitro-1,2,4-triazolyl)methyl]streptamine is dissolved in ethanol, and hydrogenated over 10% palladium on carbon catalyst at 50 psi with shaking for 72 h. The mixture was filtered through celite, and the solvent removed to afford the title compound.

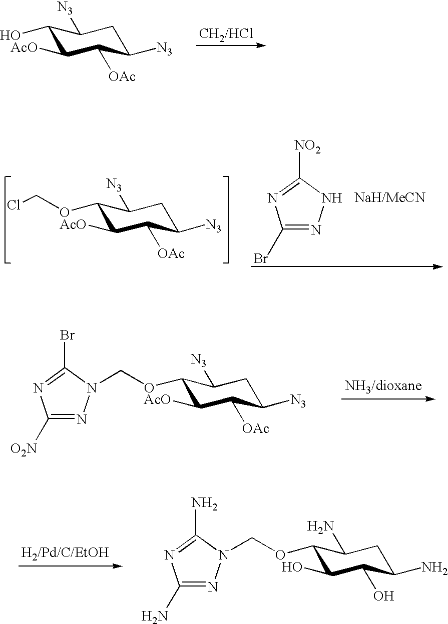

### Examples 7-24

### (Scheme I): Preparation of 1-(8-hydroxy-5-hydroxymethyl-2-methyl-3,6-dioxa-2-aza-bicyclo[3.2.1]oct-7-yl)-1H-pyrimidine-2,4-dione (1)

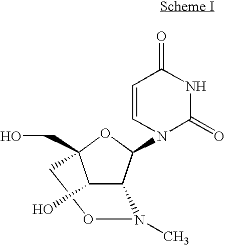

### Example 7

### 1-(3-hydroxy-5,5,7,7-tetraisopropyl-tetrahydro-1,4,6,8-tetraoxa-5,7-disila-cyclopentacycloocten-2-yl)-1H-pyrimidine-2,4-dione (4)

The 3′,5′-protected nucleoside is prepared as illustrated in Karpeisky et. al., Tetrahedron Lett. 1998, 39, 1131-1134. To a solution of arabinouridine (3, 1.0 eq., 0° C.) in anhydrous pyridine is added 1,3-dichloro-1,1,3,3-tetraisopropyldisiloxane (1.1 eq.). The resulting solution is warmed to room temperature and stirred for two hours. The reaction mixture is subsequently quenched with methanol, concentrated to an oil, dissolved in dichloromethane, washed with aqueous NaHCO₃ and saturated brine, dried over anhydrous Na₂SO₄, filtered, and evaporated. Purification by silica gel chromatography will yield Compound 4.

For the preparation of the corresponding cytidine and adenosine analogs, N⁴-benzoyl arabinocytidine and N⁶-benzoyl arabinoadenosine are used, respectively, both of which are prepared from the unprotected arabinonucleoside using the transient protection strategy as illustrated in Ti, et al., J. Am. Chem. Soc. 1982, 104, 1316-1319. Alternatively, the cytidine analog can also be prepared by conversion of the uridine analog as illustrated in Lin, et al., J. Med. Chem. 1983, 26, 1691.

### Example 8

### Acetic Acid 2-(2,4-dioxo-3,4-dihydro-2H-pyrimidin-1-yl)-5,5,7,7-tetraisopropyl-tetrahydro-1,4,6,8-tetraoxa-5,7-disila-cyclopentacycloocten-3-yl Ester (5)

Compound 4 is O-Acetylated using well known literature procedures (Protective Groups in Organic Synthesis, 3^{rd} edition, 1999, pp. 150-160 and references cited therein and in Greene, T. W. and Wuts, P. G. M., eds, Wiley-Interscience, New York.) Acetic anhydride (2 to 2.5 eq.) and triethylamine (4 eq.) is added to a solution of 4 (1 eq.) and N,N-dimethylaminopyridine (0.1 eq.) in anhydrous pyridine. After stirring at room temperature for 1 hour the mixture is treated with methanol to quench excess acetic anhydride and evaporated. The residue is redissolved in ethyl acetate, washed extensively with aqueous NaHCO₃, dried over anhydrous Na₂SO₄, filtered, and evaporated. The compound is used without further purification.

### Example 9

### Acetic Acid 2-(2,4-dioxo-3,4-dihydro-2H-pyrimidin-1-yl)-4-hydroxy-5-hydroxymethyl-tetrahydro-furan-3-yl Ester (6)

The Tips protecting group is removed from Compound 5 as illustrated in the literature (Protective Groups in Organic Synthesis, 3^{rd} edition, 1999, pp. 239 and references therein, Greene, T. W. and Wuts, P. G. M., eds, Wiley-Interscience, New York). To a solution of 5 (1 eq.) in anhydrous dichloromethane is added triethylamine (2 eq.) and triethylamine trihydrofluoride (2 eq.). The reaction mixture is monitored by thin layer chromatography until complete at which point the reaction mixture is diluted with additional dichloromethane, washed with aqueous NaHCO₃, dried over anhydrous Na₂SO₄, and evaporated. The resulting Compound 6 is optionally purified by silica gel chromatography.

### Example 10

### Acetic Acid 5-[bis-(4-methoxy-phenyl)-phenyl-methoxymethyl]-2-(2,4-dioxo-3,4-dihydro-2H-pyrimidin-1-yl)-4-hydroxy-tetrahydro-furan-3-yl Ester (7)

Dimethoxytritylation of Compound 6 is performed using known literature procedures. Formation of the primary 4,4′-dimethoxytrityl ether should be achieved using standard conditions (Nucleic Acids in Chemistry and Biology, 1992, pp. 108-110, Blackburn, Michael G., and Gait, Michael J., eds, IRL Press, New York.) Generally, a solution of 6 (1 eq.) and N,N-dimethylaminopyridine (0.1 eq.) in anhydrous pyridine is treated with 4,4′-dimethoxytrityl chloride (DMTCl, 1.2 eq.) and triethylamine (4 eq.). After several hours at room temperature, excess 4,4′-dimethoxytrityl chloride is quenched with the addition of methanol and the mixture is evaporated. The mixture is dissolved in dichloromethane and washed extensively with aqueous NaHCO₃ and dried over anhydrous Na₂SO₄. Purification by silica gel chromatography will yield Compound 7.

### Example 11

### Acetic Acid 5-[bis-(4-methoxy-phenyl)-phenyl-methoxymethyl]-4-(tert-butyl-diphenyl-silanyloxy)-2-(2,4-dioxo-3,4-dihydro-2H-pyrimidin-1-yl)-tetrahydro-furan-3-yl Ester (8)

The preparation of tert-butyldiphenylsilyl ethers is a common, routine procedure (Protective Groups in Organic Synthesis, 3^{rd} edition, 1999, pp. 141-144 and references therein, Greene, T. W. and Wuts, P. G. M., eds, Wiley-Interscience, New York). In general, a solution of one eq. of 7 and imidazole (3.5 eq.) in anhydrous N,N-dimethylformamide (DMF) is treated with tert-butyldiphenylsilyl chloride (1.2 eq.). After stirring at room temperature for several hours, the reaction mixture is poured into ethyl acetate and washed extensively with water and saturated brine solution. The resulting organic solution is dried over anhydrous sodium sulfate, filtered, evaporated, and purified by silica gel chromatography to give Compound 8.

### Example 12

### Acetic Acid 4-(tert-butyl-diphenyl-silanyloxy)-2-(2,4-dioxo-3,4-dihydro-2H-pyrimidin-1-yl)-5-hydroxymethyl-tetrahydro-furan-3-yl Ester (9)

The 5′-O-DMT group is removed as per known literature procedures 4,4′-dimethoxytrityl ethers are commonly removed under acidic conditions (Oligonucleotides and analogues, A Practical Approach, Eckstein, F., ed, IRL Press, New York.) Generally, Compound 8 (1 eq.) is dissolved in 80% aqueous acetic acid. After several hours, the mixture is evaporated, dissolved in ethyl acetate and washed with a sodium bicarbonate solution. Purification by silica gel chromatography will give compound 9.

### Example 13

### Acetic Acid 4-(tert-butyl-diphenyl-silanyloxy)-2-(2,4-dioxo-3,4-dihydro-2H-pyrimidin-1-yl)-5-formyl-tetrahydro-furan-3-yl Ester (10)

To a mixture of trichloroacetic anhydride (1.5 eq.) and dimethylsulfoxide (2.0 eq.) in dichloromethane at −78° C. is added a solution of Compound 9 in dichloromethane. After 30 minutes, triethylamine (4.5 eq.) is added. Subsequently, the mixture is poured into ethyl acetate, washed with water and brine, dried over anhydrous sodium sulfate, and evaporated to dryness. The resulting material is carried into the next step without further purification. This procedure has been used to prepare the related 4′-C-V-formyl nucleosides (Nomura, M., et. al., J. Med. Chem. 1999, 42, 2901-2908).

### Example 14

### 1-[4-(tert-butyl-diphenyl-silanyloxy)-3-hydroxy-5,5-bis-hydroxymethyl-tetrahydro-furan-2-yl]-1H-pyrimidine-2,4-dione (11)

Hydroxymethylation of the 5′-aldehyde is performed as per the method of Cannizzaro which is well documented in the literature (Jones, G. H., et. al., J. Org. Chem. 1979, 44, 1309-1317). These condisions are expected to additionally remove the 2′-O-acetyl group. Generally, Briefly, formaldehyde (2.0 eq., 37% aq.) and NaOH (1.2 eq., 2 M) is added to a solution of Compound 10 in 1,4-dioxane. After stirring at room temperature for several hours, this mixture is neutralized with acetic acid, evaporated to dryness, suspended in methanol, and evaporated onto silica gel. The resulting mixture is added to the top of a silica gel column and eluted using an appropriate solvent system to give Compound 11.

### Example 15

### 1-[5-[bis-(4-methoxy-phenyl)-phenyl-methoxymethyl]-4-(tert-butyl-diphenyl-silanyloxy)-3-hydroxy-5-hydroxymethyl-tetrahydro-furan-2-yl]-1H-pyrimidine-2,4-dione (12)

Preferential protection with DMT at the ∀-hydroxymethyl position is performed following a published literature procedure (Nomura, M., et. al., J. Med. Chem. 1999, 42, 2901-2908). Generally, a solution of Compound 11 (1 eq.) in anhydrous pyridine is treated with DMTCl (1.3 eq.), then stirred at room temperature for several hours. Subsequently, the mixture is poured into ethyl acetate, washed with water, dried over anhydrous Na₂SO₄, filtered, and evaporated. Purification by silica gel chromatography will yield Compound 12.

### Example 16

### 1-[5-[bis-(4-methoxy-phenyl)-phenyl-methoxymethyl]-4-(tert-butyl-diphenyl-silanyloxy)-5-(tert-butyl-diphenyl-silanyloxymethyl)-3-hydroxy-tetrahydrofuran-2-yl]-1H-pyrimidine-2,4-dione (13)

The 5′-hydroxyl positon is selectively protected with tert-butyldiphenylsilyl following published literature procedures (Protective Groups in Organic Synthesis, 3^{rd} edition, 1999, pp. 141-144 and references therein, Greene, T. W. and Wuts, P. G. M., eds, Wiley-Interscience, New York). Generally, a solution of Compound 12 (1 eq.) and N,N-dimethylaminopyridine (0.2 eq.) in anhydrous dichloromethane is treated with tert-butyldiphenylsilyl chloride (1.2 eq.) and triethylamine (4 eq.). After several hours at room temperature, the reaction is quenched with methanol, poured into ethyl acetate, washed with saturated NaHCO₃, saturated brine, dried over anhydrous Na₂SO₄, filtered, and evaporated. Purification by silica gel chromatography will yield Compound 13.

### Example 17

### Acetic Acid 5-[bis-(4-methoxy-phenyl)-phenyl-methoxymethyl]-4-(tert-butyl-diphenyl-silanyloxy)-5-(tert-butyl-diphenyl-silanyloxymethyl)-2-(2,4-dioxo-3,4-dihydro-2H-pyrimidin-1-yl)-tetrahydro-furan-3-yl Ester (14)

Compound 14 is prepared as per the procedure illustrated in Example 2 above.

### Example 18

### Acetic Acid 4-(tert-butyl-diphenyl-silanyloxy)-5-(tert-butyl-diphenyl-silanyloxymethyl)-2-(2,4-dioxo-3,4-dihydro-2H-pyrimidin-1-yl)-5-hydroxymethyl-tetrahydro-furan-3-yl Ester (15)

Compound 15 is prepared as per the procedure illustrated in Example 9 above.

### Example 19

### Acetic Acid 4-(tert-butyl-diphenyl-silanyloxy)-5-(tert-butyl-diphenyl-silanyloxymethyl)-5-(1,3-dioxo-1,3-dihydro-isoindol-2-yloxymethyl)-2-(2,4-dioxo-3,4-dihydro-2H-pyrimidin-1-yl)-tetrahydro-furan-3-yl Ester (16)

The use of the Mitsunobu procedure to generate the 5′-O-phthalimido nucleosides starting with the 5′-unprotected nucleosides has been reported previously (Perbost, M., et. al., J. Org. Chem. 1995, 60, 5150-5156). Generally, a mixture of Compound 15 (1 eq.), triphenylphosphine (1.15 eq.), and N-hydroxyphthalimide (PhthNOH, 1.15 eq.) in anhydrous 1,4-dioxane is treated with diethyl azodicarboxylate (DEAD, 1.15 eq.). The reaction is stirred at room temperature for several hours until complete by thin layer chromatography. The resulting mixture is evaporated, suspended in ethyl acetate, washed with both saturated NaHCO₃ and saturated brine, dried over anhydrous Na₂SO₄, filtered and evaporated. Purification by silica gel chromatography will yield Compound 16.

### Example 20

### 1-[4-(tert-butyl-diphenyl-silanyloxy)-5-(tert-butyl-diphenyl-silanyloxymethyl)-3-hydroxy-5-methyleneaminooxymethyl-tetrahydro-furan-2-yl]-1H-pyrimidine-2,4-dione (17)

This transformation is performed smoothly in high yield using published procedures (Bhat, B., et. al., J. Org. Chem. 1996, 61, 8186-8199). Generally, a portion of Compound 16 is dissolved in dichloromethane and cooled to −10° C. To this solution is added methylhydrazine (2.5 eq.). After 1-2 hours of stirring at 0° C., the mixture is diluted with dichloromethane, washed with water and brine, dried with anhydrous Na₂SO₄, filtered, and evaporated. The resulting residue is immediately redissolved in a 1:1 mixture of ethyl acetate:methanol, and treated with 20% (w/w) aqueous formaldehyde (1.1 eq.). After an hour at room temperature, the mixture is concentrated then purified by silica gel chromatography to give Compound 17.

### Example 21

### Methanesulfonic Acid 4-(tert-butyl-diphenyl-silanyloxy)-5-(tert-butyl-diphenyl-silanyloxymethyl)-2-(2,4-dioxo-3,4-dihydro-2H-pyrimidin-1-yl)-5-methyleneaminooxymethyl-tetrahydro-furan-3-yl Ester (18)

The mesylation of hydroxyl groups proceeds readily under these conditions (Protective Groups in Organic Synthesis, 3^{rd} edition, 1999, pp. 150-160 and references cited therein). Briefly, to a solution of Compound 17 in a 1:1 mixture of anhydrous dichloromethane and anhydrous pyridine is added methanesulfonyl chloride (1.2 eq.). After stirring at room temperature for several hours, this mixture is quenched with methanol, concentrated, diluted with dichloromethane, washed with aqueous NaHCO₃ and brine, dried over anhydrous Na₂SO₄, filtered and evaporated. Purification by silica gel chromatography will yield Compound 18.

### Example 22

### 1-[8-(tert-butyl-diphenyl-silanyloxy)-5-(tert-butyl-diphenyl-silanyloxymethyl)-2-methyl-3,6-dioxa-2-aza-bicyclo[3.2.1]oct-7-yl]-1H-pyrimidine-2,4-dione (19)

The reduction of the formaldoxime moiety is performed as per known literature procedures. Generally, a solution of Compound 18 in methanol is treated with sodium cyanoborohydride (1.5 eq.). This treatment will result in quantitative reduction of the formaldoxime moiety to yield the 4′-C-(aminooxymethyl) arabinonucleoside. The proximity of the methylated electron-rich amine to the activated 2′-O-mesylate will result in the spontaneous ring closing of this intermediate to yield bicyclic Compound 19. The reaction is monitored by thin layer chromatography until completion. The mixture is then poured into ethyl acetate, washed extensively with aqueous NaHCO₃ and brine, dried over anhydrous Na₂SO₄, filtered and evaporated. Purification by silica gel chromatography will yield Compound 19.

### Example 23

### 1-(8-hydroxy-5-hydroxymethyl-2-methyl-3,6-dioxa-2-aza-bicyclo [3.2.1]oct-7-yl)-1H-pyrimidine-2,4-dione (1)

The tert-butyldiphenylsilyl ether protecting groups are readily cleaved by treatment with tetrabutylammonium fluoride (Protective Groups in Organic Synthesis, 3^{rd} edition, 1999, pp. 141-144 and references therein, Greene, T. W. and Wuts, P. G. M., eds, Wiley-Interscience, New York). Briefly, a solution of Compound 19 in a minimal amount of tetrahydrofuran (THF) is treated with a 1 M solution of tetrabutylammonium fluoride (TBAF, 5-10 eq.) in THF. After several hours at room temperature, this mixture is evaporated onto silica gel and subjected to silica gel chromatography to give Compound 1.

Alternate Sythetic Route to Compound 1, Synthesis of Guanosine Analog

### Example 24

### 4-benzyloxy-5-benzyloxymethyl-5-hydroxymethyl-2-methoxy-tetrahydro-furan-3-ol (21)

The preparation of the protected 4′-C-hydroxymethylribofuranose, Compound 20, follows published literature procedures (Koshkin, A. A., et. al., Tetrahedron 1998, 54, 3607-3630). Compound 20 (1 eq.) is dissolved in anhydrous methanol and hydrogen chloride in an anhydrous solvent (either methanol or 1,4-dioxane) is added to give a final concentration of 5% (w/v). After stirring at room temperature for several hours, the mixture is concentrated to an oil, dried under vacuum, and used in the next step without further purification.

### Example 25

### 2-(3-benzyloxy-2-benzyloxymethyl-4-hydroxy-5-methoxy-tetrahydro-furan-2-ylmethoxy)-isoindole-1,3-dione (22)

The O-phthalimido compound is prepared following the reference cited and the procedures illustrated in Example 13 above. The reaction can be adjusted to preferentially react at the primary hydroxyl e.g. the 4′-C-hydroxymethyl group (Bhat, B., et. al., J. Org. Chem. 1996, 61, 8186-8199). Generally, a solution of 21 (1 eq.), N-hydroxyphthalimide (1.1 eq.), and triphenylphosphine (1.1 eq.) in anhydrous tetrahydrofuran is treated with diethyl azodicarboxylate (1.1 eq.). After several hours at room temperature, the mixture is concentrated and subjected to silica gel chromatography to give Compound 22.

### Example 26

### Formaldehyde O-(3-benzyloxy-2-benzyloxymethyl-4-hydroxy-5-methoxy-tetrahydro-furan-2-ylmethyl)-oxime (23)

Compound 23 is prepared as per the procedure illustrated in Example 14 above.

### Example 27

### Methanesulfonic Acid 4-benzyloxy-5-benzyloxymethyl-2-methoxy-5-methyleneaminooxymethyl-tetrahydro-furan-3-yl Ester (24)

Mesylation is achieved with inversion of configuration using Mitsunobu conditions (Anderson, N. G., et. al., J. Org. Chem. 1996, 60, 7955). Generyally, a mixture of Compound 23 (1 eq.), triphenylphosphine (1.2 eq.) and methanesulfonic acid (1.2 eq.) in anhydrous 1,4-dioxane is treated with diethyl azodicarboxylate (1.2 eq.). After stirring at room temperature for several hours, the resulting mixture is concentrated and subjected to silica gel chromatography to give Compound 24.

### Example 28

### 8-benzyloxy-5-benzyloxymethyl-7-methoxy-2-methyl-3,6-dioxa-2-aza-bicyclo[3.2.1]octane (25)

Compound 25 is prepared as per the procedure illustrated in Example 16 above.

### Example 29

### Acetic Acid 8-benzyloxy-5-benzyloxymethyl-2-methyl-3,6-dioxa-2-aza-bicyclo[3.2.1]oct-7-yl Ester (26)

Compound 25 is dissolved in 80% (v/v) aqueous acetic acid. After 1-2 hours at room temperature, the solution is concentrated, then dissolved in dichloromethane and washed with saturated aqueous NaHCO₃ and brine. The organic portion is subsequently dried over anhydrous Na₂SO₄, filtered, and concentrated. The resulting mixture is coevaporated from anhydrous pyridine, then dissolved in anhydrous pyridine and treated with acetic anhydride (2 eq.). The solution is stirred overnight, quenched with methanol, dissolved in ethyl acetate and washed extensively with saturated NaHCO₃. The organic portion is then dried (Na₂SO₄), filtered and evaporated without further purification.

### Example 30

### 1-(8-benzyloxy-5-benzyloxymethyl-2-methyl-3,6-dioxa-2-aza-bicyclo [3.2.1]oct-7-yl)-1H-pyrimidine-2,4-dione (27)

Compound 26 is converted to one of several N-glycosides (nucleosides) using published chemistry procedures including either Vorbrüggen chemistry or one of several other methods (Chemistry of Nucleosides and Nucleotides, Volume 1, 1988, edited by Leroy B. Townsend, Plenum Press, New York). To prepare the uradinyl analog, a mixture of Compound 26 (1 eq.) and uracil (1.3 eq.) is suspended in anhydrous acetonitrile. To the suspension is added N,O-bis-(trimethylsilyl)-acetamide (BSA, 4 eq.). The suspension is heated to 70° C. for 1 hour, then cooled to 0° C. and treated with trimethylsilyl-trifluoromethanesulfonate (TMSOTf, 1.6 eq.). The resulting solution is heated at 55° C. until the reaction appears complete by TLC. The reaction mixture is poured into ethyl acetate and washed extensively with saturated NaHCO₃, dried over anhydrous Na₂SO₄, filtered, evaporated, and purified by silica gel chromatography to give Compound 30.

In order to use the above preparation with nucleobases with reactive functional groups the reactive functional groups are protected prior to use. For example such protected nucleobases include naturally occurring nucleobases such as N⁴-benzoyl cytosine, N⁶-benzoyl adenine and N²-isobutyryl guanine.

### Example 31

### 1-(8-hydroxy-5-hydroxymethyl-2-methyl-3,6-dioxa-2-aza-bicyclo[3.2.1]oct-7-yl)-1H-pyrimidine-2,4-dione (1)

To give the desired product, Compound 1 the benzyl ethers protecting groups are removed following published literature procedures (Koshkin, A. A., et. al., Tetrahedron 1998, 54, 3607-3630). Generally, the bis-O-benzylated bicyclic Compound 27 is dissolved in methanol. To this solution is added 20% Pd(OH)₂/C, and the resulting suspension is maintained under an atmosphere of H₂ at 1-2 atm pressure. This mixture is stirred at room temperature for several hours until complete by TLC, at which point the Pd(OH)₂/C is removed by filtration, and the filtrate is concentrated and purified by silica gel chromatography, if necessary, to give Compound 1.

### Example 32

### 2′-O-tert-butyldimethylsilyl-3′-C-styryluridine (33)

Compound 28 is treated with DMTCl, in pyridine in presence of DMAP to get 5′-DMT derivative, Compound 29. Compound 29 is treated with TBDMSCl in pyridine to which yields both the 2′ and the 3′-silyl derivative. The 3′-TBDMS derivative is isolated by silica gel flash column chromatography and further heated with phenyl chlorothionoformate and N-chlorosuccinimide in a solution of pyridine in benzene 60° C. to give Compound 31. Compound 31 is treated with β-tributylstannylstyrene and AIBN in benzene give Compound 32. Compound 32 is detritylated with dichloroacetic acid in dichloromethane give compound 33.

### Example 33

### 1-[(1R,3R,8S)-8-[(2-cyanoethyl)bis(1-methylethyl)phosphoramidite)-3-[(4,4′-dimethoxytrityloxy)methyl]-5-methyl-2-oxo-5-azabicyclo[2.3.1]octane-5-methyl-2,4-(1H,3H)-pyrimidinedione (40)

Compound 33 is treated with oxalyl chloride in DMSO in the presence of ethyl diisopropylamine to give the 5′-aldehyde which is then subjected to a tandem aldol condensation and Cannizzaro reaction using aqueous formaldehyde and 1 M NaOH in 1,4-dioxane to yield the diol, Compound 34. Selective silylation with TBDMSCl in pyridine and isolation of the required isomer will give Compound 35. Compound 35 is treated with methanesulfonyl chloride in pyridine to give the methane sufonyl derivative which is treated with methanolic ammonia to give compound 36. The double bond of Compound 36 is oxidatively cleaved by oxymylation go give the diol and then by cleavage of the diol with sodium periodate to give the aldehyde, Compound 37. The amino and aldehyde groups in Compound 37 are cross coupled under reductive condition followed by methylation of the amino group with formaldehyde in the presence of sodium borohydride will give the Compound 38. Treatment of Compound 38 with triethylamine trihydrofluoride and triethylamine in THF will give Compound 39. The primary alcohol of Compound 39 is selectively titylated with DMTCl in pyridine followed by phosphytilation at 8-position to give Compound 40.

### Example 34

### 1-[(1R,3R,8S)-8-[(2-cyanoethyl)bis(1-methylethyl)phosphoramidite)-3-[(4,4′-dimethoxytrityloxy)methyl]-5-methyl-2-oxo-5-azabicyclo[3.2.1]octan-4-one-5-methyl-2,4-(1H,3H)-pyrimidinedione (20)

Compound 35 is benzylated with benzyl bromide in DMF and sodium hydride to give Compound 41. Oxidative cleavage of Compound 41 will give an aldehyde at the 2′-position which is reduced to the corresponding alcohol using sodium borohydride in methanol to give Compound 42. Compound 42 is converted into the 3′-C-aminomethyl derivative, Compound 43 by in situ generation of the methane sulfonyl derivative and treatment with ammonia. The amino group in Compound 43 is protected with an Fmoc protecting group using Fmoc-Cl and sodium bicarbonate in aqueous dioxane to give Compound 44. Deprotection of the benzyl group is achieved with BCl₃ in dichloromethane at −78° C. followed by oxidation of the alcohol with pyridinium dichromate in DMF give the corresponding carboxylic acid. The deprotection of the Fmoc group releases the amino group at the 2′-position to give Compound 45. Compound 45 is treated with TBTU (2-(1H-benzotriazole-1-yl)-1,1,3,3-tetramethyluroniumtetrafluoroborate) and triethylamine in DMF to yield Compound 46. Compound 46 is desilylated with triethylamine trihydrofluoride in triethylamine in THF followed by tritylation at 3 position to give the 3-trityloxymethyl derivative followed by phosphytilation at 8-position to give Compound 47. The DMT phosphoramidite bicyclic nucleoside, Compound 47 is purified by silica gel flash column chromatography.

### Example 35

### Synthesis of Nucleoside Phosphoramidites

The following compounds, including amidites and their intermediates were prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N-4-benzoyl-5-methylcytidine penultimate intermediate for 5-methyl dC amidite, [5′-O-(4,4′-Dimethoxytriphenylmethyl)-2′-deoxy-N⁴-benzoyl-5-methylcytidin-3′-O-yl]-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, [5′-O-(4,4′-Dimethoxytriphenylmethyl)-2′-O-(2-methoxyethyl)-5-methyluridin-3′-O-yl]-2-cyanoethyl-N,N-diisopropylphosphoramidite (MOE T amidite), 5′-O-Dimethoxytrityl-2′-O-(2-methoxyethyl)-5-methylcytidine intermediate, 5′-O-dimethoxytrityl-2′-O-(2-methoxyethyl)-N⁴-benzoyl-5-methyl-cytidine penultimate intermediate, [5′-O-(4,4′-Dimethoxytriphenylmethyl)-2′-O-(2-methoxyethyl)-N⁴-benzoyl-5-methylcytidin-3′-O-yl]-2-cyanoethyl-N,N-diisopropylphosphoramidite (MOE 5-Me-C amidite), [5′-O-(4,4′-Dimethoxytriphenylmethyl)-2′-O-(2-methoxyethyl)-N⁶-benzoyladenosin-3′-O-yl]-2-cyanoethyl-N,N-diisopropylphosphoramidite (MOE A amdite), [5′-O-(4,4′-Dimethoxytriphenylmethyl)-2′-O-(2-methoxyethyl)-N⁴-isobutyrylguanosin-3′-O-yl]-2-cyanoethyl-N,N-diisopropylphosphoramidite (MOE G amidite), 2′-O-(Aminooxyethyl) nucleoside amidites and 2′-O-(dimethylaminooxyethyl) nucleoside amidites, 2′-(Dimethylaminooxyethoxy) nucleoside amidites, 5′-O-tert-Butyldiphenylsilyl-O²-2′-anhydro-5-methyluridine, 5′-O-tert-Butyldiphenylsilyl-2′-O-(2-hydroxyethyl)-5-methyluridine, 2′-O-([2-phthalimidoxy)ethyl]-5′-t-butyldiphenylsilyl-5-methyluridine, 5′-O-tert-butyldiphenylsilyl-2′-O-[(2-formadoximinooxy)ethyl]-5-methyluridine, 5′-O-tert-Butyldiphenylsilyl-2′-O-[N,N dimethylaminooxyethyl]-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-[(2-cyanoethyl)-N,N-diisopropylphosphoramidite], 2′-(Aminooxyethoxy) nucleoside amidites, N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite], 2′-dimethylaminoethoxyethoxy(2′-DMAEOE) nucleoside amidites, 2′-O-[2(2-N,N-dimethylaminoethoxy)ethyl]-5-methyl uridine, 5′-O-dimethoxytrityl-2′-O-[2(2-N,N-dimethylaminoethoxy)-ethyl)]-5-methyl uridine and 5′-O-Dimethoxytrityl-2′-O-[2(2-N,N-dimethylaminoethoxy)-ethyl)]-5-methyl uridine-3′-O-(cyanoethyl-N,N-diisopropyl)phosphoramidite.

### Example 36

### Oligonucleotide and Oligonucleoside Synthesis

The chimeric oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH₄OAc solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. No. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in published PCT applications PCT/US94/00902 and PCT/US93/06976 (published as WO 94/17093 and WO 94/02499, respectively), herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone oligomeric compounds having, for instance, alternating MMI and P═O or P═S linkages are prepared as described in U.S. Pat. Nos. 5,378,825, 5,386,023, 5,489,677, 5,602,240 and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 37

### RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand, 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

RNA oligomeric compounds (RNA oligonucleotides) for use in the present invention can be synthesized by the methods herein or purchased from Dharmacon Research, Inc (Lafayette, Colo.). Once synthesized, complementary RNA oligomeric compounds can then be annealed by methods known in the art to form double stranded (duplexed) oligomeric compounds. For example, duplexes can be formed by combining 30 μl of each of the complementary strands of RNA oligonucleotides (50 uM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed oligomeric compounds can be used in kits, assays, screens, or other methods to investigate the role of a target nucleic acid.

### Example 38

### Synthesis of Chimeric Oligomeric Compounds

Chimeric oligomeric compounds, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

### [2′-O-Me]-[2′-deoxy]-[2′-O-Me]Chimeric Phosphorothioate Oligonucleotides

Chimeric oligomeric compounds having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

### [2′-O-(2-Methoxyethyl)]-[2′-deoxy]-[2′-O-(Methoxyethyl)]Chimeric Phosphorothioate Oligonucleotides

[2′-O-(2-methoxyethyl)]-[2′-deoxy]-[2′-O-(methoxyethyl)]chimeric phosphorothioate oligonucleotides were prepared as per the procedure above for the 2′-O-methyl chimeric oligomeric compound, with the substitution of 2′-O-(methoxyethyl) amidites for the 2′-O-methyl amidites.

### [2′-O-(2-Methoxyethyl)Phosphodiester][2′-deoxy Phosphorothioate]-[2′-O-(2-Methoxyethyl)Phosphodiester]Chimeric Oligomeric Compounds

[2′-O-(2-methoxyethyl phosphodiester]-[2′-deoxy phosphorothioate]-[2′-O-(methoxyethyl) phosphodiester]chimeric oligomeric compounds are prepared as per the above procedure for the 2′-O-methyl chimeric oligomeric compound with the substitution of 2′-O-(methoxyethyl)amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

The above methods are also applicable to the synthesis of chimeric oligomeric compounds having multiple alternating regions such as olignucleotides having the formula: T₁-(3′-endo region)-[(2′-deoxy region)-(3′-endo region)]ₙ-T₂. The use of 2′-MOE or other nucleoside amidites will enable the preparation of a myriad of different oligonucleotides.

Other chimeric oligomeric compounds, chimeric oligonucleosides and mixed chimeric oligomeric compounds/oligonucleosides are synthesized according to U.S. Pat. No. 5,623,065, herein incorporated by reference.

### Example 39

### Design and Screening of Duplexed Oliogmeric Compounds

In accordance with the present invention, a series of nucleic acid duplexes comprising the ligands and/or targets employed in the methods of the present invention and their complements can be designed to target a nucleic acid molecule, such as a pre-mRNA, processed RNA, intron, exon, and the like. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed oligomeric compounds are evaluated for their ability to modulate a target expression. When cells reached 80% confluency, they are treated with duplexed oligomeric compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex oligomeric compound at a final concentration of 200 nM. After 5 hours of treatment, the medium is replaced with fresh medium. Cells are harvested 16 hours after treatment, at which time RNA is isolated and target reduction measured by RT-PCR.

### Example 40

### Oligonucleotide and Small Noncoding RNA Isolation

Oligonucleotides and small noncoding RNA samples can be size fractionated and gel purified by methods disclosed herein or those commonly used in the art. Briefly, total RNA can be extracted using a guanidine-based denaturation solution and standard methods known in the art. Subsequently, low molecular weight RNA can be isolated by anion-exchange chromatography (RNA/DNA Midi Kit, Qiagen, Valencia, Calif.). Small RNAs can be further resolved by electrophoresis on 15% polyacrylamide (30:0.8) denaturing gels containing 7 M urea in TBE buffer (45 mM Tris-borate, pH 8.0, 1.0 mM EDTA), and a gel slice containing RNAs of approximately 15 to 35 nucleotides (based on RNA oligonucleotide size standards) can be excised and eluted in 0.3 M NaCl at 4° C. for approximately 16 hours. The eluted RNAs can be precipitated using ethanol and resuspended in diethyl pyrocarbonate-treated water.

### Example 41

### Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH₄OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 42

### Oligonucleotide Analysis—96-Well Plate Format

The concentration of oligonucleotide in each well was assessed by dilution of samples and UV absorption spectroscopy. The full-length integrity of the individual products was evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition was confirmed by mass analysis of the oligomeric compounds utilizing electrospray-mass spectroscopy. All assay test plates were diluted from the master plate using single and multi-channel robotic pipettors. Plates were judged to be acceptable if at least 85% of the oligomeric compounds on the plate were at least 85% full length.

### Example 43

### Cell Culture and Oligonucleotide Treatment

The effect of chimeric oligomeric compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 Cells:

The human transitional cell bladder carcinoma cell line T-24 is obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. For Northern blotting or other analyses, cells harvested when they reached 90% confluence. Cells were seeded into 96-well plates (Falcon-Primaria #353872) at a density of 7000 cells/well for use in real-time RT-PCR analysis.

A549 Cells:

The human lung carcinoma cell line A549 is obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

HMECs:

Normal human mammary epithelial cells (HMECs) are obtained from American Type Culture Collection (Manassus, Va.). HMECs are routinely cultured in DMEM high glucose (Invitrogen Life Technologies, Carlsbad, Calif.) supplemented with 10% fetal bovine serum (Invitrogen Life Technologies, Carlsbad, Calif.). Cells are routinely passaged by trypsinization and dilution when they reach approximately 90% confluence. HMECs are plated in 24-well plates (Falcon-Primaria # 353047, BD Biosciences, Bedford, Mass.) at a density of 50,000-60,000 cells per well, and allowed to attach overnight prior to treatment with oligomeric compounds. HMECs are plated in 96-well plates (Falcon-Primaria #353872, BD Biosciences, Bedford, Mass.) at a density of approximately 10,000 cells per well and allowed to attach overnight prior to treatment with oligomeric compounds.

MCF7 Cells:

The breast carcinoma cell line MCF7 is obtained from American Type Culture Collection (Manassus, Va.). MCF7 cells are routinely cultured in DMEM high glucose (Invitrogen Life Technologies, Carlsbad, Calif.) supplemented with 10% fetal bovine serum (Invitrogen Life Technologies, Carlsbad, Calif.). Cells are routinely passaged by trypsinization and dilution when they reach approximately 90% confluence. MCF7 cells are plated in 24-well plates (Falcon-Primaria # 353047, BD Biosciences, Bedford, Mass.) at a density of approximately 140,000 cells per well, and allowed to attach overnight prior to treatment with oligomeric compounds. MCF7 cells are plated in 96-well plates (Falcon-Primaria #353872, BD Biosciences, Bedford, Mass.) at a density of approximately 20,000 cells per well and allowed to attach overnight prior to treatment with oligomeric compounds.

T47D Cells:

The breast carcinoma cell line T47D is obtained from American Type Culture Collection (Manassus, Va.). T47D cells are deficient in expression of the tumor suppressor gene p53. T47D cells are cultured in DMEM high glucose (Invitrogen Life Technologies, Carlsbad, Calif.) supplemented with 10% fetal bovine serum (Invitrogen Life Technologies, Carlsbad, Calif.). Cells are routinely passaged by trypsinization and dilution when they reach approximately 90% confluence. T47D cells are plated in 24-well plates (Falcon-Primaria # 353047, BD Biosciences, Bedford, Mass.) at a density of approximately 170,000 cells per well, and allowed to attach overnight prior to treatment with oligomeric compounds. T47D cells are plated in 96-well plates (Falcon-Primaria #353872, BD Biosciences, Bedford, Mass.) at a density of approximately 20,000 cells per well and allowed to attach overnight prior to treatment with oligomeric compounds.

BJ Cells:

The normal human foreskin fibroblast BJ cell line was obtained from American Type Culture Collection (Manassus, Va.). BJ cells were routinely cultured in MEM high glucose with 2 mM L-glutamine and Earle's BSS adjusted to contain 1.5 g/L sodium bicarbonate and supplemented with 10% fetal bovine serum, 0.1 mM non-essential amino acids and 1.0 mM sodium pyruvate (all media and supplements from Invitrogen Life Technologies, Carlsbad, Calif.). Cells were routinely passaged by trypsinization and dilution when they reached approximately 80% confluence. Cells were plated on collagen-coated 24-well plates (Falcon-Primaria #3047, BD Biosciences, Bedford, Mass.) at approximately 50,000 cells per well, and allowed to attach to wells overnight.

B16-F10 Cells:

The mouse melanoma cell line B16-F10 was obtained from American Type Culture Collection (Manassas, Va.). B16-F10 cells were routinely cultured in DMEM high glucose (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 80% confluence. Cells were seeded into collagen-coated 24-well plates (Falcon-Primaria #3047, BD Biosciences, Bedford, Mass.) at approximately 50,000 cells per well and allowed to attach overnight.

HUVECs:

Human vascular endothelial cells (HUVECs) are obtained from American Type Culture Collection (Manassus, Va.). HUVECs are routinely cultured in EBM (Clonetics Corporation, Walkersville, Md.) supplemented with SingleQuots supplements (Clonetics Corporation, Walkersville, Md.). Cells are routinely passaged by trypsinization and dilution when they reach approximately 90% confluence and are maintained for up to 15 passages. HUVECs are plated at approximately 3000 cells/well in 96-well plates (Falcon-Primaria #353872, BD Biosciences, Bedford, Mass.) and treated with oligomeric compounds one day later.

NHDF Cells:

Human neonatal dermal fibroblast (NHDF) cells are obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) are obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

293T Cells:

The human 293T cell line is obtained from American Type Culture Collection (Manassas, Va.). 293T cells are a highly transfectable cell line constitutively expressing the simian virus 40 (SV40) large T antigen. 293T cells were maintained in Dulbeccos' Modified Medium (DMEM) (Invitrogen Corporation, Carlsbad, Calif.) supplemented with 10% fetal calf serum and antibiotics (Life Technologies).

HepG2 Cells:

The human hepatoblastoma cell line HepG2 is obtained from the American Type Culture Collection (ATCC) (Manassas, Va.). HepG2 cells are routinely cultured in Eagle's MEM supplemented with 10% fetal bovine serum, 1 mM non-essential amino acids, and 1 mM sodium pyruvate (medium and all supplements from Invitrogen Life Technologies, Carlsbad, Calif.). Cells are routinely passaged by trypsinization and dilution when they reach approximately 90% confluence. For treatment with oligomeric compounds, cells are seeded into 96-well plates (Falcon-Primaria #353872, BD Biosciences, Bedford, Mass.) at a density of approximately 7000 cells/well prior to treatment with oligomeric compounds. For the caspase assay, cells are seeded into collagen coated 96-well plates (BIOCOAT cellware, Collagen type I, B-D #354407/356,407, Becton Dickinson, Bedford, Mass.) at a density of 7500 cells/well.

Preadipocytes:

Human preadipocytes are obtained from Zen-Bio, Inc. (Research Triangle Park, N.C.). Preadipocytes were routinely maintained in Preadipocyte Medium (ZenBio, Inc., Research Triangle Park, N.C.) supplemented with antibiotics as recommended by the supplier. Cells were routinely passaged by trypsinization and dilution when they reached 90% confluence. Cells were routinely maintained for up to 5 passages as recommended by the supplier. To induce differentiation of preadipocytes, cells are then incubated with differentiation media consisting of Preadipocyte Medium further supplemented with 2% more fetal bovine serum (final total of 12%), amino acids, 100 nM insulin, 0.5 mM IBMX, 1 μM dexamethasone and 1 μM BRL49653. Cells are left in differentiation media for 3-5 days and then re-fed with adipocyte media consisting of Preadipocyte Medium supplemented with 33 μM biotin, 17 μM pantothenate, 100 nM insulin and 1 μM dexamethasone. Cells differentiate within one week. At this point cells are ready for treatment with the oligomeric compounds of the invention. One day prior to transfection, 96-well plates (Falcon-Primaria #353872, BD Biosciences, Bedford, Mass.) are seeded with approximately 3000 cells/well prior to treatment with oligomeric compounds.

Differentiated Adipocytes:

Human adipocytes are obtained from Zen-Bio, Inc. (Research Triangle Park, N.C.). Adipocytes were routinely maintained in Adipocyte Medium (ZenBio, Inc., Research Triangle Park, N.C.) supplemented with antibiotics as recommended by the supplier. Cells were routinely passaged by trypsinization and dilution when they reached 90% confluence. Cells were routinely maintained for up to 5 passages as recommended by the supplier.

NT2 Cells:

The NT2 cell line is obtained from the American Type Culture Collection (ATCC; Manassa, Va.). The NT2 cell line, which has the ATCC designation NTERA-2 cl.D 1, is a pluripotent human testicular embryonal carcinoma cell line derived by cloning the NTERA-2 cell line. The parental NTERA-2 line was established in 1980 from a nude mouse xenograft of the Tera-2 cell line (ATCC HTB-106). NT2 cells were routinely cultured in DMEM, high glucose (Invitrogen Corporation, Carlsbad, Calif.) supplemented with 10% fetal bovine serum (Invitrogen Corporation, Carlsbad, Calif.). Cells were routinely passaged by trypsinization and dilution when they reached 90% confluence. For Northern blotting or other analyses, cells harvested when they reached 90% confluence.

HeLa Cells:

The human epitheloid carcinoma cell line HeLa is obtained from the American Tissue Type Culture Collection (Manassas, Va.). HeLa cells were routinely cultured in DMEM, high glucose (Invitrogen Corporation, Carlsbad, Calif.) supplemented with 10% fetal bovine serum (Invitrogen Corporation, Carlsbad, Calif.). Cells were routinely passaged by trypsinization and dilution when they reached 90% confluence. For Northern blotting or other analyses, cells were harvested when they reached 90% confluence.

Treatment with Antisense Oligomeric Compounds:

When cells reached 65-75% confluency, they were treated with oligonucleotide. For cells grown in 96-well plates, wells were washed once with 100 μL OPTI-MEM™-1 reduced-serum medium (Invitrogen Corporation, Carlsbad, Calif.) and then treated with 130 μL of OPTI-MEM™-1 containing 3.75 μg/mL LIPOFECTIN™ (Invitrogen Corporation, Carlsbad, Calif.) and the desired concentration of oligonucleotide. Cells are treated and data are obtained in triplicate. After 4-7 hours of treatment at 37° C., the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO:64) which is targeted to human H-ras, or ISIS 18078, (GTGCGCGCGAGCCCGAAATC, SEQ ID NO:65) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO:66, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### Example 44

### Analysis of Oligonucleotide Inhibition of a Target Expression

Antisense modulation of a target expression can be assayed in a variety of ways known in the art. For example, a target mRNA levels can be quantitated by, e.g., Northern blot analysis, competitive polymerase chain reaction (PCR), or real-time PCR (RT-PCR). Real-time quantitative PCR is presently suitable. RNA analysis can be performed on total cellular RNA or poly(A)+ mRNA. One method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of a target can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### Example 45

### Design of Phenotypic Assays and In Vivo Studies for the Use of a Target Inhibitors

Phenotypic Assays

Once target inhibitors have been identified by the methods disclosed herein, the oligomeric compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of a target in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with a target inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the geneotype of the cell (measurement of the expression of one or more of the genes of the cell) after treatment is also used as an indicator of the efficacy or potency of the a target inhibitors. Hallmark genes, or those genes suspected to be associated with a specific disease state, condition, or phenotype, are measured in both treated and untreated cells.

In Vivo Studies

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study.

To account for the psychological effects of receiving treatments, volunteers are randomly given placebo or a target inhibitor. Furthermore, to prevent the doctors from being biased in treatments, they are not informed as to whether the medication they are administering is a a target inhibitor or a placebo. Using this randomization approach, each volunteer has the same chance of being given either the new treatment or the placebo.

Volunteers receive either the a target inhibitor or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding a target or a target protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and a target inhibitor treatment. In general, the volunteers treated with placebo have little or no response to treatment, whereas the volunteers treated with the a target inhibitor show positive trends in their disease state or condition index at the conclusion of the study.

### Example 46

### RNA Isolation

Poly(A)+ mRNA Isolation

Poly(A)+ mRNA was isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 47

### Real-Time Quantitative PCR Analysis of a Target mRNA Levels

Quantitation of a target mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

Probes and are designed to hybridize to a human a target sequence, using published sequence information.

### Example 48

### Northern Blot Analysis of a Target mRNA Level

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human a target, a human a target specific primer probe set is prepared by PCR To normalize for variations in loading and transfer efficiency membranes are stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 49

### Modulation of Target Expression by microRNA Ligands

In accordance with the present invention, a series of oligomeric compounds are designed to target different regions of the human target RNA. The oligomeric compounds are analyzed for their effect on human target mRNA levels by quantitative real-time PCR as described in other examples herein. Data are averages from three experiments. The target regions to which these sequences are complementary are herein referred to as “suitable target segments” and are therefore suitable for targeting by oligomeric compounds of the present invention. The sequences represent the reverse complement of the suitable chimeric oligomeric compounds.

As these “suitable target segments” have been found by experimentation to be open to, and accessible for, hybridization with the chimeric oligomeric compounds of the present invention, one of skill in the art will recognize or be able to ascertain, using no more than routine experimentation, further embodiments of the invention that encompass other oligomeric compounds that specifically hybridize to these suitable target segments and consequently inhibit the expression of a target.

According to the present invention, chimeric oligomeric compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 50

### Western Blot Analysis of a Target Protein Level

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to a target is used, with a radiolabeled or fluorescently labeled secondary antibody directed against the primary antibody species. Bands are visualized using a PHOSPHORIMAGER™ (Molecular Dynamics, Sunnyvale Calif.).

### Example 51

### Gel Shift Assay

A gel shift protocol, though nominally a low-throughput method, is an important tool for the initial selection of the assay configuration for oligonucleotide-based methods to be used with each particular target RNA. Typically, the target RNA is transcribed in vitro, using as template a DNA that contains the sequence for the T7-RNA polymerase promoter followed by a region encoding the target RNA, and [³²P]-UTP to produce radiolabelled RNA. Conditions for the T7 RNA polymerase transcription reaction using oligonucleotide templates have been described by Milligan et al., Nuc. Acids Res., 15: 8783, 1987. The [³²P]-labelled target RNA is optionally heat denatured at 90° C. for 2 min and chilled on ice for 2 min, after which aliquots are incubated in the absence and presence of test ligands and increasing concentrations of a complementary oligonucleotide. The reaction mixtures are then resolved in polyacrylamide native gels containing or lacking the test ligands.

If a test ligand binds the target RNA, it will inhibit the formation of hybrids between the target and the complementary oligonucleotide.

### Example 52

### High Throughput Assays Using Streptavidin-Biotin

In these embodiments, a biotin moiety is introduced into the complementary oligonucleotide or into the target RNA. This allows the use of capture methods that are based on the strong interaction between biotin and avidin, streptavidin (SA) or other derivatives of biotin-binding proteins (Wilchek et al., Meth. Enzymol., 184:5-45, 1990). Only those labelled RNA target molecules that are hybridized to the biotinylated oligonucleotide can become associated with the biotin binding protein.

The biotin moiety can be located at any position in the oligonucleotide, or there can be multiple biotin moieties per oligonucleotide molecule. The SA (or its derivatives or analogues) can be covalently attached to a solid support, or it can be added free in solution. The target RNA can be radiolabelled, for example, or labelled with a fluorophore such as fluorescein or rhodamine or any other label that can be readily measured.

In a typical assay, 96-well plates coated with SA, which are commercially available (Pierce, Rockford, Ill.), are used. The reactions are first set up in a regular uncoated 96-well plate by mixing the labeled RNA target with the biotinylated oligonucleotide and the ligands to be tested. After an incubation period, the reaction mixture is transferred into a SA coated plate to allow binding of the biotin moiety to SA. Target RNA:oligonucleotide hybrids and free biotinylated oligonucleotide will bind to the wall of the plate through the SA-biotin interaction. Unbound material, including target RNA that is not associated with the oligonucleotide, is washed away with an excess of buffer. The target RNA remaining in the plate after the wash is quantified by an appropriate method, depending upon the nature of the label in the target RNA.

SA coated beads: In this embodiment, the SA is covalently attached to small beads of an inert material, such as, for example, sepharose (Pharmacia, Uppsala, Sweden), agarose (Sigma Chemical Co., St. Louis, Mo.), or Affigel (BioRad, Hercules, Calif.). A fixed amount of beads is added to each well containing the reaction mixture to allow binding of the hybrids. The beads are then washed to remove unbound material before quantitation.

SA coated paramagnetic beads: The washing step required when using SA-beads can be facilitated by using paramagnetic-SA coated beads (PMP-SA). These beads are commercially available (Promega, Madison, Wis.) and can be concentrated and held by a magnet. Positioning the magnet under the plate during washing steps concentrates and retains the beads at the bottom of the well during the washing procedure thus preventing loss of beads and allowing faster operation.

SA coated SPA beads and scintillant containing plates: Scintillation proximity assay (SPA) is a technology available from Amersham Corp. (Arlington Heights, Ill.) that can be used for measurement of hybridization. SA coated SPA beads contain a solid phase scintillant that can be excited by low energy isotopes in close proximity. In this embodiment, the target RNA is labelled with ³H (whose weak radiation energy is virtually undetectable at distances of more than one micron unless the signal is amplified by a scintillant). When using SPA beads, only those radiolabelled molecules that bind to the SA-SPA-beads will be close enough to cause the scintillant to emit a detectable signal, while those molecules in solution will not contribute to the signal. Therefore, by using a biotinylated oligonucleotide and a ³H labelled RNA target, it is possible to determine the amount of hybrids formed in the reaction by adding SA-SPA beads to the reaction and then counting in a LSC-counter after a brief binding incubation period. Plates coated with SA (which contain a scintillant attached to the surface of the wells) are also commercially available (Scintiplates.RTM., Packard, Meriden, Conn.) can be used for this assay in place of SA-SPA beads.

Adsorption to nitrocellulose filters: This embodiment takes advantage of the fact that most proteins tightly adsorb to nitrocellulose filters. When using a biotinylated oligonucleotide and a labelled target RNA, free SA or a SA derivative such as SA:alkaline phosphatase conjugate (SA:AP), SA:.beta.-galactosidase (SA:BG), or other SA-conjugate or fusion protein, is added to the reaction to allow binding to the biotin moiety in the oligonucleotide. Subsequently, the reaction is filtered through 96-well nitrocellulose filter plates (Millipore, Bedford, Mass., HATF or NC). Labeled target RNA hybridized to the biotinylated oligonucleotide is retained in the filter through the adsorption of SA or its derivative to the filter, while unhybridized RNA passes through. SA has been found to bind poorly to nitrocellulose filters; however, the present inventors have discovered that the use of SA conjugates or fusion proteins increases the adsorption of the protein to nitrocellulose. Therefore, the use of SA fusion proteins or conjugates is suitable.

### Example 53

### High-Throughput Assays Using Covalently Attached Proteins

Polypeptides and proteins can be covalently attached to the 5′-end of nucleic acids that have been treated with a carbodiimide to form an activated 5′-phosphorimidazolide derivative that will readily react with amines including those in polypeptides and proteins (Chu et al., Nuc. Acids Res., 11:6513, 1983). Using this approach, any peptide or protein of choice can be covalently attached to the 5′-end of the RNA target or the complementary oligonucleotide used in the present invention. Non-limiting examples of embodiments that use this technique are described below.

Adsorption to nitrocellulose filters: With a peptide or protein covalently attached to the oligonucleotide and a labeled RNA target, or, conversely, with a peptide or protein covalently attached to the RNA target and a labeled oligonucleotide, hybridization can be quantified in essentially the same way as described above for the SA based capture of biotin containing hybrids in nitrocellulose filters. In this case, however, binding to the filter is via the peptide or protein adduct in the RNA target, or in the oligonucleotide.

Affinity binding to solid supports: All the techniques described above for the use of SA-biotin can be duplicated by using any of a number of other affinity pairs in place of SA and biotin. The adduct “Y” attached to the oligonucleotide (or target RNA) is capable of high affinity binding with a specific molecule “X” which is attached to a solid support. Activated resins (beads) and 96-well plastic plates for attachment of macromolecules or their derivatives are commercially available (Dynatech, Chantilly, Va.). X and Y can be a number of combinations including antigen-antibody, protein-protein, protein-substrate, and protein-nucleic acid pairs. Some of these pairs are shown in the following table:

| X | Y |
|---|---|
| Antigen/epitope | Specific antibody |
| Protein A | Immunoglobulin |
| Glutathione | Glutathione-S-transferase |
| Maltose | Maltose binding protein |
| RNA or DNA motif | Specific motif binding protein |

In most cases either component of the pair can be attached to the RNA target (or oligonucleotide) while the other is attached to the solid support. However, if a specific RNA or DNA binding protein is used, it is suitable to attach the protein to the solid support while the specific sequence motif that the protein binds can be incorporated during synthesis at any convenient position in the sequence of the RNA target or oligonucleotide. Finally, attachment of the protein to solid support can be omitted if adsorption to nitrocellulose filters is used instead (as described above).

### Example 54

### High-Throughput Assays Using Fluorescence Energy Transfer (FET)

Fluorophores such as fluorescein, rhodamine and coumarin have distinctive excitation and emission spectra. Fluorescence energy transfer occurs between pairs of fluorophores in which the emission spectrum of one (donor) overlaps the excitation spectrum of the other (acceptor). For appropriately chosen pairs of fluorescent molecules, emission by the donor probe is reduced by the presence of an acceptor probe in close proximity because of direct energy transfer from the donor to acceptor. Thus, upon excitation at a wavelength absorbed by the donor probe, a reduction in donor emission and increase in acceptor emission relative to the probes alone is observed if the probes are close in space. In other words, the donor's emission fluorescence is quenched by the acceptor, which in turn emits a higher wavelength fluorescence. FET, however, is effective only when donor and acceptor are in close proximity. The efficiency of energy transfer is inversely proportional to the sixth power of the distance between the donor and acceptor probes, thus the extent of these effects can be used to calculate the distance separating the probes. FET has been used to probe the structure of transfer RNA molecules (Beardsley et al., Proc. Natl. Acad. Sci. USA 65:39, 1970), as well as for detection of hybridization, for restriction enzyme assays, for DNA-unwinding assays, and for other applications).

Intramolecular FET

FET is used in the present invention to monitor a change in target RNA conformation, when the distance between the donor and acceptor probes differs significantly between the different conformations. In one embodiment an RNA molecule is used in which an internal hybridization probe sequence has been engineered as in, for example, FIG. 3. The solid region represents target RNA sequences and the hatched region represents an internal probe sequence that is complementary to a large portion of the target sequence. In conformation 1, the probe and target sequences hybridize, bringing the acceptor (A) and donor (D) fluorescent probes into close proximity. In the presence of a ligand that binds to a structured conformation of the target sequences, conformation 2 is stabilized; as a consequence, the probes are further apart. A predominance of conformation 2 is reflected in a relative increase in donor fluorescence and/or a decrease in acceptor fluorescence.

Use of Oligonucleotide Hybridization and FET

Acceptor and donor in the same strand: In this embodiment, the target RNA is designed so that the 5′- and 3′-ends of the RNA stay in close proximity when folded, allowing FET. FIG. 4 shows the model in which the formation of hybrids with another DNA or RNA oligonucleotide will result in a decrease in the FET efficiency due to the larger distance between donor and acceptor.

Acceptor and donor in separate strands: This approach is useful when the design of the RNA target does not allow the incorporation of both donor and acceptor fluorophores in the same strand. In this case, the donor and acceptor are in separate strands and come in close proximity in the target:oligonucleotide hybrid. In this embodiment, the formation of the hybrid results in an increase of FET.

### Example 55

### High-Throughput Assays Using Conformation-Specific Nucleases

In practicing the present invention the ligand-induced stabilization of a folded conformation of a target RNA by binding decreases the fraction of the target RNA present in an unfolded conformation. Conversely, in the absence of ligand, a greater fraction of the RNA is found in the unfolded state than in the presence of such a compound. Folded conformations of RNA are characterized by double-stranded regions in which base pairing between RNA strands occurs. A variety of nucleolytic enzymes, such as S1 and mung bean nucleases, preferentially digest phosphodiester bonds in single-stranded RNA relative to double stranded RNA. Such enzymes can be used to probe the conformation of RNA target molecules in the current invention.

In a typical assay, target RNA and test compound(s) are preincubated to allow binding to occur. Next, an appropriate nuclease is added, and the mixture is incubated under appropriate conditions of temperature, nuclease concentration, ionic strength and denaturant concentration to ensure that (in the absence of ligand) about 75% of the RNA is digested according to the specificity of the nuclease used, within a short incubation period (typically 30 minutes). The extent of digestion is then measured using any method well-known in the art for distinguishing between free ribonucleoside monophosphates and oligonucleotides, including, without limitation, acid precipitation and detection of labelled RNA, FET of RNA containing donor and acceptor fluorescence probes, and electrophoretic separation and detection of RNA by autoradiography, fluorescence, UV absorbance, hybridization with labeled nucleic acid probe or dye binding.

Changes in conformation between two or more alternative folded RNA conformations can also be detected using nuclease digestion. In this embodiment, each of the conformations typically contains some regions of double stranded RNA. If the alternate conformations involve differing amounts of double-stranded regions, they can be distinguished by measuring the amount of nuclease-resistant material. If the overall double-stranded content of these structures is comparable, it is necessary to distinguish between the nuclease-resistant fragments yielded by nuclease digestion of different target RNA conformations. For example, although regions B and B′ are found among the nuclease resistant fragments of both conformations 1 and 2, region A is not found after digestion of conformation 2. Specific RNA fragments may be detected and quantified by any method well-known in the art, including, without limitation, labelling of target RNA, hybridization with target-specific probes, amplification using target-specific primers and reverse-transcriptase-coupled PCR, and size determination of digestion products (if digestion products of a specific RNA conformation have characteristic sizes that distinguish them from the digestion products of other conformations).

Nucleases that are specific for different nucleic acid structures may also be used to quantify hybridization of complementary oligonucleotides.

RNAse H: RNAse H is a commercially available nuclease that specifically degrades the RNA strand of RNA:DNA hybrids. A 5′-end or 3′-end biotinylated RNA target is also labeled at the other end with a radionuclide or a fluorophore such as fluorescein, rhodamine or coumarin. RNAase H digestion of the RNA:DNA hybrids formed during the reaction results in physical separation of the biotin moiety (on one end) from the fluorophore or radionuclide (on the other end). RNA target strands not involved in hybrid formation will not be digested by RNAse H and can be quantified after streptavidin binding as described above. In this embodiment, the signal obtained will increase if the test ligand binds the target RNA.

Nuclease S1: Single stranded nucleic acids can be specifically digested with the commercially available nuclease S1 (Promega, Madison, Wis.). This enzyme can be used in the present invention if the DNA oligonucleotide carries the biotin moiety as well as the label at an internal position. Labelled strands forming hybrids resist digestion by S1 nuclease and are quantified by SA-mediated capture as described above. The label can also be in the section of RNA that participates in hybrid formation. Alternatively, the same approach can be carried out with single strand specific RNases such as RNAse T1 or RNAase ONE.™. (Promega), in which case the label must be located in the RNA target.

### Example 56

### Conformation Specific Binding

A variety of materials bind with greater affinity to one or another type of RNA structure. A prime example of this phenomenon is hydroxyapatite, which has greater affinity for double-stranded than for single-stranded nucleic acids. Nitrocellulose, by contrast, has higher affinity for single-stranded than for double-stranded RNA. These and other similar materials can be used to distinguish between different conformations of RNA, particularly where ligand binding stabilizes one conformation that differs significantly from other conformations in its single-stranded content. These methods are generally useful when ligand binding stabilizes folded RNA conformations relative to the unfolded state.

Antibodies that recognize RNA may also be used in a high-throughput mode to identify ligands according to the present invention. Useful antibodies may recognize specific RNA sequences (and/or conformations of such sequences) (Deutscher et al., Proc. Natl. Acad. Sci. USA 85:3299, 1988), may bind to double-stranded or single-stranded RNA in a sequence-independent manner (Schonborn et al., Nuc. Acids Res. 19:2993, 1991), or may bind DNA:RNA hybrids specifically (Stumph et al., Biochem. 17:5791, 1978). In these embodiments, binding of antibodies to the target RNA is measured in the presence and absence of test ligands.

### Example 57

### Biophysical Measurements

A variety of biophysical measurements can be used to examine the folded and unfolded conformation(s) of RNA molecules and detect the relative amounts of such conformations, including, without limitation, UV absorbance, CD spectrum, intrinsic fluorescence, fluorescence of extrinsic covalent or noncovalent probes, sedimentation rate, and viscosity. Each of these properties may change with changing RNA conformation. In these embodiments, measurements are performed on mixtures of target RNA and appropriate buffer, salt and denaturants in the presence and absence of test ligand(s). A change in a measurable property, particularly one that suggests conversion of unfolded to folded forms of the RNA, is indicative of ligand binding.

### Example 58

### Changes in Conformational Stability

Any of the structural measurements described above can be used to examine the stabilization of a conformation by ligand binding. The stability of such a conformation is defined as the free energy difference between that conformation and alternative (typically unfolded) conformations. Conformational stability can be measured under constant conditions, with and without test ligand(s), or over a range of conditions. For example, the effect of increasing temperature on structure, as measured by one of the methods above, can be measured in the presence and absence (control) of test ligand(s). An increase in the temperature at which structure is lost is indicative of ligand binding.

### Example 59

### Disruption of Protein Binding to Adjacent RNA

A variety of proteins are known that bind to specific RNA sequences in a manner that is dependent on the three-dimensional structure of the RNA. In these embodiments, protein binding is used as a probe of RNA structure and its alteration upon ligand binding. A target RNA sequence and an RNA sequence to which a protein binds are incorporated within the same RNA molecule. The interaction of a binding protein with its binding sequence is measured in the presence and absence of test ligands. Ligand-induced changes in the RNA conformation that alter the conformation of the protein binding site are detected by measurement of protein.

Binding to a given target RNA is a prerequisite for pharmaceuticals intended to modify directly the action of that RNA. Thus, if a test ligand is shown, through use of the present method, to bind an RNA that reflects or affects the etiology of a condition, it may indicate the potential ability of the test ligand to alter RNA function and to be an effective pharmaceutical or lead compound for the development of such a pharmaceutical. Alternatively, the ligand may serve as the basis for the construction of hybrid compounds containing an additional component that has the potential to alter the RNA's function. In this case, binding of the ligand to the target RNA serves to anchor or orient the additional component so as to effectuate its pharmaceutical effects. The fact that the present method is based on physico-chemical properties common to most RNAs gives it widespread application. The present invention can be applied to large-scale systematic high-throughput procedures that allow a cost-effective screening of many thousands of test ligands. Once a ligand has been identified by the methods of the present invention, it can be further analyzed in more detail using known methods specific to the particular target RNA used. For example, the ligand can be tested for binding to the target RNA directly, such as, for example, by incubating radiolabelled ligand with unlabelled target, and then separating RNA-bound and unbound ligand. Furthermore, the ligand can be test for its ability to influence, either positively or negatively, a known biological activity of the target RNA.

### Example 60

### Selection of CD40 as a Target

Cell-cell interactions are a feature of a variety of biological processes. In the activation of the immune response, for example, one of the earliest detectable events in a normal inflammatory response is adhesion of leukocytes to the vascular endothelium, followed by migration of leukocytes out of the vasculature to the site of infection or injury. The adhesion of leukocytes to vascular endothelium is an obligate step in their migration out of the vasculature (for a review, see Albelda et al., FASEB J., 1994, 8, 504). As is well known in the art, cell-cell interactions are also critical for propagation of both B-lymphocytes and T-lymphocytes resulting in enhanced humoral and cellular immune responses, respectively (for a reviews, see Makgoba et al., Immunol. Today, 1989, 10, 417; Janeway, Sci. Amer., 1993, 269, 72).

CD40 was first characterized as a receptor expressed on B-lymphocytes. It was later found that engagement of B-cell CD40 with CD40L expressed on activated T-cells is essential for T-cell dependent B-cell activation (i.e. proliferation, immunoglobulin secretion, and class switching) (for a review, see Gruss et al. Leuk Lymphoma, 1997, 24, 393). A full cDNA sequence for CD40 is available (GenBank accession number X60592, incorporated herein as SEQ ID NO:67).

As interest in CD40 mounted, it was subsequently revealed that functional CD40 is expressed on a variety of cell types other than B-cells, including macrophages, dendritic cells, thymic epithelial cells, Langerhans cells, and endothelial cells (Id.). These studies have led to the current belief that CD40 plays a much broader role in immune regulation by mediating interactions of T-cells with cell types other than B-cells. In support of this notion, it has been shown that stimulation of CD40 in macrophages and dendritic results is required for T-cell activation during antigen presentation (Id.). Recent evidence points to a role for CD40 in tissue inflammation as well. Production of the inflammatory mediators IL-12 and nitric oxide by macrophages have been shown to be CD40 dependent (Buhlmann et al., J. Clin. Immunol., 1996, 16, 83). In endothelial cells, stimulation of CD40 by CD40L has been found to induce surface expression of E-selectin, ICAM-1, and VCAM-1, promoting adhesion of leukocytes to sites of inflammation (Buhlmann et al., J. Clin. Immunol, 1996, 16, 83; Gruss et al., Leuk Lymphoma, 1997, 24, 393). Finally, a number of reports have documented overexpression of CD40 in epithelial and hematopoietic tumors as well as tumor infiltrating endothelial cells, indicating that CD40 may play a role in tumor growth and/or angiogenesis as well (Gruss et al., Leuk Lymphoma, 1997, 24, 393-422; Kluth et al. Cancer Res, 1997, 57, 891).

Due to the pivotal role that CD40 plays in humoral immunity, the potential exists that therapeutic strategies aimed at downregulating CD40 may provide a novel class of agents useful in treating a number of immune associated disorders, including but not limited to graft versus host disease, graft rejection, and autoimmune diseases such as multiple sclerosis, systemic lupus erythematosus, and certain forms of arthritis. Inhibition of CD40 may also prove useful as an anti-inflammatory compound, and could therefore be useful as treatment for a variety of diseases with an inflammatory component such as asthma, rheumatoid arthritis, allograft rejections, inflammatory bowel disease, various dermatological conditions, and psoriasis. Finally, as more is learned of the association between CD40 overexpression and tumor growth, inhibitors of CD40 may prove useful as anti-tumor agents as well.

Currently, there are no known therapeutic agents which effectively inhibit the synthesis of CD40. To date, strategies aimed at inhibiting CD40 function have involved the use of a variety of agents that disrupt CD40/CD40L binding. These include monoclonal antibodies directed against either CD40 or CD40L, soluble forms of CD40, and synthetic peptides derived from a second CD40 binding protein, A20. The use of neutralizing antibodies against CD40 and/or CD40L in animal models have provided evidence that inhibition of CD40 stimulation would have therapeutic benefit for GVHD, allograft rejection, rheumatoid arthritis, SLE, MS, and B-cell lymphoma (Buhlmann et al., J. Clin. Immunol, 1996, 16, 83). However, due to the expense, short half-life, and bioavailability problems associated with the use of large proteins as therapeutic agents, there is a long felt need for additional agents capable of effectively inhibiting CD40 function. Oligonucleotides compounds avoid many of the pitfalls of current agents used to block CD40/CD40L interactions and may therefore prove to be uniquely useful in a number of therapeutic applications.

### Example 61

### Generation of Virtual Oligoncueltoides Targeted to CD40

The process of the invention was used to select oligonucleotides targeted to CD40, generating the list of oligonucleotide sequences with desired properties. From the assembled CD40 sequence, the process began with determining the desired oligonucleotide length to be eighteen nucleotides. All possible oligonucleotides of this length were generated by Oligo 5.0. Desired thermodynamic properties were selected. The single parameter used was oligonucleotides of melting temperature less than or equal to 40° C. were discarded. Oligonucleotide melting temperatures were calculated by Oligo 5.0. Oligonucleotide sequences possessing an undesirable score were discarded. It is believed that oligonucleotides with melting temperatures near or below physiological and cell culture temperatures will bind poorly to target sequences. All oligonucleotide sequences remaining were exported into a spreadsheet and desired sequence properties were selected. These include discarding oligonucleotides with stretch of four guanosines in a row and stretches of six of any other nucleotide in a row. A spreadsheet macro removed all oligonucleotides containing the text string “GGGG”. Another spreadsheet macro removed all oligonucleotides containing the text strings “AAAAAA” or “CCCCCC” or “TTTTTT”. From the remaining oligonucleotide sequences, approximately 100 sequences were selected manually with the criteria of having an even distribution of oligonucleotide sequences throughout the target sequence. These oligonucleotide sequences were then passed to the next step in the process, assigning actual oligonucleotide chemistries to the sequences.

### Example 62

### Input Files for Automated Oligonucleotide Synthesis

Command File (.cmd File)

Command file for synthesis of oligonucleotide having regions of 2′-O-(methoxyethy) nucleosides and region of 2′-deoxy nucleosides each linked by phosphorothioate internucleotide linkages.

SOLID_SUPPORT_SKIP

BEGIN

Next_Sequence

END

INITIAL-WASH

BEGIN

Add ACN 300
Drain 10

END

LOOP-BEGIN
DEBLOCK

BEGIN

Prime TCA
Load Tray
Repeat 2

Add TCA 150
Wait 10
Drain 8

End_Repeat
Remove Tray

Add TCA 125
Wait 10
Drain 8

END

WASH_AFTER_DEBLOCK

BEGIN

Repeat 3

Add ACN 250 To_All
Drain 10

End_Repeat

END

COUPLING

BEGIN

if class = DEOXY_THIOATE

Nozzle wash <act1>
prime <act1>
prime <seq>
Add <act1> 70 + <seq> 70

Wait 40

Drain 5
end-if

if class = MOE_THIOATE

Nozzle wash <act1>

Prime <act1>

prime <seq>

Add <act1> 120 + <seq> 120
Wait 230
Drain 5

End_if

END

WASH_AFTER_COUPLING

BEGIN

Add ACN 200 To_All
Drain 10

END

OXIDIZE

BEGIN

if class = DEOXY_THIOATE

Add BEAU 180
Wait 40

Drain 7

end_if

if class = MOE_THIOATE

Add BEAU 200

Wait 120
Drain 7

end_if

END
CAP

BEGIN

Add CAP_B 80 + CAP_A 80
Wait 20

Drain 7

END

WASH_AFTER_CAP

BEGIN

Add ACN 150 To_All
Drain 5

Add ACN 250 To_All
Drain 11

END

BASE_COUNTER

BEGIN

Next_Sequence

END

LOOP_END
DEBLOCK_FINAL

BEGIN

Prime TCA
Load Tray
Repeat 2

Add TCA 150 To_All
Wait 10
Drain 8

End_Repeat
Remove Tray
Add TCA 125 To_All
Wait 10
Drain 10

END

FINAL_WASH

BEGIN

Repeat 4

Add ACN 300 to_All
Drain_12

End_Repeat

END

ENDALL

BEGIN

Wait 3

END

Sequence Files (.seq Files)

File for oligonucleotides having 2′-deoxy nucleosides linked by phosphorothioate internucleotide linkages.

Identity of columns: Syn #, Well, Scale, Nucleotide at particular position (identified using base identifier followed by backbone identifier where “s” is phosphorothioate). Note the columns wrap around to next line when longer than one line.

| | |
|---|---|
| 1 | A01 200 As Cs Cs As Gs Gs As Cs Gs Gs |
| | Cs Gs Gs As Cs Cs As Gs |
| 2 | A02 200 As Cs Gs Gs Cs Gs Gs As Cs Cs |
| | As Gs As Gs Ts Gs Gs As |
| 3 | A03 200 As Cs Cs As As Gs Cs As Gs As |
| | Cs Gs Gs As Gs As Cs Gs |
| 4 | A04 200 As Gs Gs As Gs As Cs Cs Cs Cs |
| | Gs As Cs Gs As As Cs Gs |
| 5 | A05 200 As Cs Cs Cs Cs Gs As Cs Gs As |
| | As Cs Gs As Cs Ts Gs Gs |
| 6 | A06 200 As Cs Gs As As Cs Gs As Cs Ts |
| | Gs Gs Cs Gs As Cs As Gs |
| 7 | A07 200 As Cs Gs As Cs Ts Gs Gs Cs Gs |
| | As Cs As Gs Gs Ts As Gs |
| 8 | A08 200 As Cs As Gs Gs Ts As Gs Gs Ts |
| | Cs Ts Ts Gs Gs Ts Gs Gs |
| 9 | A09 200 As Gs Gs Ts Cs Ts Ts Gs Gs Ts |
| | Gs Gs Gs Ts Gs As Cs Gs |
| 10 | A10 200 As Gs Ts Cs As Cs Gs As Cs As |
| | As Gs As As As Cs As Cs |
| 11 | A11 200 As Cs Gs As Cs As As Gs As As |
| | As Cs As Cs Gs Gs Ts Cs |
| 12 | A12 200 As Gs As As As Cs As Cs Gs Gs |
| | Ts Cs Gs Gs Ts Cs Cs Ts |
| 13 | B01 200 As As Cs As Cs Gs Gs Ts Cs Gs |
| | Gs Ts Cs Cs Ts Gs Ts Cs |
| 14 | B02 200 As Cs Ts Cs As Cs Ts Gs As Cs |
| | Gs Ts Gs Ts Cs Ts Cs As |
| 15 | B03 200 As Cs Gs Gs As As Gs Gs As As |
| | Cs Gs Cs Cs As Cs Ts Ts |
| 16 | B04 200 As Ts Cs Ts Gs Ts Gs Gs As Cs |
| | Cs Ts Ts Gs Ts Cs Ts Cs |
| 17 | B05 200 As Cs As Cs Ts Ts Cs Ts Ts Cs |
| | Cs Gs As Cs Cs Gs Ts Gs |
| 18 | B06 200 As Cs Ts Cs Ts Cs Gs As Cs As |
| | Cs As Gs Gs As Cs Gs Ts |
| 19 | B07 200 As As As Cs Cs Cs Cs As Gs Ts |
| | Ts Cs Gs Ts Cs Ts As As |
| 20 | B08 200 As Ts Gs Ts Cs Cs Cs Cs As As |
| | As Gs As Cs Ts As Ts Gs |
| 21 | B09 200 As Cs Gs Cs Ts Cs Gs Gs Gs As |
| | Cs Gs Gs Gs Ts Cs As Gs |
| 22 | B10 200 As Gs Cs Cs Gs As As Gs As As |
| | Gs As Gs Gs Ts Ts As Cs |
| 23 | B11 200 As Cs As Cs As Gs Ts As Gs As |
| | Cs Gs As As As Gs Cs Ts |
| 24 | B12 200 As Cs As Cs Ts Cs Ts Gs Gs Ts |
| | Ts Ts Cs Ts Gs Gs As Cs |
| 25 | C01 200 As Cs Gs As Cs Cs As Gs As As |
| | As Ts As Gs Ts Ts Ts Ts |
| 26 | C02 200 As Gs Ts Ts As As As As Gs Gs |
| | Gs Cs Ts Gs Cs Ts As Gs |
| 27 | C03 200 As Gs Gs Ts Ts Gs Ts Gs As Cs |
| | Gs As Cs Gs As Gs Gs Ts |
| 28 | C04 200 As As Ts Gs Ts As Cs Cs Ts As |
| | Cs Gs Gs Ts Ts Gs Gs Cs |
| 29 | C05 200 As Gs Ts Cs As Cs Gs Ts Cs Cs |
| | Ts Cs Ts Cs Ts Gs Ts Cs |
| 30 | C06 200 Cs Ts Gs Gs Cs Gs As Cs As Gs |
| | Gs Ts As Gs Gs Ts Cs Ts |
| 31 | C07 200 Cs Ts Cs Ts Gs Ts Gs Ts Gs As |
| | Cs Gs Gs Ts Gs Gs Ts Cs |
| 32 | C08 200 Cs As Gs Gs Ts Cs Gs Ts Cs Ts |
| | Ts Cs Cs Cs Gs Ts Gs Gs |
| 33 | C09 200 Cs Ts Gs Ts Gs Gs Ts As Gs As |
| | Cs Gs Ts Gs Gs As Cs As |
| 34 | C10 200 Cs Ts As As Cs Gs As Ts Gs Ts |
| | Cs Cs Cs Cs As As As Gs |
| 35 | C11 200 Cs Ts Gs Ts Ts Cs Gs As Cs As |
| | Cs Ts Cs Ts Gs Gs Ts Ts |
| 36 | C12 200 Cs Ts Gs Gs As Cs Cs As As Cs |
| | As Cs Gs Ts Ts Gs Ts Cs |
| 37 | D01 200 Cs Cs Gs Ts Cs Cs Gs Ts Gs Ts |
| | Ts Ts Gs Ts Ts Cs Ts Gs |
| 38 | D02 200 Cs Ts Gs As Cs Ts As Cs As As |
| | Cs As Gs As Cs As Cs Cs |
| 39 | D03 200 Cs As As Cs As Gs As Cs As Cs |
| | Cs As Gs Gs Gs Gs Ts Cs |
| 40 | D04 200 Cs As Gs Gs Gs Gs Ts Cs Cs Ts |
| | As Gs Cs Cs Gs As Cs Ts |
| 41 | D05 200 Cs Ts Cs Ts As Gs Ts Ts As As |
| | As As Gs Gs Gs Cs Ts Gs |
| 42 | D06 200 Cs Ts Gs Cs Ts As Gs As As Gs |
| | Gs As Cs Cs Gs As Gs Gs |
| 43 | D07 200 Cs Ts Gs As As As Ts Gs Ts As |
| | Cs Cs Ts As Cs Gs Gs Ts |
| 44 | D08 200 Cs As Cs Cs Cs Gs Ts Ts Ts Gs |
| | Ts Cs Cs Gs Ts Cs As As |
| 45 | D09 200 Cs Ts Cs Gs As Ts As Cs Gs Gs |
| | Gs Ts Cs As Gs Ts Cs As |
| 46 | D10 200 Gs Gs Ts As Gs Gs Ts Cs Ts Ts |
| | Gs Gs Ts Gs Gs Gs Ts Gs |
| 47 | D11 200 Gs As Cs Ts Ts Ts Gs Cs Cs Ts |
| | Ts As Cs Gs Gs As As Gs |
| 48 | D12 200 Gs Ts Gs Gs As Gs Ts Cs Ts Ts |
| | Ts Gs Ts Cs Ts Gs Ts Gs |
| 49 | E01 200 Gs Gs As Gs Ts Cs Ts Ts Ts Gs |
| | Ts Cs Ts Gs Ts Gs Gs Ts |
| 50 | E02 200 Gs Gs As Cs As Cs Ts Cs Ts Cs |
| | Gs As Cs As Cs As Gs Gs |
| 51 | E03 200 Gs As Cs As Cs As Gs Gs As Cs |
| | Gs Ts Gs Gs Cs Gs As Gs |
| 52 | E04 200 Gs As Gs Ts As Cs Gs As Gs Cs |
| | Gs Gs Gs Cs Cs Gs As As |
| 53 | E05 200 Gs As Cs Ts As Ts Gs Gs Ts As |
| | Gs As Cs Gs Cs Ts Cs Gs |
| 54 | E06 200 Gs As As Gs As Gs Gs Ts Ts As |
| | Cs As Cs As Gs Ts As Gs |
| 55 | E07 200 Gs As Gs Gs Ts Ts As Cs As Cs |
| | As Gs Ts As Gs As Cs Gs |
| 56 | E08 200 Gs Ts Ts Gs Ts Cs Cs Gs Ts Cs |
| | Cs Gs Ts Gs Ts Ts Ts Gs |
| 57 | E09 200 Gs As Cs Ts Cs Ts Cs Gs Gs Gs |
| | As Cs Cs As Cs Cs As Cs |
| 58 | E10 200 Gs Ts As Gs Gs As Gs As As Cs |
| | Cs As Cs Gs As Cs Cs As |
| 59 | E11 200 Gs Gs Ts Ts Cs Ts Ts Cs Gs Gs |
| | Ts Ts Gs Gs Ts Ts As Ts |
| 60 | E12 200 Gs Ts Gs Gs Gs Gs Ts Ts Cs Gs |
| | Ts Cs Cs Ts Ts Gs Gs Gs |
| 61 | F01 200 Gs Ts Cs As Cs Gs Ts Cs Cs Ts |
| | Cs Ts Gs As As As Ts Gs |
| 62 | F02 200 Gs Ts Cs Cs Ts Cs Cs Ts As Cs |
| | Cs Gs Ts Ts Ts Cs Ts Cs |
| 63 | F03 200 Gs Ts Cs Cs Cs Cs As Cs Gs Ts |
| | Cs Cs Gs Ts Cs Ts Ts Cs |
| 64 | F04 200 Ts Cs As Cs Cs As Gs Gs As Cs |
| | Gs Gs Cs Gs Gs As Cs Cs |
| 65 | F05 200 Ts As Cs Cs As As Gs Cs As Gs |
| | As Cs Gs Gs As Gs As Cs |
| 66 | F06 200 Ts Cs Cs Ts Gs Ts Cs Ts Ts Ts |
| | Gs As Cs Cs As Cs Ts Cs |
| 67 | F07 200 Ts Gs Ts Cs Ts Ts Ts Gs As Cs |
| | Cs As Cs Ts Cs As Cs Ts |
| 68 | F08 200 Ts Gs As Cs Cs As Cs Ts Cs As |
| | Cs Ts Gs As Cs Gs Ts Gs |
| 69 | F09 200 Ts Gs As Cs Gs Ts Gs Ts Cs Ts |
| | Cs As As Gs Ts Gs As Cs |
| 70 | F10 200 Ts Cs As As Gs Ts Gs As Cs Ts |
| | Ts Ts Gs Cs Cs Ts Ts As |
| 71 | F11 200 Ts Gs Ts Ts Ts As Ts Gs As Cs |
| | Gs Cs Ts Gs Gs Gs Gs Ts |
| 72 | F12 200 Ts Ts As Ts Gs As Cs Gs Cs Ts |
| | Gs Gs Gs Gs Ts Ts Gs Gs |
| 73 | G01 200 Ts Gs As Cs Gs Cs Ts Gs Gs Gs |
| | Gs Ts Ts Gs Gs As Ts Cs |
| 74 | G02 200 Ts Cs Gs Ts Cs Ts Ts Cs Cs Cs |
| | Gs Ts Gs Gs As Gs Ts Cs |
| 75 | G03 200 Ts Gs Gs Ts As Gs As Cs Gs Ts |
| | Gs Gs As Cs As Cs Ts Ts |
| 76 | G04 200 Ts Ts Cs Ts Ts Cs Cs Gs As Cs |
| | Cs Gs Ts Gs As Cs As Ts |
| 77 | G05 200 Ts Gs Gs Ts As Gs As Cs Gs Cs |
| | Ts Cs Gs Gs Gs As Cs Gs |
| 78 | G06 200 Ts As Gs As Cs Gs Cs Ts Cs Gs |
| | Gs Gs As Cs Gs Gs Gs Ts |
| 79 | G07 200 Ts Ts Ts Ts As Cs As Gs Ts Gs |
| | Gs Gs As As Cs Cs Ts Gs |
| 80 | G08 200 Ts Gs Gs Gs As As Cs Cs Ts Gs |
| | Ts Ts Cs Gs As Cs As Cs |
| 81 | G09 200 Ts Cs Gs Gs Gs As Cs Cs As Cs |
| | Cs As Cs Ts As Gs Gs Gs |
| 82 | G10 200 Ts As Gs Gs As Cs As As As Cs |
| | Gs Gs Ts As Gs Gs As Gs |
| 83 | G11 200 Ts Gs Cs Ts As Gs As As Gs Gs |
| | As Cs Cs Gs As Gs Gs Ts |
| 84 | G12 200 Ts Cs Ts Gs Ts Cs As Cs Ts Cs |
| | Cs Gs As Cs Gs Ts Gs Gs |

File for oligonucleotides having regions of 2′-O-(methoxyethyl)nucleosides and region of 2′-deoxy nucleosides each linked by phosphorothioate internucleotide linkages.

Identity of columns: Syn #, Well, Scale, Nucleotide at particular position (identified using base identifier followed by backbone identifier where “s” is phosphorothioate phosphorothioate and “moe” indicated a 2′-O-(methoxyethy) substituted nucleoside). The columns wrap around to next line when longer than one line.

| | |
|---|---|
| 1 | A01 200 moeAs moeCs moeCs moeAs Gs Gs As Cs Gs Gs |
| | Cs Gs Gs As moeCs moeCs moeAs moeGs |
| 2 | A02 200 moeAs moeCs moeGs moeGs Cs Gs Gs As Cs Cs |
| | As Gs As Gs moeTs moeGs moeGs moeAs |
| 3 | A03 200 moeAs moeCs moeCs moeAs As Gs Cs As Gs As |
| | Cs Gs Gs As moeGs moeAs moeCs moeGs |
| 4 | A04 200 moeAs moeGs moeGs moeAs Gs As Cs Cs Cs Cs |
| | Gs As Cs Gs moeAs moeAs moeCs moeGs |
| 5 | A05 200 moeAs moeCs moeCs moeCs Cs Gs As Cs Gs As |
| | As Cs Gs As moeCs moeTs moeGs moeGs |
| 6 | A06 200 moeAs moeCs moeGs moeAs As Cs Gs As Cs Ts |
| | Gs Gs Cs Gs moeAs moeCs moeAs moeGs |
| 7 | A07 200 moeAs moeCs moeGs moeAs Cs Ts Gs Gs Cs Gs |
| | As Cs As Gs moeGs moeTs moeAs moeGs |
| 8 | A08 200 moeAs moeCs moeAs moeGs Gs Ts As Gs Gs Ts |
| | Cs Ts Ts Gs moeGs moeTs moeGs moeGs |
| 9 | A09 200 moeAs moeGs moeGs moeTs Cs Ts Ts Gs Gs Ts |
| | Gs Gs Gs Ts moeGs moeAs moeCs moeGs |
| 10 | A10 200 moeAs moeGs moeTs moeCs As Cs Gs As Cs As |
| | As Gs As As moeAs moeCs moeAs moeCs |
| 11 | A11 200 moeAs moeCs moeGs moeAs Cs As As Gs As As |
| | As Cs As Cs moeGs moeGs moeTs moeCs |
| 12 | A12 200 moeAs moeGs moeAs moeAs As Cs As Cs Gs Gs |
| | Ts Cs Gs Gs moeTs moeCs moeCs moeTs |
| 13 | B01 200 moeAs moeAs moeCs moeAs Cs Gs Gs Ts Cs Gs |
| | Gs Ts Cs Cs moeTs moeGs moeTs moeCs |
| 14 | B02 200 moeAs moeCs moeTs moeCs As Cs Ts Gs As Cs |
| | Gs Ts Gs Ts moeCs moeTs moeCs moeAs |
| 15 | B03 200 moeAs moeCs moeGs moeGs As As Gs Gs As As |
| | Cs Gs Cs Cs moeAs moeCs moeTs moeTs |
| 16 | B04 200 moeAs moeTs moeCs moeTs Gs Ts Gs Gs As Cs |
| | Cs Ts Ts Gs moeTs moeCs moeTs moeCs |
| 17 | B05 200 moeAs moeCs moeAs moeCs Ts Ts Cs Ts Ts Cs |
| | Cs Gs As Cs moeCs moeGs moeTs moeGs |
| 18 | B06 200 moeAs moeCs moeTs moeCs Ts Cs Gs As Cs As |
| | Cs As Gs Gs moeAs moeCs moeGs moeTs |
| 19 | B07 200 moeAs moeAs moeAs moeCs Cs Cs Cs As Gs Ts |
| | Ts Cs Gs Ts moeCs moeTs moeAs moeAs |
| 20 | B08 200 moeAs moeTs moeGs moeTs Cs Cs Cs Cs As As |
| | As Gs As Cs moeTs moeAs moeTs moeGs |
| 21 | B09 200 moeAs moeCs moeGs moeCs Ts Cs Gs Gs Gs As |
| | Cs Gs Gs Gs moeTs moeCs moeAs moeGs |
| 22 | B10 200 moeAs moeGs moeCs moeCs Gs As As Gs As As |
| | Gs As Gs Gs moeTs moeTs moeAs moeCs |
| 23 | B11 200 moeAs moeCs moeAs moeCs As Gs Ts As Gs As |
| | Cs Gs As As moeAs moeGs moeCs moeTs |
| 24 | B12 200 moeAs moeCs moeAs moeCs Ts Cs Ts Gs Gs Ts |
| | Ts Ts Cs Ts moeGs moeGs moeAs moeCs |
| 25 | C01 200 moeAs moeCs moeGs moeAs Cs Cs As Gs As As |
| | As Ts As Gs moeTs moeTs moeTs moeTs |
| 26 | C02 200 moeAs moeGs moeTs moeTs As As As As Gs Gs |
| | Gs Cs Ts Gs moeCs moeTs moeAs moeGs |
| 27 | C03 200 moeAs moeGs moeGs moeTs Ts Gs Ts Gs As Cs |
| | Gs As Cs Gs moeAs moeGs moeGs moeTs |
| 28 | C04 200 moeAs moeAs moeTs moeGs Ts As Cs Cs Ts As |
| | Cs Gs Gs Ts moeTs moeGs moeGs moeCs |
| 29 | C05 200 moeAs moeGs moeTs moeCs As Cs Gs Ts Cs Cs |
| | Ts Cs Ts Cs moeTs moeGs moeTs moeCs |
| 30 | C06 200 moeCs moeTs moeGs moeGs Cs Gs As Cs As Gs |
| | Gs Ts As Gs moeGs moeTs moeCs moeTs |
| 31 | C07 200 moeCs moeTs moeCs moeTs Gs Ts Gs Ts Gs As |
| | Cs Gs Gs Ts moeGs moeGs moeTs moeCs |
| 32 | C08 200 moeCs moeAs moeGs moeGs Ts Cs Gs Ts Cs Ts |
| | Ts Cs Cs Cs moeGs moeTs moeGs moeGs |
| 33 | C09 200 moeCs moeTs moeGs moeTs Gs Gs Ts As Gs As |
| | Cs Gs Ts Gs moeGs moeAs moeCs moeAs |
| 34 | C10 200 moeCs moeTs moeAs moeAs Cs Gs As Ts Gs Ts |
| | Cs Cs Cs Cs moeAs moeAs moeAs moeGs |
| 35 | C11 200 moeCs moeTs moeGs moeTs Ts Cs Gs As Cs As |
| | Cs Ts Cs Ts moeGs moeGs moeTs moeTs |
| 36 | C12 200 moeCs moeTs moeGs moeGs As Cs Cs As As Cs |
| | As Cs Gs Ts moeTs moeGs moeTs moeCs |
| 37 | D01 200 moeCs moeCs moeGs moeTs Cs Cs Gs Ts Gs Ts |
| | Ts Ts Gs Ts moeTs moeCs moeTs moeGs |
| 38 | D02 200 moeCs moeTs moeGs moeAs Cs Ts As Cs As As |
| | Cs As Gs As moeCs moeAs moeCs moeCs |
| 39 | D03 200 moeCs moeAs moeAs moeCs As Gs As Cs As Cs |
| | Cs As Gs Gs moeGs moeGs moeTs moeCs |
| 40 | D04 200 moeCs moeAs moeGs moeGs Gs Gs Ts Cs Cs Ts |
| | As Gs Cs Cs moeGs moeAs moeCs moeTs |
| 41 | D05 200 moeCs moeTs moeCs moeTs As Gs Ts Ts As As |
| | As As Gs Gs moeGs moeCs moeTs moeGs |
| 42 | D06 200 moeCs moeTs moeGs moeCs Ts As Gs As As Gs |
| | Gs As Cs Cs moeGs moeAs moeGs moeGs |
| 43 | D07 200 moeCs moeTs moeGs moeAs As As Ts Gs Ts As |
| | Cs Cs Ts As moeCs moeGs moeGs moeTs |
| 44 | D08 200 moeCs moeAs moeCs moeCs Cs Gs Ts Ts Ts Gs |
| | Ts Cs Cs Gs moeTs moeCs moeAs moeAs |
| 45 | D09 200 moeCs moeTs moeCs moeGs As Ts As Cs Gs Gs |
| | Gs Ts Cs As moeGs moeTs moeCs moeAs |
| 46 | D10 200 moeGs moeGs moeTs moeAs Gs Gs Ts Cs Ts Ts |
| | Gs Gs Ts Gs moeGs moeGs moeTs moeGs |
| 47 | D11 200 moeGs moeAs moeCs moeTs Ts Ts Gs Cs Cs Ts |
| | Ts As Cs Gs moeGs moeAs moeAs moeGs |
| 48 | D12 200 moeGs moeTs moeGs moeGs As Gs Ts Cs Ts Ts |
| | Ts Gs Ts Cs moeTs moeGs moeTs moeGs |
| 49 | E01 200 moeGs moeGs moeAs moeGs Ts Cs Ts Ts Ts Gs |
| | Ts Cs Ts Gs moeTs moeGs moeGs moeTs |
| 50 | E02 200 moeGs moeGs moeAs moeCs As Cs Ts Cs Ts Cs |
| | Gs As Cs As moeCs moeAs moeGs moeGs |
| 51 | E03 200 moeGs moeAs moeCs moeAs Cs As Gs Gs As Cs |
| | Gs Ts Gs Gs moeCs moeGs moeAs moeGs |
| 52 | E04 200 moeGs moeAs moeGs moeTs As Cs Gs As Gs Cs |
| | Gs Gs Gs Cs moeCs moeGs moeAs moeAs |
| 53 | E05 200 moeGs moeAs moeCs moeTs As Ts Gs Gs Ts As |
| | Gs As Cs Gs moeCs moeTs moeCs moeGs |
| 54 | E06 200 moeGs moeAs moeAs moeGs As Gs Gs Ts Ts As |
| | Cs As Cs As moeGs moeTs moeAs moeGs |
| 55 | E07 200 moeGs moeAs moeGs moeGs Ts Ts As Cs As Cs |
| | As Gs Ts As moeGs moeAs moeCs moeGs |
| 56 | E08 200 moeGs moeTs moeTs moeGs Ts Cs Cs Gs Ts Cs |
| | Cs Gs Ts Gs moeTs moeTs moeTs moeGs |
| 57 | E09 200 moeGs moeAs moeCs moeTs Cs Ts Cs Gs Gs Gs |
| | As Cs Cs As moeCs moeCs moeAs moeCs |
| 58 | E10 200 moeGs moeTs moeAs moeGs Gs As Gs As As Cs |
| | Cs As Cs Gs moeAs moeCs moeCs moeAs |
| 59 | E11 200 moeGs moeGs moeTs moeTs Cs Ts Ts Cs Gs Gs |
| | Ts Ts Gs Gs moeTs moeTs moeAs moeTs |
| 60 | E12 200 moeGs moeTs moeGs moeGs Gs Gs Ts Ts Cs Gs |
| | Ts Cs Cs Ts moeTs moeGs moeGs moeGs |
| 61 | F01 200 moeGs moeTs moeCs moeAs Cs Gs Ts Cs Cs Ts |
| | Cs Ts Gs As moeAs moeAs moeTs moeGs |
| 62 | F02 200 moeGs moeTs moeCs moeCs Ts Cs Cs Ts As Cs |
| | Cs Gs Ts Ts moeTs moeCs moeTs moeCs |
| 63 | F03 200 moeGs moeTs moeCs moeCs Cs Cs As Cs Gs Ts |
| | Cs Cs Gs Ts moeCs moeTs moeTs moeCs |
| 64 | F04 200 moeTs moeCs moeAs moeCs Cs As Gs Gs As Cs |
| | Gs Gs Cs Gs moeGs moeAs moeCs moeCs |
| 65 | F05 200 moeTs moeAs moeCs moeCs As As Gs Cs As Gs |
| | As Cs Gs Gs moeAs moeGs moeAs moeCs |
| 66 | F06 200 moeTs moeCs moeCs moeTs Gs Ts Cs Ts Ts Ts |
| | Gs As Cs Cs moeAs moeCs moeTs moeCs |
| 67 | F07 200 moeTs moeGs moeTs moeCs Ts Ts Ts Gs As Cs |
| | Cs As Cs Ts moeCs moeAs moeCs moeTs |
| 68 | F08 200 moeTs moeGs moeAs moeCs Cs As Cs Ts Cs As |
| | Cs Ts Gs As moeCs moeGs moeTs moeGs |
| 69 | F09 200 moeTs moeGs moeAs moeCs Gs Ts Gs Ts Cs Ts |
| | Cs As As Gs moeTs moeGs moeAs moeCs |
| 70 | F10 200 moeTs moeCs moeAs moeAs Gs Ts Gs As Cs Ts |
| | Ts Ts Gs Cs moeCs moeTs moeTs moeAs |
| 71 | F11 200 moeTs moeGs moeTs moeTs Ts As Ts Gs As Cs |
| | Gs Cs Ts Gs moeGs moeGs moeGs moeTs |
| 72 | F12 200 moeTs moeTs moeAs moeTs Gs As Cs Gs Cs Ts |
| | Gs Gs Gs Gs moeTs moeTs moeGs moeGs |
| 73 | G01 200 moeTs moeGs moeAs moeCs Gs Cs Ts Gs Gs Gs |
| | Gs Ts Ts Gs moeGs moeAs moeTs moeCs |
| 74 | G02 200 moeTs moeCs moeGs moeTs Cs Ts Ts Cs Cs Cs |
| | Gs Ts Gs Gs moeAs moeGs moeTs moeCs |
| 75 | G03 200 moeTs moeGs moeGs moeTs As Gs As Cs Gs Ts |
| | Gs Gs As Cs moeAs moeCs moeTs moeTs |
| 76 | G04 200 moeTs moeTs moeCs moeTs Ts Cs Cs Gs As Cs |
| | Cs Gs Ts Gs moeAs moeCs moeAs moeTs |
| 77 | G05 200 moeTs moeGs moeGs moeTs As Gs As Cs Gs Cs |
| | Ts Cs Gs Gs moeGs moeAs moeCs moeGs |
| 78 | G06 200 moeTs moeAs moeGs moeAs Cs Gs Cs Ts Cs Gs |
| | Gs Gs As Cs moeGs moeGs moeGs moeTs |
| 79 | G07 200 moeTs moeTs moeTs moeTs As Cs As Gs Ts Gs |
| | Gs Gs As As moeCs moeCs moeTs moeGs |
| 80 | G08 200 moeTs moeGs moeGs moeGs As As Cs Cs Ts Gs |
| | Ts Ts Cs Gs moeAs moeCs moeAs moeCs |
| 81 | G09 200 moeTs moeCs moeGs moeGs Gs As Cs Cs As Cs |
| | Cs As Cs Ts moeAs moeGs moeGs moeGs |
| 82 | G10 200 moeTs moeAs moeGs moeGs As Cs As As As Cs |
| | Gs Gs Ts As moeGs moeGs moeAs moeGs |
| 83 | G11 200 moeTs moeGs moeCs moeTs As Gs As As Gs Gs |
| | As Cs Cs Gs moeAs moeGs moeGs moeTs |
| 84 | G12 200 moeTs moeCs moeTs moeGs Ts Cs As Cs Ts Cs |
| | Cs Gs As Cs moeGs moeTs moeGs moeGs |

Reagent File (.tab File)

File for reagents necessary for synthesizing an oligonucleotides having both 2′-O-(methoxyethy)nucleosides and 2′-deoxy nucleosides located therein.

Identity of columns: GroupName, Bottle ID, ReagentName, FlowRate, Concentration. Wherein reagent name is identified using base identifier, “moe” indicated a 2′-O-(methoxyethy) substituted nucleoside and “cpg” indicates a control pore glass solid support medium. The columns wrap around to next line when longer than one line.

SUPPORT

BEGIN

| | | | | |
|---|---|---|---|---|
| 0 | moeG | moeGcpg | 100 | 1 |
| 0 | moe5meC | moe5meCcpg | 100 | 1 |
| 0 | moeA | moeAcpg | 100 | 1 |
| 0 | moeT | moeTcpg | 100 | 1 |
| END | | | | |

DEBLOCK
BEGIN

| | | | | |
|---|---|---|---|---|
| 70 | TCA | TCA | 100 | 1 |

END

WASH
BEGIN

| | | | |
|---|---|---|---|
| 65 | ACN | ACN 190 | 1 |

END

OXIDIZERS
BEGIN

| | | | | |
|---|---|---|---|---|
| 68 | BEAU | BEAUCAGE | 320 | 1 |

END

CAPPING
BEGIN

| | | | | |
|---|---|---|---|---|
| 66 | CAP_B | CAP_B | 220 | 1 |
| 67 | CAP_A | CAP_A | 230 | 1 |

END

DEOXY THIOATE
BEGIN

| | | | | |
|---|---|---|---|---|
| 31, 32 | Gs | deoxyG | 270 | 1 |
| 39, 40 | 5meCs | 5methyldeoxyC | 270 | 1 |
| 37, 38 | As | deoxyA | 270 | 1 |
| 29, 30 | Ts | deoxyT | 270 | 1 |

END

MOE-THIOATE
BEGIN

| | | | | |
|---|---|---|---|---|
| 15, 16 | moeGs | methoxyethoxyG | 240 | 1 |
| 23, 24 | moe5meCs | methoxyethoxyC | 240 | 1 |
| 21, 22 | moeAs | methoxyethoxyA | 240 | 1 |
| 13, 14 | moeTs | methoxyethoxyT | 240 | 1 |

END

ACTIVATORS

| | BEGIN | | | |
|---|---|---|---|---|
| 5, 6, 7, 8 SET | s-ethyl-tet | 280 | 1 Activates | DEOXY_THIOATE |

MOE_THIOATE

END

### Example 63

### Output Oligonucleotides from Automated Oligonucleotide Synthesis

Using the .seq files, the .cmd files and .tab file from above, oligonucleotides were prepared as per the protocol of the 96 well format. The oligonucleotides were prepared utilizing phosphorothioate chemistry to give in one instance a first library of phosphorothioate oligonucleotides. The oligonucleotides were prepared in a second instance as a second library of hybrid oligonucleotides have phosphorothioate backbones with a first and third ‘wing’ region of 2′-O-(methoxyethyl) nucleotides on either side of a center gap region of 2′-deoxy nucleotides. In each instance, the sequences of the oligonucleotides were the same.

For illustrative purposes Table 13 shows the sequences of the library of phosphorothioate oligonucleotide of the first library. Because the sequences of the second library of compounds is the same as the first (however the chemistry is different), for brevity sake, the second library is not shown.

The sequences shown below are in a 5′ to 3′ direction. This is reverse with respect to 3′ to 5′ direction shown is the seq files of Example 3. For synthesis purposes, the seq files are generated reading from 3′ to 5′. This allows for aligning all of the 3′ most ‘A’ nucleosides together, all of the 3′ most ‘G’ nucleosides together, all of the 3′ most ‘C’ nucleosides together and all of the 3′ most ‘T’ nucleosides together. Thus when the first nucleoside of each particular oligonucleotide (attached to the solid support) is added to the wells on the plates, machine movement is reduced since an automatic pipette can move in a linear manner down one row and up another on the 96 well plate.

The locations of the well holding the particular oligonucleotides is indicated by row and column. There are 8 rows (A to G) and 12 columns in a typical 96 well format plate. Any particular well location is indicated by its ‘Well No.’ which is indicated by the combination of the row and the column, e.g. A08 is the well at row A, column 8.

TABLE 13
Sequences of Oligonucleotides Targeted to CD40

| | | |
|---|---|---|
| Well No. | Nucleobase Sequence | SEQ ID NO: |

| | | |
|---|---|---|
| A01 | GACCAGGCGGCAGGACCA | 68 |
| A02 | AGGTGAGACCAGGCGGCA | 69 |
| A03 | GCAGAGGCAGACGAACCA | 70 |
| A04 | GCAAGCAGCCCCAGAGGA | 71 |
| A05 | GGTCAGCAAGCAGCCCCA | 72 |
| A06 | GACAGCGGTCAGCAAGCA | 73 |
| A07 | GATGGACAGCGGTCAGCA | 74 |
| A08 | GGTGGTTCTGGATGGACA | 75 |
| A09 | GCAGTGGGTGGTTCTGGA | 76 |
| A10 | CACAAAGAACAGCACTGA | 77 |
| A11 | CTGGCACAAAGAACAGCA | 78 |
| A12 | TCCTGGCTGGCACAAAGA | 79 |
| B01 | CTGTCCTGGCTGGCACAA | 80 |
| B02 | ACTCTGTGCAGTCACTCA | 81 |
| B03 | TTCACCGCAAGGAAGGCA | 82 |
| B04 | CTCTGTTCCAGGTGTCTA | 83 |
| B05 | GTGCCAGCCTTCTTCACA | 84 |
| B06 | TGCAGGACACAGCTCTCA | 85 |
| B07 | AATCTGCTTGACCCCAAA | 86 |
| B08 | GTATCAGAAACCCCTGTA | 87 |
| B09 | GACTGGGCAGGGCTCGCA | 88 |
| B10 | CATTGGAGAAGAAGCCGA | 89 |
| B11 | TCGAAAGCAGATGACACA | 90 |
| B12 | CAGGTCTTTGGTCTCACA | 91 |
| C01 | TTTTGATAAAGACCAGCA | 92 |
| C02 | GATCGTCGGGAAAATTGA | 93 |
| C03 | TGGAGCAGCAGTGTTGGA | 94 |
| C04 | CGGTTGGCATCCATGTAA | 95 |
| C05 | CTGTCTCTCCTGCACTGA | 96 |
| C06 | TCTGGATGGACAGCGGTC | 97 |
| C07 | CTGGTGGCAGTGTGTCTC | 98 |
| C08 | GGTGCCCTTCTGCTGGAC | 99 |
| C09 | ACAGGTGCAGATGGTGTC | 100 |
| C10 | GAAACCCCTGTAGCAATC | 101 |
| C11 | TTGGTCTCACAGCTTGTC | 102 |
| C12 | CTGTTGCACAACCAGGTC | 103 |
| D01 | GTCTTGTTTGTGCCTGCC | 104 |
| D02 | CCACAGACAACATCAGTC | 105 |
| D03 | CTGGGGACCACAGACAAC | 106 |
| D04 | TCAGCCGATCCTGGGGAC | 107 |
| D05 | GTCGGGAAAATTGATCTC | 108 |
| D06 | GGAGCCAGGAAGATCGTC | 109 |
| D07 | TGGCATCCATGTAAAGTC | 110 |
| D08 | AACTGCCTGTTTGCCCAC | 111 |
| D09 | ACTGACTGGGCATAGCTC | 112 |
| D10 | GTGGGTGGTTCTGGATGG | 113 |
| D11 | GAAGGCATTCCGTTTCAG | 114 |
| D12 | GTGTCTGTTTCTGAGGTG | 115 |
| E01 | TGGTGTCTGTTTCTGAGG | 116 |
| E02 | GGACACAGCTCTCACAGG | 117 |
| E03 | GAGCGGTGCAGGACACAG | 118 |
| E04 | AAGCCGGGCGAGCATGAG | 119 |
| E05 | GCTCGCAGATGGTATCAG | 120 |
| E06 | GATGACACATTGGAGAAG | 121 |
| E07 | GCAGATGACACATTGGAG | 122 |
| E08 | GTTTGTGCCTGCCTGTTG | 123 |
| E09 | CACCACCAGGGCTCTCAG | 124 |
| E10 | ACCAGCACCAAGAGGATG | 125 |
| E11 | TATTGGTTGGCTTCTTGG | 126 |
| E12 | GGGTTCCTGCTTGGGGTG | 127 |
| F01 | GTAAAGTCTCCTGCACTG | 128 |
| F02 | CTCTTTGCCATCCTCCTG | 129 |
| F03 | CTTCTGCCTGCACCCCTG | 130 |
| F04 | CCAGGCGGCAGGACCACT | 131 |
| F05 | CAGAGGCAGACGAACCAT | 132 |
| F06 | CTCACCAGTTTCTGTCCT | 133 |
| F07 | TCACTCACCAGTTTCTGT | 134 |
| F08 | GTGCAGTCACTCACCAGT | 135 |
| F09 | CAGTGAACTCTGTGCAGT | 136 |
| F10 | ATTCCGTTTCAGTGAACT | 137 |
| F11 | TGGGGTCGCAGTATTTGT | 138 |
| F12 | GGTTGGGGTCGCAGTATT | 139 |
| G01 | CTAGGTTGGGGTCGCAGT | 140 |
| G02 | CTGAGGTGCCCTTCTGCT | 141 |
| G03 | TTCACAGGTGCAGATGGT | 142 |
| G04 | TACAGTGCCAGCCTTCTT | 143 |
| G05 | GCAGGGCTCGCAGATGGT | 144 |
| G06 | TGGGCAGGGCTCGCAGAT | 145 |
| G07 | GTCCAAGGGTGACATTTT | 146 |
| G08 | CACAGCTTGTCCAAGGGT | 147 |
| G09 | GGGATCACCACCAGGGCT | 148 |
| G10 | GAGGATGGCAAACAGGAT | 149 |
| G11 | TGGAGCCAGGAAGATCGT | 150 |
| G12 | GGTGCAGCCTCACTGTCT | 151 |

### Example 64

### Oligonucleotide Analysis

Oligonucleotide Analysis—96 Well Plate Format

The concentration of oligonucleotide in each well was assessed by dilution of samples and UV absorption spectroscopy. The full-length integrity of the individual products was evaluated by capillary electrophoresis (CE) in either the 96 well format (Beckman MDQ) or, for individually prepared samples, on a commercial CE apparatus (e.g., Beckman 5000, ABI 270). Base and backbone composition was confirmed by mass analysis of the compounds utilizing Electrospray-Mass Spectroscopy. All assay test plates were diluted from the master plate using single and multi-channel robotic pipettors.

Alternate Oligonucleotide Analysis

After cleavage from the controlled pore glass support (Applied Biosystems) and deblocking in concentrated ammonium hydroxide at 55° C. for 18 hours, the oligonucleotides or oligonucleosides are purified by precipitation twice out of 0.5 M NaCl with 2.5 volumes ethanol. Synthesized oligonucleotides are analyzed by polyacrylamide gel electrophoresis on denaturing gels. Oligonucleotide purity is checked by ³¹P nuclear magnetic resonance spectroscopy, and/or by HPLC, as described by Chiang et al., J. Biol. Chem. 1991, 266, 18162.

### Example 65

### Automated Assay of CD40 Oligonucleotides

Poly(A)+ mRNA Isolation

Poly(A)+ mRNA was isolated according to Miura et al. (Clin. Chem., 1996, 42, 1758). Briefly, for cells grown on 96-well plates, growth medium was removed from the cells and each well was washed with 200 ul cold PBS. 60 ul lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 ul of lysate was transferred to Oligo d(T) coated 96 well plates (AGCT Inc., Irvine, Calif.). Plates were incubated for 60 minutes at room temperature, washed 3 times with 200 ul of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 ul of elution buffer (5 mM Tris-HCl pH 7.6), preheated to 70° C. was added to each well, the plate was incubated on a 90° hot plate for 5 minutes, and the eluate then transferred to a fresh 96 well plate. Cells grown on 100 mm or other standard plates may be treated similarly, using appropriate volumes of all solutions.

RT-PCR Analysis of CD40 mRNA Levels

Quantitation of CD40 mRNA levels was determined by real-time PCR (RT-PCR) using the ABI PRISM 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time.

As opposed to standard PCR, in which amplification products are quantitated after the PCR is completed, products in RT-PCR are quantitated as they accumulate. This is accomplished by including in the PCR reaction an oligonucleotide probe that anneals specifically between the forward and reverse PCR primers, and contains two fluorescent dyes. A reporter dye (e.g., JOE or FAM, PE-Applied Biosystems, Foster City, Calif.) is attached to the 5′ end of the probe and a quencher dye (e.g., TAMRA, PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated.

With each cycle, additional reporter dye molecules are cleaved from their respective probes, and the fluorescence intensity is monitored at regular (six-second) intervals by laser optics built into the ABI PRISM 7700 Sequence Detection System. In each assay, a series of parallel reactions containing serial dilutions of mRNA from untreated control samples generates a standard curve that is used to quantitate the percent inhibition after antisense oligonucleotide treatment of test samples.

RT-PCR reagents were obtained from PE-Applied Biosystems, Foster City, Calif. RT-PCR reactions were carried out by adding 25 ul PCR cocktail (1× Taqman buffer A, 5.5 mM MgCl₂, 300 uM each of dATP, dCTP and dGTP, 600 uM of dUTP, 100 nM each of forward primer, reverse primer, and probe, 20 U RNAse inhibitor, 1.25 units AmpliTaq Gold, and 12.5 U MuLV reverse transcriptase) to 96 well plates containing 25 ul poly(A) mRNA solution. The RT reaction was carried out by incubation for 30 minutes at 48° C. following a 10 minute incubation at 95° C. to activate the AmpliTaq gold, 40 cycles of a two-step PCR protocol were carried out: 95° C. for 15 seconds (denaturation) followed by 60° C. for 1.5 minutes (annealing/extension).

For CD40, the PCR primers were:

| | |
|---|---|
| forward primer: | |
| CAGAGTTCACTGAAACGGAATGC | (SEQ ID NO:152) |
| reverse primer: | |
| GGTGGCAGTGTGTCTCTCTGTTC | (SEQ ID NO:153) |

and the PCR probe was:

| | |
|---|---|
| FAM-TTCCTTGCGGTGAAAGCGAATTCCT- | (SEQ ID NO:154) |
| TAMRA | |

where FAM (PE-Applied Biosystems, Foster City, Calif.) is the fluorescent reporter dye) and TAMRA (PE-Applied Biosystems, Foster City, Calif.) is the quencher dye.

For GAPDH the PCR primers were:

| | |
|---|---|
| forward primer: | |
| GAAGGTGAAGGTCGGAGTC | (SEQ ID NO:155) |
| reverse primer: | |
| GAAGATGGTGATGGGATTTC | (SEQ ID NO:156) |

and the PCR probe was:

| | |
|---|---|
| 5′ JOE-CAAGCTTCCCGTTCTCAGCC- | (SEQ ID No.157) |
| TAMRA 3′ | |

where JOE (PE-Applied Biosystems, Foster City, Calif.) is the fluorescent reporter dye) and TAMRA (PE-Applied Biosystems, Foster City, Calif.) is the quencher dye.

### Example 66

### Inhibition of CD40 Expression by Phosphorothioate Oligomers

In accordance with the present invention, a series of oligonucleotides complementary to mRNA were designed to target different regions of the human CD40 mRNA, using published sequences (GenBank accession number X60592, incorporated herein as SEQ ID NO:158). The oligonucleotides are shown in Table 14. Target sites are indicated by nucleotide numbers, as given in the sequence source reference (X60592), to which the oligonucleotide binds. All compounds in Table 14 are oligodeoxynucleotides with phosphorothioate backbones (intersugar linkages) throughout. Data are averages from three experiments.

TABLE 14

Inhibition of CD40 mRNA Levels by
Phosphorothioate Oligodeoxynucleotides

| | | | |
|---|---|---|---|
| ISIS# | SEQUENCE | % INHIB. | SEQ ID NO. |

| | | | |
|---|---|---|---|
| 18623 | CCAGGCGGCAGGACCACT | 30.71 | 159 |
| 18624 | GACCAGGCGGCAGGACCA | 28.09 | 160 |
| 18625 | AGGTGAGACCAGGCGGCA | 21.89 | 161 |
| 18626 | CAGAGGCAGACGAACCAT | 0.00 | 162 |
| 18627 | GCAGAGGCAGACGAACCA | 0.00 | 163 |
| 18628 | GCAAGCAGCCCCAGAGGA | 0.00 | 164 |
| 18629 | GGTCAGCAAGCAGCCCCA | 29.96 | 165 |
| 18630 | GACAGCGGTCAGCAAGCA | 0.00 | 166 |
| 18631 | GATGGACAGCGGTCAGCA | 0.00 | 167 |
| 18632 | TCTGGATGGACAGCGGTC | 0.00 | 168 |
| 18633 | GGTGGTTCTGGATGGACA | 0.00 | 169 |
| 18634 | GTGGGTGGTTCTGGATGG | 0.00 | 170 |
| 18635 | GCAGTGGGTGGTTCTGGA | 0.00 | 171 |
| 18636 | CACAAAGAACAGCACTGA | 0.00 | 172 |
| 18637 | CTGGCACAAAGAACAGCA | 0.00 | 173 |
| 18638 | TCCTGGCTGGCACAAAGA | 0.00 | 174 |
| 18639 | CTGTCCTGGCTGGCACAA | 4.99 | 175 |
| 18640 | CTCACCAGTTTCTGTCCT | 0.00 | 176 |
| 18641 | TCACTCACCAGTTTCTGT | 0.00 | 177 |
| 18642 | GTGCAGTCACTCACCAGT | 0.00 | 178 |
| 18643 | ACTCTGTGCAGTCACTCA | 0.00 | 179 |
| 18644 | CAGTGAACTCTGTGCAGT | 5.30 | 180 |
| 18645 | ATTCCGTTTCAGTGAACT | 0.00 | 181 |
| 18646 | GAAGGCATTCCGTTTCAG | 9.00 | 182 |
| 18647 | TTCACCGCAAGGAAGGCA | 0.00 | 183 |
| 18648 | CTCTGTTCCAGGTGTCTA | 0.00 | 184 |
| 18649 | CTGGTGGCAGTGTGTCTC | 0.00 | 185 |
| 18650 | TGGGGTCGCAGTATTTGT | 0.00 | 186 |
| 18651 | GGTTGGGGTCGCAGTATT | 0.00 | 187 |
| 18652 | CTAGGTTGGGGTCGCAGT | 0.00 | 188 |
| 18653 | GGTGCCCTTCTGCTGGAC | 19.67 | 189 |
| 18654 | CTGAGGTGCCCTTCTGCT | 15.63 | 190 |
| 18655 | GTGTCTGTTTCTGAGGTG | 0.00 | 191 |
| 18656 | TGGTGTCTGTTTCTGAGG | 0.00 | 192 |
| 18657 | ACAGGTGCAGATGGTGTC | 0.00 | 193 |
| 18658 | TTCACAGGTGCAGATGGT | 0.00 | 194 |
| 18659 | GTGCCAGCCTTCTTCACA | 5.67 | 195 |
| 18660 | TACAGTGCCAGCCTTCTT | 7.80 | 196 |
| 18661 | GGACACAGCTCTCACAGG | 0.00 | 197 |
| 18662 | TGCAGGACACAGCTCTCA | 0.00 | 198 |
| 18663 | GAGCGGTGCAGGACACAG | 0.00 | 199 |
| 18664 | AAGCCGGGCGAGCATGAG | 0.00 | 200 |
| 18665 | AATCTGCTTGACCCCAAA | 5.59 | 201 |
| 18666 | GAAACCCCTGTAGCAATC | 0.10 | 202 |
| 18667 | GTATCAGAAACCCCTGTA | 0.00 | 203 |
| 18668 | GCTCGCAGATGGTATCAG | 0.00 | 204 |
| 18669 | GCAGGGCTCGCAGATGGT | 34.05 | 205 |
| 18670 | TGGGCAGGGCTCGCAGAT | 0.00 | 206 |
| 18671 | GACTGGGCAGGGCTCGCA | 2.71 | 207 |
| 18672 | CATTGGAGAAGAAGCCGA | 0.00 | 208 |
| 18673 | GATGACACATTGGAGAAG | 0.00 | 209 |
| 18674 | GCAGATGACACATTGGAG | 0.00 | 210 |
| 18675 | TCGAAAGCAGATGACACA | 0.00 | 211 |
| 18676 | GTCCAAGGGTGACATTTT | 8.01 | 212 |
| 18677 | CACAGCTTGTCCAAGGGT | 0.00 | 213 |
| 18678 | TTGGTCTCACAGCTTGTC | 0.00 | 214 |
| 18679 | CAGGTCTTTGGTCTCACA | 6.98 | 215 |
| 18680 | CTGTTGCACAACCAGGTC | 18.76 | 216 |
| 18681 | GTTTGTGCCTGCCTGTTG | 2.43 | 217 |
| 18682 | GTCTTGTTTGTGCCTGCC | 0.00 | 218 |
| 18683 | CCACAGACAACATCAGTC | 0.00 | 219 |
| 18684 | CTGGGGACCACAGACAAC | 0.00 | 220 |
| 18685 | TCAGCCGATCCTGGGGAC | 0.00 | 221 |
| 18686 | CACCACCAGGGCTCTCAG | 23.31 | 222 |
| 18687 | GGGATCACCACCAGGGCT | 0.00 | 223 |
| 18688 | GAGGATGGCAAACAGGAT | 0.00 | 224 |
| 18689 | ACCAGCACCAAGAGGATG | 0.00 | 225 |
| 18690 | TTTTGATAAAGACCAGCA | 0.00 | 226 |
| 18691 | TATTGGTTGGCTTCTTGG | 0.00 | 227 |
| 18692 | GGGTTCCTGCTTGGGGTG | 0.00 | 228 |
| 18693 | GTCGGGAAAATTGATCTC | 0.00 | 229 |
| 18694 | GATCGTCGGGAAAATTGA | 0.00 | 230 |
| 18695 | GGAGCCAGGAAGATCGTC | 0.00 | 231 |
| 18696 | TGGAGCCAGGAAGATCGT | 0.00 | 232 |
| 18697 | TGGAGCAGCAGTGTTGGA | 0.00 | 233 |
| 18698 | GTAAAGTCTCCTGCACTG | 0.00 | 234 |
| 18699 | TGGCATCCATGTAAAGTC | 0.00 | 235 |
| 18700 | CGGTTGGCATCCATGTAA | 0.00 | 236 |
| 18701 | CTCTTTGCCATCCTCCTG | 4.38 | 237 |
| 18702 | CTGTCTCTCCTGCACTGA | 0.00 | 238 |
| 18703 | GGTGCAGCCTCACTGTCT | 0.00 | 239 |
| 18704 | AACTGCCTGTTTGCCCAC | 33.89 | 240 |
| 18705 | CTTCTGCCTGCACCCCTG | 0.00 | 241 |
| 18706 | ACTGACTGGGCATAGCTC | 0.00 | 242 |

As shown in Table 14, SEQ ID NOS: 159, 160, 165, 205 and 240 demonstrated at least 25% inhibition of CD40 expression and are therefore suitable compounds of the invention.

### Example 67

### Inhibition of CD40 Expression by Phosphorothioate 2′-MOE Gapmer Oligonucleotides

In accordance with the present invention, a second series of oligonucleotides complementary to mRNA were designed to target different regions of the human CD40 mRNA, using published sequence X60592. The oligonucleotides are shown in Table 15. Target sites are indicated by nucleotide numbers, as given in the sequence source reference (X60592), to which the oligonucleotide binds.

All compounds in Table 15 are chimeric oligonucleotides (“gapmers”) 18 nucleotides in length, composed of a central “gap” region consisting of ten 2′-deoxynucleotides, which is flanked on both sides (5′ and 3′ directions) by four-nucleotide “wings.” The wings are composed of 2′-methoxyethyl (2′-MOE) nucleotides. The intersugar (backbone) linkages are phosphorothioate (P═S) throughout the oligonucleotide. Cytidine residues in the 2′-MOE wings are 5-methylcytidines. Data are averaged from three experiments.

TABLE 15

Inhibition of CD40 mRNA Levels by Chimeric
Phosphorothioate Oligonucleotides

| | | | |
|---|---|---|---|
| ISIS# | SEQUENCE | % Inhibition | SEQ ID NO. |

| | | | |
|---|---|---|---|
| 19211 | CCAGGCGGCAGGACCACT | 75.71 | 159 |
| 19212 | GACCAGGCGGCAGGACCA | 77.23 | 160 |
| 19213 | AGGTGAGACCAGGCGGCA | 80.82 | 161 |
| 19214 | CAGAGGCAGACGAACCAT | 23.68 | 162 |
| 19215 | GCAGAGGCAGACGAACCA | 45.97 | 163 |
| 19216 | GCAAGCAGCCCCAGAGGA | 65.80 | 164 |
| 19217 | GGTCAGCAAGCAGCCCCA | 74.73 | 165 |
| 19218 | GACAGCGGTCAGCAAGCA | 67.21 | 166 |
| 19219 | GATGGACAGCGGTCAGCA | 65.14 | 167 |
| 19220 | TCTGGATGGACAGCGGTC | 78.71 | 168 |
| 19221 | GGTGGTTCTGGATGGACA | 81.33 | 169 |
| 19222 | GTGGGTGGTTCTGGATGG | 57.79 | 170 |
| 19223 | GCAGTGGGTGGTTCTGGA | 73.70 | 171 |
| 19224 | CACAAAGAACAGCACTGA | 40.25 | 172 |
| 19225 | CTGGCACAAAGAACAGCA | 60.11 | 173 |
| 19226 | TCCTGGCTGGCACAAAGA | 10.18 | 174 |
| 19227 | CTGTCCTGGCTGGCACAA | 24.37 | 175 |
| 19228 | CTCACCAGTTTCTGTCCT | 22.30 | 176 |
| 19229 | TCACTCACCAGTTTCTGT | 40.64 | 177 |
| 19230 | GTGCAGTCACTCACCAGT | 82.04 | 178 |
| 19231 | ACTCTGTGCAGTCACTCA | 37.59 | 179 |
| 19232 | CAGTGAACTCTGTGCAGT | 40.26 | 180 |
| 19233 | ATTCCGTTTCAGTGAACT | 56.03 | 181 |
| 19234 | GAAGGCATTCCGTTTCAG | 32.21 | 182 |
| 19235 | TTCACCGCAAGGAAGGCA | 61.03 | 183 |
| 19236 | CTCTGTTCCAGGTGTCTA | 62.19 | 184 |
| 19237 | CTGGTGGCAGTGTGTCTC | 70.32 | 185 |
| 19238 | TGGGGTCGCAGTATTTGT | 0.00 | 186 |
| 19239 | GGTTGGGGTCGCAGTATT | 19.40 | 187 |
| 19240 | CTAGGTTGGGGTCGCAGT | 36.32 | 188 |
| 19241 | GGTGCCCTTCTGCTGGAC | 78.91 | 189 |
| 19242 | CTGAGGTGCCCTTCTGCT | 69.84 | 190 |
| 19243 | GTGTCTGTTTCTGAGGTG | 63.32 | 191 |
| 19244 | TGGTGTCTGTTTCTGAGG | 42.83 | 192 |
| 19245 | ACAGGTGCAGATGGTGTC | 73.31 | 193 |
| 19246 | TTCACAGGTGCAGATGGT | 47.72 | 194 |
| 19247 | GTGCCAGCCTTCTTCACA | 61.32 | 195 |
| 19248 | TACAGTGCCAGCCTTCTT | 46.82 | 196 |
| 19249 | GGACACAGCTCTCACAGG | 0.00 | 197 |
| 19250 | TGCAGGACACAGCTCTCA | 52.05 | 198 |
| 19251 | GAGCGGTGCAGGACACAG | 50.15 | 199 |
| 19252 | AAGCCGGGCGAGCATGAG | 32.36 | 200 |
| 19253 | AATCTGCTTGACCCCAAA | 0.00 | 201 |
| 19254 | GAAACCCCTGTAGCAATC | 0.00 | 202 |
| 19255 | GTATCAGAAACCCCTGTA | 36.13 | 203 |
| 19256 | GCTCGCAGATGGTATCAG | 64.65 | 204 |
| 19257 | GCAGGGCTCGCAGATGGT | 74.95 | 205 |
| 19258 | TGGGCAGGGCTCGCAGAT | 0.00 | 206 |
| 19259 | GACTGGGCAGGGCTCGCA | 82.00 | 207 |
| 19260 | CATTGGAGAAGAAGCCGA | 41.31 | 208 |
| 19261 | GATGACACATTGGAGAAG | 13.81 | 209 |
| 19262 | GCAGATGACACATTGGAG | 78.48 | 210 |
| 19263 | TCGAAAGCAGATGACACA | 59.28 | 211 |
| 19264 | GTCCAAGGGTGACATTTT | 70.99 | 212 |
| 19265 | CACAGCTTGTCCAAGGGT | 0.00 | 213 |
| 19266 | TTGGTCTCACAGCTTGTC | 45.92 | 214 |
| 19267 | CAGGTCTTTGGTCTCACA | 63.95 | 215 |
| 19268 | CTGTTGCACAACCAGGTC | 82.32 | 216 |
| 19269 | GTTTGTGCCTGCCTGTTG | 70.10 | 217 |
| 19270 | GTCTTGTTTGTGCCTGCC | 68.95 | 218 |
| 19271 | CCACAGACAACATCAGTC | 11.22 | 219 |
| 19272 | CTGGGGACCACAGACAAC | 9.04 | 220 |
| 19273 | TCAGCCGATCCTGGGGAC | 0.00 | 221 |
| 19274 | CACCACCAGGGCTCTCAG | 23.08 | 222 |
| 19275 | GGGATCACCACCAGGGCT | 57.94 | 223 |
| 19276 | GAGGATGGCAAACAGGAT | 49.14 | 224 |
| 19277 | ACCAGCACCAAGAGGATG | 3.48 | 225 |
| 19278 | TTTTGATAAAGACCAGCA | 30.58 | 226 |
| 19279 | TATTGGTTGGCTTCTTGG | 49.26 | 227 |
| 19280 | GGGTTCCTGCTTGGGGTG | 13.95 | 228 |
| 19281 | GTCGGGAAAATTGATCTC | 54.78 | 229 |
| 19282 | GATCGTCGGGAAAATTGA | 0.00 | 230 |
| 19283 | GGAGCCAGGAAGATCGTC | 69.47 | 231 |
| 19284 | TGGAGCCAGGAAGATCGT | 54.48 | 232 |
| 19285 | TGGAGCAGCAGTGTTGGA | 15.17 | 233 |
| 19286 | GTAAAGTCTCCTGCACTG | 30.62 | 234 |
| 19287 | TGGCATCCATGTAAAGTC | 65.03 | 234 |
| 19288 | CGGTTGGCATCCATGTAA | 34.49 | 236 |
| 19289 | CTCTTTGCCATCCTCCTG | 41.84 | 237 |
| 19290 | CTGTCTCTCCTGCACTGA | 25.68 | 238 |
| 19291 | GGTGCAGCCTCACTGTCT | 76.27 | 239 |
| 19292 | AACTGCCTGTTTGCCCAC | 63.34 | 240 |
| 19293 | CTTCTGCCTGCACCCCTG | 0.00 | 241 |
| 19294 | ACTGACTGGGCATAGCTC | 11.55 | 242 |

As shown in Table 15, SEQ ID NOS: 159, 160, 161, 164, 165, 166, 167, 168, 169, 170, 171, 173, 178, 181, 183, 184, 185, 189, 190, 191, 193, 195, 198, 199, 204, 205, 207, 210, 211, 212, 215, 216, 217, 218, 223, 229, 231, 232, 235, 239 and 240 demonstrated at least 50% inhibition of CD40 expression and are therefore suitable compounds of the invention.

### Example 68

### Oligonucleotide-Sensitive Sites of the CD40 Target Nucleic Acid

As the data presented in the preceding two Examples shows, several sequences were present in suitable compounds of two distinct oligonucleotide chemistries. Specifically, compounds having SEQ ID NOS: 159, 160, 165, 205 and 240 are suitable in both instances. These compounds map to different regions of the CD40 transcript but nevertheless define accessible sites of the target nucleic acid.

For example, SEQ ID NOS: 159 and 160 overlap each other and both map to the 5-untranslated region (5′-UTR) of CD40. Accordingly, this region of CD40 is particularly suitable for modulation via sequence-based technologies. Similarly, SEQ ID NOS: 165 and 205 map to the open reading frame of CD40, whereas SEQ ID NO:240 maps to the 3′-untranslated region (3′-UTR). Thus, the ORF and 3′-UTR of CD40 may be targeted by sequence-based technologies as well.

Through multiple iterations of the process of the invention, more extensive “footprints” are generated. A library of this information is compiled and may be used by those skilled in the art in a variety of sequence-based technologies to study the molecular and biological functions of CD40 and to investigate or confirm its role in various diseases and disorders.

### Example 69

### Site Selection Program

In one embodiment of the invention, an application is deployed which facilitates the selection process for determining the target positions of the oligos to be synthesized, or “sites.” This program is written using a three-tiered object-oriented approach. All aspects of the software described, therefore, are tightly integrated with the relational database. For this reason, explicit database read and write steps are not shown. It should be assumed that each step described includes database access. The description below illustrates one way the program can be used. The actual interface allows users to skip from process to process at will, in any order.

Before running the site picking program, the target must have all relevant properties computed as described previously. When the site picking program is launched the user is presented with a panel showing targets which have previously been selected and had their properties calculated. The user selects one target to work with and proceeds to decide if any derived properties will be needed. Derived properties are calculated by performing mathematical operations on combinations of pre-calculated properties as defined by the user.

The derived properties are made available as peers with all the pre-calculated properties. The user selects one of the properties to view plotted versus target position. This graph is shown above a linear representation of the target. The horizontal or position axis of both the graph and target are linked and scalable by the user. The zoom range goes from showing the full target length to showing individual target bases as letters and individual property points. The user next selects a threshold value below or above which all sites will be eliminated from future consideration. The user decides whether to eliminate more sites based on any other properties. If they choose to eliminate more, they return to pick another property to display and threshold.

After eliminating sites, the user selects from the remaining list by choosing any property and then choosing a manual or automatic selection technique. In the automatic technique, the user decides whether they want to pick from maxima or minima and the number of maxima or minima to be selected as sites. The software automatically finds and picks the points. When picking manually the user must decide if they wish to use automatic peak finding. If the user selects automatic peak finding, then user must click on the graphed property with the mouse. The nearest maxima or minima, depending on the modifier key held down, to the selected point will be picked as the site. Without the peak finding option, the user must pick a site by clicking on its position on the linear representation of target.

Each time a site, or group of sites, is picked, a dynamic property is calculated for all possible sites (not yet eliminated). This property indicates the nearness of the site two a picked site allowing the user to pick sites in subsequent iterations based on target coverage. After new sites are picked, the user determines if the desired number of sites has been picked. If too few sites have been picked the user returns to pick more. If too many sites have been picked, the user may eliminate them by selecting and deleting them on the target display. If the correct number of sites is picked, and the user is satisfied with the set of picked sites, the user registers these sites to the database along with their name, notebook number, and page number. The database time stamps this registration event.

### Example 70

### Site Selection Program

In one embodiment of the invention, an application is deployed which facilitates the assignment of specific chemical structure to the compliment of the sequence of the sites previously picked and facilitates the registration and ordering of these now fully defined antisense compounds. This program is written using a three-tiered object-oriented approach. All aspects of the software described, therefore, are tightly integrated with the relational database. For this reason, explicit database read and write steps are not shown. It being understood that each step described also includes appropriate database read/write access.

To begin using the oligonucleotide chemistry assignment program, the user launches it. The user then selects from the previously selected sets of oligonucleotides, registered to the database in site picker's process step. Next, the user must decide whether to manually assign the chemistry a base a time, or run the sites through a template. If the user chooses to use a template, they must determine if a desired is available. If a template is not available with the desired chemistry modifications and the correct length, the user can define one.

To define a template, the user must select the length of the oligonucleotide the template is to define. This oligonucleotide is then represented as a bar with a selectable of regions. The user sets the number of regions on the oligonucleotide, and the positions and lengths of these regions by dragging them back and forth on the bar. Each region is represented by a different color.

For each region, the user must define the chemistry modifications for the sugars, the linkers, and the heterocycles at each base position in the region. Four heterocycle chemistries must be given, one for each of the four possible base types (A, G, C or T) in the site sequence the template will be applied to. A user interface is provided to select these chemistries which show the molecular structure of each component selected and its modification name. By pushing on a pop-up list next to each of the pictures, the user may choose from a list of structures and names, those possible to put in this place. For example, the heterocycle that represents the base type G is shown as a two dimensional structure diagram. If the user clicks on the pop-up list, a row of other possible structures and names is shown. The user drags the mouse to the desired chemistry and releases the mouse. Now the newly selected molecule is displayed as the choice for G type heterocycle modifications.

Once the user has created a template, or selected an existing one, the software applies the template to each of the compliments of the sites in the list. When the templates are applied, it is possible that chemistries will be defined which are impossible to make with the chemical precursors presently used on the automatic synthesizer. To check this, a database is maintained of all precursors previously designed, and their availability for automated synthesis. When the templates are applied, the resulting molecules are tested against this database to see if they are readily synthesized.

If a molecule is not readily synthesized, it is added to a list that the user inspects. The user decides whether to modify the chemistry to make compatible with the currently recognized list of available chemistries or to ignore it. To modify a chemistry, the user must use the base at a time interface. The user can also choose to go directly to this step, bypassing templates all together.

The base at a time interface is very similar to the template editor except that instead of specifying chemistries for regions, they are defined one base at a time. This interface also differs in that it dynamically checks to see if the design is readily synthesized as the user makes selections. In other words, each choice made limits the choices the software makes available on the pop-up selection lists. To accommodate this function, an additional choice is made available on each pop-up of “not defined.” For example, this allows the user to inhibiting linker choice from restricting the sugar choices by first setting the linker to “not defined.” The user would then pick the sugar, and then pick from the remaining linker choices available.

Once all of the sites on the list is assigned chemistries or dropped, they are registered at process step to the commercial chemical structure database. Registering to this database makes sure the structure is unique, assigns it a new identifier if it is unique, and allows future structure and substructure searching by creating various hash-tables. The compound definition is also stored at process step to various hash tables referred to as chemistry/position tables. These allow antisense compound searching and categorization based on oligonucleotide chemistry modification sequences and equivalent base sequences. The results of the registration are displayed to the user with the new IDs if they are new compounds and with the old IDs if they have been previously registered. The user next selects which of the compounds processed they wish to order for synthesis and registers an order list by scientist name, notebook number and page number. The database time-stamps this entry. The user may than choose to quit the program, go back to the beginning and choose a new site list to work with process step, or start the oligonucleotide ordering interface.

### Example 71

### Modifications to Account for Biologically Likely Species Variants (“cloud Algorithm”)

Base count blurring can be carried out as follows. “Electronic PCR” can be conducted on nucleotide sequences of desired bioagents to obtain the different expected base counts that could be obtained for each primer pair (i.e., primer pairs that hybridize to conserved regions that flank a variable region, wherein the variable region can be used to identify a bioagent; see, for example, International Application Publication WO 02/070664 and WO 03/001976, and U.S. Ser. No. 60/504,147, filed Sep. 17, 2003, each of which is incorporated herein by reference in its entirety). In one illustrative embodiment, one or more spreadsheets, such as Microsoft Excel workbook, that contains a plurality of worksheets. First, there is a worksheet with a name similar to the workbook name; this worksheet contains the raw electronic PCR data. Second, there is a worksheet named “filtered bioagents base count” that contains bioagent name and base count; there is a separate record for each strain after removing sequences that are not identified with a genus and species and removing all sequences for bioagents with less than 10 strains. Third, there is a worksheet, “Sheet1” that contains the frequency of substitutions, insertions, or deletions for this primer pair. This data is generated by first creating a pivot table from the data in the “filtered bioagents base count” worksheet and then executing an Excel VBA macro. A macro creates a table of differences in base counts for bioagents of the same species, but different strains. One of ordinary skill in the art may understand additional pathways for obtaining similar table differences without undo experimentation.

Application of an exemplary script, involves the user defining a threshold that specifies the fraction of the strains that are represented by the reference set of base counts for each bioagent. The reference set of base counts for each bioagent may contain as many different base counts as are needed to meet or exceed the threshold. The set of reference base counts is defined by taking the most abundant strain's base type composition and adding it to the reference set and then the next most abundant strain's base type composition is added until the threshold is met or exceeded. The current set of data was obtained using a threshold of 55%, which was obtained empirically.

For each base count not included in the reference base count set for that bioagent, the script then proceeds to determine the manner in which the current base count differs from each of the base counts in the reference set. This difference may be represented as a combination of substitutions, Si=Xi, and insertions, Ii=Yi, or deletions, Di=Zi. If there is more than one reference base count, then the reported difference is chosen using rules that aim to minimize the number of changes and, in instances with the same number of changes, minimize the number of insertions or deletions. Therefore, the primary rule is to identify the difference with the minimum sum (Xi+Yi) or (Xi+Zi), e.g., one insertion rather than two substitutions. If there are two or more differences with the minimum sum, then the one that will be reported is the one that contains the most substitutions.

Differences between a base count and a reference composition are categorized as either one, two, or more substitutions, one, two, or more insertions, one, two, or more deletions, and combinations of substitutions and insertions or deletions. Tables 16-25 illustrate these changes. The number of possible changes within each category is termed the complexity and is shown in Table 24.

The workbook contains a worksheet for each primer pair; the tables in each worksheet summarize the frequency of the types of base count changes. One worksheet can show the mean and standard deviation for each base count change type over the ten primer pairs.

The results of the above described procedure are presented in tables 16-25.

TABLE 16
Single Substitutions

| | |
|---|---|
| A → C | transversion |
| A → G | transition |
| A → T | transversion |
| C → A | transversion |
| C → G | transversion |
| C → T | transition |
| G → A | transition |
| G → C | transversion |
| G → T | transversion |
| T → A | transversion |
| T → C | transition |
| T → G | transversion |

TABLE 17
Two Substitutions

| | |
|---|---|
| A A → CC | 2 transversions |
| A A → CG | transition and transversion |
| A A → CT | 2 transversions |
| A G → CC | 2 transversions |
| A G → CT | 2 transversions |
| A T → CC | transition and transversion |
| A A → GG | 2 transitions |
| A A → GT | transition and transversion |
| A C → GG | transition and transversion |
| A C → GT | 2 transitions |
| A T → GC | 2 transitions |
| A T → GG | transition and transversion |
| A A → TT | 2 transversions |
| A C → TT | transition and transversion |
| A G → TT | 2 transversions |
| C C → AA | 2 transversions |
| C C → AG | 2 transversions |
| C C → AT | transition and transversion |
| C G → AA | transition and transversion |
| C G → AT | 2 transitions |
| C T → AA | 2 transversions |
| C T → AG | 2 transversions |
| C C → GG | 2 transversions |
| C C → GT | transition and transversion |
| C T → GG | 2 transversions |
| C C → TT | 2 transitions |
| C G → TT | transition and transversion |
| G G → AA | 2 transitions |
| G G → AC | transition and transversion |
| G G → AT | transition and transversion |
| G T → AA | transition and transversion |
| G T → AC | 2 transitions |
| G G → CC | 2 transversions |
| G G → CT | 2 transversions |
| G T → CC | transition and transversion |
| G G → TT | 2 transversions |
| T T → AA | 2 transversions |
| T T → AC | transition and transversion |
| T T → AG | 2 transversions |
| T T → CC | 2 transitions |
| T T → CG | transition and transversion |
| T T → GG | 2 transversions |

TABLE 18
Single Insertion
→ A
→ C
→ G
→ T

TABLE 19
Two Insertions
→ AA
→ AC
→ AG
→ AT
→ CC
→ CG
→ CT
→ GG
→ GT
→ TT

TABLE 20
Single Deletion
A →
C →
G →
T →

TABLE 21
Two Deletions
AA →
AC →
AG →
AT →
CC →
CG →
CT →
GG →
GT →
TT →

TABLE 22
One Substitution and One Insertion
A → CC
A → CG
A → CT
A → GG
A → GT
A → TT
C → AA
C → AG
C → AT
C → GG
C → GT
C → TT
G → AA
G → AC
G → AT
G → CC
G → CT
G → TT
T → AA
T → AC
T → AG
T → CC
T → CG
T → GG

TABLE 23
One Substitution and One Deletion
AA → C
AA → G
AA → T
AC → G
AC → T
AG → C
AG → T
AT → C
AT → G
CC → A
CC → G
CC → T
CG → A
CG → T
CT → A
CT → G
GG → A
GG → C
GG → T
GT → A
GT → C
TT → A
TT → C
TT → G

TABLE 24
Complexity of base count changes

| Type of base | |
|---|---|
| composition change | Comple [symbol] |
| Single Substitution | Purine → Purine |
| | Purine → Pyrimidine |
| | Pyrimidine → Purine |
| | Pyrimidine → Pyrimidine |
| | Single Transition |
| | Single Transversion |
| Two Substitutions | Two Transitions |
| | One Transition & One Transversion |
| | Two Transversions |
| Three Substitutions | Single Purine |
| One Insertion | Single Pyrimidine |
| Two Insertions | Two Purines |
| | One Purine & One Pyrimidine |
| | Two Pyrimidines |
| Three Insertions | Single Purine |
| One Deletion | Single Pyrimidine |
| Two Deletions | Two Purines |
| | One Purine & One Pyrimidine |
| | Two Pyrimidines |
| Three Deletions | Purine → TwoPurines |
| One Insertion & | Purine → One Purine & |
| One Substitution | One Pyrimidine |
| | Purine → TwoPyrimidines |
| | Pyrimidine → TwoPurines |
| | Pyrimidine → One Purine & One Pyrimidine |
| | Pyrimidine → TwoPyrimidines |
| | Single Transition & One Purine Insertion |
| | Single Transition & One Pyrimidine Insertion |
| | Single Transversion & One Purine Insertion |
| | Single Transversion & One Pyrimidine Insertion |
| One Deletion & | Two Purines→ Purine |
| One Substitution | One Purine & One Pyrimidine→ Purine |
| | Two Pyrimidines → Purine |
| | Two Purines → Pyrimidine |
| | One Purine & One Pyrimidine → Pyrimidine |
| | Two Pyrimidines → Pyrimidine |
| | Single Transition & One Purine Deletion |
| | Single Transition & One Pyrimidine Deletion |
| | Single Transversion & One Purine Deletion |
| | Single Transversion & One Pyrimidine Deletion |

TABLE 25
Average Frequencies of Various Base Composition Changes Deduced from Electronic PCR of 16 S Ribosomal Data

| | Strains | | Strains/Complexity | | Base Compositions | | Base Compositins/Complexity | |
|---|---|---|---|---|---|---|---|---|
| Strain Threshold = 55% | Average | Std. Dev. | Average | Std. Dev. | Average | Std. Dev. | Average | Std. Dev. |
| No Changes | 85.9% | 5.7% | 85.9% | 5.7% | 41.8% | 7.6% | 41.8% | 7.6% |
| All Changes | 14.1% | 5.7% | | | 58.2% | 7.6% | | |
| Single Substitution | 7.5% | 3.1% | 0.63% | 0.3% | 29.5% | 2.5% | 2.5% | 0.21% |
| Purine −> Purine | 2.6% | 1.6% | 1.29% | 0.8% | 8.5% | 2.5% | 4.3% | 1.23% |
| Purine −> Pyrimidine | 1.0% | 0.5% | 0.24% | 0.1% | 5.4% | 2.3% | 1.4% | 0.58% |
| Pyrimidine −> Purine | 1.1% | 0.4% | 0.28% | 0.1% | 5.8% | 2.0% | 1.5% | 0.50% |
| Pyrimidine −> Pyrimidine | 2.9% | 1.2% | 1.44% | 0.6% | 9.7% | 2.1% | 4.9% | 1.03% |
| Single Transition | 5.5% | 2.5% | 1.36% | 0.6% | 18.2% | 2.5% | 4.6% | 0.63% |
| Single Transversion | 2.1% | 0.7% | 0.26% | 0.1% | 11.2% | 2.2% | 1.4% | 0.27% |
| Two Substitutions | 2.5% | 1.2% | 0.06% | 0.0% | 9.7% | 2.9% | 0.2% | 0.07% |
| Two Transitions | 1.2% | 0.9% | 0.17% | 0.1% | 3.7% | 1.1% | 0.5% | 0.16% |
| One Transition & One Transversion | 0.6% | 0.4% | 0.04% | 0.0% | 2.8% | 1.7% | 0.2% | 0.11% |
| Two Transversions | 0.7% | 0.6% | 0.04% | 0.0% | 3.2% | 1.7% | 0.2% | 0.09% |
| Three or More Substitutions | 1.0% | 1.0% | 0.01% | 0.0% | 4.5% | 3.2% | 0.0% | 0.03% |
| One Insertion | 1.0% | 1.0% | 0.26% | 0.2% | 3.8% | 2.5% | 0.9% | 0.62% |
| Single Purine | 0.6% | 0.5% | 0.28% | 0.2% | 2.1% | 1.1% | 1.1% | 0.57% |
| Single Pyrimidine | 0.5% | 0.8% | 0.24% | 0.4% | 1.6% | 1.5% | 0.8% | 0.77% |
| Two Insertions | 0.1% | 0.2% | 0.01% | 0.0% | 0.5% | 0.6% | 0.1% | 0.06% |
| Two Purines | 0.0% | 0.0% | 0.01% | 0.0% | 0.2% | 0.3% | 0.1% | 0.08% |
| One Purine & One Pyrimidine | 0.1% | 0.1% | 0.02% | 0.0% | 0.2% | 0.3% | 0.1% | 0.08% |
| Two Pyrimidines | 0.0% | 0.0% | 0.01% | 0.0% | 0.1% | 0.2% | 0.0% | 0.06% |
| Three or More Insertions | 0.1% | 0.1% | 0.00% | 0.0% | 0.5% | 0.5% | 0.0% | 0.03% |
| One Deletion | 0.6% | 0.4% | 0.15% | 0.1% | 3.2% | 1.8% | 0.8% | 0.44% |
| Single Purine | 0.3% | 0.2% | 0.17% | 0.1% | 1.7% | 0.9% | 0.9% | 0.43% |
| Single Pyrimidine | 0.3% | 0.3% | 0.13% | 0.1% | 1.5% | 1.3% | 0.7% | 0.66% |
| Two Deletions | 0.1% | 0.2% | 0.01% | 0.0% | 0.9% | 1.0% | 0.1% | 0.10% |
| Two Purines | 0.0% | 0.1% | 0.02% | 0.0% | 0.4% | 0.5% | 0.1% | 0.15% |
| One Purine & One Pyrimidine | 0.1% | 0.1% | 0.02% | 0.0% | 0.3% | 0.6% | 0.1% | 0.14% |
| Two Pyrimidines | 0.0% | 0.0% | 0.01% | 0.0% | 0.2% | 0.3% | 0.1% | 0.08% |
| Three or More Deletions | 0.1% | 0.1% | 0.00% | 0.0% | 0.4% | 0.4% | 0.0% | 0.02% |
| One Insertion & One Substitution | 0.1% | 0.1% | 0.00% | 0.0% | 0.7% | 0.5% | 0.0% | 0.02% |
| Purine −> Two Purines | 0.0% | 0.0% | 0.00% | 0.0% | 0.0% | 0.0% | 0.0% | 0.00% |
| Purine −> One Purine & One Pyrimidine | 0.0% | 0.0% | 0.00% | 0.0% | 0.1% | 0.2% | 0.0% | 0.05% |
| Purine −> Two Pyrimidines | 0.0% | 0.0% | 0.00% | 0.0% | 0.2% | 0.2% | 0.0% | 0.03% |
| Pyrimidine −> Two Purines | 0.0% | 0.0% | 0.00% | 0.0% | 0.2% | 0.3% | 0.0% | 0.04% |
| Pyrimidine −> One Purine & One Pyrimidine | 0.0% | 0.0% | 0.01% | 0.0% | 0.2% | 0.3% | 0.0% | 0.07% |
| Pyrimidine −> Two Pyrimidines | 0.0% | 0.0% | 0.00% | 0.0% | 0.0% | 0.0% | 0.0% | 0.00% |
| One Deletion & One Substitution | 0.2% | 0.2% | 0.01% | 0.0% | 1.1% | 0.9% | 0.0% | 0.04% |
| Two Purines −> Purine | 0.0% | 0.0% | 0.00% | 0.0% | 0.0% | 0.0% | 0.0% | 0.00% |
| One Purine & One Pyrimidine −> Purine | 0.0% | 0.0% | 0.01% | 0.0% | 0.4% | 0.4% | 0.1% | 0.11% |
| Two Pyrimidines −> Purine | 0.0% | 0.1% | 0.01% | 0.0% | 0.1% | 0.2% | 0.0% | 0.04% |
| Two Purines −> Pyrimidine | 0.0% | 0.0% | 0.00% | 0.0% | 0.2% | 0.3% | 0.0% | 0.05% |
| One Purine & One Pyrimidine −> Pyrimidine | 0.0% | 0.1% | 0.01% | 0.0% | 0.2% | 0.3% | 0.1% | 0.08% |
| Two Pyrimidines −> Pyrimidine | 0.0% | 0.0% | 0.01% | 0.0% | 0.1% | 0.3% | 0.1% | 0.13% |
| >=1 Insertions/Deletions & >= 1 Substitutions | 0.8% | 1.3% | | | 3.5% | 3.7% | | |

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method for selecting a target molecule that has an affinity for a ligand that is equal to or greater than a baseline affinity comprising:
mixing an amount of a standard target with an excess amount of the ligand, wherein the standard target forms a non-covalent binding complex with the ligand and wherein unbound ligand is present in the mixture;
introducing the mixture of the standard target and the ligand into a mass spectrometer to obtain a baseline affinity;
adjusting the operating performance conditions of the mass spectrometer such that the signal strength of the standard target bound to the ligand is from 1% to about 30% of the signal strength of unbound ligand;
introducing at least one target molecule into the test mixture of the ligand and the standard target;
introducing the test mixture into a mass spectrometer; and
identifying any complexes of the target molecule and the ligand, wherein the presence of a complex is indicated by an affinity that is greater than the baseline affinity, and wherein either one or both of the target molecule and ligand, independently, is a microRNA.

2. The method of claim 1 wherein the mass spectrometer is an electrospray mass spectrometer.

3. The method of claim 1 wherein the ligand is a microRNA and the target molecule is a microRNA, a microRNA mimic, a protein, an RNA-DNA duplex, an RNA-RNA duplex, a DNA duplex, a polysaccharide, a phospholipid, or a glycolipid; or wherein the target molecule is a microRNA and the ligand is a microRNA, a microRNA mimic, a protein, an RNA-DNA duplex, an RNA-RNA duplex, a DNA duplex, a polysaccharide, a phospholipid, or a glycolipid.

4. The method of claim 3 wherein the ligand is a microRNA and the target molecule is a microRNA.

5. The method of claim 1 wherein the ligand or target molecule is a microRNA mimic.

6. The method of claim 1 wherein the baseline affinity expressed as a dissociation constant is about 50 millimolar.

7. The method of claim 1 wherein the standard target is ammonium, a primary amine, a secondary amine, a tertiary amine, an amino acid, or a nitrogen-containing heterocycle.

8. The method of claim 1 wherein the standard target is ammonium or primary amine.

9. The method of claim 1 wherein the standard target is ammonium.

10. The method of claim 2 wherein the electrospray mass spectrometer comprises a desolvation capillary or countercurrent gas and a lens element, and the adjustment of the operating performance conditions comprises adjustment of the voltage potential across the capillary and the lens element, adjustment of source voltage potential to give a stable electrospray ionization as monitored by the ion abundance of free target molecule, adjustment of the temperature of the desolvation capillary or countercurrent heating gas, or adjustment of the operating gas pressure within the mass spectrometer downstream of the desolvation capillary.

11. The method of claim 10 wherein the standard target is ammonium ion, and the adjustment of the voltage potential across the capillary and the lens element generates a signal strength of the monoammonium-microRNA complex that is from about 10% to about 20% of the signal strength of unbound microRNA.

12. The method of claim 4 wherein the microRNA ligand or microRNA target molecule is from about 10 to about 200 nucleotides in length.

13. The method of claim 4 wherein the microRNA ligand or microRNA target molecule is from about 15 to about 100 nucleotides in length.

14. The method of claim 4 wherein the microRNA ligand or microRNA target molecule comprises an isolated or purified portion of a larger RNA molecule.

15. The method of claim 4 wherein the microRNA ligand or microRNA target molecule has secondary and ternary structure.

16. The method of claim 2 wherein the electrospray mass spectrometer comprises a gated ion storage device for effecting thermolysis of the test mixture in the mass spectrometer.

17. The method of claim 2 wherein the mass spectrometer comprises mass analysis by a quadrupole, a quadrupole ion trap, a time-of-flight, a FT-ICR, or a hybrid mass detector.

18. The method of claim 2 wherein the electrospray mass spectrometer comprises Z-spray, microspray, off-axis spray, or pneumatically assisted electrospray ionization.

19. The method of claim 18 wherein the Z-spray, microspray, off-axis spray, or pneumatically assisted electrospray ionization each comprise countercurrent drying gas.

20. The method of claim 1 further comprising storing the relative abundance and stoichiometry of the complexes of the ligand and target molecule in a relational database that is cross-indexed to the structure of the target molecule.

21. The method of claim 1 wherein the target molecule is a member of a set of target molecules.

22. The method of claim 21 wherein each of the members of the set of target molecules, independently, has a molecular mass less than about 1000 Daltons and has fewer than 15 rotatable bonds.

23. The method of claim 21 wherein each of the members of the set of target molecules, independently, has a molecular mass less than about 600 Daltons and has fewer than 8 rotatable bonds.

24. The method of claim 21 wherein each of the members of the set of target molecules, independently, has a molecular mass less than about 200 Daltons, has fewer than 4 rotatable bonds or no more than one sulfur, phosphorous, or halogen atom.

25. The method of claim 1 wherein the signal strength is measured by the relative ion abundance.

26. The method of claim 1 further comprising a plurality of target molecules.

27. The method of claim 26 further comprsing a plurality of standard targets.
